SEQUENCE LISTING (3 sequences)

SEQ ID NO: 1 - RNA, 54 nt, synthetic construct
  source 1..54: mol_type = other RNA
  modified_base 6: mod_base = m1f
  modified_base 11: mod_base = m1f
  modified_base 14: mod_base = m1f
  modified_base 16..17: mod_base = m1f
  modified_base 19..20: mod_base = m1f
  modified_base 22: mod_base = m1f
  modified_base 25: mod_base = m1f
  modified_base 36: mod_base = m1f
gagaanaaacnagnanncnncnggnccccacagacncagagagaacccgccacc

SEQ ID NO: 2 - RNA, 295 nt, synthetic construct
  source 1..295: mol_type = other RNA
  modified_base 2: mod_base = m1f
  modified_base 8: mod_base = m1f
  modified_base 11: mod_base = m1f
  modified_base 14: mod_base = m1f
  modified_base 18: mod_base = m1f
  modified_base 27: mod_base = m1f
  modified_base 30: mod_base = m1f
  modified_base 34: mod_base = m1f
  modified_base 40..42: mod_base = m1f
  modified_base 47: mod_base = m1f
  modified_base 50: mod_base = m1f
  modified_base 54: mod_base = m1f
  modified_base 63: mod_base = m1f
  modified_base 65: mod_base = m1f
  modified_base 75: mod_base = m1f
  modified_base 80: mod_base = m1f
  modified_base 87: mod_base = m1f
  modified_base 89: mod_base = m1f
  modified_base 92: mod_base = m1f
  modified_base 99: mod_base = m1f
  modified_base 105: mod_base = m1f
  modified_base 113: mod_base = m1f
  modified_base 121: mod_base = m1f
  modified_base 123: mod_base = m1f
  modified_base 126: mod_base = m1f
  modified_base 129..130: mod_base = m1f
  modified_base 140: mod_base = m1f
  modified_base 157: mod_base = m1f
  modified_base 163: mod_base = m1f
  modified_base 172..173: mod_base = m1f
  modified_base 178: mod_base = m1f
  modified_base 205: mod_base = m1f
  modified_base 208..209: mod_base = m1f
  modified_base 214..216: mod_base = m1f
  modified_base 222: mod_base = m1f
  modified_base 232..234: mod_base = m1f
  modified_base 238: mod_base = m1f
  modified_base 243: mod_base = m1f
  modified_base 245: mod_base = m1f
  modified_base 248: mod_base = m1f
  modified_base 259..260: mod_base = m1f
  modified_base 263: mod_base = m1f
  modified_base 267..269: mod_base = m1f
  modified_base 272: mod_base = m1f
  modified_base 286: mod_base = m1f
  modified_base 292: mod_base = m1f
cncgagcnggnacngcangcacgcaangcnagcngccccnnncccgnccngggnaccccgagncncccccgaccncgggncccaggnangcncccaccnccaccngccccacncaccaccncngcnagnnccagacaccncccaagcacgcagcaangcagcncaaaacgcnnagccnagccacacccccacgggaaacagcagngannaaccnnnagcaanaaacgaaagnnnaacnaagcnanacnaaccccagggnnggncaannncgngccagccacacccnggagcnagc

SEQ ID NO: 3 - RNA, 110 nt, synthetic construct
  source 1..110: mol_type = other RNA
  modified_base 34: mod_base = m1f
  modified_base 36: mod_base = m1f
  modified_base 40: mod_base = m1f
aaaaaaaaaaaaaaaaaaaaaaaaaaaaaagcanangacnaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2025/0367129 A1
(43) Pub. Date: Dec. 4, 2025

(54) Lipid Compounds and Uses Thereof

(71) Applicant: Pfizer Inc., New York, NY (US)

(72) Inventors: Matthew Frank Brown, Stonington, CT (US); Daniel Paul Canterbury, Pawcatuck, CT (US); Ye Che, Niantic, CT (US); Wenhua Jiao, Salem, CT (US); Roger Hochoon Pak, Boxborough, MA (US); Mark Edward Schnute, Acton, MA (US)

(21) Appl. No.: 19/125,013

(22) PCT Filed: Nov. 1, 2023
(86) PCT No.: PCT/IB2023/061006

§ 371 Date: Apr. 28, 2025

Related U.S. Application Data
(60) Provisional application No. 63/382,389, filed on Nov. 4, 2022.
(60) Provisional application No. 63/590,756, filed on Oct. 16, 2023.

(51) Int. Cl. A61K 9/51; A61K 31/575; A61K 31/7105; A61K 47/22; C07D 221/20; C07D 471/10; C07D 487/10

(52) U.S. Cl. CPC A61K 9/5123; A61K 31/575; A61K 31/7105; A61K 47/22; C07D 221/20; C07D 471/10; C07D 487/10

(57) ABSTRACT

Compounds are provided having the following structure: (I) or a pharmaceutically acceptable salt, N-oxide, tautomer or stereoisomer thereof, wherein R¹, G¹, W and m, n, o and p are as defined herein. Use of the compounds as a component of lipid nanoparticle formulations for delivery of a nucleic acid, compositions comprising the compounds and methods for their use and preparation are also provided.

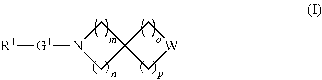

### CROSS REFERENCE TO RELATED APPLICATIONS

This application claims the benefit of U.S. Provisional Application No. 63/590,756, filed Oct. 16, 2023, and U.S. Provisional Application No. 63/382,389, filed Nov. 4, 2022. The entire content of each of the foregoing applications is incorporated herein by reference.

### REFERENCE TO SEQUENCE LISTING

This application is being filed electronically via EFS-Web and includes an electronically submitted sequence listing in.xml format. The .xml file contains a sequence listing entitled “PC072882A Sequence Listing.xml” created on Oct. 17, 2023 and having a size of 25 KB. The sequence listing contained in this .xml file is part of the specification and is incorporated herein by reference in its entirety.

### BACKGROUND OF THE INVENTION

The present invention relates to novel ionizable lipid compounds. The invention also relates to the preparation of the ionizable lipid compounds and intermediates used in the preparation, compositions containing the ionizable lipid compounds, and uses of the ionizable lipid compounds including in combination with other lipid components, such as neutral lipids, cholesterol and polymer conjugated lipids, to form lipid nanoparticles with oligonucleotides, to facilitate the intracellular delivery of therapeutic nucleic acids both in vitro and in vivo.

There are many challenges associated with the delivery of nucleic acids to affect a desired response in a biological system. Nucleic acid-based therapeutics have enormous potential but there remains a need for more effective delivery of nucleic acids to appropriate sites within a cell or organism in order to realize this potential. Therapeutic nucleic acids include, e.g., messenger RNA (mRNA), antisense oligonucleotides, ribozymes, DNAzymes, plasmids, immune stimulating nucleic acids, antagomir, antimir, mimic, supermir, and aptamers. Some nucleic acids, such as mRNA or plasmids, can be used to effect expression of specific cellular products as would be useful in the treatment of, for example, diseases related to a deficiency of a protein or enzyme, or as a vaccine. The therapeutic applications of translatable nucleotide delivery are extremely broad as constructs can be synthesized to produce any chosen protein sequence, whether or not indigenous to the system. The expression products of the nucleic acid can augment existing levels of protein, replace missing or non-functional versions of a protein, or introduce new protein and associated functionality in a cell or organism.

However, two problems currently face the use of oligonucleotides in therapeutic contexts. First, free RNAs are susceptible to nuclease digestion in plasma. Second, free RNAs have limited ability to gain access to the intracellular compartment where the relevant translation machinery resides. Lipid nanoparticles formed from ionizable lipids with other lipid components, such as neutral lipids, cholesterol, PEG, PEGylated lipids, and oligonucleotides have been used to block degradation of the RNAs in plasma and facilitate the cellular uptake of the oligonucleotides.

Accordingly, there remains a need for improved lipid compounds and lipid nanoparticles for the delivery of oligonucleotides. Preferably, these lipid nanoparticles would provide optimal drug:lipid ratios, protect the nucleic acid from degradation and clearance in serum, be suitable for systemic or local delivery, and provide intracellular delivery of the nucleic acid. In addition, these lipid-nucleic acid particles should be well-tolerated and provide an adequate therapeutic index, such that patient treatment at an effective dose of the nucleic acid is not associated with unacceptable toxicity and/or risk to the patient. In addition, there is a need to identify ionizable lipid-containing nanoparticle compositions with improved colloidal stability as well as tissue or cell specificity of oligonucleotide delivery.

### SUMMARY OF THE INVENTION

The present invention provides, in part, lipid compounds of Formula (I) and pharmaceutically acceptable salts, N-oxide, tautomers or stereoisomers thereof. Such lipid compounds, including pharmaceutically acceptable salts, N-oxide, tautomers or stereoisomers thereof, can be used alone or in combination with other lipid components such as neutral lipids, charged lipids, steroids (including for example, cholesterol) and/or their analogs, and/or polymer conjugated lipids to form lipid nanoparticles for the delivery of therapeutic agents. In some instances, the lipid nanoparticles are used to deliver nucleic acids such as antisense and/or messenger RNA. Also provided are pharmaceutical compositions, comprising the lipid compounds pharmaceutically acceptable salts, N-oxide, tautomers or stereoisomers of the invention, alone or in combination with additional therapeutic agents. The present invention also provides, in part, methods for preparing such lipid compounds, or pharmaceutically acceptable salts, N-oxide, tautomers or stereoisomers thereof and compositions of the invention, and methods of using the foregoing for treatment of various diseases or conditions, such as those caused by infectious entities and/or insufficiency of a protein. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to an embodiment of the invention there is provided a compound of Formula (I)

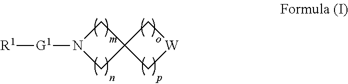

or a pharmaceutically acceptable salt, N-oxide, tautomer, or stereoisomer thereof, wherein
m, n, o, and p are each independently 1-3;
G¹ is C₁₋₁₂alkylene or C₂₋₁₂alkenylene;
R¹ is —N(R²)R³, —OR⁴, CN, —N(R⁴)(heteroaryl), —O(CH₂)_{q}OH, —(OCH₂CH₂)ᵣOH, —OC(═O)R⁵, —N(R⁴)C(═O)R⁵, —N(R⁴)S(O)₂R⁵, —N(R⁴)C(═O)N(R²)R³, —OC(═O)N(R²)R³, —N(R⁴)C(═O)OR⁵, —N(R⁴)C(═S)N(R²)R³, —N(R⁴)C(═NR⁶)N(R²)R³, or

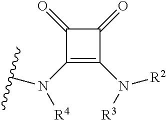

R² and R³ are each independently H, C₁₋₆alkyl, C₃₋₈cycloalkyl, or aryl; or R² and R³ together with the nitrogen atom to which they are attached form a heterocyclic ring;
R⁴ is H, C₁₋₆alkyl or C₃₋₈cycloalkyl;
R⁵ is C₁₋₆alkyl or C₃₋₈cycloalkyl optionally substituted by C₁₋₆alkyl;
R⁶ is H, CN, NO₂, C₁₋₆alkyl, OR⁵, S(O)₂R⁵, or S(O)₂N(R²)R³;
q is 2-6;
r is 1-6;

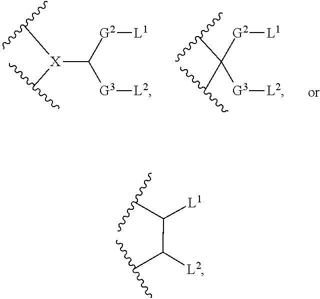

W is
X is N or CH;
G² and G³ are each independently C₁₋₁₂alkylene or C₂₋₁₂alkenylene;
L¹ and L² are each independently —C(═O)OR⁷, —OC(═O)R⁷, —OC(═O)(CH₂)ᵣC(═O)OR⁷, —OC(═O)(CH₂)ᵣOC(═O)R⁷, —OC(═O)N(R⁴)R⁷, —N(R⁴)C(═O)OR⁷, —N(R⁴)C(═O)N(R⁴)R⁷, OC(═O)OR⁷, or —S—SR⁷; and
R⁷ is C₆₋₂₄alkyl, C₆₋₂₄alkenyl, or C₆₋₂₄alkynyl where each is optionally substituted by F, C₁₋₆alkoxy, C₃₋₈cycloalkyl, or C₃₋₈cycloalkenyl, wherein the R⁷ of L¹ and L² may be the same or different.

In one aspect, the present disclosure relates to a compound having Formula (Ia)

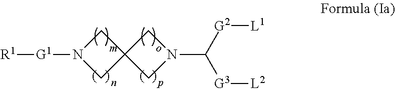

or a pharmaceutically acceptable salt, N-oxide, tautomer or stereoisomer thereof, wherein m, n, o, and p are each independently 1 or 2.

In another aspect, the present disclosure relates to a compound having Formula (Ib)

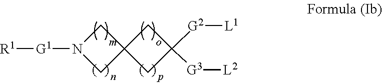

or a pharmaceutically acceptable salt, N-oxide, tautomer or stereoisomer thereof, wherein m, n, o, and p are each independently 1 or 2.

In another aspect, the present disclosure relates to a compound having Formula (Ic)

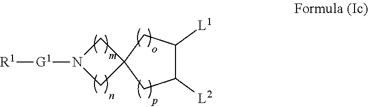

or a pharmaceutically acceptable salt, N-oxide, tautomer or stereoisomer thereof,
wherein m and n are each independently 1 or 2; and
and p are each 1.

In a further aspect, the present disclosure relates to a compound selected from the group consisting of:
(3-(4-hydroxybutyl)-3-azaspiro[5.5]undecane-9,9-diyl)bis(methylene) bis(2-heptylnonanoate);
2-(3-(4-hydroxybutyl)-3-azaspiro[5.5]undecan-9-yl)propane-1,3-diyl bis(2-heptylnonanoate);
2-(9-(4-hydroxybutyl)-3,9-diazaspiro[5.5]undecan-3-yl)propane-1,3-diyl bis(2-heptylnonanoate);
2-(9-(4-hydroxybutyl)-3,9-diazaspiro[5.5]undecan-3-yl)propane-1,3-diyl bis(2-cyclobutyldecanoate);
2-(9-(5-hydroxypentyl)-3,9-diazaspiro[5.5]undecan-3-yl)propane-1,3-diyl bis(2-hexyldecanoate);
2-(2-(4-hydroxybutyl)-2,8-diazaspiro[4.5]decan-8-yl)propane-1,3-diyl bis(2-hexyldecanoate);
3-((2-hexyldecanoyl)oxy)-2-(9-(4-hydroxybutyl)-3,9-diazaspiro[5.5]undecan-3-yl)propyl palmitate;
2-(9-(3-((2-(methylamino)-3,4-dioxocyclobut-1-en-1-yl)amino)propyl)-3,9-diazaspiro[5.5]undecan-3-yl)propane-1,3-diyl bis(2-heptylnonanoate);
2-(9-(3-(1-methylcyclopropane-1-carboxamido)propyl)-3,9-diazaspiro[5.5]undecan-3-yl)propane-1,3-diyl bis(2-hexyldecanoate);
2-(9-(3-hydroxypropyl)-3,9-diazaspiro[5.5]undecan-3-yl)propane-1,3-diyl bis(2-heptylnonanoate);
2-(9-(3-(ethylsulfonamido)propyl)-3,9-diazaspiro[5.5]undecan-3-yl)propane-1,3-diyl bis(2-hexyldecanoate);
2-(7-(4-hydroxybutyl)-2,7-diazaspiro[4.4]nonan-2-yl)propane-1,3-diyl bis(2-heptylnonanoate);
2-(7-(4-hydroxybutyl)-2,7-diazaspiro[3.5]nonan-2-yl)propane-1,3-diyl bis(2-heptylnonanoate);
2-(8-(4-hydroxybutyl)-2,8-diazaspiro[4.5]decan-2-yl)propane-1,3-diyl bis(2-heptylnonanoate);
3-(9-(4-hydroxybutyl)-3,9-diazaspiro[5.5]undecan-3-yl)pentane-1,5-diyl bis(2-hexyldecanoate);
2-(7-(5-hydroxypentyl)-2,7-diazaspiro[4.4]nonan-2-yl)propane-1,3-diyl bis(2-hexyldecanoate);
2-(2-(5-hydroxypentyl)-2,8-diazaspiro[4.5]decan-8-yl)propane-1,3-diyl bis(2-hexyldecanoate);
2-(7-(5-hydroxypentyl)-2,7-diazaspiro[3.5]nonan-2-yl)propane-1,3-diyl bis(2-heptylnonanoate);
2-(9-(4-hydroxybutyl)-3,9-diazaspiro[5.5]undecan-3-yl)pentane-1,5-diyl bis(2-hexyldecanoate);
2-(2-(4-hydroxybutyl)-2,7-diazaspiro[3.5]nonan-7-yl)propane-1,3-diyl bis(2-heptylnonanoate);
2-(9-(5-hydroxypentan-2-yl)-3,9-diazaspiro[5.5]undecan-3-yl)propane-1,3-diyl bis(2-hexyldecanoate);
2-(2-(4-hydroxybutyl)-2,8-diazaspiro[4.5]decan-8-yl)propane-1,3-diyl bis(2-heptylnonanoate);
2-(9-(4-hydroxybutyl)-3,9-diazaspiro[5.5]undecan-3-yl)propane-1,3-diyl bis(2-hexyldecanoate);
2-(9-(4-hydroxybutyl)-3,9-diazaspiro[5.5]undecan-3-yl)propane-1,3-diyl bis(2-octyldecanoate);
2-(9-(5-hydroxypentyl)-3,9-diazaspiro[5.5]undecan-3-yl)propane-1,3-diyl bis(2-heptylnonanoate);
2-(9-(4-hydroxybutyl)-3,9-diazaspiro[5.5]undecan-3-yl)propane-1,3-diyl bis(2-heptyldecanoate);
2-(9-(4-hydroxybutyl)-3,9-diazaspiro[5.5]undecan-3-yl)propane-1,3-diyl bis(2-butyldecanoate);
2-(9-(4-hydroxybutyl)-3,9-diazaspiro[5.5]undecan-3-yl)propane-1,3-diyl bis(3-hexylundecanoate);
2-(9-(4-hydroxybutyl)-3,9-diazaspiro[5.5]undecan-3-yl)propane-1,3-diyl bis(2-pentyldecanoate);
2-(9-(4-hydroxybutyl)-3,9-diazaspiro[5.5]undecan-3-yl)propane-1,3-diyl bis(2-(cyclobutylmethyl)decanoate);
2-(9-(4-hydroxybutyl)-3,9-diazaspiro[5.5]undecan-3-yl)propane-1,3-diyl bis(2-heptyltetradecanoate);
2-(9-(4-hydroxybutyl)-3,9-diazaspiro[5.5]undecan-3-yl)propane-1,3-diyl bis(2-(cyclopentylmethyl)decanoate);
2-(7-(4-hydroxybutyl)-2,7-diazaspiro[4.4]nonan-2-yl)propane-1,3-diyl bis(2-hexyldecanoate);
2-(7-(4-hydroxybutyl)-2,7-diazaspiro[3.5]nonan-2-yl)propane-1,3-diyl bis(2-hexyldecanoate);
2-(9-(4-hydroxybutyl)-3,9-diazaspiro[5.5]undecan-3-yl)propane-1,3-diyl bis(2-(cyclopent-3-en-1-ylmethyl)decanoate);
2-(9-(4-hydroxybutyl)-3,9-diazaspiro[5.5]undecan-3-yl)propane-1,3-diyl bis(2-(2-cyclobutylethyl)decanoate);
2-(9-(4-hydroxybutyl)-3,9-diazaspiro[5.5]undecan-3-yl)propane-1,3-diyl bis(2-(cyclohexylmethyl)decanoate);
2-(9-(4-hydroxybutyl)-3,9-diazaspiro[5.5]undecan-3-yl)propane-1,3-diyl bis(4,5-dibutylnonanoate);
2-(9-(4-hydroxybutyl)-3,9-diazaspiro[5.5]undecan-3-yl)propane-1,3-diyl bis(3,3-dibutylnonanoate);
2-(9-(4-hydroxybutyl)-3,9-diazaspiro[5.5]undecan-3-yl)propane-1,3-diyl bis(4-heptylundecanoate);
2-(7-(5-hydroxypentyl)-2,7-diazaspiro[4.4]nonan-2-yl)propane-1,3-diyl bis(2-heptylnonanoate);
3-(decanoyloxy)-2-(9-(4-hydroxybutyl)-3,9-diazaspiro[5.5]undecan-3-yl)propyl 2-hexyldecanoate;
3-((2-hexyldecanoyl)oxy)-2-(9-(4-hydroxybutyl)-3,9-diazaspiro[5.5]undecan-3-yl)propyl (Z)-dodec-5-enoate;
3-((2-(cyclobutylmethyl)decanoyl)oxy)-2-(9-(4-hydroxybutyl)-3,9-diazaspiro[5.5]undecan-3-yl)propyl 2-hexyldecanoate;
3-((2-hexyldecanoyl)oxy)-2-(9-(4-hydroxybutyl)-3,9-diazaspiro[5.5]undecan-3-yl)propyl 2-butylundecanoate;
3-((4,5-dibutylnonanoyl)oxy)-2-(9-(4-hydroxybutyl)-3,9-diazaspiro[5.5]undecan-3-yl)propyl palmitate;
3-(9-(4-hydroxybutyl)-3,9-diazaspiro[5.5]undecan-3-yl)pentane-1,5-diyl bis(2-heptylnonanoate);
2-(2-(5-hydroxypentyl)-2,8-diazaspiro[4.5]decan-8-yl)propane-1,3-diyl bis(2-heptylnonanoate);
2-(7-(5-hydroxypentyl)-2,7-diazaspiro[3.5]nonan-2-yl)propane-1,3-diyl bis(2-hexyldecanoate);
2-(2-(4-hydroxybutyl)-2,7-diazaspiro[3.5]nonan-7-yl)propane-1,3-diyl bis(2-hexyldecanoate);
rac-(2R,3R)-8-(4-hydroxybutyl)-8-azaspiro[4.5]decane-2,3-diyl bis(2-heptylnonanoate);
rac-(2R,3R)-8-(2-hydroxyethyl)-8-azaspiro[4.5]decane-2,3-diyl bis(2-heptylnonanoate);
rac-(7R,8R)-2-(3-hydroxypropyl)-2-azaspiro[4.4]nonane-7,8-diyl bis(2-heptylnonanoate);
rac-(2R,3R)-8-(3-hydroxypropyl)-8-azaspiro[4.5]decane-2,3-diyl bis(2-heptylnonanoate);
rac-(2R,3R)-8-(3-((2-(methylamino)-3,4-dioxocyclobut-1-en-1-yl)amino)propyl)-8-azaspiro[4.5]decane-2,3-diyl bis(2-heptylnonanoate);
rac-(2R,3R)-8-(5-hydroxypentyl)-8-azaspiro[4.5]decane-2,3-diyl bis(2-heptylnonanoate);
(2R,3R)-8-(4-hydroxybutyl)-8-azaspiro[4.5]decane-2,3-diyl bis(2-heptylnonanoate);
(2S,3S)-8-(4-hydroxybutyl)-8-azaspiro[4.5]decane-2,3-diyl bis(2-heptylnonanoate);
rac-(2R,3R)-8-(4-hydroxybutyl)-8-azaspiro[4.5]decane-2,3-diyl bis(2-octyldecanoate);
rac-(2R,3R)-8-(4-hydroxybutyl)-8-azaspiro[4.5]decane-2,3-diyl bis(2-heptyltetradecanoate);
rac-(2R,3R)-8-(4-hydroxybutyl)-8-azaspiro[4.5]decane-2,3-diyl bis(2-hexyldecanoate);
rac-O,O′-((2R,3R)-8-(4-hydroxybutyl)-8-azaspiro[4.5]decane-2,3-diyl) di(pentadecan-8-yl) disuccinate;
rac-O′¹,O¹-((2R,3R)-8-(4-hydroxybutyl)-8-azaspiro[4.5]decane-2,3-diyl) 7,7′-di(pentadecan-8-yl) di(heptanedioate);
rac-(((2R,3R)-8-(4-hydroxybutyl)-8-azaspiro[4.5]decane-2,3-diyl)bis(oxy))bis(6-oxohexane-6,1-diyl) bis(2-heptylnonanoate);
rac-(2R,3R)-8-(4-hydroxybutyl)-8-azaspiro[4.5]decane-2,3-diyl bis(2-pentyldecanoate);
rac-(2R,3R)-8-(4-hydroxybutyl)-8-azaspiro[4.5]decane-2,3-diyl bis(2-heptyldecanoate);
rac-(2R,3R)-8-(4-hydroxybutyl)-8-azaspiro[4.5]decane-2,3-diyl bis(2-hexylundecanoate);
rac-(2R,3R)-8-(4-hydroxybutyl)-8-azaspiro[4.5]decane-2,3-diyl bis(2-hexylnonanoate);
rac-(2R,3R)-8-(4-hydroxybutyl)-8-azaspiro[4.5]decane-2,3-diyl bis(2-butyldecanoate);
rac-(2R,3R)-3-((2-ethylnonanoyl)oxy)-8-(4-hydroxybutyl)-8-azaspiro[4.5]decan-2-yl 2-hexyldecanoate; and
bis(3-pentyloctyl) 3-(9-(4-hydroxybutyl)-3,9-diazaspiro[5.5]undecan-3-yl)pentanedioate;
   or a pharmaceutically acceptable salt thereof.

In another embodiment of the invention there is provided a pharmaceutical composition comprising a nucleic acid, at least one pharmaceutically acceptable excipient, and the compound described herein, or a pharmaceutically acceptable salt thereof. In a further embodiment of the invention there is provided a method for administering a nucleic acid to a subject in need thereof, the method comprising preparing or providing the pharmaceutical composition described herein, and administering the pharmaceutical composition to the subject.

It is to be understood that both the foregoing general description and the following detailed description are exemplary and explanatory only and are not restrictive of the invention, as claimed.

### BRIEF DESCRIPTION OF THE DRAWINGS

FIG. 1 shows 50% Neutralization Titer at 2 weeks post-dose 2 of 0.2 μg LNP (modRNA Flu HA/California) to Balb/c mice, geometric mean titer (GMT) ratio over ALC-0315 Control. Study #1 and Study #2 were run using the same protocol (see Example 74).

### DETAILED DESCRIPTION OF THE INVENTION

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.

Described below are embodiments of the invention, where for convenience Embodiment 1 (E1) is identical to the embodiment of Formula (I) provided above. Exemplary embodiments (E) of the invention provided herein include:
E1 A compound of Formula (I)

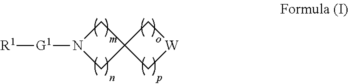

or a pharmaceutically acceptable salt, N-oxide, tautomer, or stereoisomer thereof,
wherein
m, n, o, and p are each independently 1-3;
G¹ is C₁₋₁₂alkylene or C₂₋₁₂alkenylene;
R¹ is —N(R²)R³, —OR⁴, CN, —N(R⁴)(heteroaryl), —O(CH₂)_{q}OH, —(OCH₂CH₂)ᵣOH, —OC(═O)R⁵, —N(R⁴)C(═O)R⁵, —N(R⁴)S(O)₂R⁵, —N(R⁴)C(═O)N(R²)R³, —OC(═O)N(R²)R³, —N(R⁴)C(═O)OR⁵, —N(R⁴)C(═S)N(R²)R³, —N(R⁴)C(═NR⁶)N(R²)R³, or

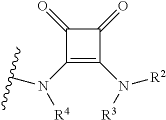

R² and R³ are each independently H, C₁₋₆alkyl, C₃₋₈cycloalkyl, or aryl; or R² and R³ together with the nitrogen atom to which they are attached form a heterocyclic ring;
R⁴ is H, C₁₋₆alkyl or C₃₋₈cycloalkyl;
R⁵ is C₁₋₆alkyl or C₃₋₈cycloalkyl optionally substituted by C₁₋₆alkyl;
R⁶ is H, CN, NO₂, C₁₋₆alkyl, OR⁵, S(O)₂R⁵, or S(O)₂N(R²)R³;
q is 2-6;
r is 1-6;
W is

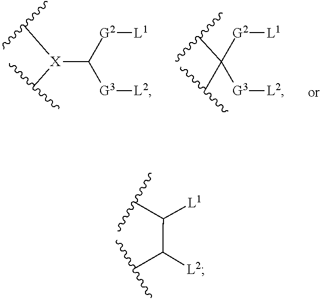

X is N or CH;
G² and G³ are each independently C₁₋₁₂alkylene or C₂₋₁₂alkenylene;
L¹ and L² are each independently —C(═O)OR⁷, —OC(═O)R⁷, —OC(═O)(CH₂)ᵣC(═O)OR⁷, —OC(═O)(CH₂)ᵣOC(═O)R⁷, —OC(═O)N(R⁴)R⁷, —N(R⁴)C(═O)OR⁷, —N(R⁴)C(═O)N(R⁴)R⁷, —OC(═O)OR⁷, or —S—SR⁷; and
R⁷ is C₆₋₂₄alkyl, C₆₋₂₄alkenyl, or C₆₋₂₄alkynyl where each is optionally substituted by F, C₁₋₆alkoxy, C₃₋₈cycloalkyl, or C₃₋₈cycloalkenyl, wherein the R⁷ of L¹ and L² may be the same or different.
E2 The compound of embodiment E1 having Formula (Ia)

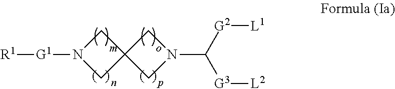

or a pharmaceutically acceptable salt, N-oxide, tautomer or stereoisomer thereof,
wherein m, n, o, and p are each independently 1 or 2.
E3 The compound of embodiment E1 having Formula (Ib)

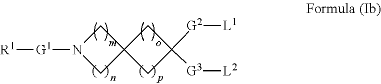

or a pharmaceutically acceptable salt, N-oxide, tautomer or stereoisomer thereof,
wherein m, n, o, and p are each independently 1 or 2.
E4 The compound of embodiment E1 having Formula (Ic)

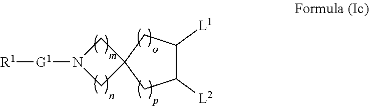

or a pharmaceutically acceptable salt, N-oxide, tautomer or stereoisomer thereof,
wherein m and n are each independently 1 or 2; and
and p are each 1.
E5 The compound of any one of embodiments E1-E4, wherein
   G¹ is C₁₋₁₂alkylene;
   R¹ is —OH or

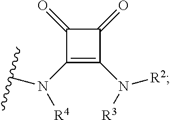

R² and R³ are each independently H, C₁₋₆alkyl, or C₃₋₈cycloalkyl; or R² and R³ together with the nitrogen atom to which they are attached form a heterocyclic ring;
R⁴ is H, C₁₋₆alkyl or C₃₋₈cycloalkyl;
G² and G³ are each independently C₁₋₁₂alkylene;
L¹ and L² are each —OC(═O)R⁷; and
R⁷ is C₆₋₂₄alkyl, C₆₋₂₄alkenyl, or C₆₋₂₄alkynyl wherein each is optionally substituted by F, C₁₋₆alkoxy, C₃₋₈cycloalkyl, or C₃₋₈cycloalkenyl, wherein the R⁷ of L¹ and L² may be the same or different.
E6 The compound of any one of embodiments E1-E5, wherein R⁷, has the following structure:

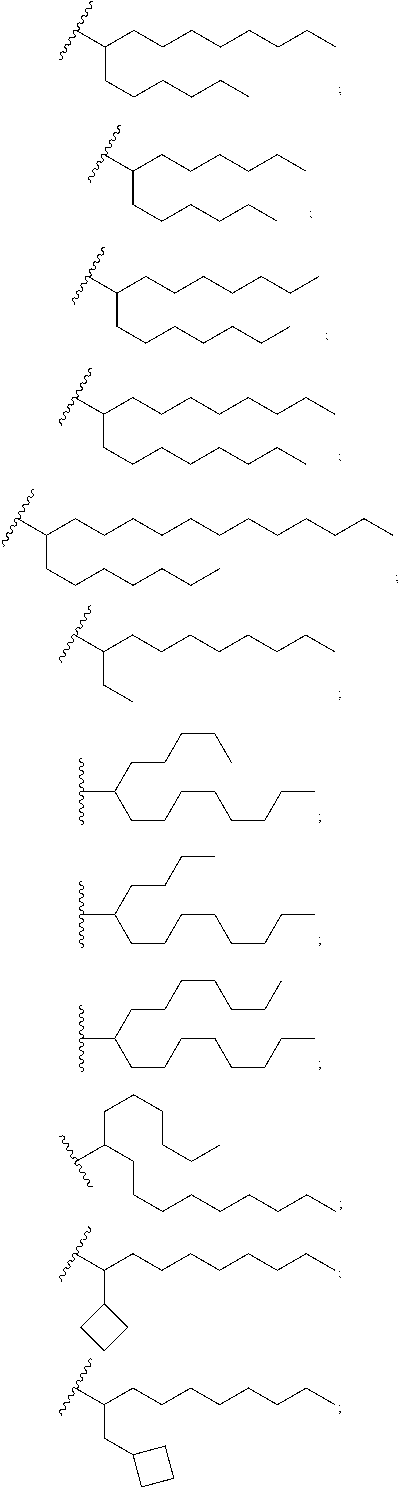

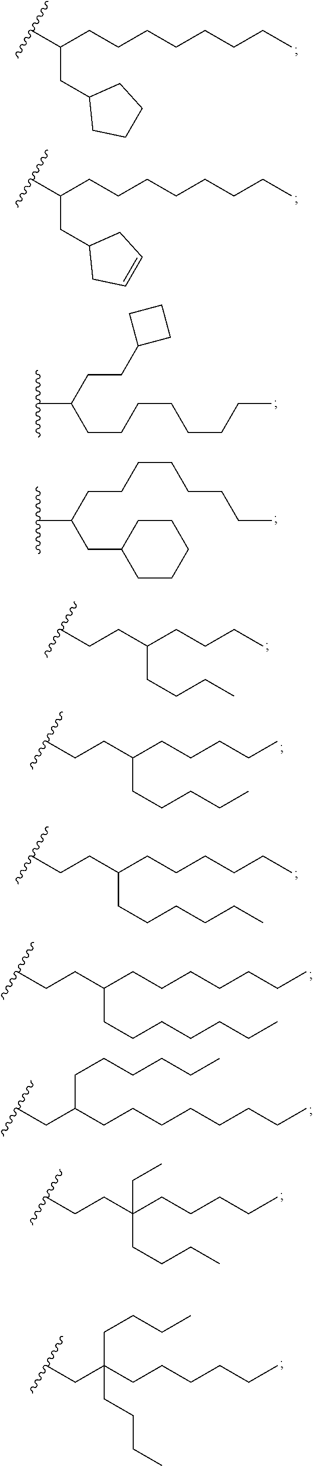

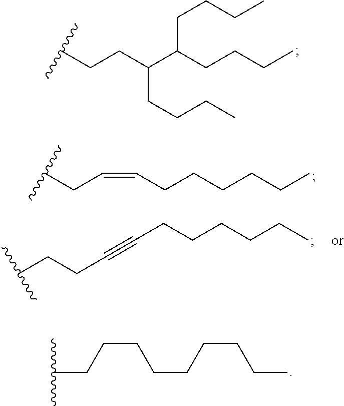

E7 The compound of any one of embodiments E1-E6, wherein R¹ is OH.
E8 The compound of any one of embodiments E1-E6, wherein R¹ is

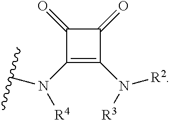

E9 The compound of any one of embodiments E1-E8, wherein G¹ is C₂-C₅ alkylene.
E10 The compound of any one of embodiments E1-E8, wherein G¹ is C₃-C₅ alkylene.
E11 The compound of embodiment E1 selected from the group consisting of:
(3-(4-hydroxybutyl)-3-azaspiro[5.5]undecane-9,9-diyl)bis(methylene) bis(2-heptylnonanoate);
2-(3-(4-hydroxybutyl)-3-azaspiro[5.5]undecan-9-yl)propane-1,3-diyl bis(2-heptylnonanoate);
2-(9-(4-hydroxybutyl)-3,9-diazaspiro[5.5]undecan-3-yl)propane-1,3-diyl bis(2-heptylnonanoate);
2-(9-(4-hydroxybutyl)-3,9-diazaspiro[5.5]undecan-3-yl)propane-1,3-diyl bis(2-cyclobutyldecanoate);
2-(9-(5-hydroxypentyl)-3,9-diazaspiro[5.5]undecan-3-yl)propane-1,3-diyl bis(2-hexyldecanoate);
2-(2-(4-hydroxybutyl)-2,8-diazaspiro[4.5]decan-8-yl)propane-1,3-diyl bis(2-hexyldecanoate);
3-((2-hexyldecanoyl)oxy)-2-(9-(4-hydroxybutyl)-3,9-diazaspiro[5.5]undecan-3-yl)propyl palmitate;
2-(9-(3-((2-(methylamino)-3,4-dioxocyclobut-1-en-1-yl)amino)propyl)-3,9-diazaspiro[5.5]undecan-3-yl)propane-1,3-diyl bis(2-heptylnonanoate);
2-(9-(3-(1-methylcyclopropane-1-carboxamido)propyl)-3,9-diazaspiro[5.5]undecan-3-yl)propane-1,3-diyl bis(2-hexyldecanoate);
2-(9-(3-hydroxypropyl)-3,9-diazaspiro[5.5]undecan-3-yl)propane-1,3-diyl bis(2-heptylnonanoate);
2-(9-(3-(ethylsulfonamido)propyl)-3,9-diazaspiro[5.5]undecan-3-yl)propane-1,3-diyl bis(2-hexyldecanoate);
2-(7-(4-hydroxybutyl)-2,7-diazaspiro[4.4]nonan-2-yl)propane-1,3-diyl bis(2-heptylnonanoate);
2-(7-(4-hydroxybutyl)-2,7-diazaspiro[3.5]nonan-2-yl)propane-1,3-diyl bis(2-heptylnonanoate);
2-(8-(4-hydroxybutyl)-2,8-diazaspiro[4.5]decan-2-yl)propane-1,3-diyl bis(2-heptylnonanoate);
3-(9-(4-hydroxybutyl)-3,9-diazaspiro[5.5]undecan-3-yl)pentane-1,5-diyl bis(2-hexyldecanoate);
2-(7-(5-hydroxypentyl)-2,7-diazaspiro[4.4]nonan-2-yl)propane-1,3-diyl bis(2-hexyldecanoate);
2-(2-(5-hydroxypentyl)-2,8-diazaspiro[4.5]decan-8-yl)propane-1,3-diyl bis(2-hexyldecanoate);
2-(7-(5-hydroxypentyl)-2,7-diazaspiro[3.5]nonan-2-yl)propane-1,3-diyl bis(2-heptylnonanoate);
2-(9-(4-hydroxybutyl)-3,9-diazaspiro[5.5]undecan-3-yl)pentane-1,5-diyl bis(2-hexyldecanoate);
2-(2-(4-hydroxybutyl)-2,7-diazaspiro[3.5]nonan-7-yl)propane-1,3-diyl bis(2-heptylnonanoate);
2-(9-(5-hydroxypentan-2-yl)-3,9-diazaspiro[5.5]undecan-3-yl)propane-1,3-diyl bis(2-hexyldecanoate);
2-(2-(4-hydroxybutyl)-2,8-diazaspiro[4.5]decan-8-yl)propane-1,3-diyl bis(2-heptylnonanoate);
2-(9-(4-hydroxybutyl)-3,9-diazaspiro[5.5]undecan-3-yl)propane-1,3-diyl bis(2-hexyldecanoate);
2-(9-(4-hydroxybutyl)-3,9-diazaspiro[5.5]undecan-3-yl)propane-1,3-diyl bis(2-octyldecanoate);
2-(9-(5-hydroxypentyl)-3,9-diazaspiro[5.5]undecan-3-yl)propane-1,3-diyl bis(2-heptylnonanoate);
2-(9-(4-hydroxybutyl)-3,9-diazaspiro[5.5]undecan-3-yl)propane-1,3-diyl bis(2-heptyldecanoate);
2-(9-(4-hydroxybutyl)-3,9-diazaspiro[5.5]undecan-3-yl)propane-1,3-diyl bis(2-butyldecanoate);
2-(9-(4-hydroxybutyl)-3,9-diazaspiro[5.5]undecan-3-yl)propane-1,3-diyl bis(3-hexylundecanoate);
2-(9-(4-hydroxybutyl)-3,9-diazaspiro[5.5]undecan-3-yl)propane-1,3-diyl bis(2-pentyldecanoate);
2-(9-(4-hydroxybutyl)-3,9-diazaspiro[5.5]undecan-3-yl)propane-1,3-diyl bis(2-(cyclobutylmethyl)decanoate);
2-(9-(4-hydroxybutyl)-3,9-diazaspiro[5.5]undecan-3-yl)propane-1,3-diyl bis(2-heptyltetradecanoate);
2-(9-(4-hydroxybutyl)-3,9-diazaspiro[5.5]undecan-3-yl)propane-1,3-diyl bis(2-(cyclopentylmethyl)decanoate);
2-(7-(4-hydroxybutyl)-2,7-diazaspiro[4.4]nonan-2-yl)propane-1,3-diyl bis(2-hexyldecanoate);
2-(7-(4-hydroxybutyl)-2,7-diazaspiro[3.5]nonan-2-yl)propane-1,3-diyl bis(2-hexyldecanoate);
2-(9-(4-hydroxybutyl)-3,9-diazaspiro[5.5]undecan-3-yl)propane-1,3-diyl bis(2-(cyclopent-3-en-1-ylmethyl)decanoate);
2-(9-(4-hydroxybutyl)-3,9-diazaspiro[5.5]undecan-3-yl)propane-1,3-diyl bis(2-(2-cyclobutylethyl)decanoate);
2-(9-(4-hydroxybutyl)-3,9-diazaspiro[5.5]undecan-3-yl)propane-1,3-diyl bis(2-(cyclohexylmethyl)decanoate);
2-(9-(4-hydroxybutyl)-3,9-diazaspiro[5.5]undecan-3-yl)propane-1,3-diyl bis(4,5-dibutylnonanoate);
2-(9-(4-hydroxybutyl)-3,9-diazaspiro[5.5]undecan-3-yl)propane-1,3-diyl bis(3,3-dibutylnonanoate);
2-(9-(4-hydroxybutyl)-3,9-diazaspiro[5.5]undecan-3-yl)propane-1,3-diyl bis(4-heptylundecanoate);
2-(7-(5-hydroxypentyl)-2,7-diazaspiro[4.4]nonan-2-yl)propane-1,3-diyl bis(2-heptylnonanoate);
3-(decanoyloxy)-2-(9-(4-hydroxybutyl)-3,9-diazaspiro[5.5]undecan-3-yl)propyl 2-hexyldecanoate;
3-((2-hexyldecanoyl)oxy)-2-(9-(4-hydroxybutyl)-3,9-diazaspiro[5.5]undecan-3-yl)propyl (Z)-dodec-5-enoate;
3-((2-(cyclobutylmethyl)decanoyl)oxy)-2-(9-(4-hydroxybutyl)-3,9-diazaspiro[5.5]undecan-3-yl)propyl 2-hexyldecanoate;
3-((2-hexyldecanoyl)oxy)-2-(9-(4-hydroxybutyl)-3,9-diazaspiro[5.5]undecan-3-yl)propyl 2-butylundecanoate;
3-((4,5-dibutylnonanoyl)oxy)-2-(9-(4-hydroxybutyl)-3,9-diazaspiro[5.5]undecan-3-yl)propyl palmitate;
3-(9-(4-hydroxybutyl)-3,9-diazaspiro[5.5]undecan-3-yl)pentane-1,5-diyl bis(2-heptylnonanoate);
2-(2-(5-hydroxypentyl)-2,8-diazaspiro[4.5]decan-8-yl)propane-1,3-diyl bis(2-heptylnonanoate);
2-(7-(5-hydroxypentyl)-2,7-diazaspiro[3.5]nonan-2-yl)propane-1,3-diyl bis(2-hexyldecanoate);
2-(2-(4-hydroxybutyl)-2,7-diazaspiro[3.5]nonan-7-yl)propane-1,3-diyl bis(2-hexyldecanoate);
rac-(2R,3R)-8-(4-hydroxybutyl)-8-azaspiro[4.5]decane-2,3-diyl bis(2-heptylnonanoate);
rac-(2R,3R)-8-(2-hydroxyethyl)-8-azaspiro[4.5]decane-2,3-diyl bis(2-heptylnonanoate);
rac-(7R,8R)-2-(3-hydroxypropyl)-2-azaspiro[4.4]nonane-7,8-diyl bis(2-heptylnonanoate);
rac-(2R,3R)-8-(3-hydroxypropyl)-8-azaspiro[4.5]decane-2,3-diyl bis(2-heptylnonanoate);
rac-(2R,3R)-8-(3-((2-(methylamino)-3,4-dioxocyclobut-1-en-1-yl)amino)propyl)-8-azaspiro[4.5]decane-2,3-diyl bis(2-heptylnonanoate);
rac-(2R,3R)-8-(5-hydroxypentyl)-8-azaspiro[4.5]decane-2,3-diyl bis(2-heptylnonanoate);
(2R,3R)-8-(4-hydroxybutyl)-8-azaspiro[4.5]decane-2,3-diyl bis(2-heptylnonanoate);
(2S,3S)-8-(4-hydroxybutyl)-8-azaspiro[4.5]decane-2,3-diyl bis(2-heptylnonanoate);
rac-(2R,3R)-8-(4-hydroxybutyl)-8-azaspiro[4.5]decane-2,3-diyl bis(2-octyldecanoate);
rac-(2R,3R)-8-(4-hydroxybutyl)-8-azaspiro[4.5]decane-2,3-diyl bis(2-heptyltetradecanoate);
rac-(2R,3R)-8-(4-hydroxybutyl)-8-azaspiro[4.5]decane-2,3-diyl bis(2-hexyldecanoate);
rac-O,O′-((2R,3R)-8-(4-hydroxybutyl)-8-azaspiro[4.5]decane-2,3-diyl) di(pentadecan-8-yl) disuccinate;
rac-O′¹,O¹-((2R,3R)-8-(4-hydroxybutyl)-8-azaspiro[4.5]decane-2,3-diyl) 7,7′-di(pentadecan-8-yl) di(heptanedioate);
rac-(((2R,3R)-8-(4-hydroxybutyl)-8-azaspiro[4.5]decane-2,3-diyl)bis(oxy))bis(6-oxohexane-6,1-diyl) bis(2-heptylnonanoate);
rac-(2R,3R)-8-(4-hydroxybutyl)-8-azaspiro[4.5]decane-2,3-diyl bis(2-pentyldecanoate);
rac-(2R,3R)-8-(4-hydroxybutyl)-8-azaspiro[4.5]decane-2,3-diyl bis(2-heptyldecanoate);
rac-(2R,3R)-8-(4-hydroxybutyl)-8-azaspiro[4.5]decane-2,3-diyl bis(2-hexylundecanoate);
rac-(2R,3R)-8-(4-hydroxybutyl)-8-azaspiro[4.5]decane-2,3-diyl bis(2-hexylnonanoate);
rac-(2R,3R)-8-(4-hydroxybutyl)-8-azaspiro[4.5]decane-2,3-diyl bis(2-butyldecanoate);
rac-(2R,3R)-3-((2-ethylnonanoyl)oxy)-8-(4-hydroxybutyl)-8-azaspiro[4.5]decan-2-yl 2-hexyldecanoate; and
bis(3-pentyloctyl) 3-(9-(4-hydroxybutyl)-3,9-diazaspiro[5.5]undecan-3-yl)pentanedioate;
   or a pharmaceutically acceptable salt thereof.
   E12 The compound of embodiment E1 selected from the group consisting of:
2-(9-(4-hydroxybutyl)-3,9-diazaspiro[5.5]undecan-3-yl)propane-1,3-diyl bis(2-heptylnonanoate);
2-(7-(4-hydroxybutyl)-2,7-diazaspiro[3.5]nonan-2-yl)propane-1,3-diyl bis(2-heptylnonanoate);
2-(2-(4-hydroxybutyl)-2,8-diazaspiro[4.5]decan-8-yl)propane-1,3-diyl bis(2-heptylnonanoate);
2-(9-(4-hydroxybutyl)-3,9-diazaspiro[5.5]undecan-3-yl)propane-1,3-diyl bis(2-hexyldecanoate);
2-(9-(5-hydroxypentyl)-3,9-diazaspiro[5.5]undecan-3-yl)propane-1,3-diyl bis(2-heptylnonanoate);
rac-(2R,3R)-8-(4-hydroxybutyl)-8-azaspiro[4.5]decane-2,3-diyl bis(2-heptylnonanoate);
rac-(2R,3R)-8-(4-hydroxybutyl)-8-azaspiro[4.5]decane-2,3-diyl bis(2-hexyldecanoate);
2-(2-(4-hydroxybutyl)-2,8-diazaspiro[4.5]decan-8-yl)propane-1,3-diyl bis(2-hexyldecanoate);
2-(9-(3-hydroxypropyl)-3,9-diazaspiro[5.5]undecan-3-yl)propane-1,3-diyl bis(2-heptylnonanoate);
2-(9-(4-hydroxybutyl)-3,9-diazaspiro[5.5]undecan-3-yl)propane-1,3-diyl bis(3-hexylundecanoate);
2-(7-(4-hydroxybutyl)-2,7-diazaspiro[4.4]nonan-2-yl)propane-1,3-diyl bis(2-hexyldecanoate);
2-(7-(5-hydroxypentyl)-2,7-diazaspiro[3.5]nonan-2-yl)propane-1,3-diyl bis(2-hexyldecanoate);
2-(2-(4-hydroxybutyl)-2,7-diazaspiro[3.5]nonan-7-yl)propane-1,3-diyl bis(2-hexyldecanoate); and
rac-(2R,3R)-8-(5-hydroxypentyl)-8-azaspiro[4.5]decane-2,3-diyl bis(2-heptylnonanoate);
   or a pharmaceutically acceptable salt thereof.
   E13 2-(9-(4-hydroxybutyl)-3,9-diazaspiro[5.5]undecan-3-yl)propane-1,3-diyl bis(2-heptylnonanoate), or a pharmaceutically acceptable salt thereof.
   E14 2-(7-(4-hydroxybutyl)-2,7-diazaspiro[3.5]nonan-2-yl)propane-1,3-diyl bis(2-heptylnonanoate), or a pharmaceutically acceptable salt thereof.
   E15 2-(2-(4-hydroxybutyl)-2,8-diazaspiro[4.5]decan-8-yl)propane-1,3-diyl bis(2-heptylnonanoate), or a pharmaceutically acceptable salt thereof.
   E16 2-(9-(4-hydroxybutyl)-3,9-diazaspiro[5.5]undecan-3-yl)propane-1,3-diyl bis(2-hexyldecanoate), or a pharmaceutically acceptable salt thereof.
   E17 2-(9-(5-hydroxypentyl)-3,9-diazaspiro[5.5]undecan-3-yl)propane-1,3-diyl bis(2-heptylnonanoate), or a pharmaceutically acceptable salt thereof.
   E18 rac-(2R,3R)-8-(4-hydroxybutyl)-8-azaspiro[4.5]decane-2,3-diyl bis(2-heptylnonanoate), or a pharmaceutically acceptable salt thereof.
   E19 rac-(2R,3R)-8-(4-hydroxybutyl)-8-azaspiro[4.5]decane-2,3-diyl bis(2-hexyldecanoate), or a pharmaceutically acceptable salt thereof.
   E20 2-(2-(4-hydroxybutyl)-2,8-diazaspiro[4.5]decan-8-yl)propane-1,3-diyl bis(2-hexyldecanoate), or a pharmaceutically acceptable salt thereof.
   E21 2-(9-(3-hydroxypropyl)-3,9-diazaspiro[5.5]undecan-3-yl)propane-1,3-diyl bis(2-heptylnonanoate), or a pharmaceutically acceptable salt thereof.
   E22 2-(9-(4-hydroxybutyl)-3,9-diazaspiro[5.5]undecan-3-yl)propane-1,3-diyl bis(3-hexylundecanoate), or a pharmaceutically acceptable salt thereof.
   E23 2-(7-(4-hydroxybutyl)-2,7-diazaspiro[4.4]nonan-2-yl)propane-1,3-diyl bis(2-hexyldecanoate), or a pharmaceutically acceptable salt thereof.
   E24 2-(7-(5-hydroxypentyl)-2,7-diazaspiro[3.5]nonan-2-yl)propane-1,3-diyl bis(2-hexyldecanoate), or a pharmaceutically acceptable salt thereof.
   E25 2-(2-(4-hydroxybutyl)-2,7-diazaspiro[3.5]nonan-7-yl)propane-1,3-diyl bis(2-hexyldecanoate), or a pharmaceutically acceptable salt thereof.
   E26 rac-(2R,3R)-8-(5-hydroxypentyl)-8-azaspiro[4.5]decane-2,3-diyl bis(2-heptylnonanoate), or a pharmaceutically acceptable salt thereof.
   E27 2-(9-(4-hydroxybutyl)-3,9-diazaspiro[5.5]undecan-3-yl)propane-1,3-diyl bis(2-heptylnonanoate).
   E28 2-(7-(4-hydroxybutyl)-2,7-diazaspiro[3.5]nonan-2-yl)propane-1,3-diyl bis(2-heptylnonanoate).
   E29 2-(2-(4-hydroxybutyl)-2,8-diazaspiro[4.5]decan-8-yl)propane-1,3-diyl bis(2-heptylnonanoate).
   E30 2-(9-(4-hydroxybutyl)-3,9-diazaspiro[5.5]undecan-3-yl)propane-1,3-diyl bis(2-hexyldecanoate).
   E31 2-(9-(5-hydroxypentyl)-3,9-diazaspiro[5.5]undecan-3-yl)propane-1,3-diyl bis(2-heptylnonanoate).
   E32 rac-(2R,3R)-8-(4-hydroxybutyl)-8-azaspiro[4.5]decane-2,3-diyl bis(2-heptylnonanoate).
   E33 rac-(2R,3R)-8-(4-hydroxybutyl)-8-azaspiro[4.5]decane-2,3-diyl bis(2-hexyldecanoate).
   E34 2-(2-(4-hydroxybutyl)-2,8-diazaspiro[4.5]decan-8-yl)propane-1,3-diyl bis(2-hexyldecanoate).
   E35 2-(9-(3-hydroxypropyl)-3,9-diazaspiro[5.5]undecan-3-yl)propane-1,3-diyl bis(2-heptylnonanoate).
   E36 2-(9-(4-hydroxybutyl)-3,9-diazaspiro[5.5]undecan-3-yl)propane-1,3-diyl bis(3-hexylundecanoate).
   E37 2-(7-(4-hydroxybutyl)-2,7-diazaspiro[4.4]nonan-2-yl)propane-1,3-diyl bis(2-hexyldecanoate).
   E38 2-(7-(5-hydroxypentyl)-2,7-diazaspiro[3.5]nonan-2-yl)propane-1,3-diyl bis(2-hexyldecanoate).
   E39 2-(2-(4-hydroxybutyl)-2,7-diazaspiro[3.5]nonan-7-yl)propane-1,3-diyl bis(2-hexyldecanoate).
   E40 rac-(2R,3R)-8-(5-hydroxypentyl)-8-azaspiro[4.5]decane-2,3-diyl bis(2-heptylnonanoate).
   E41 A pharmaceutical composition comprising a nucleic acid, at least one pharmaceutically acceptable excipient, and the compound according to any one of embodiments E1-E40, or a pharmaceutically acceptable salt thereof.
   E42 The pharmaceutical composition of embodiment E41, wherein the pharmaceutically acceptable excipient is selected from the group consisting of neutral lipids, steroids and polymer conjugated lipids.
   E43 The pharmaceutical composition of any one of embodiments E41-E42, wherein the pharmaceutical composition comprises 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), phophatidylethanolamines such as 1,2-Dioleoyl-sn-glycero-3-phosphoethanolamine (DOPE), sphingomyelins (SM), or a combination thereof.
   E44 The pharmaceutical composition of any one of embodiments E42 to E43, wherein the steroid is cholesterol.
   E45 The pharmaceutical composition of any one of embodiments E42 to E44, wherein the polymer conjugated lipid is a pegylated lipid.
   E46 The pharmaceutical composition of embodiment E45, wherein the pegylated lipid is PEG-DAG, PEG-PE, PEG-S-DAG, PEG-cer, PEG-DMG, 2-[(polyethylene glycol)-2000]-N,N-ditetradecylacetamide (ALC-0159) or a PEG dialkyoxypropylcarbamate.
   E47 The pharmaceutical composition of any one of embodiments E41 to E46, wherein the nucleic acid is RNA.
   E48 The pharmaceutical composition of embodiment E47, wherein the RNA is messenger RNA.
   E49 The pharmaceutical composition of any one of embodiments E47-E48, wherein the RNA is modRNA or saRNA.
   E50 A method for administering a nucleic acid to a subject in need thereof, the method comprising preparing or providing the pharmaceutical composition of any one of embodiments E41-E49, and administering the pharmaceutical composition to the subject.
   E51 A method of making the compound of any one of embodiments E1-E40, the method comprising any of the methods set forth in the Examples described herein.
   E52 A method of making the pharmaceutical composition of any one of embodiments E41-E49, the method comprising combining the nucleic acid, at least one pharmaceutically acceptable excipient, and the compound according to any one of embodiments E1-E40.
   E53 A compound according to any one of embodiments E1 to E40 for use as a component of a medicament.
   E54 The compound of embodiment E53, wherein the medicament is a vaccine.
   E55 Use of a compound according to any one of embodiments E1 to E40 for the manufacture of a medicament.
   E56 The use of embodiment E55, wherein the medicament is a vaccine.

Each of the embodiments described herein may be combined with any other embodiment(s) described herein not inconsistent with the embodiment(s) with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the embodiment(s) described herein.

Furthermore, each of the embodiments described herein envisions within its scope pharmaceutically acceptable salts of the compounds described herein.

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

### Definitions

Unless otherwise defined herein, scientific and technical terms used in connection with the present invention have the meanings that are commonly understood by those of ordinary skill in the art.

The invention described herein suitably may be practiced in the absence of any element(s) not specifically disclosed herein.

“Compounds of the invention” include compounds of Formula I, I(a), I(b), and/or I(c), pharmaceutically acceptable salts, N-oxide, tautomers or stereoisomers thereof, and the novel intermediates used in the preparation thereof. One of ordinary skill in the art will appreciate that compounds of the invention include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the invention include solvates, hydrates, isomorphs, polymorphs, esters, salt forms, prodrugs, and isotopically labelled versions thereof (including deuterium substitutions), where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of XXX) means that the parameter may vary by as much as 10% below or above the stated numerical value for that parameter. For example, a dose of about 5 mg means 5%±10%, e.g., it may be 4.5 mg and 5.5 mg or any number therebetween.

As used herein, the term “aqueous solution” refers to a composition comprising water.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (e.g., unsubstituted), or the group may have one or more non-hydrogen substituents (e.g., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Halogen” or “halo” refers to fluoro, chloro, bromo and iodo (F, Cl, Br, I).

“Cyano” refers to a substituent having a carbon atom joined to a nitrogen atom by a triple bond, e.g., —C≡N.

“Hydroxy” refers to an —OH group.

“Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 12 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted or substituted, as further defined herein.

“Alkylene” or “alkylene chain” refers to a straight or branched divalent hydrocarbon chain linking the rest of the molecule to a radical group, consisting solely of carbon and hydrogen, which is saturated, and having, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₈alkylene), one to six carbon atoms (C₁-C₆ alkylene), two to four carbon atoms (C₂-C₄ alkylene), one to two carbon atoms (C₁-C₂ alkylene), e.g., methylene, ethylene, propylene, n-butylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Fluoroalkyl” refers to an alkyl group, as defined herein, wherein from one to all of the hydrogen atoms of the alkyl group are replaced by fluoro atoms. Examples include, but are not limited to, fluoromethyl, difluoromethyl, 2-fluoroethyl, 2,2-difluoroethyl, 2,2,2-trifluoroethyl, and 1,2,2,2-tetrafluoroethyl. Examples of fully substituted fluoroalkyl groups (also referred to as perfluoroalkyl groups) include trifluoromethyl (—CF₃) and pentafluoroethyl (—C₂F₅).

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain, but are not limited to, 1 to 8 carbon atoms (“C₁-C₈alkoxy”), 1 to 6 carbon atoms (“C₁-C₆ alkoxy”), 1 to 4 carbon atoms (“C₁-C₄ alkoxy”), or 1 to 3 carbon atoms (“C₁-C₃ alkoxy”). Alkoxy groups include, but are not limited to, methoxy, ethoxy, n-propoxy, isobutoxy, and the like.

“Alkoxyalkyl” refers to an alkyl group, as defined herein, that is substituted by an alkoxy group, as defined herein. Examples include, but are not limited to, CH₃OCH₂— and CH₃CH₂OCH₂—

“Alkenyl” refers to a monovalent aliphatic hydrocarbon radical, including straight chain or branched chain groups, consisting of at least two carbon atoms and at least one carbon-carbon double bond. For example, as used herein, the term “C₂-C₆ alkenyl” means straight or branched chain unsaturated radicals of 2 to 6 carbon atoms, including, but not limited to, ethenyl, 1-propenyl, 2-propenyl, 1-, 2-, or 3-butenyl, and the like.

“Alkenylene” or “alkenylene chain” refers to a straight or branched divalent hydrocarbon chain linking the rest of the molecule to a radical group, consisting solely of carbon and hydrogen, consisting of at least two carbon atoms and at least one carbon-carbon double bond, and having, for example, from one to twenty-four carbon atoms (C₁-C₂₄ alkenylene), one to fifteen carbon atoms (C₁-C₁₅ alkenylene), one to twelve carbon atoms (C₁-C₁₂ alkenylene), one to eight carbon atoms (C₁-C₈ alkenylene), one to six carbon atoms (C₁-C₆ alkenylene), two to four carbon atoms (C₂-C₄ alkenylene), one to two carbon atoms (C₁-C₂ alkenylene), e.g., ethenylene, propenylene, n-butenylene, and the like. The alkenylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Alkynyl” refers to a monovalent aliphatic hydrocarbon radical, including straight chain or branched chain groups, consisting of at least two carbon atoms and at least one carbon-carbon triple bond. Examples include, but are not limited to, ethynyl, 1-propynyl, 2-propynyl, 1-, 2-, or 3-butynyl, and the like.

“Cycloalkyl” or “carbocyclic ring” refers to a fully saturated hydrocarbon ring system that has the specified number of carbon atoms, which may be a monocyclic, bridged, spirocyclic, or fused bicyclic or polycyclic ring system that is connected to the base molecule through a carbon atom of the cycloalkyl ring. Cycloalkyl groups may contain, but are not limited to, 3 to 12 carbon atoms (“C₃-C₁₂ cycloalkyl”), 3 to 8 carbon atoms (“C₃-C₈ cycloalkyl”), 3 to 6 carbon atoms (“C₃-C₆ cycloalkyl”), 3 to 5 carbon atoms (“C₃-C₅ cycloalkyl”) or 3 to 4 carbon atoms (“C₃-C₄ cycloalkyl”). Examples include, but are not limited to, cyclopropyl, cyclobutyl, cyclopentyl, cyclohexyl, cycloheptyl, adamantanyl, and the like. Cycloalkyl groups may be optionally substituted, unsubstituted or substituted, as further defined herein.

Illustrative examples of cycloalkyl rings include, but are not limited to, the following:

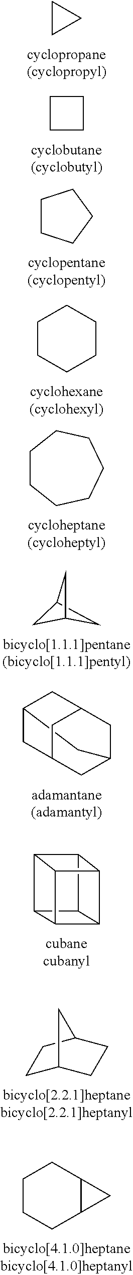

“Cycloalkenyl” refers to a hydrocarbon ring system that has the specified number of carbon atoms containing at least one carbon-carbon double bond, which may be a monocyclic, bridged, spirocyclic, or fused bicyclic or polycyclic ring system that is connected to the base molecule through a carbon atom of the cycloalkenyl ring. Cycloalkenyl groups may contain, but are not limited to, 3 to 12 carbon atoms (“C₃-C₁₂ cycloalkenyl”), 3 to 8 carbon atoms (“C₃-C₈ cycloalkenyl”), 3 to 6 carbon atoms (“C₃-C₆ cycloalkenyl”), 3 to 5 carbon atoms (“C₃-C₅ cycloalkenyl”) or 3 to 4 carbon atoms (“C₃-C₄ cycloalkenyl”). Examples include, but are not limited to, cyclopentenyl, cyclohexenyl, cycloheptenyl, and the like. Cycloalkenyl groups may be optionally substituted, unsubstituted or substituted, as further defined herein.

“Cycloalkoxy” refers to a cycloalkyl group, as defined herein, that is single bonded to an oxygen atom. The attachment point of a cycloalkoxy radical to a molecule is through the oxygen atom. A cycloalkoxy radical may be depicted as cycloalkyl-O—. Cycloalkoxy groups may contain, but are not limited to, 3 to 8 carbon atoms (“C₃-C₈ cycloalkoxy”), 3 to 6 carbon atoms (“C₃-C₆ cycloalkoxy”), and 3 to 4 carbon atoms (“C₃-C₄ cycloalkoxy”). Cycloalkoxy groups include, but are not limited to, cyclopropoxy, cyclobutoxy, cyclopentoxy and the like.

“Heterocycloalkyl” refers to a fully saturated ring system containing the specified number of ring atoms and containing at least one heteroatom selected from N, O and S as a ring member, where ring S atoms are optionally substituted by one or two oxo groups (e.g., S(O)_{q}, where q is 0, 1 or 2) and where the heterocycloalkyl ring is connected to the base molecule via a ring atom, which may be C or N. Heterocycloalkyl rings include rings which are spirocyclic, bridged, or fused to one or more other heterocycloalkyl or carbocyclic rings, where such spirocyclic, bridged, or fused rings may themselves be saturated, partially unsaturated or aromatic to the extent unsaturation or aromaticity makes chemical sense, provided the point of attachment to the base molecule is an atom of the heterocycloalkyl portion of the ring system. Heterocycloalkyl rings may contain 1 to 4 heteroatoms selected from N, O, and S(O)_{q} as ring members, or 1 to 2 ring heteroatoms, provided that such heterocycloalkyl rings do not contain two contiguous oxygen or sulfur atoms. Heterocycloalkyl rings may be optionally substituted, unsubstituted or substituted, as further defined herein. Such substituents may be present on the heterocyclic ring attached to the base molecule, or on a spirocyclic, bridged or fused ring attached thereto. Heterocycloalkyl rings may include, but are not limited to, 3-8 membered heterocyclyl groups, for example 4-7 or 4-6 membered heterocycloalkyl groups, in accordance with the definition herein.

Illustrative examples of heterocycloalkyl rings include, but are not limited, to a monovalent radical of:

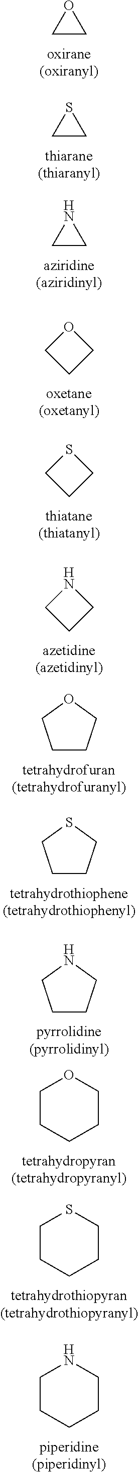

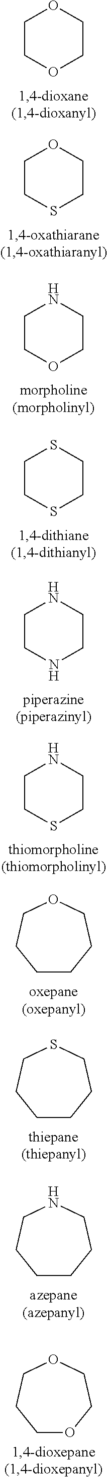

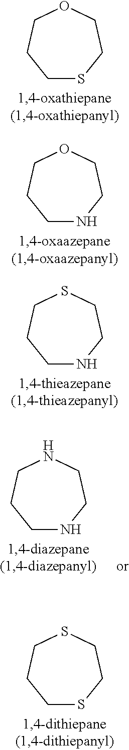

Illustrative examples of bridged and fused heterocycloalkyl groups include, but are not limited to a monovalent radical of:

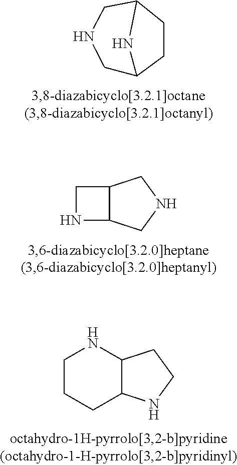

As used herein, “spirocyclic” refers to a bicyclic residue where each ring is independently a cycloalkyl, cycloalkenyl, or heterocycloalkyl ring as defined herein such that both rings share only one common carbon atom. The spirocyclic ring may be attached to the molecule by a carbon or nitrogen atom.

“Aryl” or “aromatic” refers to monocyclic, bicyclic (e.g., biaryl, fused) or polycyclic ring systems that contain the specified number of ring atoms, in which all carbon atoms in the ring are of sp² hybridization and in which the pi electrons are in conjugation. Aryl groups may contain, but are not limited to, 6 to 20 carbon atoms (“C₆-C₂₀ aryl”), 6 to 14 carbon atoms (“C₆-C₁₄ aryl”), 6 to 12 carbon atoms (“C₆-C₁₂ aryl”), or 6 to 10 carbon atoms (“C₆-C₁₀ aryl”). Fused aryl groups may include an aryl ring (e.g., a phenyl ring) fused to another aryl ring. Examples include, but are not limited to, phenyl, biphenyl, naphthyl, anthracenyl, phenanthrenyl, indanyl, and indenyl. Aryl groups may be optionally substituted, unsubstituted or substituted, as further defined herein.

Similarly, “heteroaryl” or “heteroaromatic” refer to monocyclic, bicyclic (e.g., heterobiaryl, fused) or polycyclic ring systems that contain the specified number of ring atoms and include at least one heteroatom selected from N, O and S as a ring member in a ring in which all carbon atoms in the ring are of sp² hybridization and in which the pi electrons are in conjugation. Heteroaryl groups may contain, but are not limited to, 5 to 20 ring atoms (“5-20 membered heteroaryl”), 5 to 14 ring atoms (“5-14 membered heteroaryl”), 5 to 12 ring atoms (“5-12 membered heteroaryl”), 5 to 10 ring atoms (“5-10 membered heteroaryl”), 5 to 9 ring atoms (“5-9 membered heteroaryl”), or 5 to 6 ring atoms (“5-6 membered heteroaryl”). Heteroaryl rings are attached to the base molecule via a ring atom of the heteroaromatic ring. Thus, either 5- or 6-membered heteroaryl rings, alone or in a fused structure, may be attached to the base molecule via a ring C or N atom. Examples of heteroaryl groups include, but are not limited to, pyrrolyl, furanyl, thiophenyl, pyrazolyl, imidazolyl, isoxazolyl, oxazolyl, isothiazolyl, thiazolyl, triazolyl, oxadiazolyl, thiadiazolyl, tetrazolyl, pyridinyl, pyridizinyl, pyrimidinyl, pyrazinyl, benzofuranyl, benzothiophenyl, indolyl, benzamidazolyl, indazolyl, quinolinyl, isoquinolinyl, purinyl, triazinyl, naphthyridinyl, cinnolinyl, quinazolinyl, quinoxalinyl and carbazolyl. Examples of 5- or 6-membered heteroaryl groups include, but are not limited to, pyrrolyl, furanyl, thiophenyl, pyrazolyl, imidazolyl, isoxazolyl, oxazolyl, isothiazolyl, thiazolyl, triazolyl, pyridinyl, pyrimidinyl, pyrazinyl and pyridazinyl rings. Heteroaryl groups may be optionally substituted, unsubstituted or substituted, as further defined herein.

Illustrative examples of monocyclic heteroaryl groups include, but are not limited to a monovalent radical of:

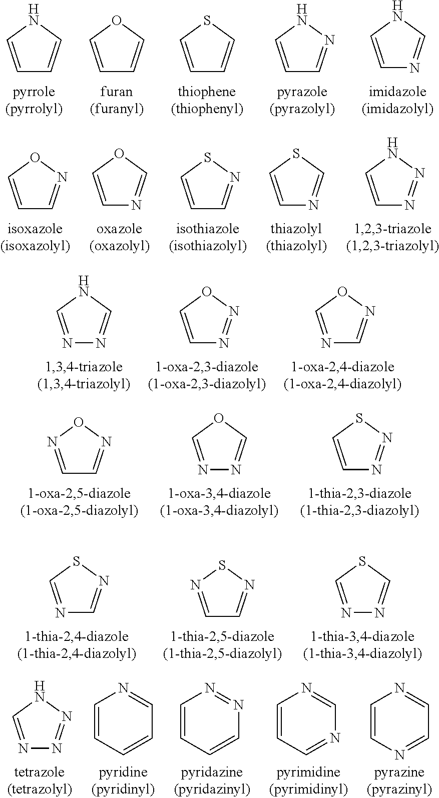

Illustrative examples of fused ring heteroaryl groups include, but are not limited to:

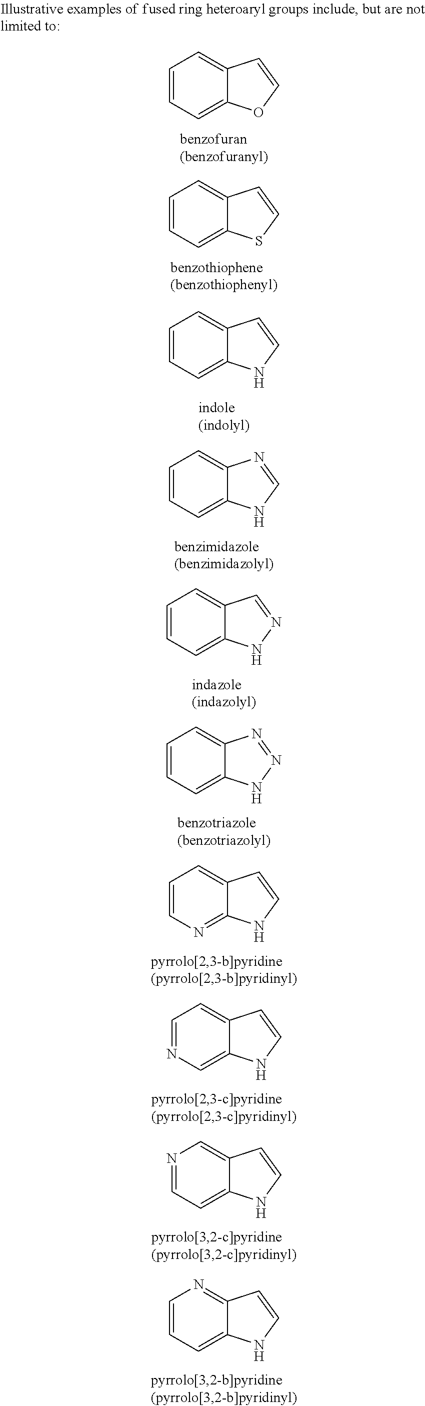

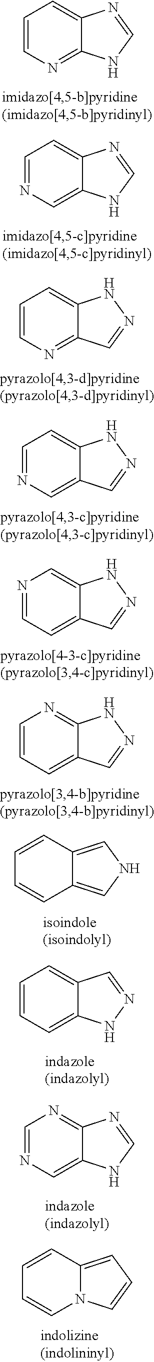

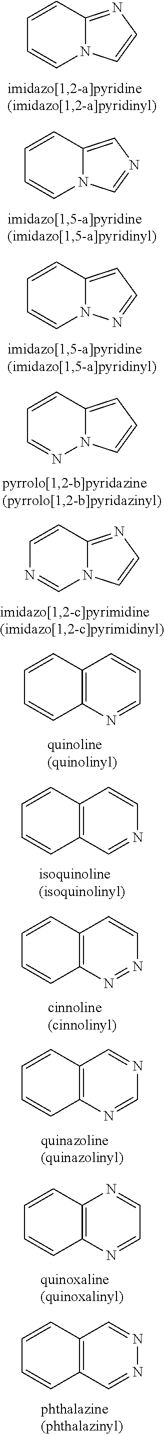

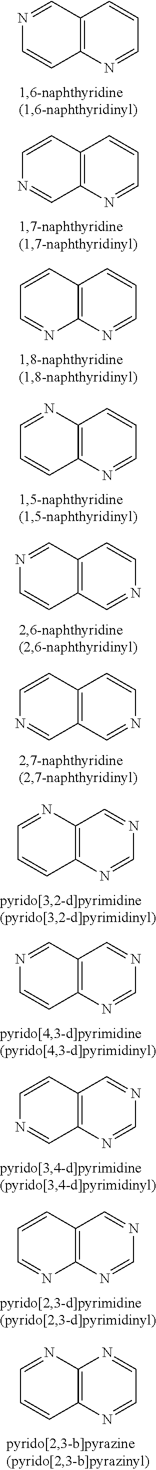

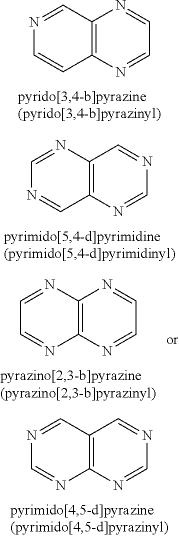 [SMILES: c1cc2nccnc2cn1 ; c1ncc2ncncc2n1 ; c1cnc2nccnc2n1 ; c1ncc2cncnc2n1]

“Amino” refers to a group —NH₂, which is unsubstituted. Where the amino is described as substituted or optionally substituted, the term includes groups of the form —NR′R″, where each of R′ and R″ is defined as further described herein. For example, “alkylamino” refers to a group-NR′R″, wherein one of R′ and R″ is an alkyl moiety and the other is H, and “dialkylamino” refers to —NR′R″ wherein both of R′ and R″ are alkyl moieties, where the alkyl moieties have the specified number of carbon atoms (e.g., —NH(C₁-C₄ alkyl) or —N(C₁-C₄ alkyl)₂).

“Aminoalkyl” refers to an alkyl group, as defined above, that is substituted by 1, 2, or 3 amino groups, as defined herein.

The term “pharmaceutically acceptable” means the substance (e.g., the compounds described herein) and any salt thereof, or composition containing the substance or salt of the invention is suitable for administration to a subject or patient.

“Deuterium enrichment factor” as used herein means the ratio between the deuterium abundance and the natural abundance of deuterium, each relative to hydrogen abundance. An atomic position designated as having deuterium typically has a deuterium enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Salts

Salts encompassed within the term “pharmaceutically acceptable salts” refer to compounds which are generally prepared by reacting the free base or free acid with a suitable organic or inorganic acid, or a suitable organic or inorganic base, respectively, to provide a salt of the compound of the invention that is suitable for administration to a subject or patient.

In addition, the compounds of Formula I may also include other salts of such compounds which are not necessarily pharmaceutically acceptable salts, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

Suitable acid addition salts for pharmaceutically acceptable salts may be formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of compounds of the invention may be prepared by methods well known to one skilled in the art, including but not limited to the following procedures
(i) by reacting a compound of the invention with the desired acid;
(ii) by removing an acid- or base-labile protecting group from a suitable precursor of a compound of the invention or by ring-opening a suitable cyclic precursor, for example, a lactone or lactam, using the desired acid; or
(iii) by converting one salt of a compound of the invention to another. This may be accomplished by reaction with an appropriate acid or by means of a suitable ion exchange procedure.

These procedures are typically carried out in solution. The resulting salt may precipitate out and be collected by filtration or may be recovered by evaporation of the solvent.

### Solvates

The compounds of the invention, and pharmaceutically acceptable salts thereof, may exist in unsolvated and solvated forms. The term ‘solvate’ is used herein to describe a molecular complex comprising a compound and one or more solvent molecules, for example, ethanol. The term ‘hydrate’ may be employed when the solvent is water.

The compounds of Formula I may include solvates of such compounds that are pharmaceutically acceptable. In addition, the compounds of Formula I may also include other solvates of such compounds which are not necessarily pharmaceutically acceptable solvates, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Solid Form

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

### Stereoisomers

Some compounds of the invention may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the invention containing one or more asymmetric carbon atoms may exist as two or more stereoisomers. Where a compound of the invention contains an alkenyl or alkenylene group, geometric cis/trans (or Z/E) isomers are possible. Cis/trans isomers may also exist for saturated rings.

The salts of compounds of the invention may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the invention contains an acidic or basic moiety, a base or acid such as 1-phenylethylamine or tartaric acid. The resulting diastereomeric mixture may be separated by chromatography, fractional crystallization, or by using both of the techniques, and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

### Tautomerism

Where structural isomers are interconvertible via a low energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism. In particular, the bis(amino)cyclobut-3-ene-1,2-dione moiety contained within the compounds of the present invention may tautomerize as shown below and are included within the scope of the present invention.

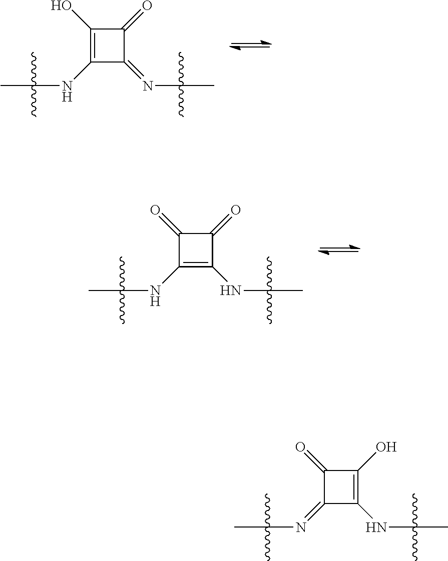 [SMILES: */N=c1/c(N*)c(O)c1=O ; *Nc1c(N*)c(=O)c1=O ; */N=c1/c(N*)c(O)c1=O]

It must be emphasized that while, for conciseness, the compounds of the invention have been drawn herein in a single tautomeric form, all possible tautomeric forms are included within the scope of the invention.

### Isotopes

The present invention includes all isotopically-labeled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention may include isotopes of hydrogen, such as ²H (D, deuterium) and ³H (T, tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the invention, for example those incorporating a radioactive isotope, are useful in one or both of drug or substrate tissue distribution studies. The radioactive isotopes tritium, e.g., ³H, and carbon-14, e.g., ¹⁴C, are particularly useful for this purpose in view of their ease of incorporation and ready means of detection.

Substitution with deuterium, e.g., ²H, may afford certain therapeutic advantages resulting from greater metabolic stability.

Substitution with positron emitting isotopes, such as ¹¹C, ¹⁸F, ¹⁵O and ¹³N, may be useful in Positron Emission Topography (PET) studies for examining substrate receptor occupancy. Substitution with deuterium may afford certain therapeutic advantages resulting from greater metabolic stability, for example increased in vivo half-life, reduced dosage requirements, reduced CYP450 inhibition (competitive or time dependent), or an improvement in therapeutic index or tolerability.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the invention may be defined by the deuterium enrichment factor. It is understood that one or more deuterium may exchange with hydrogen under physiological conditions.

In some embodiments, the deuterium compound is selected from any one of the compounds set forth in Tables 6A-6G shown in the Examples section.

In some embodiments, one or more hydrogen atoms on certain metabolic sites on the compounds of the invention are deuterated.

Isotopically-labeled compounds of the invention may generally be prepared by conventional techniques known to those skilled in the art or by processes analogous to those described in the accompanying Examples and Preparations using an appropriate isotopically-labeled reagent in place of the non-labeled reagent previously employed.

Solvates in accordance with the invention include those wherein the solvent of crystallization may be isotopically substituted, e.g., D₂O, d₆-acetone, d₆-DMSO.

### Metabolites

Also included within the scope of the invention are active metabolites of compounds of the invention, that is, compounds formed in vivo upon administration of the drug, often by oxidation or dealkylation. Some examples of metabolites in accordance with the invention include, but are not limited to,
(i) where the compound of the invention contains an alkyl group, a hydroxyalkyl derivative thereof (—CH>—COH):
(ii) where the compound of the invention contains an alkoxy group, a hydroxy derivative thereof (—OR->—OH);
(iii) where the compound of the invention contains a tertiary amino group, a secondary amino derivative thereof (—NRR′->—NHR or —NHR);
(iv) where the compound of the invention contains a tertiary amino group, an N-oxide derivative thereof (—NRR′->—N(O)RR′);
(v) where the compound of the invention contains a secondary amino group, a primary derivative thereof (—NHR->—NH₂);
(vi) where the compound of the invention contains a phenyl moiety, a phenol derivative thereof (-Ph->-PhOH);
(vii) where the compound of the invention contains an amide group, a carboxylic acid derivative thereof (—CONH₂->COOH); and
(viii) where the compound contains a hydroxy or carboxylic acid group, the compound may be metabolized by conjugation, for example with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

### Lipid Nanoparticles

Novel ionizable lipids are disclosed herein that provide advantages when used in lipid nanoparticles for the delivery of an active or therapeutic agent such as a nucleic acid into a cell of a mammal. In particular, embodiments of the present invention provide nucleic acid-lipid nanoparticle compositions comprising one or more of the novel ionizable lipids described herein that provide increased activity of the nucleic acid and improved tolerability of the compositions in vivo, resulting in an increase in the therapeutic index as compared to nucleic acid-lipid nanoparticle compositions previously described.

In particular embodiments, the present invention provides novel ionizable lipids that enable the formulation of improved compositions for the in vitro and in vivo delivery of mRNA and/or other oligonucleotides. In some embodiments, these improved lipid nanoparticle compositions are useful for expression of protein encoded by mRNA. In other embodiments, these improved lipid nanoparticles compositions are useful for upregulation of endogenous protein expression by delivering miRNA inhibitors targeting one specific miRNA or a group of miRNA regulating one target mRNA or several mRNA. In other embodiments, these improved lipid nanoparticle compositions are useful for down-regulating (e.g., silencing) the protein levels and/or mRNA levels of target genes. In some other embodiments, the lipid nanoparticles are also useful for delivery of mRNA and plasmids for expression of transgenes. In yet other embodiments, the lipid nanoparticle compositions are useful for inducing a pharmacological effect resulting from expression of a protein, e.g., increased production of red blood cells through the delivery of a suitable erythropoietin mRNA, or protection against infection through delivery of mRNA encoding for a suitable antigen or antibody. The present disclosure further provides for RNA molecules that are messenger-RNA (mRNA), which can be either nucleoside-modified RNA (modRNA) or self-amplifying RNA (saRNA). In some aspects, the RNA is a mRNA. In some aspects, the RNA is a modRNA. In other aspects, the RNA is a saRNA.

The lipid nanoparticles and compositions of the present invention may be used for a variety of purposes, including the delivery of encapsulated or associated (e.g., complexed) therapeutic agents such as nucleic acids to cells, both in vitro and in vivo. Accordingly, embodiments of the present invention provide methods of treating or preventing diseases or disorders in a subject in need thereof by contacting the subject with a lipid nanoparticle that encapsulates or is associated with a suitable therapeutic agent, wherein the lipid nanoparticle comprises one or more of the novel ionizable lipids described herein.

As described herein, lipid nanoparticles are particularly useful for the delivery of nucleic acids, including, e.g., mRNA, antisense oligonucleotide, plasmid DNA, microRNA (miRNA), miRNA inhibitors (antagomirs/antimirs), messenger-RNA-interfering complementary RNA (micRNA), DNA, multivalent RNA, dicer substrate RNA, complementary DNA (cDNA), circular DNA (ceDNA), short interfering RNA (SIRNA), etc. Therefore, the lipid nanoparticles and compositions of the present invention may be used to induce expression of a desired protein both in vitro and in vivo by contacting cells with a lipid nanoparticle comprising one or more novel ionizable lipids described herein, wherein the lipid nanoparticle encapsulates or is associated with a nucleic acid that is expressed to produce the desired protein (e.g., a messenger RNA or plasmid encoding the desired protein) or inhibit processes that terminate expression of mRNA (e.g., miRNA inhibitors). Alternatively, the lipid nanoparticles and compositions may be used to decrease the expression of target genes and proteins both in vitro and in vivo by contacting cells with a lipid nanoparticle comprising one or more novel ionizable lipids described herein, wherein the lipid nanoparticle encapsulates or is associated with a nucleic acid that reduces target gene expression (e.g., an antisense oligonucleotide or small interfering RNA (siRNA)). The lipid nanoparticles and compositions of the present invention may also be used for co-delivery of different nucleic acids (e.g. mRNA and plasmid DNA) separately or in combination, such as may be useful to provide an effect requiring colocalization of different nucleic acids (e.g. mRNA encoding for a suitable gene modifying enzyme and DNA segment(s) for incorporation into the host genome).

Nucleic acids for use with this invention may be prepared according to any available technique. For mRNA, the primary methodology of preparation is, but not limited to, enzymatic synthesis (also termed in vitro transcription) which currently represents the most efficient method to produce long sequence-specific mRNA. In vitro transcription describes a process of template-directed synthesis of RNA molecules from an engineered DNA template comprised of an upstream bacteriophage promoter sequence (e.g. including but not limited to that from the T7, T3 and SP6 coliphage) linked to a downstream sequence encoding the gene of interest. Template DNA can be prepared for in vitro transcription from a number of sources with appropriate techniques which are well known in the art including, but not limited to, plasmid DNA and polymerase chain reaction amplification (see Linpinsel, J. L and Conn, G. L., General protocols for preparation of plasmid DNA template and Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012)

Transcription of the RNA occurs in vitro using the linearized DNA template in the presence of the corresponding RNA polymerase and adenosine, guanosine, uridine and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well known in the art. (see e.g. Losick, R., 1972, In vitro transcription, Ann Rev Biochem v. 41 409-46; Kamakaka, R. T. and Kraus, W. L. 2001. In Vitro Transcription. Current Protocols in Cell Biology. 2:11.6:11.6.1-11.6.17; Beckert, B. And Masquida, B., (2010) Synthesis of RNA by In Vitro Transcription in RNA in Methods in Molecular Biology v. 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology v. 530, 101-114; all of which are incorporated herein by reference).

The desired in vitro transcribed mRNA is then purified from the undesired components of the transcription or associated reactions (including unincorporated rNTPs, protein enzyme, salts, short RNA oligos, etc.). Techniques for the isolation of the mRNA transcripts are well known in the art. Well known procedures include phenol/chloroform extraction or precipitation with either alcohol (ethanol, isopropanol) in the presence of monovalent cations or lithium chloride. Additional, non-limiting examples of purification procedures which can be used include size exclusion chromatography (Lukavsky, P. J. and Puglisi, J. D., 2004, Large-scale preparation and purification of polyacrylamide-free RNA oligonucleotides, RNA v. 10, 889-893), silica-based affinity chromatography and polyacrylamide gel electrophoresis (Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012). Purification can be performed using a variety of commercially available kits including, but not limited to SV Total Isolation System (Promega) and In Vitro Transcription Cleanup and Concentration Kit (Norgen Biotek).

Furthermore, while reverse transcription can yield large quantities of mRNA, the products can contain a number of aberrant RNA impurities associated with undesired polymerase activity which may need to be removed from the full-length mRNA preparation. These include short RNAs that result from abortive transcription initiation as well as double-stranded RNA (dsRNA) generated by RNA-dependent RNA polymerase activity, RNA-primed transcription from RNA templates and self-complementary 3′ extension. It has been demonstrated that these contaminants with dsRNA structures can lead to undesired immunostimulatory activity through interaction with various innate immune sensors in eukaryotic cells that function to recognize specific nucleic acid structures and induce potent immune responses. This in turn, can dramatically reduce mRNA translation since protein synthesis is reduced during the innate cellular immune response. Therefore, additional techniques to remove these dsRNA contaminants have been developed and are known in the art including but not limited to scaleable HPLC purification (see e.g. Kariko, K., Muramatsu, H., Ludwig, J. And Weissman, D., 2011, Generating the optimal mRNA for therapy: HPLC purification eliminates immune activation and improves translation of nucleoside-modified, protein-encoding mRNA, Nucl Acid Res, v. 39 e142; Weissman, D., Pardi, N., Muramatsu, H., and Kariko, K., HPLC Purification of in vitro transcribed long RNA in Synthetic Messenger RNA and Cell Metabolism Modulation in Methods in Molecular Biology v. 969 (Rabinovich, P. H. Ed), 2013). HPLC purified mRNA has been reported to be translated at much greater levels, particularly in primary cells and in vivo.

A significant variety of modifications have been described in the art which are used to alter specific properties of in vitro transcribed mRNA, and improve its utility. These include, but are not limited to modifications to the 5′ and 3′ termini of the mRNA. Endogenous eukaryotic mRNA typically contain a cap structure on the 5′-end of a mature molecule which plays an important role in mediating binding of the mRNA Cap Binding Protein (CBP), which is in turn responsible for enhancing mRNA stability in the cell and efficiency of mRNA translation. Therefore, highest levels of protein expression are achieved with capped mRNA transcripts. The 5′-cap contains a 5′-5′-triphosphate linkage between the 5′-most nucleotide and guanine nucleotide. The conjugated guanine nucleotide is methylated at the N7 position. Additional modifications include methylation of the ultimate and penultimate most 5′-nucleotides on the 2′-hydroxyl group.

Multiple distinct cap structures can be used to generate the 5′-cap of in vitro transcribed synthetic mRNA. 5′-capping of synthetic mRNA can be performed co-transcriptionally with chemical cap analogs (e.g. capping during in vitro transcription). For example, the Anti-Reverse Cap Analog (ARCA) cap contains a 5′-5′-triphosphate guanine-guanine linkage where one guanine contains an N7 methyl group as well as a 3′-O-methyl group. However, up to 20% of transcripts remain uncapped during this co-transcriptional process and the synthetic cap analog is not identical to the 5′-cap structure of an authentic cellular mRNA, potentially reducing translatability and cellular stability. Alternatively, synthetic mRNA molecules may also be enzymatically capped post-transcriptionally. These may generate a more authentic 5′-cap structure that more closely mimics, either structurally or functionally, the endogenous 5′-cap which have enhanced binding of cap binding proteins, increased half-life, reduced susceptibility to 5′ endonucleases and/or reduced 5′ decapping. Numerous synthetic 5′-cap analogs have been developed and are known in the art to enhance mRNA stability and translatability (see e.g. Grudzien-Nogalska, E., Kowalska, J., Su, W., Kuhn, A. N., Slepenkov, S. V., Darynkiewicz, E., Sahin, U., Jemielity, J., and Rhoads, R. E., Synthetic mRNAs with superior translation and stability properties in Synthetic Messenger RNA and Cell Metabolism Modulation in Methods in Molecular Biology v. 969 (Rabinovich, P. H. Ed), 2013).

On the 3′-terminus, a long chain of adenine nucleotides (poly-A tail) is normally added to mRNA molecules during RNA processing. Immediately after transcription, the 3′ end of the transcript is cleaved to free a 3′ hydroxyl to which poly-A polymerase adds a chain of adenine nucleotides to the RNA in a process called polyadenylation. The poly-A tail has been extensively shown to enhance both translational efficiency and stability of mRNA (see Bernstein, P. and Ross, J., 1989, Poly (A), poly (A) binding protein and the regulation of mRNA stability, Trends Bio Sci v. 14 373-377; Guhaniyogi, J. And Brewer, G., 2001, Regulation of mRNA stability in mammalian cells, Gene, v. 265, 11-23; Dreyfus, M. And Regnier, P., 2002, The poly (A) tail of mRNAs: Bodyguard in eukaryotes, scavenger in bacteria, Cell, v. 111, 611-613).

Poly (A) tailing of in vitro transcribed mRNA can be achieved using various approaches including, but not limited to, cloning of a poly (T) tract into the DNA template or by post-transcriptional addition using Poly (A) polymerase. The first case allows in vitro transcription of mRNA with poly (A) tails of defined length, depending on the size of the poly (T) tract, but requires additional manipulation of the template. The latter case involves the enzymatic addition of a poly (A) tail to in vitro transcribed mRNA using poly (A) polymerase which catalyzes the incorporation of adenine residues onto the 3′ termini of RNA, requiring no additional manipulation of the DNA template, but results in mRNA with poly(A) tails of heterogeneous length. 5′-capping and 3′-poly (A) tailing can be performed using a variety of commercially available kits including, but not limited to Poly (A) Polymerase Tailing kit (EpiCenter), mMESSAGE mMACHINE T7 Ultra kit and Poly (A) Tailing kit (Life Technologies) as well as with commercially available reagents, various ARCA caps, Poly (A) polymerase, etc.

In addition to 5′ cap and 3′ poly adenylation, other modifications of the in vitro transcripts have been reported to provide benefits as related to efficiency of translation and stability. It is well known in the art that pathogenic DNA and RNA can be recognized by a variety of sensors within eukaryotes and trigger potent innate immune responses. The ability to discriminate between pathogenic and self DNA and RNA has been shown to be based, at least in part, on structure and nucleoside modifications since most nucleic acids from natural sources contain modified nucleosides In contrast, in vitro synthesized RNA lacks these modifications, thus rendering it immunostimulatory which in turn can inhibit effective mRNA translation as outlined above. The introduction of modified nucleosides into in vitro transcribed mRNA can be used to prevent recognition and activation of RNA sensors, thus mitigating this undesired immunostimulatory activity and enhancing translation capacity (see e.g. Kariko, K. And Weissman, D. 2007, Naturally occurring nucleoside modifications suppress the immunostimulatory activity of RNA: implication for therapeutic RNA development, Curr Opin Drug Discov Devel, v. 10 523-532; Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v. 969 (Rabinovich, P. H. Ed), 2013); Kariko, K., Muramatsu, H., Welsh, F. A., Ludwig, J., Kato, H., Akira, S., Weissman, D., 2008, Incorporation of Pseudouridine Into mRNA Yields Superior Nonimmunogenic Vector With Increased Translational Capacity and Biological Stability, Mol Ther v. 16, 1833-1840. The modified nucleosides and nucleotides used in the synthesis of modified RNAs can be prepared monitored and utilized using general methods and procedures known in the art. A large variety of nucleoside modifications are available that may be incorporated alone or in combination with other modified nucleosides to some extent into the in vitro transcribed mRNA (see e.g. US2012/0251618). In vitro synthesis of nucleoside-modified mRNA have been reported to have reduced ability to activate immune sensors with a concomitant enhanced translational capacity.

Other components of mRNA which can be modified (modRNA) to provide benefit in terms of translatability and stability include the 5′ and 3′ untranslated regions (UTR). Optimization of the UTRs (favorable 5′ and 3′ UTRs can be obtained from cellular or viral RNAs), either both or independently, have been shown to increase mRNA stability and translational efficiency of in vitro transcribed mRNA (see e.g. Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v. 969 (Rabinovich, P. H. Ed), 2013).

A “modified RNA” or “modRNA” refers to an RNA molecule having at least one addition, deletion, substitution, and/or alteration of one or more nucleotides to form a nucleotide structure that is not naturally occurring (e.g., not A, C, T, G, or U). Such alterations may refer to the addition of non-nucleotide material to internal RNA nucleotides, or to the 5′ and/or 3′ end(s) of RNA. In one aspect, such modRNA contains at least one modified nucleotide, such as an alteration to the base of the nucleotide. For example, a modified nucleotide may replace one or more uridine and/or cytidine nucleotides. For example, these replacements may occur for every instance of uridine and/or cytidine in the RNA sequence, or may occur for only select uridine and/or cytidine nucleotides. Such alterations to the standard nucleotides in RNA may include non-standard nucleotides, such as chemically synthesized nucleotides or deoxynucleotides. For example, at least one uridine nucleotide may be replaced with N1-methylpseudouridine in an RNA sequence. Other such altered nucleotides are known to those of skill in the art. Such altered RNA molecules are considered analogs of naturally-occurring RNA. In some aspects, the RNA is produced by in vitro transcription using a DNA template, where DNA refers to a nucleic acid that contains deoxyribonucleotides.

In some aspects, the RNA molecule may be an saRNA. “Self-amplifying RNA,” “self-amplifying RNA,” and “replicon” refer to RNA with the ability to replicate itself. Self-amplifying RNA molecules may be produced by using replication elements derived from, e.g. alphaviruses, and substituting the structural viral polypeptides with a nucleotide sequence encoding a polypeptide of interest. A self-amplifying RNA molecule is typically a positive-strand molecule that may be directly translated after delivery to a cell, and this translation provides an RNA-dependent RNA polymerase which then produces both antisense and sense transcripts from the delivered RNA. The delivered RNA leads to the production of multiple daughter RNA molecules. These daughter RNA molecules, as well as collinear subgenomic transcripts, may be translated themselves to provide in situ expression of an encoded gene of interest, e.g., a viral antigen, or may be transcribed to provide further transcripts with the same sense as the delivered RNA which are translated to provide in situ expression of the antigen. The overall result of this sequence of transcriptions is an amplification in the number of the introduced saRNA molecules and so the encoded gene of interest, e.g., a viral antigen, becomes a major polypeptide product of the cells.

In some aspects, the self-amplifying RNA includes at least one or more genes including any one of viral replicases, viral proteases, viral helicases and other nonstructural viral proteins, or combination thereof. In some aspects, the self-amplifying RNA may also include 5′- and 3′-end tractive replication sequences, and optionally a heterologous sequence that encodes a desired amino acid sequence (e.g., an antigen of interest). A subgenomic promoter that directs expression of the heterologous sequence may be included in the self-amplifying RNA. Optionally, the heterologous sequence (e.g., an antigen of interest) may be fused in frame to other coding regions in the self-amplifying RNA and/or may be under the control of an internal ribosome entry site (IRES).

In addition to mRNA, other nucleic acid payloads may be used for this invention. For oligonucleotides, methods of preparation include but are not limited to chemical synthesis and enzymatic, chemical cleavage of a longer precursor, in vitro transcription as described above, etc. Methods of synthesizing DNA and RNA nucleotides are widely used and well known in the art (see e.g. Gait, M. J. (ed.) Oligonucleotide synthesis: a practical approach, Oxford [Oxfordshire], Washington, D. C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

For plasmid DNA, preparation for use with this invention commonly utilizes but is not limited to expansion and isolation of the plasmid DNA in vitro in a liquid culture of bacteria containing the plasmid of interest. The presence of a gene in the plasmid of interest that encodes resistance to a particular antibiotic (penicillin, kanamycin, etc.) allows those bacteria containing the plasmid of interest to selectively grow in antibiotic-containing cultures. Methods of isolating plasmid DNA are widely used and well known in the art (see e.g. Heilig, J., Elbing, K. L. and Brent, R (2001) Large-Scale Preparation of Plasmid DNA. Current Protocols in Molecular Biology. 41:II:1.7:1.7.1-1.7.16; Rozkov, A., Larsson, B., Gillstrom, S., Bjornestedt, R. and Schmidt, S. R. (2008), Large-scale production of endotoxin-free plasmids for transient expression in mammalian cell culture. Biotechnol. Bioeng., 99:557-566; and U.S. Pat. No. 6,197,553B1). Plasmid isolation can be performed using a variety of commercially available kits including, but not limited to Plasmid Plus (Qiagen), GenJET plasmid MaxiPrep (Thermo) and PureYield MaxiPrep (Promega) kits as well as with commercially available reagents.

The term “nucleic acid” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), antisense RNA, miRNA, micRNA, multivalent RNA, dicer substrate RNA or viral RNA (VRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAS). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The term “lipid” refers to a group of organic compounds that include, but are not limited to, esters of fatty acids and are generally characterized by being poorly soluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

A “steroid” is a compound comprising the following carbon skeleton:

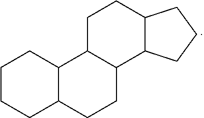 [SMILES: C1CCC2C(C1)CCC1C3CCCC3CCC21]

Non-limiting examples of steroids include cholesterol, and the like.

An “ionizable lipid” refers to a lipid capable of being positively charged. Exemplary ionizable lipids include one or more amine group(s) which bear the positive charge. Preferred ionizable lipids are ionizable such that they can exist in a positively charged or neutral form depending on pH. The ionization of the ionizable lipid affects the surface charge of the lipid nanoparticle under different pH conditions. This charge state can influence plasma protein absorption, blood clearance and tissue distribution (Semple, S. C., et al., Adv. Drug Deliv Rev 32:3-17 (1998)) as well as the ability to form endosomolytic non-bilayer structures (Hafez, I. M., et al., Gene Ther 8:1188-1196 (2001)) critical to the intracellular delivery of nucleic acids. As used herein, an “ionizable lipid” may also include, but is not limited to, a “cationic lipid”.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-DMG) and the like.

The term “neutral lipid” refers to any of a number of lipid species that exist either in an uncharged or neutral zwitterionic form at a selected pH. At physiological pH, such lipids include, but are not limited to, phosphotidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), phophatidylethanolamines such as 1,2-Dioleoyl-sn-glycero-3-phosphoethanolamine (DOPE), sphingomyelins (SM), ceramides, steroids such as sterols and their derivatives. Neutral lipids may be synthetic or naturally derived.

The term “charged lipid” refers to any of a number of lipid species that exist in either a positively charged or negatively charged form independent of the pH within a useful physiological range e.g. pH ^{˜}3 to pH ^{˜}9. Charged lipids may be synthetic or naturally derived. Examples of charged lipids include phosphatidylserines, phosphatidic acids, phosphatidylglycerols, phosphatidylinositols, sterol hemisuccinates, dialkyl trimethylammonium-propanes, (e.g. DOTAP, DOTMA), dialkyl dimethylaminopropanes, ethyl phosphocholines, dimethylaminoethane carbamoyl sterols (e.g. DC-Chol).

The term “lipid nanoparticle” refers to particles having at least one dimension on the order of nanometers (e.g., 1-1,000 nm) which include one or more of the compounds of structure (I) or other specified ionizable lipids. In some embodiments, lipid nanoparticles are included in a formulation that can be used to deliver an active agent or therapeutic agent, such as a nucleic acid (e.g., mRNA) to a target site of interest (e.g., cell, tissue, organ, tumor, and the like). In some embodiments, the lipid nanoparticles of the invention comprise a nucleic acid. Such lipid nanoparticles typically comprise a compound of structure (I) and one or more excipient selected from neutral lipids, charged lipids, steroids and polymer conjugated lipids. In some embodiments, the active agent or therapeutic agent, such as a nucleic acid, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and any size or range thereof or therebetween. In various embodiments, the lipid nanoparticles are substantially non-toxic. In certain embodiments, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

As used herein, the “polydispersity index,” or “PDI” is a ratio that describes the homogeneity of the particle size distribution of a system. A value of less than 0.3 indicates a relatively narrow particle size distribution.

As used herein, “lipid encapsulated” refers to a lipid nanoparticle that provides an active agent or therapeutic agent, such as a nucleic acid (e.g., mRNA), with full encapsulation, partial encapsulation, or both. In an embodiment, the nucleic acid (e.g., mRNA) is fully encapsulated in the lipid nanoparticle

As used herein, “encapsulation efficiency” refers to the percentage of a therapeutic agent that becomes part of a nanoparticle composition, relative to the initial total amount of therapeutic agent used in the preparation. Encapsulation efficiency (EE %) is calculated by (total therapeutic agent added—free non-entrapped therapeutic agent) divided by the total therapeutic agent added.

As used herein, “size” or “mean size” in the context of lipid nanoparticles refers to the mean diameter of a nanoparticle composition.

“Serum-stable” in relation to nucleic acid-lipid nanoparticles means that the nucleotide is not significantly degraded after exposure to a serum or nuclease assay that would significantly degrade free DNA or RNA. Suitable assays include, for example, a standard serum assay, a DNAse assay, or an RNAse assay.

“Stable compound” and “stable structure” are meant to indicate a compound that is sufficiently robust to survive isolation to a useful degree of purity from a reaction mixture, and formulation into an efficacious therapeutic agent.

In an aspect, the compositions disclosed herein comprise lipids. For example, compositions can include lipids and mRNA (e.g., modRNA or saRNA), and the lipids and mRNA (e.g., modRNA or saRNA) can together form nanoparticles, thereby producing mRNA-containing nanoparticles comprising lipids. The lipids can encapsulate or associate with the mRNA in the form of a lipid nanoparticle (LNP) to aid stability, cell entry, and intracellular release of the RNA/lipid nanoparticles.

In some instances, a LNP comprises a micelle, a solid lipid nanoparticle, a nanoemulsion, a liposome, etc., or a combination thereof.

The lipid component of a LNP may include, for example, an ionizable lipid, a neutral lipid such as a phospholipid (such as an unsaturated lipid, e.g., DOPE or DSPC), a polymer-lipid conjugate (e.g., a PEGylated lipid), a structural lipid or any combination thereof. The elements of the lipid component may be provided in specific fractions. Suitable ionizable lipids, polymer-lipid conjugates, structural lipids, and neutral lipids for the methods of the present disclosure are further disclosed herein.

In certain aspects, the lipid component of the lipid nanoparticle includes about 0 mol % to about 60 mol % ionizable lipid (e.g., at least about, at most about, between any two of, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol % ionizable lipid); about 0 mol % to about 60 mol % phospholipid (e.g., at least about, at most about, between any two of, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol % phospholipid); about 0 mol % to about 60 mol % structural lipid (e.g., at least about, at most about, between any two of, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 mol % structural lipid); about 0 mol % to about 60 mol % of polymer-lipid conjugate (e.g., at least about, at most about, between any two of, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol % polymer-lipid conjugate); about 0 mol % to about 60 mol % ionizable lipid (e.g., at least about, at most about, between any two of, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol % ionizable lipid); and/or about 0 mol % to about 60 mol % neutral lipid (e.g., at least about, at most about, between any two of, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol % neutral lipid). The LNP can have any amount of the foregoing lipid components, provided that the total mole % does not exceed 100%. As used herein, “mol percent” or “mol %” refers to a component's molar percentage relative to total moles of all lipid components in the LNP (e.g., total mols of ionizable lipid(s), the neutral lipid, the steroid and the polymer conjugated lipid).

In some aspects, the lipid component of the lipid nanoparticle includes about 35 mol % to about 55 mol % compound of ionizable lipid, about 5 mol % to about 25 mol % phospholipid, about 30 mol % to about 50 mol % structural lipid, and about 0 mol % to about 10 mol % of polymer-lipid conjugate. In a particular aspect, the lipid component includes about 50 mol % of the ionizable lipid, about 10 mol % phospholipid, about 40 mol % structural lipid, and about 1.5 mol % of polymer-lipid conjugate. In another particular aspect, the lipid component includes about 40 mol % of the ionizable lipid, about 20 mol % phospholipid, about 40 mol % structural lipid, and about 1.5 mol % of polymer-lipid conjugate. In a particular aspect, the lipid component of the lipid nanoparticle includes ionizable lipid, phospholipid, structural lipid, and polymer-lipid conjugate at a molar ratio of about 47.5:10:40.7:1.8.

In some aspects, the lipid component of the lipid nanoparticle includes about 0 mol % to about 10 mol % compound of ionizable lipid, about 40 mol % to about 60 mol % phospholipid, and about 40 mol % to about 60 mol % structural lipid. In a particular aspect, the lipid component includes about 2 mol % of the ionizable lipid, about 49 mol % phospholipid, and about 49 mol % structural lipid. In a particular aspect, the lipid component of the lipid nanoparticle includes ionizable lipid, phospholipid, and structural lipid at a molar ratio of about 1.8:49.1:49.1.

In some aspects, the phospholipid may be DOPE or DSPC. In other aspects, the polymer-lipid conjugate may be PEG-DMG and/or the structural lipid may be cholesterol. In other aspects, the polymer-lipid conjugate may be PEG-2000 DMG and/or the structural lipid may be cholesterol.

In some aspects, the lipid nanoparticle includes:
i) between 40 and 50 mol percent of an ionizable lipid;
ii) a phospholipid and/or a neutral lipid;
iii) a structural lipid;
iv) a polymer conjugated lipid; and
v) a therapeutic agent (namely, RNA) encapsulated within or associated with the lipid nanoparticle.

In some aspects, the lipid nanoparticle includes:
i) between 0 and 10 mol % of an ionizable lipid;
ii) a phospholipid and/or a neutral lipid; and
iii) a steroid.

In some aspects, the lipid nanoparticle comprises from 41 to 50 mol percent, from 42 to 50 mol percent, from 43 to 50 mol percent, from 44 to 50 mol percent, from 45 to 50 mol percent, from 46 to 50 mol percent, or from 47 to 50 mol percent of the ionizable lipid, or any mol percent or range thereof or therebetween. In certain specific aspects, the lipid nanoparticle comprises at least about, at most about, between any two of, or exactly 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, or 50 mol percent of the ionizable lipid.

In other aspects, the lipid nanoparticle comprises from 0 to 10 mol percent of the ionizable lipid. In certain specific aspects, the lipid nanoparticle comprises at least about, at most about, between any two of, or exactly 0, 1, 2, 3, 4, 5, 6, 7, 8, 9, or 10 mol percent of the ionizable lipid.

In some aspects, the phospholipid and/or neutral lipid is present in a concentration ranging from 5 to 15 mol percent, 7 to 13 mol percent, or 9 to 11 mol percent, or any mol percent or range thereof or therebetween. In certain aspects, the phospholipid and/or neutral lipid is present in a concentration of at least about, at most about, in between any two of, or exactly 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, or 15 mol percent. In certain specific aspects, the phospholipid and/or neutral lipid is present in a concentration of about 9.5, 10 or 10.5 mol percent.

In other aspects, the phospholipid and/or neutral lipid is present in a concentration ranging from 40 to 60 mol %, or any mol percent or range thereof or therebetween. In certain aspects, the phospholipid and/or neutral lipid is present in a concentration of at least about, at most about, in between any two of, or exactly 40, 41, 42, 43, 44, 45, 46, 47, 48, 49, 50, 51, 52, 53, 54, 55, 56, 57, 58, 59, or 60 mol percent. In certain specific aspects, the phospholipid and/or neutral lipid is present in a concentration of about 48, 49, or 50 mol percent.

In some aspects, the molar ratio of the ionizable lipid to the phospholipid and/or neutral lipid ranges from about 4.1:1.0 to about 4.9:1.0, from about 4.5:1.0 to about 4.8:1.0, or from about 4.7:1.0 to 4.8:1.0, or any molar ratio or range thereof or therebetween. In other aspects, the molar ratio of the phospholipid and/or neutral lipid to the ionizable lipid is 1:4.1, 1:4.2, 1:4.3, 1:4.4, 1:4.5, 1:4.6, 1:4.7, 1:4.8, or 1:4.9.

In some aspects, the structural lipid is a steroid. In some aspects, the steroid is cholesterol. In some aspects, the structural lipid is present in a concentration ranging from 39 to 49 molar percent, 40 to 46 molar percent, from 40 to 44 molar percent, from 40 to 42 molar percent, from 42 to 44 molar percent, or from 44 to 46 molar percent, or any molar percent or range thereof or therebetween. In certain specific aspects, the structural lipid is present in a concentration of at least about, at most about, in between any two of, or exactly 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, or 49 mol percent. In certain specific aspects, the structural lipid is present in a concentration of 40, 41, 42, 43, 44, 45, or 46 molar percent.

In other aspects, the structural lipid is present in a concentration ranging from 40 to 60 mol %. In certain aspects, the structural lipid is present in a concentration of at least about, at most about, in between any two of, or exactly 40, 41, 42, 43, 44, 45, 46, 47, 48, 49, 50, 51, 52, 53, 54, 55, 56, 57, 58, 59, or 60 mol percent. In certain specific aspects, the structural lipid is present in a concentration of about 48, 49, or 50 mol percent.

In certain aspects, the molar ratio of ionizable lipid to the structural lipid ranges from 1.0:0.9 to 1.0:1.2, or from 1.0:1.0 to 1.0:1.2, e.g., 1:0.9, 1:1, 1:1.1, or 1:1.2.

In preferred aspects, the ionizable lipid is a compound having the following structure (I):

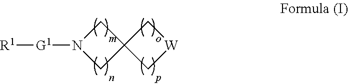

or a pharmaceutically acceptable salt, N-oxide, tautomer, or stereoisomer thereof, wherein
m, n, o, and p are each independently 1-3;
G¹ is C₁₋₁₂alkylene or C₂₋₁₂alkenylene;
R¹ is —N(R²)R³, —OR⁴, CN, —N(R⁴)(heteroaryl), —O(CH₂)_{q}OH, —(OCH₂CH₂)ᵣOH, —OC(═O)R⁵, —N(R⁴)C(═O)R⁵, —N(R⁴)S(O)₂R⁵, —N(R⁴)C(═O)N(R²)R³, —OC(═O)N(R²)R³, —N(R⁴)C(═O)OR⁵, —N(R⁴)C(═S)N(R²)R³, —N(R⁴)C(═NR⁶)N(R²)R³, or

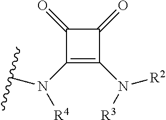

R² and R³ are each independently H, C₁₋₆alkyl, C₃₋₈cycloalkyl, or aryl; or R² and R³ together with the nitrogen atom to which they are attached form a heterocyclic ring;
R⁴ is H, C₁₋₆alkyl or C₃₋₈cycloalkyl;
R⁵ is C₁₋₆alkyl or C₃₋₈cycloalkyl optionally substituted by C₁₋₆alkyl;
R⁶ is H, CN, NO₂, C₁₋₆alkyl, OR⁵, S(O)₂R⁵, or S(O)₂N(R²)R³;
q is 2-6;
r is 1-6;
W is

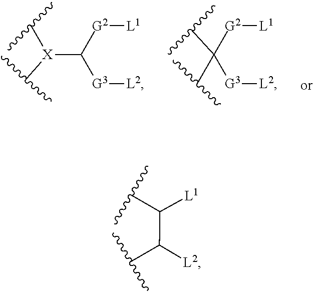

X is N or CH;
G² and G³ are each independently C₁₋₁₂alkylene or C₂₋₁₂alkenylene;
L¹ and L² are each independently —C(═O)OR⁷, —OC(═O)R⁷, —OC(═O)(CH₂)ᵣC(═O)OR⁷, —OC(═O)(CH₂)ᵣOC(═O)R⁷, —OC(═O)N(R⁴)R⁷, —N(R⁴)C(═O)OR⁷, —N(R⁴)C(═O)N(R⁴)R⁷, —OC(═O)OR⁷, or —S—SR⁷; and
R⁷ is C₆₋₂₄alkyl, C₆₋₂₄alkenyl, or C₆₋₂₄alkynyl where each is optionally substituted by F, C₁₋₆ alkoxy, C₃₋₈cycloalkyl, or C₃₋₈cycloalkenyl, wherein the R⁷ of L¹ and L² may be the same or different.

The lipid component of a lipid nanoparticle composition may include one or more molecules comprising a polymer such as a polyethylene glycol, e.g., PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some aspects, a PEG lipid may be PEG-c-DOMG, PEG-DMG, PEG-DLPE, PEG-DMPE, PEG-DPPC, or a PEG-DSPE lipid. As used herein, the term “PEG lipid” refers to polyethylene glycol (PEG)-modified lipids. Non-limiting examples of PEG lipids include PEG-modified phosphatidylethanolamine and phosphatidic acid, PEG-ceramide conjugates (e.g., PEG-CerCI4 or PEG-CerC20), PEG-modified dialkylamines and PEG-modified 1,2-diacyloxypropan-3-amines. Such lipids are also referred to as PEGylated lipids. In some aspects, a PEG lipid can be PEG-c-DOMG, PEG-DMG, PEG-DLPE, PEG-DMPE, PEG-DPPC, or a PEG-DSPE lipid. In some aspects, the PEG-modified lipids are a modified form of PEG DMG.

In some aspects, the PEG-modified lipid is PEG lipid with the formula (IV):

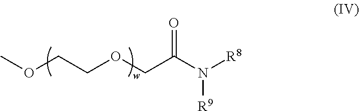

wherein R8 and R9 are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

In some aspects, the polymer-conjugated lipid is a polyoxazoline (POZ) lipid comprising the formula (IV):

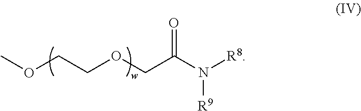

POZ is known in the art and is described in WO/2020/264505, PCT/US2020/040140, filed on Jun. 29, 2020.

In some aspects, the PEGylated lipid has the following structure (II):

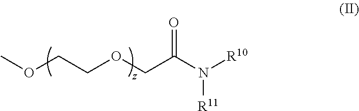

or a pharmaceutically acceptable salt, N-oxide, tautomer or stereoisomer thereof, wherein: R¹⁰ and R¹¹ are each independently a straight or branched, saturated or unsaturated alkyl chain containing from 10 to 30 carbon atoms, wherein the alkyl chain is optionally interrupted by one or more ester bonds; and z has a mean value ranging from 30 to 60; provided that R¹⁰ and R¹¹ are not both n-octadecyl when z is 42. In some aspects of the PEGylated lipid, R¹⁰ and R¹¹ are each independently straight, saturated alkyl chains containing from 12 to 16 carbon atoms. In some aspects, of the pegylated lipid z is about 45.

In some aspects, the PEGylated lipid has one of the following structures:

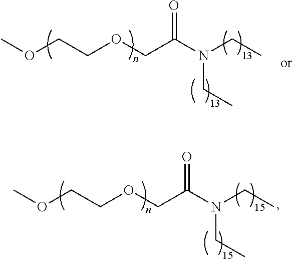

wherein n has a mean value ranging from 40 to 50. In a preferred aspect, the composition comprises the ionizable lipid described herein and a PEGylated lipid having one of the following structures:

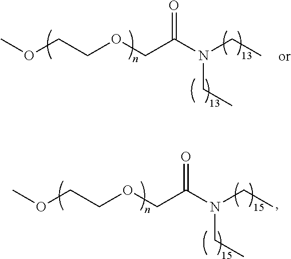

In some aspects of the PEGylated lipid described above, R¹⁰ and R¹¹ are each independently a straight or branched, saturated or unsaturated alkyl chain containing 12 carbon atoms. In some aspects of the PEGylated lipid described above, R¹⁰ and R¹¹ are each independently a straight or branched, saturated or unsaturated alkyl chain containing 14 carbon atoms. In some aspects of the PEGylated lipid described above, R¹⁰ and R¹¹ are each independently a straight or branched, saturated or unsaturated alkyl chain containing 16 carbon atoms. Further exemplary lipids and related formulations thereof are disclosed for example, in U.S. Pat. No. 9,737,619, filed Feb. 14, 2017, U.S. Pat. No. 10,166,298, filed Oct. 28, 2016, and International Patent Application No. PCT/US2017/058619, filed Oct. 26, 2017, the disclosures of which are incorporated herein by reference in their entirety.

In preferred aspects, the composition further includes a nucleic acid. In preferred aspects, the nucleic acid comprises messenger RNA. In some aspects, the composition further includes one or more excipients selected from neutral lipids and steroids. In some aspects, the composition comprises one or more neutral lipids selected from DSPC, DPPC, DMPC, DOPC, POPC, DOPE and SM. Preferably, in some aspects, the neutral lipid is DSPC. Preferably, in some aspects, the steroid is cholesterol.

A LNP may include one or more components described herein. In some aspects, the LNP formulation of the disclosure includes at least one lipid nanoparticle component. Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combinations of elements. The efficacy and tolerability of a LNP formulation may be affected by the stability of the formulation.

Lipid nanoparticles may be designed for one or more specific applications or targets. For example, a LNP may be designed to deliver a therapeutic and/or prophylactic such as an RNA to a particular cell, tissue, organ, or system or group thereof in a mammal's body. Physiochemical properties of lipid nanoparticles may be altered in order to increase selectivity for particular bodily targets. For instance, particle sizes may be adjusted based on the fenestration sizes of different organs. The therapeutic and/or prophylactic included in a LNP may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain aspects, a LNP may include an mRNA encoding a polypeptide of interest capable of being translated within a cell to produce the polypeptide of interest. Such a composition may be designed to be specifically delivered to a particular organ. In some aspects, a composition may be designed to be specifically delivered to a mammalian liver. In some aspects, a composition may be designed to be specifically delivered to a lymph node. In some aspects, a composition may be designed to be specifically delivered to a mammalian spleen.

In some aspects, a polymer may be included in and/or used to encapsulate or partially encapsulate a LNP. A polymer may be biodegradable and/or biocompatible. A polymer may be selected from, but is not limited to, polyamines, polyethers, polyamides, polyesters, poly carbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and/or polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly (hydroxy acids), polyanhydrides, polyorthoesters, poly (ester amides), polyamides, poly (ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly (vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PACM), poly(2-methyl-2-oxazoline) (PMOX), poly(2-ethyl-2-oxazoline) (PEOZ), and/or polyglycerol.

In some aspects, a surface altering agent may be included in and/or used to encapsulate or partially encapsulate a LNP. Surface altering agents may include, but are not limited to, anionic proteins (e.g., bovine serum albumin), surfactants (e.g., ionizable surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin B4, dornase alfa, neltenexine, and erdosteine), and/or DNases (e.g., rhDNase). A surface altering agent may be disposed within a nanoparticle and/or on the surface of a LNP (e.g., by coating, adsorption, covalent linkage, or other process).

A LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxy ethylene sorbitan monolaurate [TWEEN®20], polyoxy ethylene sorbitan [TWEENR® 60], polyoxy ethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly (vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, free radical scavengers, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®. An exemplary free radical scavenger includes butylated hydroxytoluene (BHT or butylhydroxytoluene) and/or deferoxamine. In some preferred aspects, the composition does not include a preservative.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. In some aspects, the concentration of the buffer in the composition is about 10 mM. In some aspects, the buffer concentration can be equal to any one of, at least any one of, at most any one of, or between any two of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM, or any range or value derivable therein. In specific aspects, the buffer concentration is 10 mM. The buffer can be at a neutral pH, pH 6.5 to 8.5, pH 7.0 to pH 8.0, or pH 7.2 to pH 7.6. In some aspects, the buffer can be at pH 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein. In specific aspects, the buffer is at pH 7.4.

In some aspects, the formulation including a LNP may further include a salt, such as a chloride salt. In some aspects, the formulation including a LNP may further includes a sugar such as a disaccharide. In some aspects, the formulation further includes a sugar but not a salt, such as a chloride salt. In some aspects, a LNP may further include one or more small hydrophobic molecules such as a vitamin (e.g., vitamin A or vitamin E) and/or a sterol. Carbohydrates may include simple sugars (e.g., glucose) and/or polysaccharides (e.g., glycogen and derivatives and analogs thereof).

The characteristics of a LNP may depend on the components thereof. For example, a LNP including cholesterol as a structural lipid may have different characteristics than a LNP that includes a different structural lipid. As used herein, the term “structural lipid” refers to sterols and also to lipids containing sterol moieties. As defined herein, “sterols” are a subgroup of steroids consisting of steroid alcohols. In some aspects, the structural lipid is a steroid. In some aspects, the structural lipid is cholesterol. In some aspects, the structural lipid is an analog of cholesterol. In some aspects, the structural lipid is alpha-tocopherol.

In some aspects, the characteristics of a LNP may depend on the absolute or relative amounts of its components. For instance, a LNP including a higher molar fraction of a phospholipid may have different characteristics than a LNP including a lower molar fraction of a phospholipid. Characteristics may also vary depending on the method and conditions of preparation of the lipid nanoparticle. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and/or a sphingomyelin. A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and/or docosahexaenoic acid. Particular phospholipids can facilitate fusion to a membrane. In some aspects, a ionizable phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue. Non-natural phospholipid species including natural species with modifications and substitutions including branching, oxidation, cyclization, and alkynes are also contemplated. In some aspects, a phospholipid can be functionalized with and/or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye). Phospholipids include, but are not limited to, glycerophospholipids such as phosphatidylcholines, phosphatidyl-ethanolamines, phosphatidylserines, phosphatidylinositols, phosphatidy glycerols, and/or phosphatidic acids. Phospholipids also include phosphosphingolipid, such as sphingomyelin. In some aspects, a phospholipid useful or potentially useful in the present invention is an analog and/or variant of DSPC.

Formulations comprising amphiphilic polymers and/or lipid nanoparticles may be formulated in whole or in part as pharmaceutical compositions. Pharmaceutical compositions may include one or more amphiphilic polymers and one or more lipid nanoparticles. For example, a pharmaceutical composition may include one or more amphiphilic polymers and one or more lipid nanoparticles and include one or more different therapeutics and/or prophylactics. Pharmaceutical compositions may further include one or more pharmaceutically acceptable excipients and/or accessory ingredients such as those described herein. General guidelines for the formulation and manufacture of pharmaceutical compositions and agents are available, for example, in Remington's The Science and Practice of Pharmacy, 21 st Edition, A. R. Gennaro; Lippincott, Williams & Wilkins, Baltimore, MD, 2006. Conventional excipients and/or accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some aspects, the composition may comprise a pharmaceutically acceptable carrier and/or vehicle. In some aspects, the composition may further include pyrogen-free water, isotonic saline and/or buffered (aqueous) solutions, e.g., phosphate, citrate etc. buffered solutions. In some aspects, the composition may include water and/or a buffer containing a sodium salt, such as at least 50 mM of a sodium salt, a calcium salt, in some aspects at least 0.01 mM of a calcium salt, and optionally a potassium salt, in some aspects at least 3 mM of a potassium salt. In some aspects the sodium, calcium and/or, optionally, potassium salts may occur in the form of their halogenides, e.g., chlorides, iodides, or bromides, in the form of their hydroxides, carbonates, hydrogen carbonates, and/or sulfates, etc. Examples of sodium salts include e.g., NaCl, NaI, NaBr, Na₂CO₃, NaHCO₃, Na₂SO₄, examples of the potassium salts include e.g., KCl, Kl, KBr, K₂CO₃, KHCO₃, K₂SO₄, and examples of calcium salts include e.g., CaCl₂), Cal₂, CaBr₂, CaCO₃, CaSO₄, Ca(OH)₂. In some aspects, organic anions of the aforementioned cations may be contained in the buffer. In some aspects, the composition may include salts selected from sodium chloride (NaCl), calcium chloride (CaCl₂)), and/or potassium chloride (KCl), wherein further anions may be present additional to the chlorides. CaCl₂) can also be replaced by another salt, such as KCl. In some aspects, the injection buffer may be hypertonic, isotonic or hypotonic with reference to the specific reference medium, e.g. the buffer may have a higher, identical or lower salt content with reference to the specific reference medium, wherein such concentrations of the afore mentioned salts may be used, which may minimize damage of cells due to osmosis or other concentration effects. The concentration of the salts in the composition can be about 70 mM to about 140 mM. For example, the salt concentration can be equal to any one of, at least any one of, at most any one of, or between any two of 50 mM, 60 mM, 70 mM, 80 mM, 90 mM, 100 mM, 120 mM, 130 mM, 140 mM, 150 mM, 160 mM, 170 mM, 180 mM, 190 mM, or 200 mM, or any range or value derivable therein. The salt can be at a neutral pH, pH 6.5 to 8.5, pH 7.0 to pH 8.0, or pH 7.2 to pH 7.6. For example, the salt can be at a pH equal to any one of, at least any one of, at most any one of, or between any two of 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein.

In some aspects, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some aspects, a pharmaceutically acceptable excipient is at least 95%, at least 96%, at least 97%, at least 98%, at least 99%, or 100% pure. Examples of excipients, which refer to ingredients in the compositions that are not active ingredients, include but are not limited to carriers, binders, diluents, lubricants, thickeners, surface active agents, preservatives, stabilizers, emulsifiers, buffers, flavoring agents, disintegrants, coatings, plasticizers, compression agents, wet granulation agents, and/or colorants. Preservatives for use in the compositions disclosed herein include but are not limited to benzalkonium chloride, chlorobutanol, paraben and/or thimerosal. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. Diluents, or diluting or thinning agents, include but are not limited to ethanol, glycerol, water, sugars such as lactose, sucrose, mannitol, and sorbitol, and starches derived from wheat, corn rice, and potato, and/or celluloses such as microcrystalline cellulose. The amount of diluent in the composition can range from about 10% to about 90% by weight of the total composition, about 25% to about 75%, about 30% to about 60% by weight, or about 12% to about 60%.

In some aspects, an excipient is approved for use in humans and for veterinary use. In some aspects, an excipient is approved by United States Food and Drug Administration. In some aspects, an excipient is pharmaceutical grade. In some aspects, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/V).

The pH and exact concentration of the various components in a pharmaceutical composition are adjusted according to well-known parameters. The use of such media and agents for pharmaceutical active substances is well known in the art. Except insofar as any conventional media or agent is incompatible with the active ingredients, its use in immunogenic and therapeutic compositions is contemplated.

In certain aspects, the lipid nanoparticles and/or pharmaceutical compositions of the disclosure are refrigerated or frozen for storage and/or shipment (e.g., being stored at a temperature of 10° C. or lower, such as a temperature at about 4°, a temperature between about −150° C. and about 10° C. (e.g., about 10° C., 9° C., 8° C., 7° C., 6° C., 5° C., 4° C., 3° C., 2° C., 1° C., 0° C., −1° C., −2° C., −3° C., −4° C., −5° C., −6° C., −7° C., −8° C., −9° C., −10° C., −15° C., −20° C., −25° C., −30° C., −40° C., −50° C., −60° C., −70° C., −80° C., −90° C., −130° C. or −150° C.) or a temperature between about −80° C. and about −20° C. (e.g., about −20° C., −25° C., −30° C., −40° C., −50° C., −60° C., −70° C., or −80° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., −80° C. or −90° C.

In certain aspects, the disclosure also relates to a method of increasing stability of the lipid nanoparticles by adding an effective amount of an amphiphilic polymer and by storing the lipid nanoparticles and/or pharmaceutical compositions thereof at a temperature of 10° C. or lower, such as a temperature at about 4° C., a temperature between about −150° C. and about 10° C. (e.g., about 10° C., 9° C., 8° C., 7° C., 6° C., 5° C., 4° C., 3° C., 2° C., 1° C., 0° C., −1° C., −2° C., −3° C., −4° C., −5° C., −6° C., −7° C., −8° C., −9° C., −10° C., −15° C., −20° C., −25° C., −30° C., −40° C., −50° C., −60° C., −70° C., −80° C., −90° C., −130° C. or −150° C.) or a temperature between about −80° C. and about −20° C. (e.g., about −20° C., −25° C., −30° C., −40° C., −50° C., −60° C., −70° C., or −80° C.).

The chemical properties of the LNP, LNP suspension, lyophilized LNP composition, or LNP formulation of the present disclosure may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and/or zeta potential.

The mean size of a LNP may be between 10 s of nm and 100 s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some aspects, the mean size of a LNP may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain aspects, the mean size of a LNP may be from about 70 nm to about 100 nm. In a particular aspect, the mean size may be about 80 nm. In other aspects, the mean size may be about 100 nm.

A LNP may be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some aspects, the polydispersity index of a LNP may be from about 0.10 to about 0.20.

The zeta potential of a LNP may be used to indicate the electrokinetic potential of the composition. For example, the zeta potential may describe the surface charge of a LNP. Lipid nanoparticles with relatively low charges, positive or negative, are generally desirable, as more highly charged species may interact undesirably with cells, tissues, and other elements in the body. In some aspects, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a LNP after preparation, relative to the initial amount provided. The encapsulation efficiency is desirably high (e.g., close to 100%). The encapsulation efficiency may be measured, for example, by comparing the amount of therapeutic and/or prophylactic in a solution containing the lipid nanoparticle before and after breaking up the lipid nanoparticle with one or more organic solvents or detergents. Fluorescence may be used to measure the amount of free therapeutic and/or prophylactic (e.g., RNA) in a solution. For the lipid nanoparticles described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some aspects, the encapsulation efficiency may be at least 80%. In certain aspects, the encapsulation efficiency may be at least 90%. In some aspects, the LNP encapsulation efficiency of the LNP, LNP suspension, lyophilized LNP composition, or LNP formulation of the present disclosure produced in the presence of blank LNPs (e.g., lipid nanoparticles comprising the lipids listed herein but not encapsulating any nucleic acid) is about 50% or higher, about 55% or higher, about 60% or higher, about 65% or higher, about 70% or higher, about 75% or higher, about 80% or higher, about 8% or higher, about 90% or higher, about 91% or higher, about 92% or higher, about 93% or higher, about 94% or higher, about 95% or higher, about 96% or higher, about 97% or higher, about 98% or higher, or about 99% or higher than the LNP encapsulation efficiency of the LNP, LNP suspension, lyophilized LNP composition, or LNP formulation produced by a comparable method in the presence of a lesser concentration of blank LNPs or in the absence of blank LNPs.

In some aspects, electrophoresis (e.g., capillary electrophoresis) and/or chromatography (e.g., reverse phase liquid chromatography) may be used to examine the mRNA integrity.

In some aspects, the LNP integrity of the LNP, LNP suspension, lyophilized LNP composition, or LNP formulation of the present disclosure produced in the presence of blank LNPs (e.g., lipid nanoparticles comprising the lipids listed in herein but not encapsulating any nucleic acid) is about 20% or higher, about 25% or higher, about 30% or higher, about 35% or higher, about 40% or higher, about 45% or higher, about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, or about 99% or higher than the LNP integrity of the LNP, LNP suspension, lyophilized LNP composition, or LNP formulation produced by a comparable method in the presence of a lesser concentration of blank LNPs or in the absence of blank LNPs.

In some aspects, the LNP integrity of the LNP, LNP suspension, lyophilized LNP composition, and/or LNP formulation of the present disclosure produced in the presence of blank LNPs (e.g., lipid nanoparticles comprising the lipids listed herein but not encapsulating any nucleic acid) is higher than the LNP integrity of the LNP, LNP suspension, lyophilized LNP composition, or LNP formulation produced by a comparable method in the presence of a lesser concentration of blank LNPs or in the absence of blank LNPs by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some aspects, the Tx0% of the LNP, LNP suspension, lyophilized LNP composition, and/or LNP formulation of the present disclosure produced in the presence of blank LNPs (e.g., lipid nanoparticles comprising the lipids listed herein but not encapsulating any nucleic acid) is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer than the Tx0% of the LNP, LNP suspension, lyophilized LNP composition, and/or LNP formulation produced by a comparable method in the presence of a lesser concentration of blank LNPs or in the absence of blank LNPs. In some aspects, the Tx0% of the LNP, LNP suspension, lyophilized LNP composition, and/or LNP formulation of the present disclosure produced in the presence of blank LNPs (e.g., lipid nanoparticles comprising the lipids listed herein but not encapsulating any nucleic acid) is longer than the Tx0% of the LNP, LNP suspension, lyophilized LNP composition, and/or LNP formulation produced by a comparable method in the presence of a lesser concentration of blank LNPs or in the absence of blank LNPs by about 5% or more, about 10% or more, about 15% or more, about 20% or more, about 30% or more, about 40% or more, about 50% or more, about 60% or more, about 70% or more, about 80% or more, about 90% or more, about 1 fold or more, about 2 folds or more, about 3 folds or more, about 4 folds or more, or about 5 folds or more.

In some aspects, the T1/2 of the LNP, LNP suspension, lyophilized LNP composition, or LNP formulation of the present disclosure produced in the presence of blank LNPs (e.g., lipid nanoparticles comprising the lipids listed herein but not encapsulating any nucleic acid) is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, or about 120 months or longer than the T1/2 of the LNP, LNP suspension, lyophilized LNP composition, or LNP formulation produced by a comparable method in the presence of a lesser concentration of blank LNPs or in the absence of blank LNPs.

In some aspects, the T1/2 of the LNP, LNP suspension, lyophilized LNP composition, or LNP formulation of the present disclosure produced in the presence of blank LNPs (e.g., lipid nanoparticles comprising the lipids listed herein but not encapsulating any nucleic acid) is longer than the T1/2 of the LNP, LNP suspension, lyophilized LNP composition, or LNP formulation produced by a comparable method in the presence of a lesser concentration of blank LNPs or in the absence of blank LNPs by about 5% or higher, about 10% or more, about 15% or more, about 20% or more, about 30% or more, about 40% or more, about 50% or more, about 60% or more, about 70% or more, about 80% or more, about 90% or more, about 1 fold or more, about 2 folds or more, about 3 folds or more, about 4 folds or more, or about 5 folds or more.

As used herein, “Tx” refers to the amount of time lasted for the nucleic acid integrity (e.g., mRNA integrity) of a LNP, LNP suspension, lyophilized LNP composition, or LNP formulation to degrade to about X of the initial integrity of the nucleic acid (e.g., mRNA) used for the preparation of the LNP, LNP suspension, lyophilized LNP composition, or LNP formulation. For example, “T80” refers to the amount of time lasted for the nucleic acid integrity (e.g., mRNA integrity) of a LNP, LNP suspension, lyophilized LNP composition, or LNP formulation to degrade to about 80% of the initial integrity of the nucleic acid (e.g., mRNA) used for the preparation of the LNP, LNP suspension, lyophilized LNP composition, or LNP formulation. For another example, “T1/2” refers to the amount of time lasted for the nucleic acid integrity (e.g., mRNA integrity) of a LNP, LNP suspension, lyophilized LNP composition, or LNP formulation to degrade to about ½ of the initial integrity of the nucleic acid (e.g., mRNA) used for the preparation of the LNP, LNP suspension, lyophilized LNP composition, or LNP formulation.

The amount of a therapeutic and/or prophylactic in a LNP may depend on the size, composition, desired target and/or application, or other properties of the lipid nanoparticle as well as on the properties of the therapeutic and/or prophylactic. For example, the amount of an RNA useful in a LNP may depend on the size, sequence, and other characteristics of the RNA. The relative amounts of a therapeutic and/or prophylactic (e.g., pharmaceutical substance) and other elements (e.g., lipids) in a LNP may also vary. In some aspects, the wt/wt ratio of the lipid component to a therapeutic and/or prophylactic in a LNP may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain aspects, the wt/wt ratio is about 20:1. The amount of a therapeutic and/or prophylactic in a LNP may, for example, be measured using absorption spectroscopy (e.g., ultraviolet-visible spectroscopy).

In some aspects, the mRNA to lipid ratio in the LNP (e.g., N:P, where N represents the moles of ionizable lipid and P represents the moles of phosphate present as part of the nucleic acid backbone) range from 2:1 to 30:1, for example 3:1 to 22:1. In other aspects, N: P ranges from 6:1 to 20:1 or 2:1 to 12:1. Exemplary N: P ranges include about 3:1, about 6:1, about 12:1 and about 22:1.

Various exemplary embodiments of the ionizable lipids of the present invention, lipid nanoparticles and compositions comprising the same, and their use to deliver active (e.g., therapeutic agents), such as nucleic acids, to modulate gene and protein expression, are described in further detail below.

### RNA Encapsulation

The RNA in an RNA product solution may be encapsulated, and the RNA solution may further comprise at least one encapsulating agent. In one aspect, the encapsulating agent comprises a lipid, a lipid nanoparticle (LNP), lipoplexes, polymeric particles, polyplexes, monolithic delivery systems, or a combination thereof. In some aspects, 1, 2, 3, 4, 5, or more of the foregoing elements may be excluded as an encapsulating agent.

In one aspect, the encapsulating agent is a lipid, and produced is lipid nanoparticle (LNP)-encapsulated RNA. Without intending to be bound by any theory, it is believed that the cationic or cationically ionizable lipid or lipid-like material and/or the cationic polymer combine together with the nucleic acid to form aggregates, and this aggregation results in colloidally stable particles.

A lipid may be a naturally occurring lipid or a synthetic lipid. However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof. A lipid is a substance that is insoluble in water and extractable with an organic solvent. Compounds other than those specifically described herein are understood by one of skill in the art as lipids and are encompassed by the compositions and methods of the present disclosure. A lipid component and a non-lipid may be attached to one another, either covalently or non-covalently.

In some aspects, LNPs may be designed to protect RNA molecules (e.g., saRNA, mRNA) from extracellular Rnases and/or may be engineered for systemic delivery of the RNA to target cells. In some aspects, such LNPs may be particularly useful to deliver RNA molecules (e.g., saRNA, mRNA) when RNA molecules are intravenously administered to a subject in need thereof. In some aspects, such LNPs may be particularly useful to deliver RNA molecules (e.g., saRNA, mRNA) when RNA molecules are intramuscularly administered to a subject in need thereof. In some aspects, such LNPs may be particularly useful to deliver RNA molecules (e.g., saRNA, mRNA) when RNA molecules are intradermally administered to a subject in need thereof. In some aspects, such LNPs may be particularly useful to deliver RNA molecules (e.g., saRNA, mRNA) when RNA molecules are intranasally administered to a subject in need thereof.

In one aspect, the RNA in the RNA product solution is at a concentration of <1 mg/mL. In another aspect, the RNA is at a concentration of at least or at least about 0.05 mg/ml. In another aspect, the RNA is at a concentration of at least or at least about 0.5 mg/mL. In another aspect, the RNA is at a concentration of at least or at least about 1 mg/mL. In another aspect, the RNA concentration is from or from about 0.05 mg/mL to about 0.5 mg/mL. In another aspect, the RNA is at a concentration of at least 10 mg/mL. In another aspect, the RNA is at a concentration of at least 50 mg/mL. In some aspects, the RNA is or is not at a concentration of at least, at most, exactly, between (inclusive or exclusive) any two of, or about 0.05 mg/ml, 0.5 mg/mL, 1 mg/mL, 10 mg/mL, 50 mg/mL, 75 mg/mL, 100 mg/mL, 150 mg/mL, 200 mg/mL, 250 mg/mL, 300 mg/mL, 400 mg/mL, or more.

The present disclosure provides for an RNA product solution and a lipid preparation mixture or compositions thereof comprising at least one RNA encoding, e.g., an antigen complexed with, encapsulated in, and/or formulated with one or more lipids, and forming lipid nanoparticles (LNPs), liposomes, lipoplexes and/or nanoliposomes. In some aspects, the composition comprises a lipid nanoparticle.

A lipid nanoparticle or LNP refers to particles of any morphology generated when a cationic lipid and optionally one or more further lipids are combined, e.g., in an aqueous environment and/or in the presence of RNA. In some aspects, lipid nanoparticles are included in a formulation that may be used to deliver an active agent or therapeutic agent, such as a nucleic acid (e.g., mRNA) to a target site of interest (e.g., cell, tissue, organ, tumor, and the like). In some aspects, the lipid nanoparticles of the present disclosure comprise a nucleic acid (e.g., mRNA). Such lipid nanoparticles typically comprise a cationic lipid and one or more excipients, e.g., one or more neutral lipids, charged lipids, steroids, polymer conjugated lipids, or combinations thereof. In some aspects, the LNPs comprise at least one cationic (e.g., ionizable) lipid, at least one neutral (e.g., non-cationic) lipid, at least one structural lipid (e.g., a steroid), and/or at least one polymer conjugated lipid (e.g., a polyethylene glycol (PEG)-modified lipid). In some aspects, 1, 2, 3, or more of the foregoing excipients may be excluded from the LNPs.

In some aspects, the LNPs comprise 20-60 mol % cationic (e.g., ionizable) lipid(s). For example, the LNPs may comprise 20-50 mol %, 20-40 mol %, 20-30 mol %, 30-60 mol %, 30-50 mol %, 30-40 mol %, 40-60 mol %, 40-50 mol %, or 50-60 mol % cationic (e.g., ionizable) lipid(s). In some aspects, the LNPs comprise or do not comprise at least, at most, exactly, or between (inclusive or exclusive) any two of 20 mol %, 30 mol %, 40 mol %, 50, or 60 mol % cationic (e.g., ionizable) lipid(s). In some aspects, the LNPs comprise 45 to 55 mole percent (mol %) cationic (e.g., ionizable) lipid(s). For example, LNPs may comprise or not comprise at least, at most, exactly, or between (inclusive or exclusive) any two of 45, 46, 47, 48, 49, 50, 51, 52, 53, 54, or 55 mol % cationic (e.g., ionizable) lipid(s).

In some aspects, the LNPs comprise 5-25 mol % neutral (e.g., non-cationic) lipid(s). For example, the LNPs may comprise 5-20 mol %, 5-15 mol %, 5-10 mol %, 10-25 mol %, 10-20 mol %, 10-25 mol %, 15-25 mol %, 15-20 mol %, or 20-25 mol % neutral (e.g., non-cationic) lipid(s). In some aspects, the LNPs are or are not at least, at most, exactly, or between (inclusive or exclusive) any two of 5 mol %, 10 mol %, 15 mol %, 20 mol %, or 25 mol % neutral (e.g., non-cationic) lipid(s). In some aspects, the LNPs comprise 5 to 15 mol % neutral (e.g., non-cationic) lipid(s). For example, LNPs may comprise at least, at most, exactly, or between (inclusive or exclusive) any two of 5, 6, 7, 8, 9, 10, 11, 12, 13, 14, or 15 mol % neutral (e.g., non-cationic) lipid(s).

In some aspects, the LNPs comprise 25-55 mol % structural lipid(s) (e.g., a steroid). For example, the LNPs may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % structural lipid(s) (e.g., a steroid). In some aspects, the LNPs are or are not at least, at most, exactly, or between (inclusive or exclusive) any two of 25 mol %, 30 mol %, 35 mol %, 40 mol %, 45 mol %, 50 mol %, or 55 mol % structural lipid(s) (e.g., a steroid). In some aspects, the LNPs comprise 35 to 40 mol % structural lipid(s) (e.g., a steroid). For example, LNPs may comprise at least, at most, exactly, or between (inclusive or exclusive) any two of 35, 36, 37, 38, 39, or 40 mol % structural lipid(s) (e.g., a steroid).

In some aspects, the LNPs comprise 0.5-15 mol % polymer conjugated lipid(s) (e.g., a polyethylene glycol (PEG)-modified lipid). For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol % polymer conjugated lipid(s) (e.g., a polyethylene glycol (PEG)-modified lipid). In some aspects, the lipid LNPs are or are not at least, at most, exactly, or between (inclusive or exclusive) any two of 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % polymer conjugated lipid(s) (e.g., a polyethylene glycol (PEG)-modified lipid). In some aspects, the LNPs comprise 1 to 2 mol % polymer conjugated lipid(s) (e.g., a polyethylene glycol (PEG)-modified lipid). For example, LNPs may comprise at least, at most, exactly, or between (inclusive or exclusive) any two of 1, 1.5, or 2 mol % polymer conjugated lipid(s) (e.g., a polyethylene glycol (PEG)-modified lipid).

In some aspects, the LNPs comprise 20-75 mol % cationic (e.g., ionizable) lipid(s) (e.g., at least, at most, exactly, or between (inclusive or exclusive) any two of 20%, 25%, 30%, 35%, 40%, 45%, 50%, 55%, 60%, 65%, 70%, and 75%), 0.5-25 mol % neutral (e.g., non-cationic) lipid(s) (e.g., at least, at most, exactly, or between (inclusive or exclusive) of 0.5%, 2.25%, 4%, 5.75%, 7.5%, 9.25%, 11%, 12.75%, 14.5%, 16.25%, 18%, 19.75%, 21.5%, 23.25%, and 25%), 5-55 mol % structural lipid(s) (e.g., a sterol) e.g., non-cationic) lipid(s) (e.g., at least, at most, exactly, or between (inclusive or exclusive) of 5%, 10%, 15%, 20%, 25%, 30%, 35%, 40%, 45%, 50%, and 55%), and 0.5-20 mol % polymer conjugated lipid(s) (e.g., a polyethylene glycol (PEG)-modified lipid) (e.g., at least, at most, exactly, or between (inclusive or exclusive) of 0.5%, 2%, 3.5%, 5%, 6.5%, 8%, 9.5%, 11%, 12.5%, 14%, 15.5%, 17%, 18.5%, and 20%). In some aspects, 1, 2, 3, or more of the lipids may be excluded from the LNPs.

In some non-limiting aspects, the molar lipid ratio is 50/10/38.5/1.5 (mol % cationic lipid/neutral lipid/structural lipid/polymer conjugated lipid), 60/7.5/31/1.5 (mol % cationic lipid/neutral lipid/structural lipid/polymer conjugated lipid), 57.5/7.5/31.5/3.5 (mol % cationic lipid/neutral lipid/structural lipid/polymer conjugated lipid), 57.2/7.1/34.3/1.4 (mol % cationic lipid/neutral lipid/structural lipid/polymer conjugated lipid), 40/15/40/5 (mol % cationic lipid/neutral lipid/structural lipid/polymer conjugated lipid), 50/10/35/4.5/0.5 (mol % cationic lipid/neutral lipid/structural lipid/polymer conjugated lipid), 50/10/35/5 (mol % cationic lipid/neutral lipid/structural lipid/polymer conjugated lipid), 40/10/40/10 (mol % cationic lipid/neutral lipid/structural lipid/polymer conjugated lipid), 35/15/40/10 (mol % cationic lipid/neutral lipid/structural lipid/polymer conjugated lipid), or 52/13/30/5 (mol % cationic lipid/neutral lipid/structural lipid/polymer conjugated lipid).

In some aspects, the active agent or therapeutic agent, such as a nucleic acid (e.g., mRNA), may be encapsulated in the lipid portion of the lipid nanoparticle and/or an aqueous space enveloped by some or all of the lipid portion of the lipid nanoparticle, thereby protecting it from enzymatic degradation or other undesirable effects induced by the mechanisms of the host organism or cells, e.g., an adverse immune response. The nucleic acid (e.g., mRNA) or a portion thereof may also be associated and complexed with the lipid nanoparticle. A lipid nanoparticle may comprise any lipid capable of forming a particle to which the nucleic acids are attached, and/or in which the one or more nucleic acids are encapsulated.

In some aspects, provided RNA molecules (e.g., saRNA, mRNA) may be formulated with LNPs. In some aspects, the lipid nanoparticles may or may not have a mean diameter of or of about 1 to 500 nm (e.g., at least, at most, exactly, or between (inclusive or exclusive) of 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 nm). In some aspects, the lipid nanoparticles have a mean diameter of or of from about 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, about 70 nm to about 80 nm, or at least, at most, exactly, or between (inclusive or exclusive) of 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321). Here, “mean diameter,” “diameter,” or “size” for particles is used synonymously with the value of the Z-average.

LNPs described herein may exhibit a polydispersity index less than or less than about 0.5, 0.4, 0.3, or 0.2 or less. By way of example, the LNPs may or may not exhibit a polydispersity index of at least, at most, exactly, or between (inclusive or exclusive) of 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5. The polydispersity index is, in some aspects, calculated based on dynamic light scattering measurements by the so-called cumulant analysis referred to in the definition of “average diameter.” Under certain prerequisites, it may be taken as a measure of the size distribution of an ensemble of nanoparticles.

In some aspects, an LNP of the disclosure comprises or does not comprise an N: P ratio of or of from about 2:1 to about 30:1, e.g., at least, at most, exactly, or between (inclusive or exclusive) of 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1. In some aspects, an LNP of the disclosure comprises an N: P ratio of or of about 6:1. In some aspects, an LNP of the disclosure comprises an N: P ratio of or of about 3:1.

In some aspects, an LNP of the disclosure comprises or does not comprise a wt/wt ratio of the cationic lipid component to the RNA of or of from about 5:1 to about 100:1, e.g., at least, at most, exactly, or between (inclusive or exclusive) of 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, or 100:1. In some aspects, an LNP of the disclosure comprises a wt/wt ratio of the ionizable cationic lipid component to the RNA of or of about 20:1. In some aspects, an LNP of the disclosure comprises a wt/wt ratio of the ionizable cationic lipid component to the RNA of or of about 10:1.

In certain aspects, nucleic acids (e.g., RNA molecules), when present in provided LNPs, are resistant in aqueous solution to degradation with a nuclease. In some aspects, LNPs are liver-targeting lipid nanoparticles. In some aspects, LNPs are cationic lipid nanoparticles comprising one or more cationic lipids (e.g., those described herein). In some aspects, cationic LNPs may comprise at least one cationic lipid, at least one polymer conjugated lipid, and at least one helper lipid (e.g., at least one neutral lipid).

In certain aspects, the RNA solution and lipid preparation mixture or compositions thereof may have at least, at most, exactly, between (inclusive or exclusive) of, or about 1%, 2%, 3%, 4% 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of a particular lipid, lipid type, or non-lipid component such as lipid-like materials and/or cationic polymers and/or an adjuvant, antigen, peptide, polypeptide, sugar, nucleic acid or other material disclosed herein or as would be known to one of skill in the art.

LNPs described herein can be generated using components, compositions, and methods as are generally known in the art, see, e.g., PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety. Other non-limiting examples of methods for preparing LNPs can be found in, e.g., WO 2022/032154, the disclosure of which is incorporated by reference herein in its entirety.

For example, methods of preparing LNPs may involve obtaining a colloid from at least one cationic or cationically ionizable lipid or lipid-like material and/or at least one cationic polymer and mixing the colloid with nucleic acid to obtain nucleic acid particles. The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” refers only to the particles in the mixture and not the entire suspension.

For the preparation of colloids comprising at least one cationic or cationically ionizable lipid or lipid-like material and/or at least one cationic polymer, methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are first dissolved in a suitable organic solvent and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that subsequently turns into a liposomal suspension.

The term “ethanol injection technique” refers to a process in which an ethanol solution comprising lipids is rapidly injected into an aqueous solution through a needle. This action disperses the lipids throughout the solution and promotes lipid structure formation, for example, lipid vesicle formation such as liposome formation. Generally, the RNA lipoplex particles described herein are obtainable by adding RNA to a colloidal liposome dispersion. Using the ethanol injection technique, such colloidal liposome dispersion is, in some aspects, formed as follows: an ethanol solution comprising lipids, such as cationic lipids and additional lipids, is injected into an aqueous solution under stirring. In some aspects, the RNA lipoplex particles described herein are obtainable without a step of extrusion. The term “extruding” or “extrusion” refers to the creation of particles having a fixed, cross-sectional profile. In particular, it refers to the downsizing of a particle, whereby the particle is forced through filters with defined pores.

Other methods for preparing a colloid having organic solvent free characteristics may also be used according to the present disclosure.

In some aspects, LNP-encapsulated RNA may be produced by rapid mixing of an RNA solution (e.g., the RNA product solution) and a lipid preparation described herein (comprising, e.g., at least one cationic lipid and optionally one or more other lipid components, in an organic solvent) under conditions such that a sudden change in solubility of lipid component(s) is triggered, which drives the lipids towards self-assembly in the form of LNPs. In some aspects, suitable buffering agents comprise tris, histidine, citrate, acetate, phosphate, and/or succinate. In some aspects, 1, 2, 3, or more of the foregoing buffering agents are excluded. The pH of a liquid formulation relates to the pKa of the encapsulating agent (e.g., cationic lipid). The pH of the acidifying buffer may be at least half a pH scale less than the pKa of the encapsulating agent (e.g., cationic lipid), and the pH of the final buffer may be at least half a pH scale greater than the pKa of the encapsulating agent (e.g., cationic lipid). In some aspects, properties of a cationic lipid are chosen such that nascent formation of particles occurs by association with an oppositely charged backbone of a nucleic acid (e.g., RNA). In this way, particles are formed around the nucleic acid, which, for example, in some aspects, may result in much higher encapsulation efficiency than is achieved in the absence of interactions between nucleic acids and at least one of the lipid components. In certain aspects, nucleic acids, when present in the lipid nanoparticles, are resistant in aqueous solution to degradation with a nuclease.

Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

Some aspects described herein relate to compositions, methods and uses involving more than one, e.g., 2, 3, 4, 5, 6 or even more nucleic acid species, such as RNA species. In an LNP formulation, it is possible that each nucleic acid species is separately formulated as an individual LNP formulation. In that case, each individual LNP formulation will comprise one nucleic acid species. The individual LNP formulations may be present as separate entities, e.g., in separate containers. Such formulations are obtainable by providing each nucleic acid species separately (typically each in the form of a nucleic acid-containing solution) together with suitable cationic or cationically ionizable lipids or lipid-like materials and cationic polymers that allow the formation of LNPs. Respective particles will contain exclusively the specific nucleic acid species that is being provided when the particles are formed (individual particulate formulations).

In some aspects, a composition such as a pharmaceutical composition comprises more than one individual LNP formulation. Respective pharmaceutical compositions are referred to as mixed LNP formulations. Mixed LNP formulations according to the invention are obtainable by forming, separately, individual LNP formulations, as described above, followed by a step of mixing of the individual LNP formulations. By the step of mixing, a formulation comprising a mixed population of nucleic acid-containing LNPs is obtainable. Individual LNP populations may be together in one container, comprising a mixed population of individual LNP formulations.

Alternatively, it is possible that different nucleic acid species are formulated together as a combined LNP formulation. Such formulations are obtainable by providing a combined formulation (typically combined solution) of different RNA species together with suitable cationic or cationically ionizable lipids or lipid-like materials and cationic polymers that allow the formation of LNPs. As opposed to a mixed LNP formulation, a combined LNP formulation will typically comprise LNPs that comprise more than one RNA species. In a combined LNP composition, different RNA species are typically present together in a single particle.

### A. Cationic Polymeric Materials

Given their high degree of chemical flexibility, polymeric materials are commonly used for nanoparticle-based delivery. Typically, cationic materials are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic materials useful in some aspects herein. In addition, some investigators have synthesized polymeric materials specifically for nucleic acid delivery. Poly (P-amino esters), in particular, have gained widespread use in nucleic acid delivery owing to their ease of synthesis and biodegradability. In some aspects, such synthetic materials may be suitable for use as cationic materials herein.

A “polymeric material,” as used herein, is given its ordinary meaning, e.g., a molecular structure comprising one or more repeat units (monomers), connected by covalent bonds. In some aspects, such repeat units may all be identical; alternatively, in some cases, there may be more than one type of repeat unit present within the polymeric material. In some cases, a polymeric material is biologically derived, e.g., a biopolymer such as a protein. In some cases, additional moieties may also be present in the polymeric material, for example targeting moieties such as those described herein.

Those skilled in the art are aware that, when more than one type of repeat unit is present within a polymer (or polymeric moiety), then the polymer (or polymeric moiety) is said to be a “copolymer.” In some aspects, a polymer (or polymeric moiety) utilized in accordance with the present disclosure may be a copolymer. Repeat units forming the copolymer may be arranged in any fashion. For example, in some aspects, repeat units may be arranged in a random order; alternatively or additionally, in some aspects, repeat units may be arranged in an alternating order, or as a “block” copolymer, e.g., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

In certain aspects, a polymeric material for use in accordance with the present disclosure is biocompatible. Biocompatible materials are those that typically do not result in significant cell death at moderate concentrations. In certain aspects, a biocompatible material is biodegradable, e.g., is able to degrade, chemically and/or biologically, within a physiological environment, such as within the body. In certain aspects, a polymeric material may be or comprise protamine or polyalkyleneimine, in particular protamine.

As those skilled in the art are aware term “protamine” is often used to refer to any of various strongly basic proteins of relatively low molecular weight that are rich in arginine and are found associated especially with DNA in place of somatic histones in the sperm cells of various animals (as fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some aspects, the term “protamine” as used herein is refers to a protamine amino acid sequence obtained or derived from natural or biological sources, including fragments thereof and/or multimeric forms of said amino acid sequence or fragment thereof, as well as (synthesized) polypeptides which are artificial and specifically designed for specific purposes and cannot be isolated from native or biological sources.

In some aspects, a polyalkyleneimine comprises polyethylenimine and/or polypropylenimine. In some aspects, the polyalkyleneimine is polyethyleneimine (PEI). In some aspects, the polyalkyleneimine is a linear polyalkyleneimine, e.g., linear polyethyleneimine (PEI).

Cationic materials (e.g., polymeric materials, including polycationic polymers) contemplated for use herein include those which are able to electrostatically bind nucleic acid. In some aspects, cationic polymeric materials contemplated for use herein include any cationic polymeric materials with which nucleic acid may be associated, e.g. by forming complexes with the nucleic acid or forming vesicles in which the nucleic acid is enclosed or encapsulated.

In some aspects, particles described herein may comprise polymers other than cationic polymers, e.g., non-cationic polymeric materials and/or anionic polymeric materials. Collectively, anionic and neutral polymeric materials are referred to herein as non-cationic polymeric materials.

### B. Lipids & Lipid-Like Materials

The terms “lipid” and “lipid-like material” are used herein to refer to molecules which comprise one or more hydrophobic moieties or groups and optionally also one or more hydrophilic moieties or groups. According to the disclosure, lipids and lipid-like materials may be cationic, anionic or neutral. Neutral lipids or lipid-like materials exist in an uncharged or neutral zwitterionic form at a selected pH.

The term “lipid” refers to a group of organic compounds that are characterized by being insoluble in water but soluble in many organic solvents. Generally, lipids may be divided into eight categories: fatty acids and their derivatives (including tri-, di-, monoglycerides, and phospholipids), glycerolipids, glycerophospholipids, sphingolipids, saccharolipids, polyketides, sterol lipids as well as sterol-containing metabolites such as cholesterol, and prenol lipids. Examples of fatty acids include, but are not limited to, fatty esters and fatty amides. Examples of glycerolipids include, but are not limited to, glycosylglycerols and glycerophospholipids (e.g., phosphatidylcholine, phosphatidylethanolamine, phosphatidylserine). Examples of sphingolipids include, but are not limited to, ceramides phosphosphingolipids (e.g., sphingomyelins, phosphocholine), and glycosphingolipids (e.g., cerebrosides, gangliosides). Examples of sterol lipids include, but are not limited to, cholesterol and its derivatives and tocopherol and its derivatives. In some aspects, 1, 2, 3, 4, 5, or more of the lipids may be excluded from the LNPs of the present disclosure.

The term “lipid-like material,” “lipid-like compound,” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

In some aspects, the RNA solution and lipid preparation mixture or compositions thereof may comprise cationic lipids, neutral lipids, cholesterol, and/or polymer (e.g., polyethylene glycol) conjugated lipids which form lipid nanoparticles that encompass the RNA molecules. Therefore, in some aspects, the LNP may comprise a cationic lipid and one or more excipients, e.g., one or more neutral lipids, charged lipids, steroids or steroid analogs (e.g., cholesterol), polymer conjugated lipids (e.g. PEG-lipid), or combinations thereof. In some aspects, 1, 2, 3, or more of the foregoing excipients may be excluded from the LNPs of the present disclosure. In some aspects, the lipids are present in a composition in an amount that is effective to form a lipid nanoparticle and deliver a therapeutic agent, e.g., an RNA molecule, for treating a particular disease or condition of interest. In some aspects, the LNPs encompass, or encapsulate, the nucleic acid molecules.

i. Cationic Lipids

Cationic or cationically ionizable lipids or lipid-like materials refer to a lipid or lipid-like material capable of being positively charged and able to electrostatically bind nucleic acid. As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl, or more acyl chains, and the head group of the lipid typically carries the positive charge. Exemplary cationic lipids include one or more amine group(s) which bear the positive charge. Cationic lipids may encapsulate negatively charged RNA.

In some aspects, cationic lipids are ionizable such that they may exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “cationically ionizable” lipids or lipid-like materials are comprised by the term “cationic lipid” or “cationic lipid-like material” unless contradicted by the circumstances.

In some aspects, a cationic lipid may comprise from or from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle. In some aspects, a cationic lipid may or may not be at least, at most, exactly, or between (inclusive or exclusive) of 10 mol %, 20 mol %, 30 mol %, 40 mol %, 50 mol %, 60 mol %, 70 mol %, 80 mol %, 90 mol %, or 100 mol %, or any range or value derivable therein, of the total lipid present in the particle.

In preferred aspects, the ionizable lipid is a compound having the following structure (I):

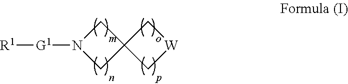

or a pharmaceutically acceptable salt, N-oxide, tautomer, or stereoisomer thereof, wherein
m, n, o, and p are each independently 1-3;
G¹ is C₁₋₁₂alkylene or C₂₋₁₂alkenylene;
R¹ is —N(R²)R³, —OR⁴, CN, —N(R⁴)(heteroaryl), —O(CH₂)_{q}OH, —(OCH₂CH₂)ᵣOH, —OC(═O)R⁵, —N(R⁴)C(═O)R⁵, —N(R⁴)S(O)₂R⁵, —N(R⁴)C(═O)N(R²)R³, —OC(═O)N(R²)R³, —N(R⁴)C(═O)OR⁵, —N(R⁴)C(═S)N(R²)R³, —N(R⁴)C(═NR⁶)N(R²)R³, or

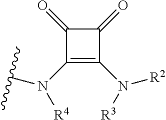

R² and R³ are each independently H, C₁₋₆alkyl, C₃₋₈cycloalkyl, or aryl; or R² and R³ together with the nitrogen atom to which they are attached form a heterocyclic ring;
R⁴ is H, C₁₋₆alkyl or C₃₋₈cycloalkyl;
R⁵ is C₁₋₆alkyl or C₃₋₈cycloalkyl optionally substituted by C₁₋₆alkyl;
R⁶ is H, CN, NO₂, C₁₋₆alkyl, OR⁵, S(O)₂R⁵, or S(O)₂N(R²)R³;
q is 2-6;
r is 1-6;
W is

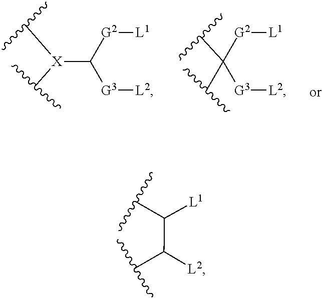

X is N or CH;
G² and G³ are each independently C₁₋₁₂alkylene or C₂₋₁₂alkenylene;
L¹ and L² are each independently —C(═O)OR⁷, —OC(═O)R⁷, —OC(═O)(CH₂)ᵣC(═O)OR⁷, —OC(═O)(CH₂)ᵣOC(═O)R⁷, —OC(═O)N(R⁴)R⁷, —N(R⁴)C(═O)OR⁷, —N(R⁴)C(═O)N(R⁴)R⁷, OC(═O)OR⁷, or —S—SR⁷; and
R⁷ is C₆₋₂₄alkyl, C₆₋₂₄alkenyl, or C₆₋₂₄alkynyl where each is optionally substituted by F, C₁₋₆alkoxy, C₃₋₈cycloalkyl, or C₃₋₈cycloalkenyl, wherein the R⁷ of L¹ and L² may be the same or different.

In some aspects, the RNA-LNPs comprise a cationic/ionizable lipid as described herein, a RNA molecule and one or more of neutral lipids, steroids, pegylated lipids, or combinations thereof. If more than one cationic lipid is incorporated within the LNP, such percentages apply to the combined cationic lipids. In one aspect, the cationic lipid is present in the LNP in an amount such as at least, at most, exactly, or between (inclusive or exclusive) of, or about 40, 41, 42, 43, 44, 45, 46, 47, 48, 49, 50, 51, 52, 53, 54, 55, 56, 57, 58, 59 or 60 mole percent (mol %). In some aspects, two or more cationic lipids are incorporated within the LNP. If more than one cationic lipid is incorporated within the LNP, the foregoing percentages apply to the combined cationic lipids.

ii. Polymer Conjugated Lipid

In some aspects, the LNPs comprise a polymer conjugated lipid. The term “polymer conjugated lipid” refers to a molecule comprising both a lipid portion and a polymer portion. An example of a polymer conjugated lipid is a pegylated lipid (e.g., polyethylene glycol-lipid, PEG-lipid). In certain aspects, the LNP comprises an additional, stabilizing lipid that is a pegylated lipid. The term “pegylated lipid” refers to a molecule comprising both a lipid portion and a polyethylene glycol portion.

Pegylated lipids are known in the art and include, but are not limited to, PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols, 2-[(polyethylene glycol)-2000]-N,N-ditetradecylacetamide, and mixtures thereof. Representative polyethylene glycol-lipids include PEG-c-DOMG, PEG-c-DMA, PEG-DSG, PEG-DPG, and PEG-s-DMG (1-(monomethoxy-polyethyleneglycol)-2,3-dimyristoylglycerol). In one aspect, the polyethylene glycol-lipid is N-[(methoxy polyethylene glycol)2000)carbamoyl]-1,2-dimyristyloxlpropyl-3-amine (PEG-c-DMA). In one aspect, the polyethylene glycol-lipid is PEG-2000-DMG. In one aspect, the polyethylene glycol-lipid is PEG-c-DOMG. In other aspects, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanolamine (PEG-PE), a PEG succinate diacylglycerol (PEG-S-DAG) such as 4-O-(2′,3′-di(tetradecanoyloxy)propyl-1-O—((O-methoxy(polyethoxy)ethyl)butanedioate (PEG-S-DMG), a PEGylated ceramide (PEG-cer), or a PEG dialkoxypropylcarbamate such as co-methoxy (polyethoxy)ethyl-N-(2,3di(tetradecanoxy)propyl)carbamate or 2,3-di(tetradecanoxy)propyl-N-(ω-methoxy(polyethoxy)ethyl)carbamate. PEG-lipids are disclosed in, e.g., U.S. Pat. No. 9,737,619, the full disclosures of which is herein incorporated by reference in its entirety for all purposes. In some aspects, 1, 2, 3, 4, 5, or more of the foregoing pegylated lipids may be excluded from the LNPs of the present disclosure.

In some aspects, the composition comprises a pegylated lipid having the following structure:

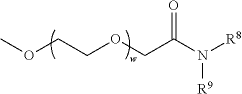

or a pharmaceutically acceptable salt, tautomer, or stereoisomer thereof, wherein:

R⁸ and R⁹ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some aspects, R⁸ and R⁹ are each independently straight, saturated alkyl chains containing from 12 to 16 carbon atoms. In some aspects, w has a mean value ranging from 43 to 53. In other aspects, the average w is or is about 45. In other different embodiments, the average w is or is about 49.

In some aspects, the lipid nanoparticles comprise a polymer conjugated lipid. In one aspect, the lipid nanoparticle comprises 2-[(polyethylene glycol)-2000]-N,N-ditetradecylacetamide (ALC-0159), having the formula:

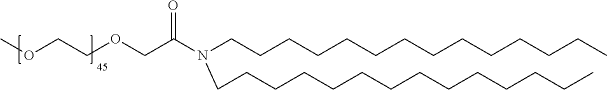

In various aspects, the molar ratio of the cationic lipid to the pegylated lipid ranges from or from about 100:1 to about 20:1, e.g., 20:1, 25:1, 30:1, 35:1, 40:1, 45:1, 50:1, 55:1, 60:1, 65:1, 70:1, 75:1, 80:1, 85:1, 90:1, 95:1, or 100:1, or any range or value derivable therein.

In certain aspects, the PEG-lipid is or is not present in the LNP in an amount from or from about 1 to about 10 mole percent (mol %) (e.g., at least, at most, exactly, or between (inclusive or exclusive) of 1, 2, 3, 4, 5, 6, 7, 8, 9, or 10 mol %), relative to the total lipid content of the nanoparticle.

In some aspects, the ratio of PEG in the lipid nanoparticle formulations may be increased or decreased and/or the carbon chain length of the PEG lipid may be modified to alter the pharmacokinetics and/or biodistribution of the lipid nanoparticle formulations.

iii. Additional Lipids

In certain aspects, the LNP comprises one or more additional lipids or lipid-like materials that stabilize particles during their formation. Suitable stabilizing or structural lipids include non-cationic lipids, e.g., neutral lipids and anionic lipids. Without being bound by any theory, optimizing the formulation of LNPs by addition of other hydrophobic moieties, such as cholesterol and lipids, in addition to an ionizable/cationic lipid or lipid-like material may enhance particle stability and efficacy of nucleic acid delivery.

As used herein, an “anionic lipid” refers to any lipid that is negatively charged at a selected pH. The term “neutral lipid” refers to any one of a number of lipid species that exist in either an uncharged or neutral zwitterionic form at physiological pH. In some aspects, additional lipids comprise one of the following neutral lipid components: (1) a phospholipid, (2) cholesterol or a derivative thereof; or (3) a mixture of a phospholipid and cholesterol or a derivative thereof.

Representative neutral lipids include phosphatidylcholines, phosphatidylethanolamines, phosphatidylglycerols, phosphatidic acids, phosphatidylserines, ceramides, sphingomyelins, dihydro-sphingomyelins, cephalins, and cerebrosides. Exemplary phospholipids include, for example, phosphatidylcholines, e.g., diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), dioleoylphosphatidylglycerol (DOPG), dipalmitoylphosphatidylglycerol (DPPG), (DAPC), dibehenoylphosphatidylcholine (DBPC), diarachidoylphosphatidylcholine ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), and 1-hexadecyl-sn-glycero-3-phosphocholine (C16 Lyso PC); and phosphatidylethanolamines, e.g., diacylphosphatidylethanolamines, such as dioleoyl-phosphatidylethanolamine (DOPE), 1,2-diundecanoyl-sn-glycero-phosphocholine (DUPC), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), dilauroyl-phosphatidylethanolamine (DLPE), distearoyl-phosphatidylethanolamine (DSPE), 1-phytanoyl-phosphatidylethanolamine (DpyPE), 16-O-monomethyl PE, 16-O-dimethyl PE, 18-1-trans PE, 1-stearoyl-2-oleoylphosphatidyethanolamine (SOPE), 1,2-dielaidoyl-sn-glycero-3-phosphoethanolamine (transDOPE), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In some aspects, 1, 2, 3, 4, 5, or more of the foregoing neutral lipids may be excluded from the LNPs of the present disclosure.

In one aspect, the neutral lipid is 1,2-distearoyl-sn-glycero-3-phosphocholine (DSPC), having the formula:

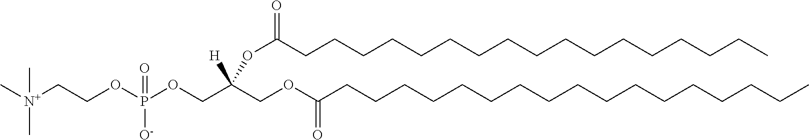 [SMILES: CCCCCCCCCCCCCCCCCC(=O)OC[C@H](COP(=O)([O-])OCC[N+](C)(C)C)OC(=O)CCCCCCCCCCCCCCCCC]

In some aspects, the LNPs comprise a neutral lipid, and the neutral lipid comprises one or more of DSPC, DPPC, DMPC, DOPC, POPC, DOPE, and/or SM. In some aspects, 1, 2, 3, 4, 5, or more of the foregoing neutral lipids may be excluded from the LNPs of the present disclosure.

In various aspects, the LNPs further comprise a steroid or steroid analogue. A “steroid” is a compound comprising the following carbon skeleton:

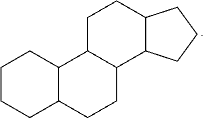 [SMILES: C1CCC2C(C1)CCC1C3CCCC3CCC21]

In certain aspects, the steroid or steroid analogue is cholesterol, fecosterol, sitosterol, ergosterol, campesterol, stigmasterol, brassicasterol, tomatidine, ursolic acid, alpha-tocopherol, and mixtures thereof. In some aspects, 1, 2, 3, 4, 5, or more of the foregoing steroid or steroid analogues may be excluded from the LNPs of the present disclosure. In certain aspects, the steroid or steroid analogue is cholesterol. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof. In some aspects, 1, 2, 3, 4, 5, or more of the foregoing cholesterol derivatives may be excluded from the LNPs of the present disclosure. In one aspect, the cholesterol has the formula:

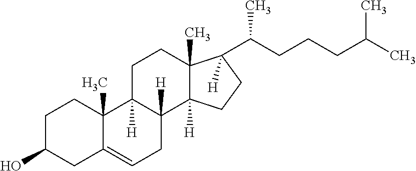

Without being bound by any theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some aspects, the molar ratio of the cationic lipid to the neutral lipid ranges from or from about 2:1 to about 8:1, or from or from about 10:0 to about 1:9, about 4:1 to about 1:2, or about 3:1 to about 1:1.

In some aspects, the non-cationic lipid, e.g., neutral lipid (e.g., one or more phospholipids and/or cholesterol), may comprise from or from about 0 mol % to about 90 mol %, from or from about 0 mol % to about 80 mol %, from or from about 0 mol % to about 70 mol %, from or from about 0 mol % to about 60 mol %, or from or from about 0 mol % to about 50 mol %, of the total lipid present in the particle. In some aspects, the non-cationic lipid, e.g., neutral lipid (e.g., one or more phospholipids and/or cholesterol), may or may not be at least, at most, exactly, or between (inclusive or exclusive) of 0 mol %, 10 mol %, 20 mol %, 30 mol %, 40 mol %, 50 mol %, 60 mol %, 70 mol %, 80 mol %, or 90 mol % of the total lipid present in the particle.

### Pharmaceutical Compositions

In another embodiment, the invention comprises pharmaceutical compositions. For pharmaceutical composition purposes, the compound per se or pharmaceutically acceptable salt thereof will simply be referred to as the compounds of the invention.

A “pharmaceutical composition” refers to a mixture of one or more of the compounds of the invention, or a pharmaceutically acceptable salt, solvate, hydrate or prodrug thereof as an active ingredient, and at least one pharmaceutically acceptable excipient.

The term ‘excipient’ is used herein to describe any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The compositions of this invention may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dosage form is sub-lingual, such as, for example, a lozenge. In such solid dosage forms, the compounds of the invention are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the invention comprises a parenteral dosage form. “Parenteral administration” includes, for example, subcutaneous injections, intravenous injections, intraperitoneally, intramuscular injections, intrasternal injections, and infusion. Injectable preparations (e.g., sterile injectable aqueous or oleaginous suspensions) may be formulated according to the known art using one or more of suitable dispersing, wetting agents, or suspending agents.

In another embodiment, the invention comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of the invention are administered by a transdermal device, administration may be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated-see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (e.g., absorbable gel sponges, collagen) and non-biodegradable (e.g., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250 or 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.01 to about 10 mg/kg/minute during a constant rate infusion.

Liposome containing compounds of the invention may be prepared by methods known in the art (see, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly (2-hydroxyethyl-methacrylate), or poly (vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the invention with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the invention may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the invention isolated and stabilized in the amorphous state (for example, amorphous solid dispersions (ASDs)). There are many techniques known in the art to manufacture ASDs that produce material suitable for integration into a bulk drug product, for example, spray dried dispersions (SDDs), melt extrudates (often referred to as HMEs), co-precipitates, amorphous drug nanoparticles, and nano-adsorbates. In one embodiment amorphous solid dispersions comprise a compound of the invention and a polymer excipient. Other excipients as well as concentrations of the excipients and the compound of the invention are well known in the art and are described in standard textbooks. See, for example, “Amorphous Solid Dispersions Theory and Practice” by Navnit Shah et al.

“Systemic delivery,” as used herein, refers to delivery of a therapeutic product that can result in a broad exposure of an active agent within an organism. Some techniques of administration can lead to the systemic delivery of certain agents, but not others. Systemic delivery means that a useful, preferably therapeutic, amount of an agent is exposed to most parts of the body. Systemic delivery of lipid nanoparticles can be by any means known in the art including, for example, intravenous, intraarterial, subcutaneous, and intraperitoneal delivery. In some embodiments, systemic delivery of lipid nanoparticles is by intravenous delivery.

“Local delivery,” as used herein, refers to delivery of an active agent directly to a target site within an organism. For example, an agent can be locally delivered by direct injection into a disease site such as a tumor, other target site such as a site of inflammation, or a target organ such as the liver, heart, pancreas, kidney, and the like. Local delivery can also include topical applications or localized injection techniques such as intramuscular, subcutaneous or intradermal injection. Local delivery does not preclude a systemic pharmacological effect.

### Administration and Dosing

The term “treating”, “treat” or “treatment” as used herein embraces both preventative, e.g., prophylactic, and palliative treatment, e.g., relieve, alleviate, or slow the progression of the patient's disease (or condition) or any tissue damage associated with the disease.

As used herein, the terms, “subject, “individual” or “patient,” used interchangeably, refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” or “effective amount” refers to the amount of active compound or pharmaceutical agent, such as a nucleic acid, that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (e.g., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (e.g., reversing the pathology or symptomatology or both).

An “effective amount” or “therapeutically effective amount” of nucleic acid is an amount sufficient to produce the desired effect, e.g., an increase or inhibition of expression of a target sequence in comparison to the normal expression level detected in the absence of the nucleic acid. An increase in expression of a target sequence is achieved when any measurable level is detected in the case of an expression product that is not present in the absence of the nucleic acid. In the case where the expression product is present at some level prior to contact with the nucleic acid, an in increase in expression is achieved when the fold increase in value obtained with a nucleic acid such as mRNA relative to control is about 1.05, 1.1, 1.2, 1.3, 1.4, 1.5, 1.75, 2, 2.5, 3, 4, 5, 6, 7, 8, 9, 10, 15, 20, 25, 30, 40, 50, 75, 100, 250, 500, 750, 1000, 5000, 10000 or greater. Inhibition of expression of a target gene or target sequence is achieved when the value obtained with a nucleic acid such as antisense oligonucleotide relative to the control is about 95%, 90%, 85%, 80%, 75%, 70%, 65%, 60%, 55%, 50%, 45%, 40%, 35%, 30%, 25%, 20%, 15%, 10%, 5%, or 0%. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, fluorescence or luminescence of suitable reporter proteins, as well as phenotypic assays known to those of skill in the art.

The phrase “induce expression of a desired protein” refers to the ability of a nucleic acid to increase expression of the desired protein. To examine the extent of protein expression, a test sample (e.g., a sample of cells in culture expressing the desired protein) or a test mammal (e.g., a mammal such as a human or an animal model such as a rodent (e.g., mouse) or a non-human primate (e.g., monkey) model) is contacted with a nucleic acid (e.g., nucleic acid in combination with a lipid of the present invention). Expression of the desired protein in the test sample or test animal is compared to expression of the desired protein in a control sample (e.g., a sample of cells in culture expressing the desired protein) or a control mammal (e.g., a mammal such as a human or an animal model such as a rodent (e.g., mouse) or non-human primate (e.g., monkey) model) that is not contacted with or administered the nucleic acid. When the desired protein is present in a control sample or a control mammal, the expression of a desired protein in a control sample or a control mammal may be assigned a value of 1.0. In particular embodiments, inducing expression of a desired protein is achieved when the ratio of desired protein expression in the test sample or the test mammal to the level of desired protein expression in the control sample or the control mammal is greater than 1, for example, about 1.1, 1.5, 2.0. 5.0 or 10.0. When a desired protein is not present in a control sample or a control mammal, inducing expression of a desired protein is achieved when any measurable level of the desired protein in the test sample or the test mammal is detected. One of ordinary skill in the art will understand appropriate assays to determine the level of protein expression in a sample, for example dot blots, northern blots, in situ hybridization, ELISA, immunoprecipitation, enzyme function, and phenotypic assays, or assays based on reporter proteins that can produce fluorescence or luminescence under appropriate conditions.

The phrase “inhibiting expression of a target gene” refers to the ability of a nucleic acid to silence, reduce, or inhibit the expression of a target gene. To examine the extent of gene silencing, a test sample (e.g., a sample of cells in culture expressing the target gene) or a test mammal (e.g., a mammal such as a human or an animal model such as a rodent (e.g., mouse) or a non-human primate (e.g., monkey) model) is contacted with a nucleic acid that silences, reduces, or inhibits expression of the target gene. Expression of the target gene in the test sample or test animal is compared to expression of the target gene in a control sample (e.g., a sample of cells in culture expressing the target gene) or a control mammal (e.g., a mammal such as a human or an animal model such as a rodent (e.g., mouse) or non-human primate (e.g., monkey) model) that is not contacted with or administered the nucleic acid. The expression of the target gene in a control sample or a control mammal may be assigned a value of 100%. In particular embodiments, silencing, inhibition, or reduction of expression of a target gene is achieved when the level of target gene expression in the test sample or the test mammal relative to the level of target gene expression in the control sample or the control mammal is about 95%, 90%, 85%, 80%, 75%, 70%, 65%, 60%, 55%, 50%, 45%, 40%, 35%, 30%, 25%, 20%, 15%, 10%, 5%, or 0%. In other words, the nucleic acids are capable of silencing, reducing, or inhibiting the expression of a target gene by at least about 5%, 10%, 15%, 20%, 25%, 30%, 35%, 40%, 45%, 50%, 55%, 60%, 65%, 70%, 75%, 80%, 85%, 90%, 95%, or 100% in a test sample or a test mammal relative to the level of target gene expression in a control sample or a control mammal not contacted with or administered the nucleic acid. Suitable assays for determining the level of target gene expression include, without limitation, examination of protein or mRNA levels using techniques known to those of skill in the art, such as, e.g., dot blots, northern blots, in situ hybridization, ELISA, immunoprecipitation, enzyme function, as well as phenotypic assays known to those of skill in the art.

Typically, a compound of the invention is administered in an amount effective to treat a condition as described herein or to deliver an active agent to treat a condition as described herein. The compounds of the invention may be administered as compound per se, or alternatively, as a pharmaceutically acceptable salt. For administration and dosing purposes, the compound per se or pharmaceutically acceptable salt thereof will simply be referred to as the compounds of the invention.

The compounds of the invention are administered by any suitable route in the form of a pharmaceutical composition adapted to such a route, and in a dose effective for the treatment intended. The compounds of the invention may be administered orally, rectally, vaginally, parenterally, topically, intranasally, or by inhalation.

The compounds of the invention may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention may also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention or compositions containing the compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the invention is typically from about 0.01 to about 100 mg/kg (e.g., mg compound of the invention per kg body weight) for the treatment of the indicated conditions discussed herein or to deliver an active agent by using the compounds of the invention. In another embodiment, total daily dose of the compound of the invention is from about 0.00001 to about 50 mg/kg, and in another embodiment, from about 0.0001 to about 30 mg/kg. It is not uncommon that the administration of the compounds of the invention will be repeated a plurality of times in a day (typically no greater than 4 times). Multiple doses per day typically may be used to increase the total daily dose, if desired.

Therapeutic Methods and Uses The compounds of the invention may be useful for treating or preventing a disease, disorder, or condition or delivering an active agent to treat or prevent a disease, disorder, or condition. In particular, such compositions may be useful in treating or preventing a disease, disorder, or condition characterized by missing or aberrant protein or polypeptide activity. Diseases, disorders, and/or conditions characterized by dysfunctional or aberrant protein or polypeptide activity for which a composition may be administered include, but are not limited to rare diseases, infectious diseases (as both vaccines and therapeutics), cancer and proliferative diseases, genetic diseases, autoimmune diseases, diabetes, neurodegenerative diseases, cardio- and reno-vascular diseases, and metabolic diseases.

### Co-Administration

The compounds of the invention may be used alone, or in combination with one or more other therapeutic agents. The invention provides any of the uses, methods or compositions as defined herein wherein the compound of the invention, or pharmaceutically acceptable salt thereof, is used in combination with one or more other therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the invention and the one or more other therapeutic agents may be administered as a fixed or non-fixed combination of the active ingredients. The term “fixed combination” means a compound of the invention, or a pharmaceutically acceptable salt thereof, and the one or more therapeutic agents, are both administered to a subject simultaneously in a single composition or dosage. The term “non-fixed combination” means that a compound of the invention, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

These agents and compounds of the invention may be combined with pharmaceutically acceptable vehicles such as saline, Ringer's solution, dextrose solution, and the like. The particular dosage regimen, e.g., dose, timing and repetition, will depend on the particular individual and that individual's medical history.

### Kits

Another aspect of the invention provides kits comprising the compound of the invention or pharmaceutical compositions comprising the compound of the invention. A kit may include, in addition to the compound of the invention or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent.

In yet another embodiment, the invention comprises kits that are suitable for use in performing the methods of treatment described herein. In one embodiment, the kit contains a first dosage form comprising one or more of the compounds of the invention in quantities sufficient to carry out the methods of the invention. In another embodiment, the kit comprises one or more compounds of the invention in quantities sufficient to carry out the methods of the invention and a container for the dosage.

### Synthetic Methods

Compounds of the present invention may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the invention. It will be further appreciated that it may be necessary or desirable to carry out the transformations in a different order from that described in the schemes, or to modify one or more of the transformations, to provide the desired compound of the invention.

In the preparation of compounds of the invention it is noted that some of the preparation methods useful for the preparation of the compounds described herein may require protection of remote functionality (e.g., a primary amine, secondary amine, carboxyl, etc. in a precursor of a compound of the invention). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition; or Green's Protective Groups in Organic Synthesis, 5th Edition, Wuts, P.G.M. Ed.

For example, if a compound contains an amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the invention.

### General Experimental Details

In the non-limiting Examples and Preparations that illustrate the invention and that are set out in the description, and in the following Schemes, the following abbreviations, definitions and analytical procedures may be referred to:

¹H NMR spectra were recorded on a Bruker 400 MHz spectrometer. The chemical shifts are reported in parts per million (ppm) and all spectra are referenced to their residual non-deuterated solvent peaks as follows: CHCl₃ (7.26 ppm), CD₃OD (3.31 ppm), DMSO-d6 (2.50 ppm), D₂O (4.75 ppm). Coupling constants (J) are reported to the nearest 0.1 Hz. Multiplicities are reported as follow: singlet(s), doublet (d), triplet (t), quartet (q), multiplet (m), and broad singlet (br s). Exchangeable protons are not always observed.

LCMS data were acquired on an Agilent Prime-6125B instrument, Agilent Poroshell 120 EC-C18 3.0×30 mm column, 2.7 μm, acetonitrile/water gradients with TFA modifiers, using evaporative light scattering detector (ELSD) (see method A and B). Preparative chiral supercritical fluid chromatography (prep-SFC) was performed using DAICEL ChiralPAK-AD, -AS, -IC, DAICEL ChiraICEL-OJ, or -OD columns; gradient eluting with CO₂ mixtures with 0.1% NH₃H₂O in EtOH and UV detection was used to trigger fraction collection. The LCMS-ELSD purity was verified by the following analytical methods and noted as area %: instrument=Agilent 1260 Infinity with 6150 MSD; Column=Waters XBridge C8 100×2.1 mm, 3.5 μm; Mobile phase A, 0.05% DFA in water; Mobile phase B, 0.05% DFA in acetonitrile; Gradient=50%-100% (solvent B) over 5 minutes and holding at 100% for 2 minutes at a flow rate of 1.0 mL/min, total time of 7.0 min, 40° C. The LC-CAD purity was verified by the following analytical methods and noted as area %: instrument=Thermo Vanquish F; Column=Waters XBridge C8 4.6×150 mm, 3.5 μm; Mobile phase A, 1 L water+0.05% TFA; Mobile phase B, 1 L acetonitrile+0.05% TFA; Gradient=50%-100% (solvent B) over 10 minutes and holding at 100% for 5 minutes at a flow rate of 1.0 mL/min, total time of 15.0 min, 40° C. The charged aerosol detection (CAD) data collection rate: 10 Hz; evaporator temperature: 35° C.

The LCMS-ELSD methods used to monitor the reactions:
Method A: Analytical LCMS data collected on instrument=Agilent Prime-6125B; Column=Agilent Poroshell 120 EC-C18 3.0×30 mm, 2.7 μm; Mobile phase A, water (4 L)+TFA (1.5 mL); Mobile phase B, acetonitrile (4 L)+TFA (0.75 mL); Gradient=5%-95% (solvent B) over 0.4 minutes and holding at 100% for 0.3 minutes at a flow rate of 2.0 mL/min, total time of 1.0 min, 50° C.
Method B: Analytical LCMS data collected on instrument=Agilent Prime-6125B; Column=Agilent Poroshell 120 EC-C18 3.0×30 mm, 2.7 μm; Mobile phase A, water (4 L)+TFA (1.5 mL); Mobile phase B, acetonitrile (4 L)+TFA (0.75 mL); Gradient=95%-100% (solvent B) over 0.4 minutes and holding at 100% for 0.3 minutes at a flow rate of 2.0 mL/min, total time of 1.0 min, 50° C.

### Abbreviations

° 2θ is degrees 2-theta;
AcCl is acetyl chloride;
AcOH is acetic acid;
APCI is atmospheric pressure chemical ionization;
aq is aqueous;
Bn is benzyl;
Boc is tert-butoxycarbonyl;
Boc₂O is di-tert-butyl dicarbonate;
br is broad;
tBu is tert-butyl;
tBuOH is tert-butanol;
tBuOK is potassium tert-butoxide;
° C. is degrees celcius;
CDCl₃ is deutero-chloroform;
CD₃OD or MeOD_d₄ is deuterated methanol;
CDI is 1,1′-carbonyldiimidazole;
is chemical shift;
d is doublet;
dd is doublet of doublets;
ddd is doublet of doublet of doublets;
dt is doublet of triplets;
DCE is 1,2-dichloroethane;
DCM is dichloromethane; methylene chloride;
DIAD is diisopropyl azodicarboxylate;
DIPEA is N-ethyldiisopropylamine, also known as N,N-diisopropylethylamine;
DMA is N,N-dimethylacetamide;
DME is 1,2-dimethoxyethane;
DMAP is 4-dimethylaminopyridine;
DMF is N, N-dimethylformamide;
DMSO is dimethyl sulfoxide;
DMSO-d₆ is deuterodimethylsulfoxide;
EDC is N-(3-dimethylaminopropyl)-N′-ethylcarbodiimide;
EDC·HCl is N-(3-dimethylaminopropyl)-N′-ethylcarbodiimide hydrochloride;
ESI is electrospray ionization;
Et₂O is diethyl ether;
EtOAc is ethyl acetate;
EtOH is ethanol;
Et₃N is triethylamine;
g is gram;
HATU is 1-[bis(dimethylamino)methylene]-1H-1,2,3-triazolo[4,5-b]pyridinium 3-oxid hexafluorophosphate;
HPLC is high pressure liquid chromatography;
HOBt is 1-hydroxybenzotriazole hydrate;
hr(s) is hour(s);
IPA is isopropyl alcohol;
iPrOAc is isopropyl acetate;
L is liter;
LCMS is liquid chromatography mass spectrometry;
m is multiplet;
M is molar;
m-CPBA is 3-chloroperbenzoic acid;
MeCN is acetonitrile;
MeMgBr is methylmagnesium bromide;
MeNHOMe HCl is N, O-dimethylhydroxylamine hydrochloride;
MeOH is methanol;
2-MeTHF is 2-methyl tetrahydrofuran;
mg is milligram;
MHz is mega Hertz;
min(s) is minute(s);
mL is milliliter;
mmol is millimole;
mol is mole;
MS (m/z) is mass spectrum peak;
MsCl is mesyl chloride;
MTBE is tert-butyl methyl ether;
NMR is nuclear magnetic resonance;
Pd/C is palladium on carbon;
Pd₂(dba)₃ is palladium tris(dibenzylideneacetone)dipalladium(0);
Pd(dppf)Cl₂ is [1,1′-bis(diphenylphophino)ferrocene]dichloropalladium(II);
Pd(PPh₃)₄ is tetrakis(triphenylphosphine)palladium(0);
Pet. ether is the petroleum fraction consisting of aliphatic hydrocarbons and boiling in the range 35-60° C.;
PMB is para-methoxybenzyl;
PMB-NH₂ is para-methoxybenzylamine;
PPh₃ is triphenylphosphine;
pH is power of hydrogen;
ppm is parts per million;
PSD is position sensitive detector;
psi is pounds per square inch;
PXRD is powder X-ray diffraction;
q is quartet;
rt is room temperature;
RT is retention time;
s is singlet;
SEM-Cl is 2-(trimethylsilyl) ethoxymethyl chloride;
SFC is supercritical fluid chromatography;
t is triplet;
TBAF is tert-butyl ammonium fluoride;
TBDMSCl is tert-butyldimethylsilyl chloride;
TFA is trifluoroacetic acid;
THF is tetrahydrofuran;
TLC is thin layer chromatography;
TMEDA is N,N,N′N′-tetramethylethylenediamine;
TMSCl is trimethylsilyl chloride;
TMSCN is trimethylsilyl cyanide;
TMSCHN₂ is (diazomethyl) trimethylsilane;
TsCl is p-toluenesulfonyl chloride;
Ts₂O is p-toluenesulfonic anhydride;
μL is microliter;
μmol is micromole; and

The Schemes described below are intended to provide a general description of the methodology employed in the preparation of the compounds of the present invention. Some of the compounds of the present invention contain one or more chiral centers. In the following Schemes, the general methods for the preparation of the compounds are shown either in racemic or enantioenriched form. It will be apparent to one skilled in the art that all of the synthetic transformations may be conducted in a precisely similar manner whether the materials are enantioenriched or racemic. Moreover, the resolution to the desired optically active material may take place at any desired point in the sequence using well known methods such as described herein and in the chemistry literature.

### General Methods:

Unless stated otherwise, the variables in Schemes A-K have the same meanings as defined herein.

In some cases, compounds described in General Schemes A-J or having Formula I, I (a), I(b) or I(c) may contain protecting groups, which may be appended or removed by additional steps in the synthetic sequence using conditions known in the art (March's Advanced Organic Chemistry: Reactions, Mechanisms, and Structure 8th Edition or Green's Protective Groups in Organic Synthesis, 5th Edition, Wuts, P.G.M. Ed.). Compounds at every step may be purified by standard techniques, such as column chromatography, crystallization, or reverse phase SFC or HPLC. Variables m, n, o, p, s ort are as defined in the embodiments, schemes, examples, and claims herein.

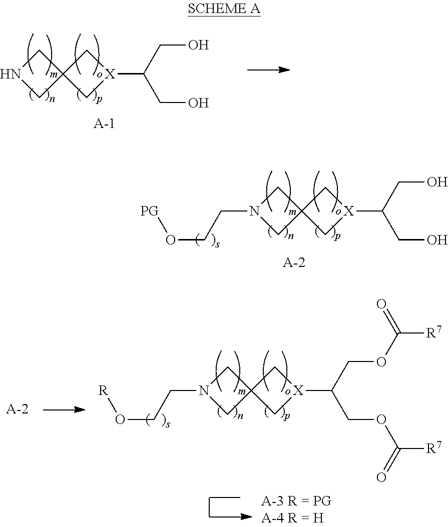

Compounds of Formula A-4 can be prepared from spirocyclic amino diols comprised of the general structure A-1 as described in Scheme A, where X can be either carbon or nitrogen. The amine can be alkylated with an appropriate alkyl halide (or an equivalent alkylating agent such as an alkyl sulfonate) bearing a pendant protected hydroxyl group to deliver compounds of Formula A-2 (s=1-11). The free hydroxyl groups can be acylated using an acid chloride or carboxylic acid to install the additional molecular framework and cleavage of the alcohol protecting group delivers compounds of Formula A-4.

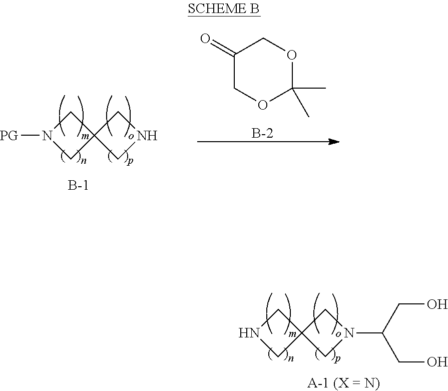

Compounds of Formula A-1 (X═N) which are employed in Scheme A can be prepared from readily available spirocyclic diamines with the general Formula B-1 as described in Scheme B. Reductive amination with commercially available protected dihydroxy acetone (B-2) followed by acid hydrolysis delivers the diol. When an acid-sensitive nitrogen protecting group is not used, an additional deprotection step can deliver compounds of general Formula A-1 (where X═N).

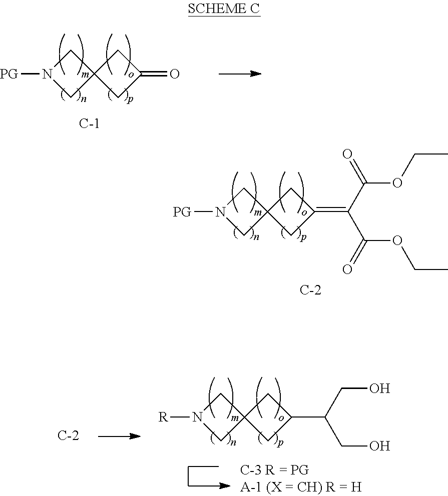

Alternatively, compounds of Formula A-1 which are employed in Scheme A (where X═CH) can be prepared from spirocyclic amino ketones of general Formula C-1 as described in Scheme C. A homologation to deliver the vinyl diester (C-2) followed by reduction of the esters and the olefin can deliver compounds of Formula C-3. Removal of the nitrogen protecting group affords compounds of Formula A-1 (where X═CH).

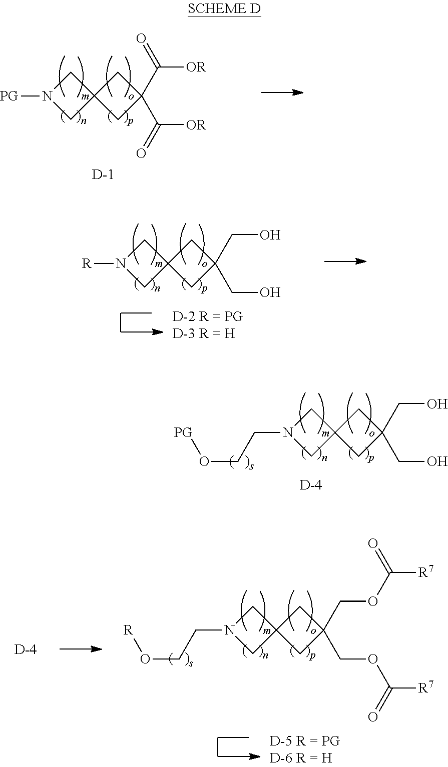

Compounds of Formula D-6 can be prepared from spirocyclic amino diesters of general Formula D-1 as shown in Scheme D. Reduction of the esters generates the diols of Formula D-2, whereupon deprotection provides amino diols of general Formula D-3. The amine can be alkylated with an appropriate alkyl halide (or an equivalent alkylating agent such as an alkyl sulfonate) bearing a pendant protected hydroxyl group to deliver compounds of Formula D-4 (s=1-11). The free hydroxyl groups can be acylated using an acid chloride or carboxylic acid to install the additional molecular framework and cleavage of the alcohol protecting group delivers compounds of Formula D-6 (s=1-11).

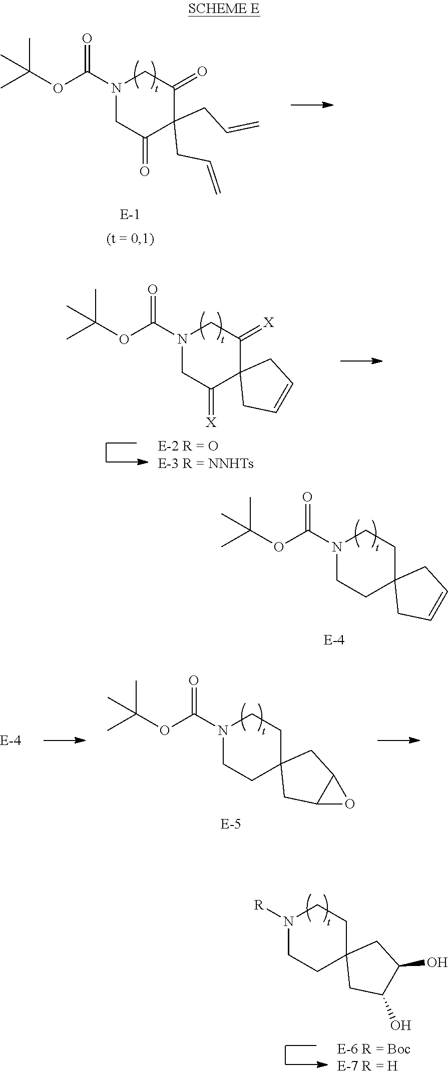

Compounds of general Formula E-7 can be prepared from readily available dienes of Formula E-1 (t=0-1) as shown in Scheme E. Compounds of general Formula E-1 can be prepared by bis-allylation of a 1,3-dione as described in Tetrahedron 2015, 71, 129; Tetrahedron Lett, 2011, 52, 4204. Spirocyclic amino olefins of Formula E-2 can be readily prepared by ring closing metathesis of dienes. Subsequent reduction of the dione to the alkanes can afford amino spirocycles of general Formula E-4. Epoxidation of the cyclic olefin followed by hydrolytic ring opening can provide spirocyclic diols with general structure E-6. Final cleavage of the nitrogen protecting group liberates spirocyclic amino diols with general Formula E-7 (t=0-1).

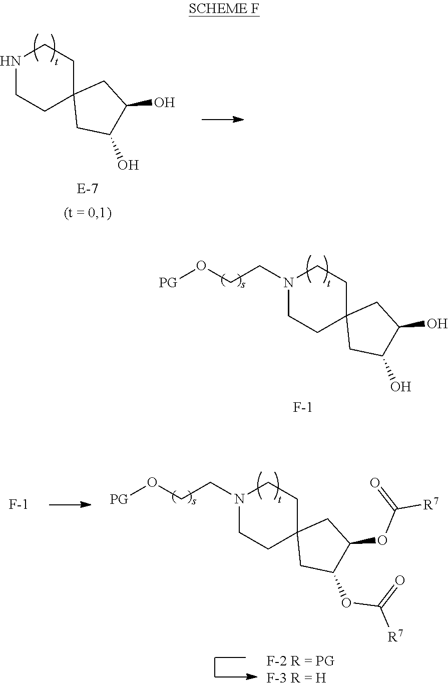

Compounds with general structure F-3 can be readily prepared from spirocyclic amino diols of Formula E-7 (t=0-1) by the methods shown in Scheme F. The amine can be alkylated with an appropriate alkyl halide (or an equivalent alkylating agent such as an alkyl sulfonate) bearing a pendant protected hydroxyl group to deliver compounds of Formula F-1 (s=1-11). The free hydroxyl groups can be acylated using an acid chloride or carboxylic acid to install the additional molecular framework and cleavage of the alcohol protecting group delivers compounds of Formula F-3 (s=1-11, t=0-1).

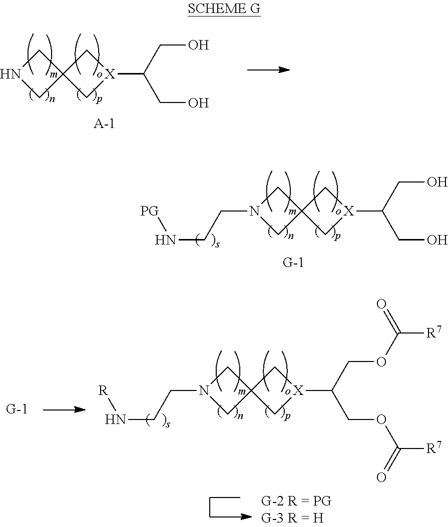

Compounds with general structure G-3 can be readily prepared from spirocyclic amino diols of Formula A-1 by the methods shown in Scheme G. The amine can be alkylated with an appropriate alkyl halide (or an equivalent alkylating agent such as an alkyl sulfonate) bearing a pendant protected nitrogen to deliver compounds of Formula G-1 (s=1-11). The free hydroxyl groups of the diol can be acylated using an acid chloride or carboxylic acid to install the additional molecular framework and cleavage of the nitrogen protecting group delivers compounds of Formula G-3 (s=1-11).

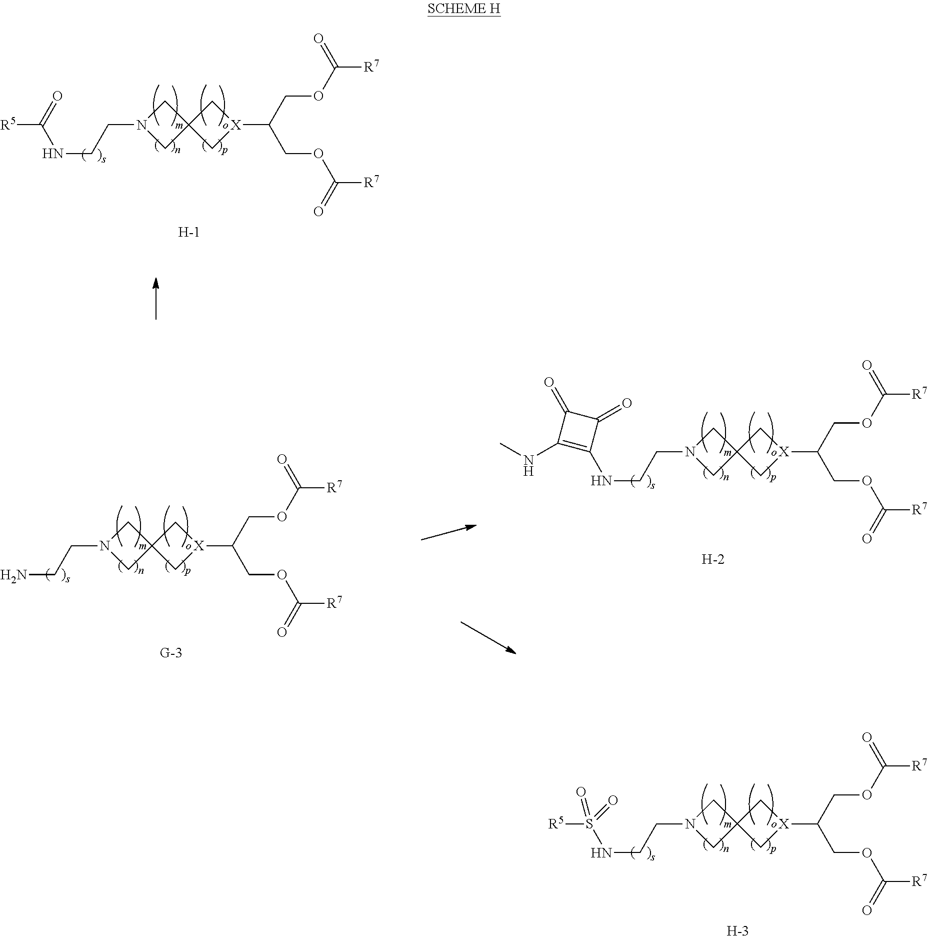

Additional compounds of general Formula (I) may be prepared according to Scheme H wherein (s=1-11). Compounds of the formula G-3 can be reacted with carboxylic acids or acid chlorides to afford compounds of the formula H-1. Compounds of formula H-2 may be prepared by a displacement reaction between compounds of the formula G-3 and commercially available 3-methoxy-4-(methylamino) cyclobut-3-ene-1,2-dione. Compounds of the formula G-3 may also be reacted with sulfonyl chlorides to provide compounds of the general formula H-3.

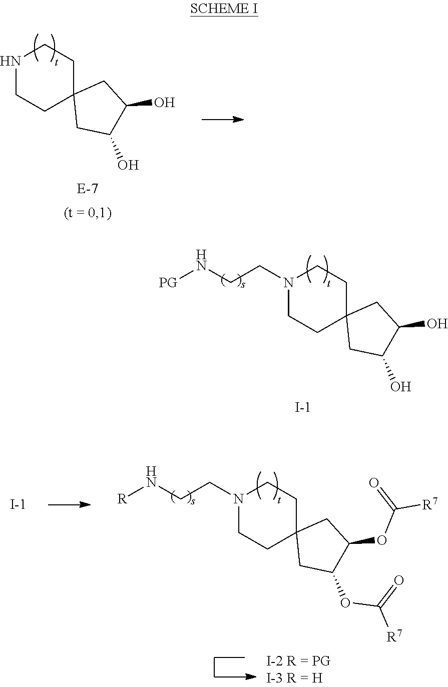

Compounds with general structure I-3 can be readily prepared from spirocyclic amino diols of Formula E-7 (t=0-1) by the methods shown in Scheme I. An amine can be alkylated with an appropriate alkyl halide (or an equivalent alkylating agent such as an alkyl sulfonate) bearing a pendant protected nitrogen to deliver compounds of Formula 1-1 (s=1-11). The free hydroxyl groups of the diol can be acylated using an acid chloride or carboxylic acid to install the additional molecular framework and subsequent cleavage of the nitrogen protecting group delivers compounds of Formula 1-3 (s=1-11, t=0-1).

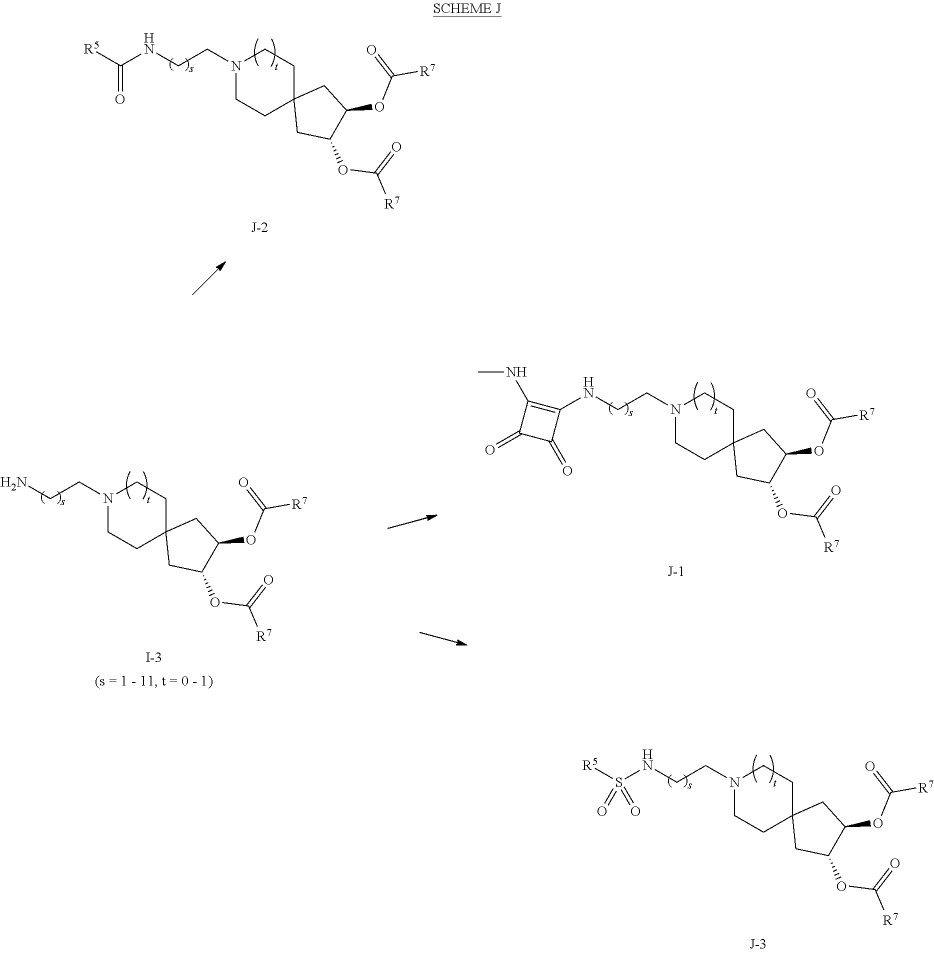

Additional compounds of general Formula (I) may be prepared according to Scheme J wherein (s=1-11, t=0-1). Compounds of general Formula J-1 can be prepared by a displacement reaction between 1-3 and commercially available 3-methoxy-4-(methylamino) cyclobut-3-ene-1,2-dione. Compounds of the formula 1-3 can be reacted with carboxylic acids or acid chlorides to afford compounds of the formula J-2. Compounds of the formula 1-3 may also be reacted with sulfonyl chlorides to provide compounds of the general formula J-3.

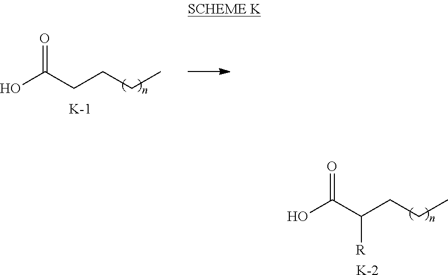

Carboxylic acids of general structure R⁷CO₂H may be obtained from commercial sources (eg. 2-octyldecanoic acid, 2-heptylnonanoic acid, and 2-hexyldecanoic acid), may be prepared by procedures known in the literature, or may be prepared as described in Scheme K wherein n=1-20. Carboxylic acids of the formula K-1 which may be obtained from commercial sources or may be prepared by procedures known in the literature are sequentially reacted with 1 equivalent of NaH followed by an additional strong based such as LDA. The resulting dianion is then reacted with an alkyl halide RX (where X═I or Br: R═C₁₋₈ alkyl, C₃₋₈ cycloalkyl, and C₁₋₆ alkyl substituted by C₃₋₈ cycloalkyl) to afford carboxylic acids of the formula K-2. The required alkyl halide RX may be obtained from commercial sources or may be prepared by procedures known in the literature.

### EXAMPLES

In order that this invention may be better understood, the following examples are set forth. These examples are for purposes of illustration only and are not to be construed as limiting the scope of the invention in any manner.

The compounds and intermediates described below were named using the naming convention provided with ChemDraw, Version 20.1.1.125 (Perkin Elmer). The naming convention provided with ChemDraw, Version 20.1.1.125 is well known by those skilled in the art and it is believed that the naming convention provided with ChemDraw, Version 20.1.1.125 generally comports with the IUPAC (International Union for Pure and Applied Chemistry) recommendations on Nomenclature of Organic Chemistry or the CAS Index rules. Unless noted otherwise, all reactants were obtained commercially without further purifications or were prepared using methods known in the literature.

### Synthesis of Carboxylic Acid Tails

### A1

### 7-oxo-7-(pentadecan-8-yloxy)heptanoic acid

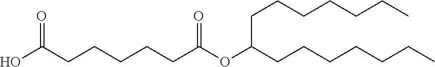 [SMILES: CCCCCCCC(CCCCCCC)OC(=O)CCCCCC(=O)O]

Pentadecan-8-ol (4.28 g, 18.7 mmol) and EDCl (3.95 g, 20.6 mmol) was added to a solution of heptanedioic acid (3.0 g, 18.7 mmol) in DCM (208 mL). DMAP (343 mg, 2.81 mmol) was added, and the reaction mixture was stirred at 45° C. After 30 hours, the mixture was partitioned between saturated NaHCO₃ and DCM. The organic layer was dried over Na₂SO₄ and concentrated in vacuo. The residue was purified by silica gel chromatography to provide 7-oxo-7-(pentadecan-8-yloxy) heptanoic acid (2.07 g, 30% yield) as a colorless oil. ¹H NMR (400 MHZ, CDCl₃) δ 4.88-4.85 (m, 1H), 2.37 (t, J=7.5 Hz, 2H), 2.30 (t, J=7.5 Hz, 2H), 1.66 (h, J=7.4 Hz, 4H), 1.55-1.51 (m, 4H), 1.43-1.37 (m, 2H), 1.26 (m, 20H), 0.87 (t, J=6.7 Hz, 6H).

### A2

### 4-oxo-4-(pentadecan-8-yloxy)butanoic acid

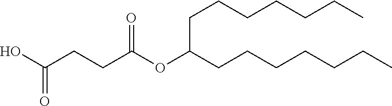 [SMILES: CCCCCCCC(CCCCCCC)OC(=O)CCC(=O)O]

DMAP (244 mg, 2.0 mmol) and pentadecan-8-ol (2.74 g, 12.0 mmol) were added to a solution of dihydrofuran-2,5-dione (1.0 g, 10.0 mmol) in toluene (20.0 mL). Triethylamine (303 mg, 3.0 mmol) was added, and the mixture was stirred at 110° C. for 16 hours. The reaction mixture was cooled to room temperature, filtered, and concentrated in vacuo. The residue was extracted with DCM. The combined organic layers were dried over Na₂SO₄ and filtered. The filtrate was concentrated in vacuo. The crude product was purified by silica gel chromatography to provide 4-oxo-4-(pentadecan-8-yloxy) butanoic acid (1.20 g, 73% yield) as a white solid. ¹H NMR (400 MHZ, CDCl₃) δ 4.89 (p, J=6.3 Hz, 1H), 2.73-2.57 (m, 4H), 1.52-1.49 (m, 4H), 1.27 (m, 21H), 0.88 (t, J=6.8 Hz, 6H).

### A3

### 6-((2-heptylnonanoyl)oxy)hexanoic acid

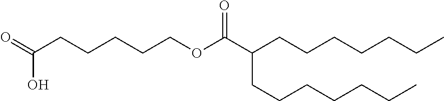 [SMILES: CCCCCCCC(CCCCCCC)C(=O)OCCCCCC(=O)O]

### Step 1: 6-(tert-butoxy)-6-oxohexyl 2-heptylnonanoate

DCC (9.13 g, 44.3 mmol) was added to a solution of tert-butyl 6-hydroxyhexanoate (5.0 g, 26.6 mmol), 2-heptylnonanoic acid (A12) (5.68 g, 22.1 mmol), and DMAP (1.35 g, 11.1 mmol) in dichloroethane (100 mL). The reaction mixture was stirred at 85° C. for 16 hours. After that time the mixture was filtered to remove solids (with the aid of pet ether). The filtrate was dried over Na₂SO₄ and concentrated in vacuo. The residue was purified by silica gel chromatography to afford 6-(tert-butoxy)-6-oxohexyl 2-heptylnonanoate (3.6 g, 32%) as a light-yellow oil. ¹H NMR (400 MHZ, CDCl₃) δ 4.06 (t, J=6.6 Hz, 2H), 2.37-2.25 (m, 1H), 2.22 (t, J=7.4 Hz, 2H), 1.69-1.51 (m, 6H), 1.44 (s, 9H), 1.40-1.35 (m, 4H), 1.29-1.20 (m, 20H), 0.87 (t, J=6.8 Hz, 6H).

### Step 2: 6-((2-heptylnonanoyl)oxy)hexanoic acid

Trifluoroacetic acid (967 mg, 8.48 mmol) was added to a solution of 6-(tert-butoxy)-6-oxohexyl 2-heptylnonanoate (3.62 g, 8.48 mmol) in DMC (25 mL), and the reaction mixture was stirred at room temperature for 2 hours. The mixture was concentrated, and the residue was purified by silica gel chromatography to afford 6-((2-heptylnonanoyl)oxy) hexanoic acid as a light-yellow oil. LCMS (ESI): Calc'd for C₂₂H₄₃O₄ [M+H]⁺ 371.3; found 371.4.

### Representative Procedure for the Synthesis of Carboxylic Acid Tails by Alkylation of Carboxylic Acids Using Alkyl Halides

### A4

### 2-(cyclobutylmethyl)decanoic acid

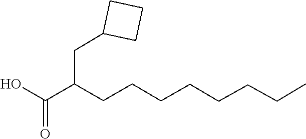 [SMILES: CCCCCCCCC(CC1CCC1)C(=O)O]

At 0° C., a solution of decanoic acid (A18) (30.0 g, 174 mmol) in THF (500.0 mL) was added dropwise to a solution of NaH (11.5 g, 287 mmol) in THF (500.0 mL). LDA (37.3 g, 348 mmol) was then added dropwise to the mixture. The mixture was stirred at room temperature for 2.5 hours resulting in a light-yellow suspension. To the suspension was added dropwise (bromomethyl) cyclobutene (38.9 g, 261 mmol). The yellow mixture changed to a white suspension immediately. The reaction mixture was stirred at 75° C. for another 14 hours. After stirring for the designating time, the reaction mixture was quenched with HCl (2N, 500 mL) resulting in a clear yellow solution. The organic layer was separated, and the aqueous layer was extracted with EtOAc (300 mL×5). The combined organic layers were dried over Na₂SO₄ and filtered. The filtrate was concentrated in vacuo, and the residue was purified by silica gel column chromatography to provide 2-(cyclobutylmethyl)decanoic acid (26 g, 63% yield) as a light-yellow oil. ¹H NMR (400 MHZ, CDCl₃) δ 2.40-2.20 (m, 2H), 2.12-1.99 (m, 2H), 1.94-1.72 (m, 3H), 1.69-1.53 (m, 5H), 1.51-1.40 (m, 1H), 1.36-1.26 (m, 12H), 0.90 (t, J=6.7 Hz, 3H).

The following Carboxylic Acid A5-A11 were prepared according to the representative procedure as described for A4.

TABLE 1

| | Structure | Analytical data | Alkyl halide |
|---|---|---|---|
| A5 | 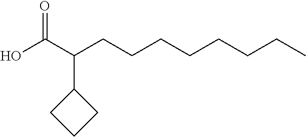 [SMILES: CCCCCCCCC(C(=O)O)C1CCC1] | ¹H NMR (400 MHz, CDCl₃) δ 2.54- 2.40 (m, 1H), 2.34 (td, J = 9.7, 4.2 Hz, 1H), 2.14-2.00 (m, 2H), 1.95- 1.65 (m, 4H), 1.57-1.38 (m, 2H), 1.35-1.22 (m, 13H), 0.92-0.83 (m, 3H). | 1-iodooctane |
| A6 | 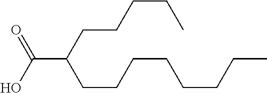 [SMILES: CCCCCCCCC(CCCCC)C(=O)O] | ¹H NMR (400 MHz, CDCl₃) δ 2.40- 2031 (m, 1H), 1.66-1.57 (m, 2H), 1.51-1.43 (m, 2H), 1.33-1.24 (m, 18H), 0.88 (t, J = 6.6 Hz, 6H) | 1-iodopentane |
| A7 | 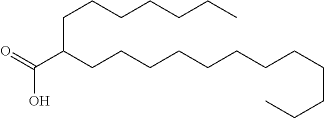 [SMILES: CCCCCCCCCCCCC(CCCCCCC)C(=O)O] | ¹H NMR (400 MHz, CDCl₃) δ 2.34 (tt, J = 8.7, 5.6 Hz, 1H), 1.67-1.54 (m, 2H), 1.53-1.41 (m, 2H), 1.35- 1.20 (m, 31H), 0.90-0.84 (m, 6H) | 1-iodododecane |
| A8 | 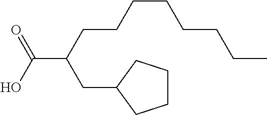 [SMILES: CCCCCCCCC(CC1CCCC1)C(=O)O] | ¹H NMR (400 MHz, CDCl₃) δ 2.46- 2.36 (m, 1H), 1.88-1.70 (m, 4H), 1.68-1.39 (m, 8H), 1.36-1.24 (m, 12H), 1.13-1.03 (m, 2H), 0.90 (t, J = 6.7 Hz, 3H) | (bromomethyl) cyclopentane |
| A9 | 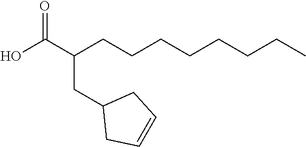 [SMILES: CCCCCCCCC(CC1CC=CC1)C(=O)O] | ¹H NMR (400 MHz, CD₃OD) δ 5.68-5.65 (m, 2H), 2.52-2.37 (m, 4H), 2.07-1.95 (m, 2H), 1.76-1.68 (m, 1H), 1.51-1.47 (m, 3H), 1.31 (m, 12H), 0.94-0.90 (m, 3H) | 4-(iodomethyl)- cyclopent-1-ene |
| A10 | 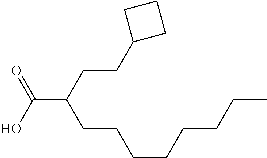 [SMILES: CCCCCCCCC(CCC1CCC1)C(=O)O] | ¹H NMR (400 MHz, CDCl₃) δ 2.38- 2.28 (m, 2H), 2.03-1.99 (m, 2H), 1.83-1.75 (m, 2H), 1.60-1.27 (m, 20H), 0.88 (m, 3H) | 1-iodooctane |
| A11 | 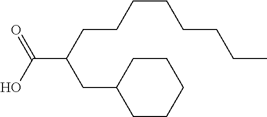 [SMILES: CCCCCCCCC(CC1CCCCC1)C(=O)O] | ¹H NMR (400 MHz, CDCl₃) δ 2.49- 2.42 (m, 1H), 1.83-1.07 (m, 27H), 1.72-1.00 (m, 24H), 0.87 (t, J = 6.7 Hz, 3H), LCMS cal'd for C₁₇H₃₃O₂ [M + H]⁺ 269.2 found 269.3. | 1-iodooctane |

The following Carboxylic Acids A12-A26 are either commercially available or readily prepared by one skilled in the art.

TABLE 2

| | Structure | CAS Registry Number |
|---|---|---|
| A12 | 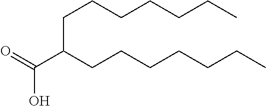 [SMILES: CCCCCCCC(CCCCCCC)C(=O)O] | 619-38-5 |
| A13 | 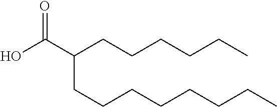 [SMILES: CCCCCCCCC(CCCCCC)C(=O)O] | 25354-97-6 |
| A14 | 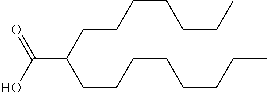 [SMILES: CCCCCCCCC(CCCCCCC)C(=O)O] | 53705-90-1 |
| A15 | 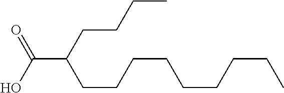 [SMILES: CCCCCCCCCC(CCCC)C(=O)O] | 66241-54-1 |
| A16 | 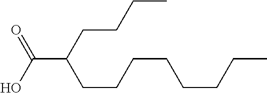 [SMILES: CCCCCCCCC(CCCC)C(=O)O] | 52304-09-3 |
| A17 | 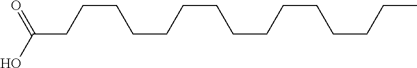 [SMILES: CCCCCCCCCCCCCCCC(=O)O] | 57-10-3 |
| A18 | 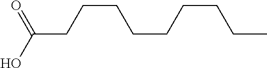 | 334-48-5 |
| A19 | 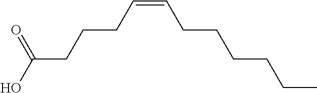 | 2430-94-6 |
| A20 | 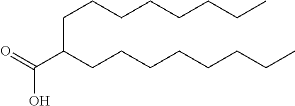 [SMILES: CCCCCCCCC(CCCCCCCC)C(=O)O] | 619-39-6 |
| A21 | 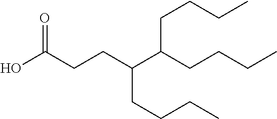 [SMILES: CCCCC(CCCC)C(CCCC)CCC(=O)O] | 2410382-83-9 |
| A22 | 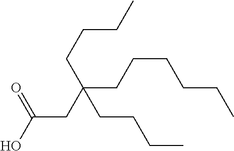 [SMILES: CCCCCCC(CCCC)(CCCC)CC(=O)O] | 318293-68-4 |
| A23 | 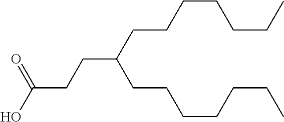 [SMILES: CCCCCCCC(CCCCCCC)CCC(=O)O] | 874516-59-3 |
| A24 | 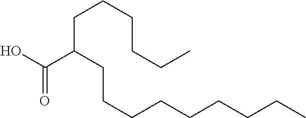 [SMILES: CCCCCCCCCC(CCCCCC)C(=O)O] | 72056-18-9 |
| A25 | 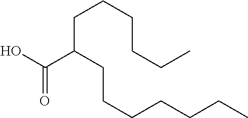 [SMILES: CCCCCCCC(CCCCCC)C(=O)O] | 37165-63-2 |
| A26 | 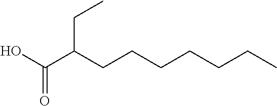 | 14276-84-7 |

### Example 1

### (3-(4-hydroxybutyl)-3-azaspiro[5.5]undecane-9,9-diyl)bis(methylene) bis(2-heptylnonanoate) (1)

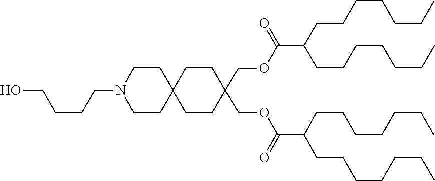 [SMILES: CCCCCCCC(CCCCCCC)C(=O)OCC1(COC(=O)C(CCCCCCC)CCCCCCC)CCC2(CCN(CCCCO)CC2)CC1]

### Step 1: tert-butyl 9,9-bis (hydroxymethyl)-3-azaspiro[5.5]undecane-3-carboxylate

To a solution of 3-(tert-butyl) 9,9-diethyl 3-azaspiro[5.5]undecane-3,9,9-tricarboxylate (6.0 g, 20 mmol) in EtOH (60 mL) at 0° C. was added NaBH₄ (3.43 g, 90.6 mmol) and the mixture was stirred at this temperature for 1 hour then heated to 60° C. and stirred for 8 hours. The solution was cooled to room temperature and slowly quenched with H₂O (10 mL) and acetone (20 mL). Then the mixture was stirred for 1 hour at room temperature resulting in a yellow precipitate. The solid was collected by filtration. The filter cake was washed with H₂O (3 mL×5) and dried to provide tert-butyl 9,9-bis(hydroxymethyl)-3-azaspiro[5.5]undecane-3-carboxylate (4.50 g, 90% yield) as a yellow solid. The crude product was used directly in next step without further purification. ¹H NMR (400 MHZ, CD₃OD) δ 3.48-3.45 (m, 4H), 3.38-3.34 (m, 4H), 1.45 (s, 9H), 1.42-1.36 (m, 12H). LCMS (ESI): Calc'd for C₁₃H₂₄NO₄ [M+H-tBu]⁺ 258.16, found 258.0.

### Step 2: (3-azaspiro[5.5]undecane-9,9-diyl)dimethanol

To a solution of tert-butyl 9,9-bis (hydroxymethyl)-3-azaspiro[5.5]undecane-3-carboxylate (4.50 g, 14.36 mmol) in DCM (20 mL) at room temperature was added HCl (4M/dioxane, 20 mL, 80 mmol). The solution was stirred at room temperature for 1 hour, the solvent was removed in vacuo, and the resulting solid was triturated with MTBE to provide (3-azaspiro[5.5]undecane-9,9-diyl) dimethanol (3.0 g, 98% yield) as a yellow solid. ¹H NMR (400 MHZ, CD₃OD) δ 3.46 (m, 4H), 3.16-3.14 (m, 4H), 1.69 (m, 4H), 1.50-1.36 (m, 8H). LCMS (ESI): Calc'd for C₁₂H₂₄NO₂ [M+H]^{+ 214.17}, found 214.1.

### Step 3: (3-(4-((tert-butyldimethylsilyl)oxy)butyl)-3-azaspiro[5.5]undecane-9,9-diyl)dimethanol

### General Alkylation Procedure

To a solution of (3-azaspiro[5.5]undecane-9,9-diyl)dimethanol (3.0 g, 10 mmol) in DMF (30 mL) and MeOH (5 mL) at room temperature was added 4-bromobutoxy-tert-butyl-dimethylsilane (4.51 g, 16.9 mmol), followed by K₂CO₃ (11.7 g, 84.4 mmol). The mixture was stirred at 70° C. for 6 hours, quenched with H₂O (25 mL), and extracted with EtOAc (40 mL×3). The organic layers were combined, washed with brine (20 mL×3) to remove DMF and concentrated in vacuo. The residual material was purified by column chromatography to give (3-(4-((tert-butyldimethylsilyl)oxy)butyl)-3-azaspiro[5.5]undecane-9,9-diyl)dimethanol (2.20 g, 40% yield) as a white solid. ¹H NMR (400 MHZ, CDCl₃) δ 3.59-3.56 (m, 6H), 2.67 (br s, 2H), 2.40 (br s, 5H), 1.66-1.40 (m, 8H), 1.31 (s, 8H), 0.84 (s, 9H), 0.00 (s, 6H). LCMS (ESI): Calc'd for C₂₂H₄₆NO₃Si [M+H]⁺ 400.32, found 400.2.

### Step 4: (3-(4-((tert-butyldimethylsilyl)oxy)butyl)-3-azaspiro[5.5]undecane-9,9-diyl)bis(methylene) bis(2-heptylnonanoate)

### General Acylation Procedure

To a solution of (3-(4-((tert-butyldimethylsilyl)oxy)butyl)-3-azaspiro[5.5]undecane-9,9-diyl) dimethanol (2.20 g, 5.50 mmol) in DCM (30 mL) was added 2-heptylnonanoic acid (A12) (2.89 g, 11.3 mmol), DCC (2.84 g, 13.8 mmol), followed by DMAP (202 mg, 1.65 mmol). The reaction mixture was heated to reflux and stirred for 40 hours. After this time the reaction mixture was filtered over celite and concentrated in vacuo. The residue was purified by silica gel column chromatography to give (3-(4-((tert-butyldimethylsilyl)oxy)butyl)-3-azaspiro[5.5]undecane-9,9-diyl)bis(methylene) bis(2-heptylnonanoate) (3.0 g, 62.2% yield) as a light-yellow oil. ¹H NMR (400 MHz, CDCl₃) δ 3.97 (s, 4H), 3.65-3.55 (m, 2H), 2.38-2.28 (m, 8H), 1.65-1.32 (m, 24H), 1.24 (s, 40H), 0.91-0.84 (m, 21H), 0.03 (s, 6H). LCMS (ESI): Calc'd for C₅₄H₁₀₆O₅Si [M+H]⁺ 876.78, found 876.6.

### Step 5: (3-(4-hydroxybutyl)-3-azaspiro[5.5]undecane-9,9-diyl)bis(methylene)bis(2-heptylnonanoate)

### General HCl-Mediated Tertbutyldimethyl Silyl Deprotection

To a solution of (3-(4-((tert-butyldimethylsilyl)oxy)butyl)-3-azaspiro[5.5]undecane-9,9-diyl)bis(methylene) bis(2-heptylnonanoate) (3.0 g, 3.42 mmol) in DCM (15 mL) was added HCl (4M dioxane, 25 mL, 50 mmol) and the resulting solution was stirred at room temperature for 1 hour. After this time the mixture was concentrated in vacuo and the residue was neutralized by saturated NaHCO₃ (60 mL). The mixture was extracted with DCM (35 mL×4) and the combined organic layers were washed with brine (70 mL), dried over Na₂SO₄, filtered, and concentrated in vacuo. The crude material was purified by silica gel column chromatography followed by additional super critical fluid chromatography to give (3-(4-hydroxybutyl)-3-azaspiro[5.5]undecane-9,9-diyl)bis(methylene) bis(2-heptylnonanoate) (1.21 g, 46.3% yield) as a light-yellow oil. ¹H NMR (400 MHZ, CDCl₃) δ 3.96 (s, 4H), 3.57-3.54 (m, 2H), 2.49-2.43 (m, 4H), 2.41-2.36 (m, 3H), 2.35-2.27 (m, 2H), 1.70-1.64 (m, 4H), 1.63-1.48 (m, 8H), 1.46-1.32 (m, 12H), 1.32-1.12 (m, 40H), 0.86 (t, J=6.8 Hz, 12H). LCMS (ESI): Calc'd for C₄₈H₉₂O₅ [M+H]⁺ 762.69, found 762.5.

### Example 2

### 2-(3-(4-hydroxybutyl)-3-azaspiro[5.5]undecan-9-yl)propane-1,3-diyl bis(2-heptylnonanoate) (2)

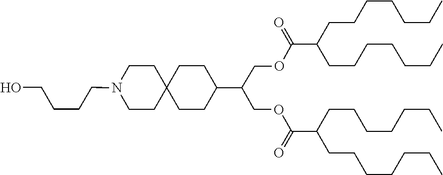 [SMILES: CCCCCCCC(CCCCCCC)C(=O)OCC(COC(=O)C(CCCCCCC)CCCCCCC)C1CCC2(CC1)CCN(CCCCO)CC2]

### Step 1: diethyl 2-(3-(tert-butoxycarbonyl)-3-azaspiro[5.5]undecan-9-ylidene)malonate

To a flask containing THF (50.0 mL) at 0° C. was slowly added TiCl₄ (6.26 g, 33.0 mmol) followed by dropwise addition of a mixture of tert-butyl 9-oxo-3-azaspiro[5.5]undecane-3-carboxylate (4.2 g, 16 mmol), diethyl malonate (2.64 g, 16.5 mmol) and pyridine (4.97 g, 62.8 mmol) in THF (40.0 mL). The red suspension was allowed to slowly warm to room temperature and was stirred for 16 hours. The mixture was then quenched with saturated NaHCO₃ (120 mL) until it turned to a clear orange liquid. The aqueous phase was extracted with ethyl acetate (60 mL×5), the combined organic phases were dried over Na₂SO₄ and concentrated in vacuo. Purification by column chromatography provided diethyl 2-(3-(tert-butoxycarbonyl)-3-azaspiro[5.5]undecan-9-ylidene) malonate (4.16 g, 65% yield) as a light-yellow oil. ¹H NMR (400 MHz, CDCl₃) δ 4.22 (q, J=7.1 Hz, 4H), 3.41-3.34 (m, 4H), 2.58-2.50 (m, 4H), 1.62-1.54 (m, 4H), 1.44 (br s, 13H), 1.28 (t, J=7.1 Hz, 6H). LCMS (ESI): Calc'd for C₁₈H₂₈NO₆ [M-tBu+H]⁺ 354.2; found 354.1.

### Step 2: tert-butyl 9-(1,3-dihydroxypropan-2-yl)-3-azaspiro[5.5]undecane-3-carboxylate

To a solution of diethyl 2-(3-(tert-butoxycarbonyl)-3-azaspiro[5.5]undecan-9-ylidene) malonate (6.40 g, 15.63 mmol) in EtOH (100.0 mL) was added NaBH₄ (5.0 g, 132.2 mmol), and the mixture was stirred for 4 hours at room temperature then heated to 65° C. and stirred for an additional 12 hours. At this point the mixture was slowly quenched with saturated NH₄Cl (100 mL), then concentrated in vacuo to remove EtOH. The resulting aqueous layer was extracted with DCM (50 mL×5). The combined organic layers were dried over Na₂SO₄, filtered, and concentrated in vacuo. Purification using silica gel column chromatography afforded tert-butyl 9-(1,3-dihydroxypropan-2-yl)-3-azaspiro[5.5]undecane-3-carboxylate (3.10 g, 60.7% yield) as a colorless solid. ¹H NMR (400 MHZ, CDCl₃) δ 4.06 (dd, J=10.8, 4.5 Hz, 1H), 3.89-3.66 (m, 3H), 3.36-3.29 (m, 4H), 1.82-1.48 (m, 5H), 1.44 (s, 11H), 1.31-0.99 (m, 7H). LCMS (ESI): Calc'd for C₁₈H₃₄NO₄ [M+H]⁺ 328.2; found 328.1.

### Step 3: 2-(3-azaspiro[5.5]undecan-9-yl)propane-1,3-diol

To a solution of tert-butyl 9-(1,3-dihydroxypropan-2-yl)-3-azaspiro[5.5]undecane-3-carboxylate (3.10 g, 9.48 mmol) in MeOH (25.0 mL) at room temperature was added 2M HCl (25.0 mL, 50.0 mmol), and the mixture was allowed to stir at room temperature for 14 hours. The mixture was concentrated in vacuo to provide 2-(3-azaspiro[5.5]undecan-9-yl)propane-1,3-diol (2.5 g, 99.9% crude) as white solid. The material was used as is in the subsequent transformation. LCMS (ESI): Calc'd for C₁₃H₂₆NO₂ [M+H]⁺ 228.2; found 228.1.

### Step 4: 2-(3-(4-((tert-butyldimethylsilyl)oxy)butyl)-3-azaspiro[5.5]undecan-9-yl)propane-1,3-diol

This compound was prepared according to the general alkylation procedure as described in Example 1 (Step 3). 2-(3-azaspiro[5.5]undecan-9-yl)propane-1,3-diol (2.50 g, 9.48 mmol) provided 2-(3-(4-((tert-butyldimethylsilyl)oxy)butyl)-3-azaspiro[5.5]undecan-9-yl)propane-1,3-diol (3.02 g, 77% yield) as a light-yellow oil. ¹H NMR (400 MHZ, CDCl₃) δ 3.90-3.69 (m, 4H), 3.61-3.58 (t, J=5.9 Hz, 2H), 2.39-2.31 (m, 6H), 1.68-1.63 (m, 2H), 1.60-1.42 (m, 9H), 1.40-1.32 (m, 3H), 1.25-0.96 (m, 4H), 0.87 (s, 9H), 0.02 (s, 6H). LCMS (ESI): Calc'd for C₂₃H₄₈NO₃Si [M+H]⁺ 414.3; found 414.3.

### Step 5: 2-(3-(4-((tert-butyldimethylsilyl)oxy)butyl)-3-azaspiro[5.5]undecan-9-yl)propane-1,3-diyl bis(2-heptylnonanoate)

This compound was prepared according to the general acylation procedure as described in Example 1 (Step 4). 2-(3-(4-((tert-Butyldimethylsilyl)oxy)butyl)-3-azaspiro[5.5]undecan-9-yl)propane-1,3-diol (3.02 g, 7.30 mmol) afforded 2-(3-(4-((tert-butyldimethylsilyl)oxy)butyl)-3-azaspiro[5.5]undecan-9-yl)propane-1,3-diyl bis(2-heptylnonanoate) (4.97 g, 76.4% yield) as a light-yellow oil.

### Step 6: 2-(3-(4-hydroxybutyl)-3-azaspiro[5.5]undecan-9-yl)propane-1,3-diyl bis(2-heptylnonanoate)

This compound was prepared according to the general tert-butyldimethylsilyl deprotection procedure as described in Example 1 (Step 5). 2-(3-(4-((tert-Butyldimethylsilyl)oxy)butyl)-3-azaspiro[5.5]undecan-9-yl)propane-1,3-diyl bis(2-heptylnonanoate) (4.97 g, 5.58 mmol) gave 2-(3-(4-hydroxybutyl)-3-azaspiro[5.5]undecan-9-yl)propane-1,3-diyl bis(2-heptylnonanoate) (1.70 g, 39.3% yield) as a light-yellow oil. ¹H NMR (400 MHZ, CDCl₃) δ 4.18 (dd, J=11.2, 4.9 Hz, 2H), 4.04 (dd, J=11.2, 6.3 Hz, 2H), 3.56 (m, 2H), 2.57-2.37 (m, 6H), 2.34-2.25 (m, 2H), 1.83-1.79 (m, 1H), 1.74-1.63 (m, 6H), 1.61-1.52 (m, 7H), 1.46-1.36 (m, 7H), 1.24 (br s, 43H), 1.06-0.99 (m, 2H), 0.86 (t, J=6.7 Hz, 12H). LCMS (ESI): Calc'd for C₄₉H₉₄NO₅ [M+H]⁺ 776.7; found 776.9.

### Example 3

### 2-(9-(4-Hydroxybutyl)-3,9-diazaspiro[5.5]undecan-3-yl)propane-1,3-diyl bis(2-heptylnonanoate) (3)

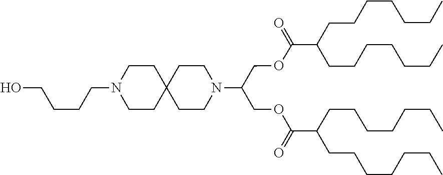 [SMILES: CCCCCCCC(CCCCCCC)C(=O)OCC(COC(=O)C(CCCCCCC)CCCCCCC)N1CCC2(CCN(CCCCO)CC2)CC1]

### Step 1: tert-butyl 9-(2,2-dimethyl-1,3-dioxan-5-yl)-3,9-diazaspiro[5.5]undecane-3-carboxylate

### General Reductive Amination Procedure

To a solution of tert-butyl 3,9-diazaspiro[5.5]undecane-3-carboxylate hydrochloride (9.13 g, 31.39 mmol) in DCM (150 mL) was added Et₃N (3.18 g 31.4 mmol). After stirring at room temperature for 10 minutes, 2,2-dimethyl-1,3-dioxan-5-one (4.09 g, 31.4 mmol) was added, and the mixture was stirred at room temperature for 30 min. The mixture was cooled to 0° C., Na(OAc)₃BH (6.65 g, 31.4 mmol) was added slowly, and the mixture was warmed to room temperature and stirred for an additional 26 hours. At this point LCMS showed that the desired product was detected as major peak and the reaction was quenched with sat. NaHCO₃ until no gas was generated. The mixture was extracted with DCM (50 mL×5), the combined organic layers were dried over Na₂SO₄, filtered, and concentrated in vacuo. The residue was purified by silica gel column chromatography to give tert-butyl 9-(2,2-dimethyl-1,3-dioxan-5-yl)-3,9-diazaspiro[5.5]undecane-3-carboxylate (8.28 g, 71.6% yield) as a light-yellow solid. ¹H NMR (400 MHz, CDCl₃) δ 3.96 (dd, J=11.7, 5.2 Hz, 2H), 3.81 (dd, J=11.6, 8.4 Hz, 2H), 3.42-3.31 (m, 5H), 2.65-2.48 (m, 5H), 1.57-1.26 (m, 24H). LCMS (ESI): Calc'd for C₂₀H₃₇N₂O₄ [M+H]⁺ 369.27, found 370.3.

### Step 2: 2-(3,9-diazaspiro[5.5]undecan-3-yl)propane-1,3-diol

### General Acetal Hydrolysis/Boc Deprotection Procedure

To a solution of tert-butyl 9-(2,2-dimethyl-1,3-dioxan-5-yl)-3,9-diazaspiro[5.5]undecane-3-carboxylate (8.28 g, 22.46 mmol) in MeOH (30 mL) was added aqueous HCl (30 mL, 30 mmol), and the mixture was stirred at room temperature for 16 hours. The mixture was dried under lyophilization to provide 2-(3,9-diazaspiro[5.5]undecan-3-yl)propane-1,3-diol (5.13 g, 100% crude) as a yellow solid. The material was used as is in the subsequent transformation. LCMS (ESI): Calc'd for C₁₂H₂₅N₂O₂ [M+H]⁺ 229.18, found 229.2.

### Step 3: 2-(9-(4-((tert-butyldimethylsilyl)oxy)butyl)-3,9-diazaspiro[5.5]undecan-3-yl)propane-1,3-diol

This compound was prepared according to general alkylation procedure as described in Example 1 (Step 3). 2-(3,9-diazaspiro[5.5]undecan-3-yl)propane-1,3-diol (5.03 g 22.03 mmol) yielded 2-(9-(4-((tert-butyldimethylsilyl)oxy)butyl)-3,9-diazaspiro[5.5]undecan-3-yl)propane-1,3-diol (2.25 g, 25% yield) as a yellow solid. ¹H NMR (400 MHZ, CDCl₃) δ 3.63-3.59 (m, 6H), 2.76 (p, J=6.5 Hz, 1H), 2.66 (t, J=5.5 Hz, 4H), 2.44-2.32 (m, 6H), 1.55-1.48 (m, 13H), 0.88 (s, 9H), 0.04 (s, 6H). LCMS (ESI): Calc'd for C₂₂H₄₇N₂O₃Si [M+H]⁺ 415.33, found 415.3.

### Step 4: 2-(9-(4-((tert-butyldimethylsilyl)oxy)butyl)-3,9-diazaspiro[5.5]undecan-3-yl)propane-1,3-diyl bis(2-heptylnonanoate)

This compound was prepared according to the general acylation procedure as described in Example 1 (Step 4). 2-(9-(4-((tert-Butyldimethylsilyl)oxy)butyl)-3,9-diazaspiro[5.5]undecan-3-yl)propane-1,3-diol (1.88 g, 4.53 mmol) and 2-heptylnonanoic acid (A12) (2.56 g, 9.97 mmol) provided 2-(9-(4-((tert-butyldimethylsilyl)oxy)butyl)-3,9-diazaspiro[5.5]undecan-3-yl)propane-1,3-diyl bis(2-heptylnonanoate) (3.55 g, 87.9% yield) as a yellow oil. ¹H NMR (400 MHZ, CDCl₃) δ 4.29 (dd, J=11.5, 6.3 Hz, 2H), 4.07 (dd, J=11.5, 5.5 Hz, 2H), 3.60 (t, J=6.2 Hz, 2H), 2.98 (p, J=5.8 Hz, 1H), 2.60 (t, J=5.3 Hz, 4H), 2.55-2.40 (m, 6H), 2.36-2.28 (m, 2H), 1.69-1.35 (m, 20H), 1.26 (d, J=8.6 Hz, 40H), 0.91-0.83 (m, 21H), 0.03 (s, 6H).

### Step 5: 2-(9-(4-hydroxybutyl)-3,9-diazaspiro[5.5]undecan-3-yl)propane-1,3-diyl bis(2-heptylnonanoate)

This compound was prepared according to the general tert-butyldimethylsilyl deprotection procedure as described in Example 1 (Step 5). 2-(9-(4-((tert-Butyldimethylsilyl)oxy)butyl)-3,9-diazaspiro[5.5]undecan-3-yl)propane-1,3-diyl bis(2-heptylnonanoate) (3.55 g, 3.98 mmol) provided 2-(9-(4-hydroxybutyl)-3,9-diazaspiro[5.5]undecan-3-yl)propane-1,3-diyl bis(2-heptyl-nonanoate (1.64 g, 53% yield) as a yellow oil. ¹H NMR (400 MHZ, CDCl₃) δ 4.29 (dd, J=11.5, 6.3 Hz, 2H), 4.06 (dd, J=11.5, 5.5 Hz, 2H), 3.56-3.35 (m, 2H), 2.98-2.95 (m, 1H), 2.60-2.57 (m, 4H), 2.52-2.37 (m, 7H), 2.35-2.28 (m, 2H), 1.68-1.64 (m, 5H), 1.63-1.49 (m, 7H), 1.47-1.37 (m, 8H), 1.27-1.23 (m, 40H), 0.87 (t, J=6.7 Hz, 12H). LCMS (ESI): Calc'd for C₄₈H₉₃N₂O₅ [M+H]⁺ 777.70, found 777.7.

### Example 4

### 2-(9-(4-hydroxybutyl)-3,9-diazaspiro[5.5]undecan-3-yl)propane-1,3-diyl bis(2-cyclobutyldecanoate) (4)

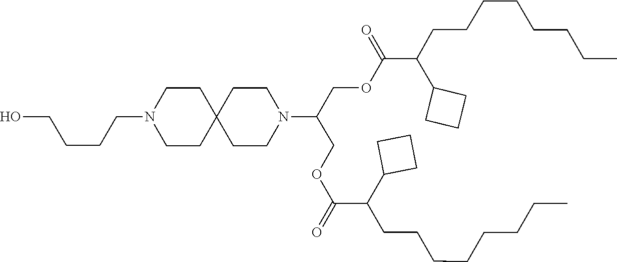 [SMILES: CCCCCCCCC(C(=O)OCC(COC(=O)C(CCCCCCCC)C1CCC1)N1CCC2(CCN(CCCCO)CC2)CC1)C1CCC1]

### Step 1: 2-(9-(4-((tert-butyldimethylsilyl)oxy)butyl)-3,9-diazaspiro[5.5]undecan-3-yl)propane-1,3-diyl bis(2-cyclobutyldecanoate)

This compound was prepared according to the general acylation procedure as described in Example 1 (Step 4). 2-(9-(4-((tert-butyldimethylsilyl)oxy)butyl)-3,9-diazaspiro[5.5]undecan-3-yl)propane-1,3-diol (Example 3, Step 3) (0.500 g, 1.21 mmol) and 2-cyclobutyldecanoic acid (A5) (0.764 g, 3.38 mmol) provided 2-(9-(4-((tert-butyldimethylsilyl)oxy)butyl)-3,9-diazaspiro[5.5]-undecan-3-yl)propane-1,3-diyl bis(2-cyclobutyldecanoate) (0.95 g, 95% yield) as a light-yellow oil. ¹H NMR (400 MHZ, CDCl₃) δ 4.29 (dd, J=11.6, 6.0 Hz, 2H), 4.08 (dd, J=11.5, 5.5 Hz, 2H), 3.62 (t, J=6.1 Hz, 2H), 2.99-2.93 (m, 1H), 2.61 (t, J=5.5 Hz, 4H), 2.53-2.40 (m, 8H), 2.37-2.34 (m, 3H), 2.11-1.92 (m, 5H), 1.90-1.64 (m, 10H), 1.62-1.36 (m, 16H), 1.33-1.20 (m, 20H), 0.92-0.86 (m, 15H), 0.06 (s, 6H).

### Step 2: 2-(9-(4-hydroxybutyl)-3,9-diazaspiro[5.5]undecan-3-yl)propane-1,3-diyl bis(2-cyclobutyldecanoate)

### General TBAF-Mediated Tertbutyldimethyl Silyl Deprotection Procedure

TBAF (1.81 g, 6.92 mmol) was added to a solution of 2-(9-(4-((tert-butyldimethylsilyl)-oxy)butyl)-3,9-diazaspiro[5.5]undecan-3-yl)propane-1,3-diyl bis(2-cyclobutyldecanoate) (1.15 g, 1.38 mmol) in THF (12.0 mL). The mixture was stirred at room temperature for 3 hours. The reaction mixture was diluted with EtOAc (10 mL) and washed with H₂O (15 mL×3) and brine (15 mL×2). The combined organic layers were dried with Na₂SO₄ and filtered. The filtrate was concentrated in vacuo, and the residue that was purified by silica gel column chromatography to provide 2-(9-(4-hydroxybutyl)-3,9-diazaspiro[5.5]undecan-3-yl)propane-1,3-diyl bis(2-cyclobutyl-decanoate) (0.35 g, 35% yield) as a light-yellow oil. ¹H NMR (400 MHZ, CDCl₃) δ 4.29 (dd, J=11.6, 6.1 Hz, 2H), 4.07 (dd, J=11.5, 5.5 Hz, 2H), 3.64-3.58 (m, 2H), 2.96 (t, J=5.9 Hz, 1H), 2.65-2.51 (m, 6H), 2.49-2.39 (m, 3H), 2.32 (td, J=9.8, 4.2 Hz, 3H), 2.12-2.02 (m, 2H), 2.01-1.59 (m, 21H), 1.54-1.37 (m, 8H), 1.33-1.21 (m, 24H), 0.89 (t, J=6.7 Hz, 6H). LCMS (ESI): Calc'd for C₄₄H₈₁N₂O₅ [M+H]⁺ 717.61, found 717.4.

### Example 5

### 2-(9-(5-hydroxypentyl)-3,9-diazaspiro[5.5]undecan-3-yl)propane-1,3-diyl bis(2-hexyldecanoate) (5)

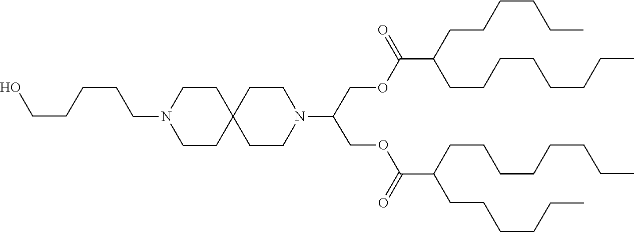 [SMILES: CCCCCCCCC(CCCCCC)C(=O)OCC(COC(=O)C(CCCCCC)CCCCCCCC)N1CCC2(CCN(CCCCCO)CC2)CC1]

### Step 1: 2-(9-(5-((tert-butyldimethylsilyl)oxy) pentyl)-3,9-diazaspiro[5.5]undecan-3-yl)propane-1,3-diol

K₂CO₃ (11.7 g, 84.4 mmol) was added to a solution of 2-(3,9-diazaspiro[5.5]undecan-3-yl)propane-1,3-diol (Example 3, Step 2) (3.0 g, 10 mmol) in DMF (50 mL) and MeOH (15 mL), and the mixture was stirred for 30 minutes. ((5-Bromopentyl)oxy) (tert-butyl)dimethylsilane (2.8 g, 9.95 mmol) was added. The reaction mixture was stirred at 65° C. for 16 hours and then filtered. The filtrate was concentrated in vacuo. The residue was diloved in EtOAc (30 mL) and washed with H₂O (20 mL×4). The organic layer was dried over Na₂SO₄ and filtered. The filtrate was concentrated in vacuo and purified by column chromatography to afford 2-(9-(5-((tert-butyldimethylsilyl)oxy) pentyl)-3,9-diazaspiro[5.5]undecan-3-yl)propane-1,3-diol (1.75 g, 40% yield) as a light-yellow oil. ¹H NMR (400 MHZ, CDCl₃) δ 3.69-3.58 (m, 6H), 2.82 (p, J=6.4 Hz, 1H), 2.72 (t, J=5.5 Hz, 4H), 2.58-2.43 (m, 6H), 1.68-1.51 (m, 12H), 1.36 (q, J=7.9 Hz, 2H), 0.90 (s, 9H), 0.06 (s, 6H). LCMS (ESI): Calc'd for C₂₃H₄₉N₂O₃Si [M+H]⁺ 429.35, found 429.3.

### Step 2: 2-(9-(5-((tert-butyldimethylsilyl)oxy)pentyl)-3,9-diazaspiro[5.5]undecan-3-yl)propane-1,3-diyl bis(2-hexyldecanoate)

This compound was prepared according to the general acylation procedure as described in Example 1 (Step 4). 2-(9-(5-((tert-butyldimethylsilyl)oxy) pentyl)-3,9-diazaspiro[5.5]undecan-3-yl)propane-1,3-diol (0.800 g, 1.87 mmol) and 2-hexyldecanoic acid (A13) (1.20 g, 3.18 mmol) provided 2-(9-(5-((tert-butyldimethylsilyl)oxy) pentyl)-3,9-diazaspiro[5.5]undecan-3-yl)propane-1,3-diyl bis(2-hexyldecanoate) (1.0 g, 59% yield) as a light-yellow oil. ¹H NMR (400 MHZ, CDCl₃) δ 4.31 (dd, J=11.5, 6.3 Hz, 2H), 4.08 (dd, J=11.5, 5.5 Hz, 2H), 3.60 (t, J=6.4 Hz, 2H), 3.00 (q, J=5.9 Hz, 1H), 2.65-2.48 (m, 8H), 2.40-2.22 (m, 3H), 1.98-1.91 (m, 1H), 1.74-1.40 (m, 24H), 1.36-1.19 (m, 38H), 0.92-0.84 (m, 21H), 0.05 (s, 6H).

### Step 3: 2-(9-(5-hydroxypentyl)-3,9-diazaspiro[5.5]undecan-3-yl)propane-1,3-diyl bis(2-hexyldecanoate)

This compound was prepared according to the general tert-butyldimethylsilyl deprotection using TBAF as described in Example 5 (Step 2). 2-(9-(5-((tert-butyldimethylsilyl)oxy) pentyl)-3,9-diazaspiro[5.5]undecan-3-yl)propane-1,3-diyl bis(2-hexyldecanoate) (0.950 g, 1.05 mmol) provided 2-(9-(5-hydroxypentyl)-3,9-diazaspiro[5.5]undecan-3-yl)propane-1,3-diyl bis(2-hexyldecanoate) (0.13 g, 15% yield) as a light-yellow oil. ¹H NMR (400 MHZ, CD₃OD) δ 4.32 (ddd, J=11.6, 5.9, 2.0 Hz, 2H), 4.16 (ddd, J=11.5, 5.5 1.6 Hz, 2H), 3.57 (t, J=6.4 Hz, 2H), 3.02 (q, J=5.8 Hz, 1H), 2.69-2.67 (m, 8H), 2.58 (m, 2H), 2.37 (tt, J=9.3, 5.1 Hz, 2H), 1.70-1.53 (m, 16H), 1.51-1.22 (m, 6H), 1.36-1.22 (m, 40H), 0.88 (t, J=6.7 Hz, 12H). LCMS (ESI): Calc'd for C₄₉H₉₅N₂O₅ [M+H]⁺ 791.72, found 791.7.

### Example 6

### 2-(2-(4-hydroxybutyl)-2,8-diazaspiro[4.5]decan-8-yl)propane-1,3-diyl bis(2-hexyldecanoate) (6)

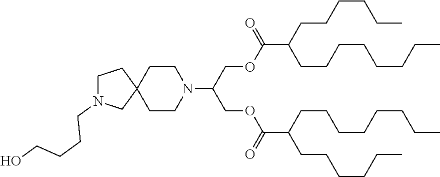 [SMILES: CCCCCCCCC(CCCCCC)C(=O)OCC(COC(=O)C(CCCCCC)CCCCCCCC)N1CCC2(CCN(CCCCO)C2)CC1]

### Step 1: tert-butyl 8-(2,2-dimethyl-1,3-dioxan-5-yl)-2,8-diazaspiro[4.5]decane-2-carboxylate

This compound was prepared according to the general reductive amination procedure as described in Example 3 (step 1). tert-Butyl 2,8-diazaspiro[4.5]decane-2-carboxylate (5.00 g, 18.1 mmol) provided tert-butyl 8-(2,2-dimethyl-1,3-dioxan-5-yl)-2,8-diazaspiro[4.5]decane-2-carboxylate (4.1 g, 64% yield) as light-yellow oil. ¹H NMR (400 MHZ, CDCl₃) δ 3.96 (dt, J=11.8, 5.9 Hz, 2H), 3.82 (dt, J=11.5, 7.2 Hz, 2H), 3.35 (dt, J=21.8, 7.2 Hz, 2H), 3.18 (s, 1H), 3.08 (s, 1H), 2.65-2.55 (m, 3H), 2.51-2.41 (m, 2H), 1.72-1.63 (m, 3H), 1.59-1.50 (m, 3H), 1.48-1.35 (m, 15H). LCMS (ESI): Calc'd for C₁₉H₃₅N₂O₄ [M+H]⁺ 355.25; found 355.1.

### Step 2: 2-(2,8-diazaspiro[4.5]decan-8-yl)propane-1,3-diol

To a solution of tert-butyl 8-(2,2-dimethyl-1,3-dioxan-5-yl)-2,8-diazaspiro[4.5]decane-2-carboxylate (4.10 g, 11.6 mmol) in MeOH (50 mL) was added HCl (0.12 M, 50 mL). The mixture was stirred at 20° C. for 16 hours. The mixture was dried under lyophilization to remove solvents to give 2-(2,8-diazaspiro[4.5]decan-8-yl)propane-1,3-diol (3.32 g, 100% yield) as a yellow oil. LCMS (ESI): Calc'd for C₁₁H₂₃N₂O₂ [M+H]⁺ 215.17; found 215.1.

### Step 3: 2-(2-(4-((tert-butyldimethylsilyl)oxy)butyl)-2,8-diazaspiro[4.5]decan-8-yl)propane-1,3-diol

This compound was prepared according to the general alkylation procedure as described in Example 1 (Step 3). 2-(2,8-diazaspiro[4.5]decan-8-yl)propane-1,3-diol (3.32 g, 11.6 mmol) and 1-bromo-4-(t-butyldimethylsilyloxy) butane (3.09 g, 11.6 mmol) provided 2-(2-(4-((tert-butyldimethylsilyl)oxy)butyl)-2,8-diazaspiro[4.5]decan-8-yl)propane-1,3-diol (2.7 g, 59% yield) as a light-yellow oil. LCMS (ESI): Calc'd for C₂₁H₄₅N₂O₃Si [M+H]⁺ 401.31; found 401.2.

### Step 4: 2-(2-(4-((tert-butyldimethylsilyl)oxy)butyl)-2,8-diazaspiro[4.5]decan-8-yl)propane-1,3-diyl bis(2-hexyldecanoate)

This compound was prepared according to the general acylation procedure as described in Example 1 (Step 4). 2-(2-(4-((tert-butyldimethylsilyl)oxy)butyl)-2,8-diazaspiro[4.5]decan-8-yl)propane-1,3-diol (0.500 g, 1.25 mmol) and 2-hexyldecanoic acid (A13) (0.800 g, 3.12 mmol) delivered 2-(2-(4-((tert-butyldimethylsilyl)oxy)butyl)-2,8-diazaspiro[4.5]decan-8-yl)propane-1,3-diyl bis(2-hexyldecanoate) (0.85 g, 78% yield) as a light-yellow oil. ¹H NMR (400 MHZ, CDCl₃) δ 4.31 (dd, J=11.5, 6.3 Hz, 2H), 4.07 (dd, J=11.5, 5.5 Hz, 2H), 3.65-3.60 (m, 2H), 2.99 (p, J=5.9 Hz, 1H), 2.63-2.56 (m, 6H), 2.47-2.29 (m, 6H), 1.64-1.52 (m, 14H), 1.49-1.39 (m, 5H), 1.34-1.21 (m, 39H), 0.93-0.85 (m, 21H), 0.06 (s, 6H).

### Step 5: 2-(2-(4-hydroxybutyl)-2,8-diazaspiro[4.5]decan-8-yl)propane-1,3-diyl bis(2-hexyldecanoate)

This compound was prepared according to the general tert-butyldimethylsilyl deprotection using TBAF as described in Example 5 (Step 2). 2-(2-(4-((tert-butyldimethylsilyl)oxy)butyl)-2,8-diazaspiro[4.5]decan-8-yl)propane-1,3-diyl bis(2-hexyldecanoate) (0.800 g, 0.912 mmol) delivered 2-(2-(4-hydroxybutyl)-2,8-diazaspiro[4.5]decan-8-yl)propane-1,3-diyl bis(2-hexyldecanoate) (0.17 g, 25% yield) as a light-yellow oil. ¹H NMR (400 MHZ, CDCl₃) δ 4.30 (dd, J=11.5, 6.4 Hz, 2H), 4.05 (dd, J=11.5, 5.5 Hz, 2H), 3.64-3.59 (m, 2H), 3.00 (p, J=5.9 Hz, 1H), 2.84-2.73 (br s, 1H), 2.61 (t, J=5.4 Hz, 6H), 2.34 (tt, J=8.6, 5.4 Hz, 2H), 1.80-1.67 (m, 6H), 1.65-1.53 (m, 8H), 1.50-1.38 (m, 5H), 1.34-1.02 (m, 43H), 0.89 (t, J=6.6 Hz, 12H). LCMS (ESI): Calc'd for C₄₇H₉₁N₂O₅ [M+H]⁺ 763.68, found 763.5.

### Example 7

### 3-((2-hexyldecanoyl)oxy)-2-(9-(4-hydroxybutyl)-3,9-diazaspiro[5.5]undecan-3-yl)propyl palmitate (7)

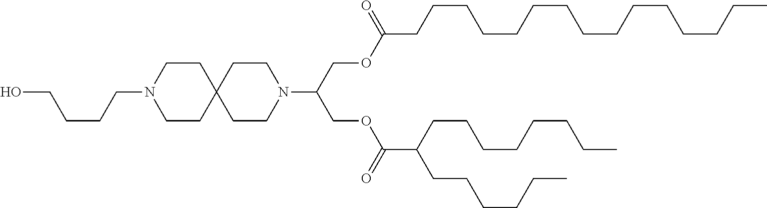 [SMILES: CCCCCCCCCCCCCCCC(=O)OCC(COC(=O)C(CCCCCC)CCCCCCCC)N1CCC2(CCN(CCCCO)CC2)CC1]

### Step 1: 2-(9-(4-((tert-butyldimethylsilyl)oxy)butyl)-3,9-diazaspiro[5.5]undecan-3-yl)-3-hydroxypropyl 2-hexyldecanoate

DCC (1.49 g, 7.23 mmol), DMAP (0.118 g, 0.965 mmol) and 2-hexyldecanoic acid (A13) (1.30 g, 5.06 mmol) were added to a solution of 2-(9-(4-((tert-butyldimethylsilyl)oxy)butyl)-3,9-diazaspiro[5.5]undecan-3-yl)propane-1,3-diol (Example 3, Step 3) (2.0 g, 4.8 mmol) in DCM (60.3 mL), and the mixture was stirred at room temperature for 16 hours. The reaction mixture was filtered over celite. The filtrate was concentrated in vacuo, and the residue was purified by silica gel column chromatography to provide 2-(9-(4-((tert-butyldimethylsilyl)oxy)butyl)-3,9-diazaspiro[5.5]undecan-3-yl)-3-hydroxypropyl 2-hexyldecanoate (2.6 g, 84% yield) as a yellow oil. ¹H NMR (400 MHZ, CDCl₃) δ 4.25 (dd, J=11.6, 6.4 Hz, 1H), 4.01 (dd, J=11.7, 5.6 Hz, 1H), 3.66-3.60 (m, 2H), 3.52 (dd, J=10.4, 5.1 Hz, 1H), 3.39 (t, J=10.3 Hz, 1H), 2.96 (dq, J=11.4, 5.9 Hz, 1H), 2.83-2.74 (m, 2H), 2.52-2.29 (m, 9H), 1.63-1.41 (m, 17H), 1.35-1.19 (m, 20H), 0.93-0.86 (m, 15H), 0.05 (s, J=1.3 Hz, 6H).

### Step 2: 2-(9-(4-((tert-butyldimethylsilyl)oxy)butyl)-3,9-diazaspiro[5.5]undecan-3-yl)-3-((2-hexyldecanoyl)oxy)propyl palmitate

DCC (0.237 g, 1.15 mmol), DMAP (0.019 g, 0.153 mmol) and palmitic acid (A17) (0.245 g, 0.957 mmol) were added to a solution of 2-(9-(4-((tert-butyldimethylsilyl)oxy)butyl)-3,9-diazaspiro[5.5]undecan-3-yl)-3-hydroxypropyl 2-hexyldecanoate (0.500 g, 0.766 mmol) in DCM (3.83 mL), and the mixture was stirred at room temperature for 5 hours. The reaction mixture was filtered over celite. The filtrate was concentrated in vacuo, and the residue was purified by silica gel column chromatography to provide 2-(9-(4-((tert-butyldimethylsilyl)oxy)butyl)-3,9-diazaspiro[5.5]undecan-3-yl)-3-((2-hexyldecanoyl)oxy) propyl palmitate (0.54 g, 79% yield) as a yellow oil. ¹H NMR (400 MHZ, CDCl₃) δ 4.30 (ddd, J=10.9, 6.2, 4.2 Hz, 2H), 4.11 (ddd, J=11.7, 5.7, 2.7 Hz, 2H), 3.63 (t, J=5.9 Hz, 2H), 3.05-2.96 (m, 2H), 2.61 (t, J=5.4 Hz, 4H), 2.48-2.24 (m, 12H), 1.67-1.39 (m, 20H), 1.36-1.20 (m, 38H), 0.94-0.86 (m, 18H), 0.06 (s, 6H).

### Step 3: 3-((2-hexyldecanoyl)oxy)-2-(9-(4-hydroxybutyl)-3,9-diazaspiro[5.5]undecan-3-yl)propyl palmitate

TBAF (1.17 g, 4.85 mmol) was added to a solution of 2-(9-(4-((tert-butyldimethylsilyl)-oxy)butyl)-3,9-diazaspiro[5.5]undecan-3-yl)-3-((2-hexyldecanoyl)oxy) propyl palmitate (0.540 g, 0.606 mmol) in THF (3.0 mL). The mixture was stirred at room temperature for 3 hours. The above sequence was repeated with an additional batch of 2-(9-(4-((tert-butyldimethylsilyl)-oxy)butyl)-3,9-diazaspiro[5.5]undecan-3-yl)-3-((2-hexyldecanoyl)oxy) propyl palmitate (0.300 g, 0.337 mmol), and the reaction mixtures were combined. The combined reaction mixtures were washed with H₂O (20 mL) and extracted with EtOAc (10 mL×3). The combined organic layer was dried with Na₂SO₄ and filtered. The filtrate was concentrated in vacuo, and the residue was purified by silica gel column chromatography to provide 3-((2-hexyldecanoyl)oxy)-2-(9-(4-hydroxybutyl)-3,9-diazaspiro[5.5]undecan-3-yl)propyl palmitate (0.21 g, 42% yield) as a colorless oil. ¹H NMR (400 MHZ, CD₃OD) δ 4.36-4.27 (m, 2H), 4.21-4.11 (m, 2H), 3.56 (t, J=5.9 Hz, 2H), 3.05-2.98 (m, 1H), 2.67 (t, J=5.6 Hz, 4H), 2.58-2.51 (m, 4H), 2.45 (t, J=7.2 Hz, 2H), 2.41-2.29 (m, 3H), 1.69-1.42 (m, 18H), 1.40-1.20 (m, 44H), 0.90 (t, J=6.7 Hz, 9H). LCMS (ESI): Calc'd for C₄₈H₉₃N₂O₅ [M+H]⁺ 777.70, found 777.5.

### Example 8

### 2-(9-(3-((2-(methylamino)-3,4-dioxocyclobut-1-en-1-yl)amino)propyl)-3,9-diazaspiro[5.5]undecan-3-yl)propane-1,3-diyl bis(2-heptylnonanoate) (8)

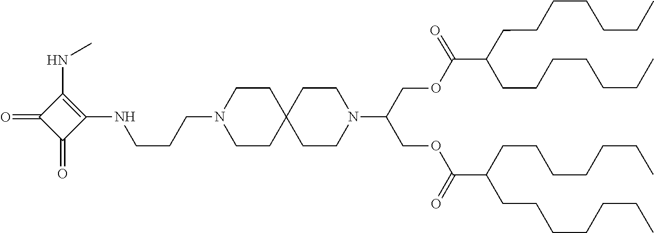 [SMILES: CCCCCCCC(CCCCCCC)C(=O)OCC(COC(=O)C(CCCCCCC)CCCCCCC)N1CCC2(CCN(CCCNc3c(NC)c(=O)c3=O)CC2)CC1]

### Step 1: tert-butyl (3-(9-(1,3-dihydroxypropan-2-yl)-3,9-diazaspiro[5.5]undecan-3-yl)propyl)carbamate

### General Alkylation Procedure Using 3-(Boc-Amino)Propyl Bromide

K₂CO₃ (4.5 g, 33 mmol) was added to a solution of 2-(3,9-diazaspiro[5.5]undecan-3-yl)propane-1,3-diol (Example 3, Step 2) (1.63 g, 5.43 mmol) in DMF (60 mL) and MeOH (3.0 mL). The mixture was stirred at room temperature for 1 hour. 3-(Boc-amino) propyl bromide) (1.29 g, 5.43 mmol) was added. The mixture was stirred at 65° C. for an additional 3 hours. The mixture was filtered, and the filtrate was concentrated in vacuo. The residue was partitioned between DCM/H₂O (50 mL each). The organic layer was washed with brine, dried over Na₂SO₄, and filtered. The filtrate was concentrated in vacuo to provide tert-butyl (3-(9-(1,3-dihydroxypropan-2-yl)-3,9-diazaspiro[5.5]undecan-3-yl)propyl) carbamate (1.9 g, 91% yield) as a yellow oil. LCMS (ESI): Calc'd for C₂₀H₄₀N₃O₄ [M+H]⁺ 386.29; found 386.2.

### Step 2: 2-(9-(3-((tert-butoxycarbonyl)amino)propyl)-3,9-diazaspiro[5.5]undecan-3-yl)propane-1,3-diyl bis(2-heptylnonanoate)

To a solution of tert-butyl (3-(9-(1,3-dihydroxypropan-2-yl)-3,9-diazaspiro[5.5]undecan-3-yl)propyl) carbamate (1.0 g, 2.5 mmol) and 2-heptylnonanoic acid (A12) (1.4 g, 5.7 mmol) in DCM (20 mL) was added DMAP (0.063 g, 0.52 mmol) followed by DCC (1.6 g, 7.8 mmol). The reaction mixture was stirred at room temperature for 16 hours. The mixture was filtered, and the filtrate was concentrated in vacuo. The residue was purified by column chromatography to provide 2-(9-(3-((tert-butoxycarbonyl)amino)propyl)-3,9-diazaspiro[5.5]undecan-3-yl)propane-1,3-diyl bis(2-heptylnonanoate) (1.7 g, 75% yield) as a light-yellow oil.

### Step 3: 2-(9-(3-aminopropyl)-3,9-diazaspiro[5.5]undecan-3-yl)propane-1,3-diyl bis(2-heptylnonanoate)

A solution of 2-(9-(3-((tert-butoxycarbonyl)amino)propyl)-3,9-diazaspiro[5.5]undecan-3-yl)propane-1,3-diyl bis(2-heptylnonanoate) (1.68 g, 1.95 mmol) in HCl/dioxane (4M, 10 mL) was stirred at room temperature for 12 hours. The mixture was quenched with saturated Na₂CO₃ until no gas was generated, and then the phases were separated. The aqueous layer was extracted with DCM (20 mL×4). The combined organic layers were dried over Na₂SO₄ and filtered. The filtrate was concentrated in vacuo. The residue was purified by column chromatography to provide 2-(9-(3-aminopropyl)-3,9-diazaspiro[5.5]undecan-3-yl)propane-1,3-diyl bis(2-heptylnonanoate) (1.2 g, 83% yield) as a yellow oil. LCMS (ESI): Calc'd for C₄₇H₉₂N₃O₄ [M+H]⁺ 762.70; found 762.6.

### Step 4: 2-(9-(3-((2-(methylamino)-3,4-dioxocyclobut-1-en-1-yl)amino)propyl)-3,9-diazaspiro[5.5]undecan-3-yl)propane-1,3-diyl bis(2-heptylnonanoate)

### General Squaramide Formation Using 3-Ethoxy-4-(Methylamino) Cyclobut-3-Ene-1,2-Dione

TEA (0.494 g, 4.88 mmol) was added to a solution of 2-(9-(3-aminopropyl)-3,9-diazaspiro[5.5]undecan-3-yl)propane-1,3-diyl bis(2-heptylnonanoate) (1.24 g, 1.63 mmol) in 1,4-dioxane (25 mL), and the mixture was stirred for 15 minutes at 20° C. 3-Ethoxy-4-(methylamino) cyclobut-3-ene-1,2-dione (0.315 g, 2.03 mmol) was added, and the mixture was stirred for an additional 16 hours at 80° C. The mixture was concentrated in vacuo, and the residue was purified by column chromatography followed by prep-SFC to provide 2-(9-(3-((2-(methylamino)-3,4-dioxocyclobut-1-en-1-yl)amino)propyl)-3,9-diazaspiro[5.5]undecan-3-yl)propane-1,3-diyl bis(2-heptylnonanoate) (0.20 g, 14% yield) as a light-yellow oil. ¹H NMR (400 MHz, CDCl₃) δ 4.24 (dd, J=11.5, 6.3 Hz, 2H), 3.99 (dd, J=11.5, 5.5 Hz, 2H), 3.53 (br s, 2H), 3.23 (t, J=4.5 Hz, 3H), 2.91 (p, J=5.9 Hz, 1H), 2.54 (t, J=5.4 Hz, 4H), 2.45 (t, J=6.2 Hz, 2H), 2.37 (br s, 4H), 2.26 (tt, J=8.6, 5.3 Hz, 2H), 1.76-1.64 (m, 7H), 1.56-1.46 (m, 4H), 1.45-1.32 (m, 11H), 1.26-1.12 (m, 38H), 0.80 (t, J=6.7 Hz, 12H). LCMS (ESI): Calc'd for C₅₂H₉₅N₄O₆ [M+H]⁺ 871.72; found 871.7.

### Example 9

### 2-(9-(3-(1-methylcyclopropane-1-carboxamido)propyl)-3,9-diazaspiro[5.5]undecan-3-yl)propane-1,3-diyl bis(2-hexyldecanoate) (9)

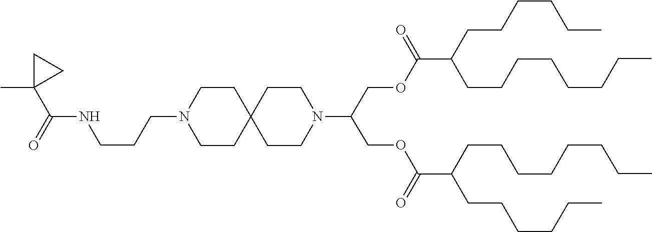 [SMILES: CCCCCCCCC(CCCCCC)C(=O)OCC(COC(=O)C(CCCCCC)CCCCCCCC)N1CCC2(CCN(CCCNC(=O)C3(C)CC3)CC2)CC1]

### Step 1: tert-butyl (tert-butoxycarbonyl) (3-(9-(1,3-dihydroxypropan-2-yl)-3,9-diazaspiro[5.5]undecan-3-yl)propyl) carbamate

General Alkylation Procedure Using tert-butyl (3-bromopropyl) (tert-butoxycarbonyl)carbamate

K₂CO₃ (3.75 g, 27.1 mmol) was added to a solution of 2-(3,9-diazaspiro[5.5]undecan-3-yl)propane-1,3-diol (Example 3, Step 2) (1.63 g, 5.43 mmol) in DMF (50 mL) and MeOH (5 mL). The mixture was stirred at room temperature for 1 hour. tert-Butyl (3-bromopropyl) (tert-butoxycarbonyl) carbamate (2.02 g, 5.97 mmol) was added, and the mixture was stirred at 70° C. for an 16 hours. The mixture was filtered, and the filtrate was concentrated in vacuo. The residue was partitioned between DCM and H₂O. The organic layer was washed with brine, dried over Na₂SO₄, and filtered. The filtrate was concentrated in vacuo, and the residue was purified by silica gel chromatography to provided tert-butyl (tert-butoxycarbonyl) (3-(9-(1,3-dihydroxypropan-2-yl)-3,9-diazaspiro[5.5]undecan-3-yl)propyl) carbamate (1.10 g, 42% yield) as a white solid. ¹H NMR (400 MHZ, CD₃OD) δ 3.75-3.56 (m, 6H), 2.74 (m, 4H), 2.70-2.63 (m, 1H), 2.47 (m, 4H), 2.44-2.35 (m, 2H), 1.84-1.76 (m, 2H), 1.51 (s, 26H). LCMS (ESI): Calc'd for C₂₅H₄₈N₃O₆ [M+H]⁺ 486.4; found 486.4.

### Step 2: 2-(9-(3-(bis(tert-butoxycarbonyl)amino)propyl)-3,9-diazaspiro[5.5]undecan-3-yl)propane-1,3-diyl bis(2-hexyldecanoate)

This compound was prepared according to the general acylation procedure as described in Example 1 (Step 4). tert-Butyl (tert-butoxycarbonyl) (3-(9-(1,3-dihydroxypropan-2-yl)-3,9-diazaspiro[5.5]undecan-3-yl)propyl) carbamate (1.10 g, 2.27 mmol) and 2-hexyldecanoic acid (A13) (1.45 g, 5.66 mmol) afforded 2-(9-(3-(bis(tert-butoxycarbonyl)amino)propyl)-3,9-diazaspiro[5.5]undecan-3-yl)propane-1,3-diyl bis(2-hexyldecanoate) (1.93 g, 89% yield) as a colorless oil. ¹H NMR (400 MHZ, CDCl₃) δ 4.29 (dd, J=11.5, 6.2 Hz, 2H), 4.07 (dd, J=11.6, 5.5 Hz, 2H), 3.57 (t, J=7.3 Hz, 2H), 2.98-2.96 (m, 1H), 2.59 (m, 4H), 2.40-2.26 (m, 8H), 1.81-1.75 (m, 2H), 1.65-1.35 (m, 34H), 1.24 (m, 40H), 0.87 (m, 12H). LCMS (ESI): Calc'd for C₅₇H₁₀₈N₃O₈ [M+H] 962.8; found 962.8.

### Step 3: 2-(9-(3-aminopropyl)-3,9-diazaspiro[5.5]undecan-3-yl)propane-1,3-diyl bis(2-hexyldecanoate)

To a solution of 2-(9-(3-(bis(tert-butoxycarbonyl)amino)propyl)-3,9-diazaspiro[5.5]undecan-3-yl)propane-1,3-diyl bis(2-hexyldecanoate) (1.90 g, 1.97 mmol) in 1,4 dioxane (20.0 mL) at room temperature was added trimethylsilyl trifluoromethanesulfonate (4.39 g, 19.7 mmol) followed by pyridine (1.56 g, 19.7 mmol) and the mixture was stirred at room temperature for 2 hours. At this time the mixture was quenched with sat. NaHCO₃, extracted with EtOAc, the combined organic phases were dried over Na₂SO₄, filtered, and concentrated to give the crude mixture. The residue was purified by silica gel chromatography to give 2-(9-(3-aminopropyl)-3,9-diazaspiro[5.5]undecan-3-yl)propane-1,3-diyl bis(2-hexyldecanoate) (1.50 g, 99% yield) as a yellow gum. ¹H NMR (400 MHZ, CDCl₃) δ 4.37-4.28 (m, 2H), 4.11-4.04 (m, 2H), 3.20 (m, 2H), 3.05-2.87 (m, 7H), 2.63 (m, 4H), 2.39-2.29 (m, 2H), 2.07 (m, 2H), 1.72-1.37 (m, 17H), 1.27 (s, 46H), 0.89 (m, 12H). LCMS (ESI): Calc'd for C₄₇H₉₂N₃O₄ [M+H] 762.7; found 762.7.

### Step 4: 2-(9-(3-(1-methylcyclopropane-1-carboxamido)propyl)-3,9-diazaspiro[5.5]undecan-3-yl)propane-1,3-diyl bis(2-hexyldecanoate)

To a solution of 2-(9-(3-aminopropyl)-3,9-diazaspiro[5.5]undecan-3-yl)propane-1,3-diyl bis(2-hexyldecanoate) (300 mg, 0.394 mmol) in DCM (6.0 mL) was added HATU (150 mg, 0.394 mmol), HOBt (5.32 mg, 0.039 mmol) followed by 1-methylcyclopropane-1-carboxylic acid (39 mg, 0.394 mmol) and the mixture was stirred at room temperature for 16 hours. At this time the mixture was partitioned between DCM/sat NaHCO₃, the organic layer was washed with brine, H₂O, dried over Na₂SO₄ and concentrated. The residue was purified by chromatography to deliver 2-(9-(3-(1-methylcyclopropane-1-carboxamido)propyl)-3,9-diazaspiro[5.5]undecan-3-yl)propane-1,3-diyl bis(2-hexyldecanoate) (90.0 mg, 27% yield). ¹H NMR (400 MHZ, CDCl₃) δ 7.32 (brs, 1H), 4.30 (dd, J=11.5, 6.3 Hz, 2H), 4.06 (dd, J=11.6, 5.5 Hz, 2H), 3.36 (m, 2H), 2.98 (m, 1H), 2.78-2.40 (m, 8H), 2.36-2.29 (m, 3H), 1.85-1.1.02 (m, 64H), 0.93-0.82 (m, 12H), 0.54 (m, 2H). LCMS (ESI): Calc'd for C₅₂H₉₈N₃O₅ [M+H]⁺ 844.7; found 844.7.

### Example 10

### 2-(9-(3-hydroxypropyl)-3,9-diazaspiro[5.5]undecan-3-yl)propane-1,3-diyl bis(2-heptylnonanoate) (10)

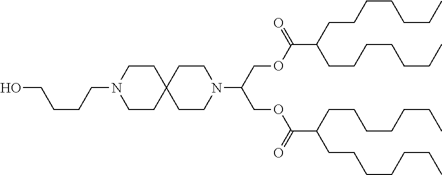 [SMILES: CCCCCCCC(CCCCCCC)C(=O)OCC(COC(=O)C(CCCCCCC)CCCCCCC)N1CCC2(CCN(CCCCO)CC2)CC1]

### Step 1: 2-(9-(3-((tert-butyldimethylsilyl)oxy) propyl)-3,9-diazaspiro[5.5]undecan-3-yl)propane-1,3-diol

This compound was prepared according to the general alkylation procedure as described in Example 1 (Step 3). 2-(3,9-Diazaspiro[5.5]undecan-3-yl)propane-1,3-diol (Example 3, Step 2) (2.0 g, 8.75 mmol) and (3-bromopropoxy) (tert-butyl)dimethylsilane (2.4 g, 9.63 mmol) afforded 2-(9-(3-((tert-butyldimethylsilyl)oxy) propyl)-3,9-diazaspiro[5.5]undecan-3-yl)propane-1,3-diol (1.81 g, 51% yield) as a yellow oil. ¹H NMR (400 MHZ, CDCl₃) δ 3.68-3.58 (m, 6H), 2.76 (p, J=6.5 Hz, 1H), 2.67-2.65 (m, 4H), 2.46-2.38 (m, 6H), 1.78-1.67 (m, 2H), 1.51 (dt, J=17.1, 5.6 Hz, 9H), 0.88 (s, 9H), 0.03 (s, 6H).

### Step 2:2-(9-(3-((tert-butyldimethylsilyl)oxy) propyl)-3,9-diazaspiro[5.5]undecan-3-yl)propane-1,3-diyl bis(2-heptylnonanoate)

This compound was prepared according to the general acylation procedure as described in Example 1 (Step 4). 2-(9-(3-((tert-butyldimethylsilyl)oxy) propyl)-3,9-diazaspiro[5.5]undecan-3-yl)propane-1,3-diol (1.81 g, 4.36 mmol) and 2-heptylnonanoic acid (A12) (2.5 g, 9.59 mmol) delivered 2-(9-(3-((tert-butyldimethylsilyl)oxy) propyl)-3,9-diazaspiro[5.5]undecan-3-yl)propane-1,3-diyl bis(2-heptylnonanoate) (1.80 g, 45% yield) as a yellow oil.

### Step 3:2-(9-(3-hydroxypropyl)-3,9-diazaspiro[5.5]undecan-3-yl)propane-1,3-diyl bis(2-heptylnonanoate)

This compound was prepared according to the general HCl-mediated tert-butyldimethylsilyl deprotection as described in Example 1 (Step 5). 2-(9-(3-((tert-butyldimethylsilyl)oxy) propyl)-3,9-diazaspiro[5.5]undecan-3-yl)propane-1,3-diyl bis(2-heptyl-nonanoate) 1.81 g, 2.01 mmol) afforded 2-(9-(3-hydroxypropyl)-3,9-diazaspiro[5.5]undecan-3-yl)propane-1,3-diyl bis(2-heptylnonanoate) (740 mg, 44% yield) as a yellow oil. ¹H NMR (400 MHz, CDCl₃) δ 4.29 (dd, J=11.5, 6.3 Hz, 2H), 4.06 (dd, J=11.5, 5.5 Hz, 2H), 3.79 (t, J=5.1 Hz, 2H), 2.97 (p, J=5.9 Hz, 1H), 2.62-2.58 (m, 6H), 2.46-2.28 (m, 6H), 1.73-1.69 (m, 2H), 1.58-1.53 (m, 4H), 1.49-1.39 (m, 12H), 1.25 (m, 41H), 0.87 (m, 12H). LCMS (ESI): Calc'd for C₄₇H₉₁N₂O₅ [M+H]⁺ 763.7; found 763.7.

### Example 11

### 2-(9-(3-(ethylsulfonamido)propyl)-3,9-diazaspiro[5.5]undecan-3-yl)propane-1,3-diyl bis(2-hexyldecanoate) (11)

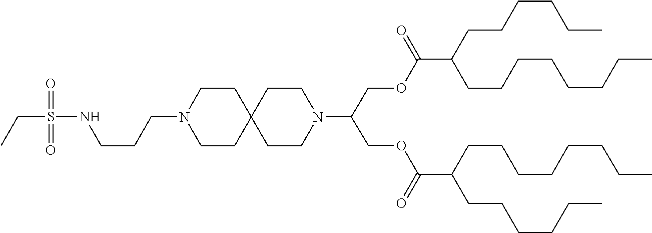 [SMILES: CCCCCCCCC(CCCCCC)C(=O)OCC(COC(=O)C(CCCCCC)CCCCCCCC)N1CCC2(CCN(CCCNS(=O)(=O)CC)CC2)CC1]

Ethanesulfonyl chloride (40.5 mg, 0.315 mmol) followed by DIPEA (85 mg, 0.656 mmol) were added to a solution of 2-(9-(3-aminopropyl)-3,9-diazaspiro[5.5]undecan-3-yl)propane-1,3-diyl bis(2-hexyldecanoate) (Example 9, step 3) (200 mg, 0.262 mmol) in THF (2.62 mL). The mixture was stirred at room temperature for 16 hours and then partitioned between EtOAc and H₂O. The organic layer was washed with brine, dried over Na₂SO₄, and filtered. The filtrate was concentrated in vacuo, and the crude product was purified by silica gel chromatography to afford 2-(9-(3-(ethylsulfonamido)propyl)-3,9-diazaspiro[5.5]undecan-3-yl)propane-1,3-diyl bis(2-hexyldecanoate) (118 mg, 53%). ¹H NMR (400 MHZ, CDCl₃) δ 4.25-4.41 (m, 2H), 4.25-4.37 (m, 2H), 3.98-4.13 (m, 2H), 3.23-3.35 (m, 2H), 2.95-3.10 (m, 4H), 2.59-2.71 (m, 4H), 2.27-2.44 (m, 2H), 1.90-2.04 (m, 2H), 1.68-1.82 (m, 4H), 1.53-1.64 (m, 6H), 1.42-1.53 (m, 8H), 1.35-1.40 (m, 4H), 1.17-1.32 (m, 41H), 0.83-0.96 (m, 12H). LCMS (ESI): Calc'd for C₄₉H₉₆N₃O₆S [M+H]⁺ 854.69; found 854.8.

### Example 12

### 2-(7-(4-hydroxybutyl)-2,7-diazaspiro[4.4]nonan-2-yl)propane-1,3-diyl bis(2-heptylnonanoate) (12)

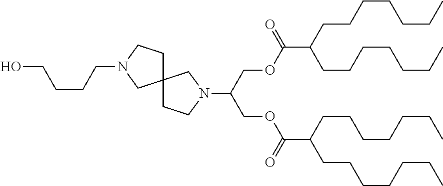 [SMILES: CCCCCCCC(CCCCCCC)C(=O)OCC(COC(=O)C(CCCCCCC)CCCCCCC)N1CCC2(CCN(CCCCO)C2)C1]

### Step 1: tert-butyl 7-(2,2-dimethyl-1,3-dioxan-5-yl)-2,7-diazaspiro[4.4]nonane-2-carboxylate

This compound was prepared according to the general reductive amination as described in Example 3 (Step 1). tert-Butyl 2,7-diazaspiro[4.4]nonane-2-carboxylate (5.0 g, 22.1 mmol) and 2,2-dimethyl-1,3-dioxan-5-one (3.16 g, 24.3 mmol) provided tert-butyl 7-(2,2-dimethyl-1,3-dioxan-5-yl)-2,7-diazaspiro[4.4]nonane-2-carboxylate (5.0 g, 66% yield) as a colorless oil. LCMS (ESI): Calc'd for C₁₈H₃₃N₂O₄ [M+H]⁺ 341.24; found 341.1.

### Step 2:2-(2,7-diazaspiro[4.4]nonan-2-yl)propane-1,3-diol

This compound was prepared according to the general acetal hydrolysis/boc-deprotection as described in example 3 (Step 2). tert-Butyl 7-(2,2-dimethyl-1,3-dioxan-5-yl)-2,7-diazaspiro[4.4]nonane-2-carboxylate (1.8 g, 5.3 mmol) afforded 2-(2,7-diazaspiro[4.4]nonan-2-yl)propane-1,3-diol (1.40 g, 100%) as a yellow oil which was used crude in subsequent transformations. LCMS (ESI): Calc'd for C₁₀H₂₁N₂O₂ [M+H]⁺ 201.15; found 201.1.

### Step 3:2-(7-(4-((tert-butyldiphenylsilyl)oxy)butyl)-2,7-diazaspiro[4.4]nonan-2-yl)propane-1,3-diol

This compound was prepared according to the general alkylation procedure as described in Example 1 (step 3). 2-(2,7-Diazaspiro[4.4]nonan-2-yl)propane-1,3-diol (1.44 g, 5.29 mmol) and (4-bromobutoxy) (tert-butyl) diphenylsilane (3.65 g, 5.29 mmol) provided 2-(7-(4-((tert-butyldimethylsilyl)oxy)butyl)-2,7-diazaspiro[4.4]nonan-2-yl)propane-1,3-diol (0.50 g, 18% yield) as a yellow oil. LCMS (ESI): Calc'd for C₃₀H₄₇N₂O₃Si [M+H]⁺ 511.33; found 511.3.

### Step 4:2-(7-(4-((tert-butyldiphenylsilyl)oxy)butyl)-2,7-diazaspiro[4.4]nonan-2-yl)propane-1,3-diyl bis(2-heptylnonanoate)

This compound was prepared according to the general acylation procedure as described in Example 1 (Step 4). 2-(7-(4-((tert-Butyldimethylsilyl)oxy)butyl)-2,7-diazaspiro[4.4]nonan-2-yl)propane-1,3-diol (0.90 g, 1.8 mmol) and 2-heptylnonanoic acid (0.994 g, 3.88 mmol) provided 2-(7-(4-((tert-butyldimethylsilyl)oxy)butyl)-2,7-diazaspiro[4.4]nonan-2-yl)propane-1,3-diyl bis(2-heptylnonanoate) (0.90 g, 52% yield) as a yellow solid. LCMS (ESI): Calc'd for C₆₂H₁₀₇N₂O₅Si [M+H]⁺ 987.79; found 988.6.

### Step 5:2-(7-(4-hydroxybutyl)-2,7-diazaspiro[4.4]nonan-2-yl)propane-1,3-diyl bis(2-heptylnonanoate)

A solution of 2-(7-(4-((tert-butyldimethylsilyl)oxy)butyl)-2,7-diazaspiro[4.4]nonan-2-yl)propane-1,3-diyl bis(2-heptylnonanoate) (0.90 g, 0.91 mmol) in HCl (4M/dioxane, 10 mL) was stirred at room temperature for 12 hours. The mixture was quenched with saturated Na₂CO₃ until no gas was generated. The aqueous layer was separated and then extracted by DCM (20 ml×4). The combined organic layers were dried over Na₂SO₄ and filtered. The filtrate was concentrated in vacuo, and the residue was purified by column chromatography to provide 2-(7-(4-hydroxybutyl)-2,7-diazaspiro[4.4]nonan-2-yl)propane-1,3-diyl bis(2-heptylnonanoate) (0.24 g, 35% yield) as a yellow oil. ¹H NMR (400 MHZ, CDCl₃) δ 4.26-4.11 (m, 4H), 3.58 (br s, 2H), 2.87-2.80 (m, 2H), 2.79-2.71 (m, 3H), 2.69-2.50 (m, 6H) 2.32 (tt, J=8.8, 5.4 Hz, 2H), 1.92-1.81 (m, 3H), 1.81-1.72 (m, 2H), 1.68 (br s, 4H), 1.62-1.50 (m, 4H), 1.48-1.37 (m, 4H), 1.31-1.19 (m, 40H), 0.87 (t, J=6.8 Hz, 12H). LCMS (ESI): Calc'd for C₄₆H₈₉N₂O₅ [M+H]⁺ 749.67; found 749.7.

### Example 13

### 2-(7-(4-hydroxybutyl)-2,7-diazaspiro[3.5]nonan-2-yl)propane-1,3-diyl bis(2-heptylnonanoate) (13)

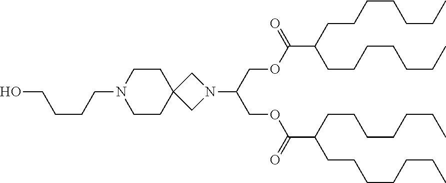 [SMILES: CCCCCCCC(CCCCCCC)C(=O)OCC(COC(=O)C(CCCCCCC)CCCCCCC)N1CC2(CCN(CCCCO)CC2)C1]

### Step 1: tert-butyl 2-(2,2-dimethyl-1,3-dioxan-5-yl)-2,7-diazaspiro[3.5]nonane-7-carboxylate

This compound was prepared according to the general reductive amination as described in Example 3 (Step 1). tert-Butyl 2,7-diazaspiro[3.5]nonane-7-carboxylate hydrochloride (10 g, 31 mmol) and 2,2-dimethyl-1,3-dioxan-5-one (8.05 g, 61.9 mmol) afforded tert-butyl 2-(2,2-dimethyl-1,3-dioxan-5-yl)-2,7-diazaspiro[3.5]nonane-7-carboxylate (5.0 g, 47% yield) as a light-yellow solid. ¹H NMR (400 MHz, CDCl₃) δ 3.76 (dd, J=11.9, 4.7 Hz, 2H), 3.55 (dd, J=11.8, 8.7 Hz, 2H), 3.32-3.27 (m, 4H), 3.04 (s, 4H), 2.50 (tt, J=9.0, 4.7 Hz, 1H), 1.69-1.64 (m, 4H), 1.44-1.36 (m, 15H). LCMS (ESI): Calc'd for C₁₈H₃₃N₂O₄ [M+H]⁺ 341.24; found 341.2.

### Step 2:2-(2,7-diazaspiro[3.5]nonan-2-yl)propane-1,3-diol

This compound was prepared according to the general acetal hydrolysis/boc-deprotection as described in Example 3 (Step 2). tert-Butyl 2-(2,2-dimethyl-1,3-dioxan-5-yl)-2,7-diazaspiro[3.5]nonane-7-carboxylate (5.0 g, 15 mmol) provided 2-(2,7-diazaspiro[3.5]nonan-2-yl)propane-1,3-diol (3.00 g, 100% yield, crude) as yellow solid. The material was used in the subsequent transformation without further purification. LCMS (ESI): Calc'd for C₁₀H₂₁N₂O₂ [M+H]⁺ 201.15; found 201.1.

### Step 3:2-(7-(4-((tert-butyldimethylsilyl)oxy)butyl)-2,7-diazaspiro[3.5]nonan-2-yl)propane-1,3-diol

This compound was prepared according to the general alkylation procedure as described in Example 1 (Step 3). 2-(2,7-Diazaspiro[3.5]nonan-2-yl)propane-1,3-diol (3.0 g, 15 mmol) and (4-bromobutoxy) (tert-butyl)dimethylsilane (4.08 g, 15.2 mmol) afforded 2-(7-(4-((tert-butyldimethylsilyl)oxy)butyl)-2,7-diazaspiro[3.5]nonan-2-yl)propane-1,3-diol (4.0 g, 69% yield) as a yellow oil. LCMS (ESI): Calc'd for C₂₀H₄₃N₂O₃Si [M+H]⁺ 387.30; found 387.6.

### Step 4:2-(7-(4-((tert-butyldimethylsilyl)oxy)butyl)-2,7-diazaspiro[3.5]nonan-2-yl)propane-1,3-diyl bis(2-heptylnonanoate)

This compound was prepared according to the general acylation procedure as described in Example 1 (Step 4). 2-(7-(4-((tert-Butyldimethylsilyl)oxy)butyl)-2,7-diazaspiro[3.5]nonan-2-yl)propane-1,3-diol (2.0 g, 5.2 mmol) and 2-heptylnonanoic acid (A12) (2.92 g, 11.4 mmol) provided 2-(7-(4-((tert-butyldimethylsilyl)oxy)butyl)-2,7-diazaspiro[3.5]nonan-2-yl)propane-1,3-diyl bis(2-heptylnonanoate) (2.0 g, 44% yield) as a yellow oil.

### Step 5:2-(7-(4-hydroxybutyl)-2,7-diazaspiro[3.5]nonan-2-yl)propane-1,3-diyl bis(2-heptylnonanoate)

This compound was prepared according to the general HCl-mediated tert-butyldimethylsilyl deprotection as described in Example 1 (Step 5). 2-(7-(4-((tert-Butyldimethylsilyl)oxy)butyl)-2,7-diazaspiro[3.5]nonan-2-yl)propane-1,3-diyl bis(2-heptylnonanoate) (1.8 g, 2.1 mmol) provided 2-(7-(4-hydroxybutyl)-2,7-diazaspiro[3.5]nonan-2-yl)propane-1,3-diyl bis(2-heptylnonanoate) (0.43 g, 48% yield) as a colorless oil. ¹H NMR (400 MHz, CDCl₃) δ 4.10 (dd, J=11.4, 5.4 Hz, 2H), 3.98 (dd, J=11.4, 4.6 Hz, 2H), 3.51-3.62 (m, 2H), 3.07 (s, 4H), 2.61 (p, J=5.0 Hz, 1H), 2.47-2.27 (m, 6H), 1.82-1.77 (m, 4H), 1.67 (s, 5H), 1.62-1.52 (m, 5H), 1.48-1.38 (m, 5H), 1.33-1.21 (m, 40H), 0.87 (t, J=6.7 Hz, 12H). LCMS (ESI): Calc'd for C₄₆H₈₉N₂O₅ [M+H]⁺ 749.67; found 749.7.

### Example 14

### 2-(8-(4-hydroxybutyl)-2,8-diazaspiro[4.5]decan-2-yl)propane-1,3-diyl bis(2-heptylnonanoate) (14)

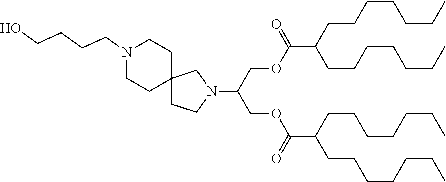 [SMILES: CCCCCCCC(CCCCCCC)C(=O)OCC(COC(=O)C(CCCCCCC)CCCCCCC)N1CCC2(CCN(CCCCO)CC2)C1]

### Step 1: tert-butyl 2-(2,2-dimethyl-1,3-dioxan-5-yl)-2,8-diazaspiro[4.5]decane-8-carboxylate

This compound was prepared according to the general reductive amination procedure as described in Example 3 (Step 1). tert-Butyl 2,8-diazaspiro[4.5]decane-8-carboxylate (5.00 g, 14.1 mmol) and 2,2-dimethyl-1,3-dioxan-5-one (2.02 g, 15.5 mmol) provided tert-butyl 2-(2,2-dimethyl-1,3-dioxan-5-yl)-2,8-diazaspiro[4.5]decane-8-carboxylate (4.5 g, 90% yield) as a yellow oil. LCMS (ESI): Calc'd for C₁₉H₃N₂O₄ [M+H]⁺ 355.25; found 355.2.

### Step 2:2-(2,8-diazaspiro[4.5]decan-2-yl)propane-1,3-diol

This compound was prepared according to the general acetal deprotection/boc-deprotection protocol as described in Example 3 (Step 2). tert-Butyl 2-(2,2-dimethyl-1,3-dioxan-5-yl)-2,8-diazaspiro[4.5]decane-8-carboxylate (4.50 g, 12.7 mmol) provided 2-(2,8-diazaspiro[4.5]decan-2-yl)propane-1,3-diol (2.72 g, 100% yield) as a yellow oil. The material was used in the subsequent transformation without further purification. LCMS (ESI): Calc'd for C₁₁H₂₃N₂O₂ [M+H]⁺ 215.17; found 215.1.

### Step 3: 2-(8-(4-((tert-butyldimethylsilyl)oxy)butyl)-2,8-diazaspiro[4.5]decan-2-yl)propane-1,3-diol

This compound was prepared according to the general alkylation procedure as described in example 1 (Step 3). 2-(2,8-Diazaspiro[4.5]decan-2-yl)propane-1,3-diol (2.72 g, 12.7 mmol) afforded 2-(8-(4-((tert-butyldimethylsilyl)oxy)butyl)-2,8-diazaspiro[4.5]decan-2-yl)propane-1,3-diol (2.6 g, 52% yield) as a yellow oil. LCMS (ESI): Calc'd for C₂₁H₄₅N₂O₃Si [M+H]⁺ 401.31; found 401.2.

### Step 4:2-(8-(4-((tert-butyldimethylsilyl)oxy)butyl)-2,8-diazaspiro[4.5]decan-2-yl)propane-1,3-diyl bis(2-heptylnonanoate)

This compound was prepared according to the general acylation procedure as described in Example 1 (Step 4). 2-(8-(4-((tert-Butyldimethylsilyl)oxy)butyl)-2,8-diazaspiro[4.5]decan-2-yl)propane-1,3-diol and 2-heptylnonanoic acid (A12) (1.60 g, 6.24 mmol) delivered 2-(8-(4-((tert-butyldimethylsilyl)oxy)butyl)-2,8-diazaspiro[4.5]decan-2-yl)propane-1,3-diyl bis(2-heptylnonanoate) (1.8 g, 82% yield) as a colorless oil. ¹H NMR (400 MHZ, CDCl₃) δ 4.25-4.14 (m, 4H), 3.60 (t, J=6.0 Hz, 2H), 2.78-2.69 (m, 4H), 2.52 (s, 2H), 2.39-2.29 (m, 5H), 1.62-1.49 (m, 14H), 1.46-1.37 (m, 4H), 1.29-1.20 (m, 42H), 0.89-0.85 (m, 21H), 0.04 (s, 6H).

### Step 5:2-(8-(4-hydroxybutyl)-2,8-diazaspiro[4.5]decan-2-yl)propane-1,3-diyl bis(2-heptylnonanoate)

This compound was prepared according to the general HCl-mediated tertbutyldimethyl silyl deprotection procedure as described in Example 1 (Step 5). 2-(8-(4-((tert-Butyldimethylsilyl)oxy)butyl)-2,8-diazaspiro[4.5]decan-2-yl)propane-1,3-diyl bis(2-heptylnonanoate) (1.79 g, 2.04 mmol) provided 2-(8-(4-hydroxybutyl)-2,8-diazaspiro[4.5]decan-2-yl)propane-1,3-diyl bis(2-heptylnonanoate) (0.31 g, 20% yield) as a light-yellow oil. ¹H NMR (400 MHZ, CDCl₃) δ 4.26-4.14 (m, 4H), 3.59 (br s, 2H), 2.74 (dt, J=17.7, 6.0 Hz, 4H), 2.56 (s, 2H), 2.49 (br s, 1H), 2.33 (tt, J=8.7, 5.4 Hz, 3H), 1.79-1.64 (m, 8H), 1.62-1.52 (m, 7H), 1.49-1.38 (m, 5H), 1.31-1.20 (m, 42H), 0.87 (t, J=6.8 Hz, 12H). LCMS (ESI): Calc'd for C₄₇H₉₁N₂O₅ [M+H]⁺ 763.68; found 763.8.

### Example 15

### 3-(9-(4-hydroxybutyl)-3,9-diazaspiro[5.5]undecan-3-yl)pentane-1,5-diyl bis(2-hexyldecanoate) (15)

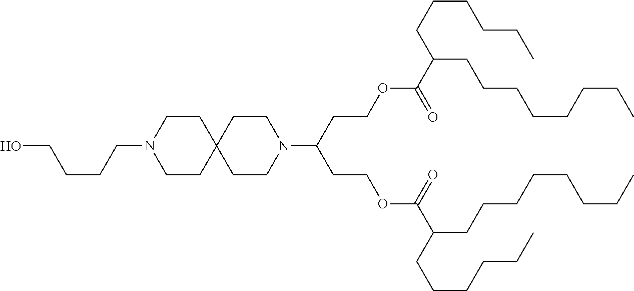 [SMILES: CCCCCCCCC(CCCCCC)C(=O)OCCC(CCOC(=O)C(CCCCCC)CCCCCCCC)N1CCC2(CCN(CCCCO)CC2)CC1]

### Step 1: tert-butyl 9-(5-hydroxy-1-methoxy-1-oxopentan-3-yl)-3,9-diazaspiro[5.5]undecane-3-carboxylate

5,6-Dihydro-2H-pyran-2-one (3.10 g, 31.5 mmol) was added to a solution of tert-butyl 3,9-diazaspiro[5.5]undecane-3-carboxylate (4.0 g, 15.7 mmol) in MeOH (120 mL) at room temperature, and the mixture was stirred at room temperature for 16 hours. The mixture was concentrated, and the residue was purified by silica gel chromatography to provide tert-butyl 9-(5-hydroxy-1-methoxy-1-oxopentan-3-yl)-3,9-diazaspiro[5.5]undecane-3-carboxylate (4.8 g, 79% yield) as a yellow gum. LCMS (ESI): Calc'd for C₂₀H₃₇N₂O₅ [M+H]⁺ 385.3; found 385.3.

### Step 2: tert-butyl 9-(1,5-dihydroxypentan-3-yl)-3,9-diazaspiro[5.5]undecane-3-carboxylate

Calcium chloride (1.39 g, 12.5 mmol) was added to a solution of tert-butyl 9-(5-hydroxy-1-methoxy-1-oxopentan-3-yl)-3,9-diazaspiro[5.5]undecane-3-carboxylate (4.0 g, 10.40 mmol) in THF (20 mL) and EtOH (20 mL) at room temperature followed by the slow addition of sodium borohydride (1.0 g, 25.4 mmol). After stirring at room temperature for 16 hours, the mixture was slowly added into saturated NH₄Cl and concentrated in vacuo to dryness. The residue was partitioned between DCM:iPrOH (4:1) and H₂O. The organic layer was separated, washed with brine, and dried over Na₂SO₄. The filtrate was concentrated in vacuo, and the residue was purified by silica gel chromatography to afford tert-butyl 9-(1,5-dihydroxypentan-3-yl)-3,9-diazaspiro[5.5]undecane-3-carboxylate (2.9 g, 78% yield) as a white gum. LCMS (ESI): Calc'd for C₁₉H₃₇N₂O₄ [M+H]⁺ 357.3; found 357.3.

### Step 3:3-(3,9-diazaspiro[5.5]undecane-3-yl)pentane-1,5-diol

A solution of tert-butyl 9-(1,5-dihydroxypentan-3-yl)-3,9-diazaspiro[5.5]undecane-3-carboxylate (500 mg, 1.40 mmol) in MeOH (5.0 mL) and HCl (4M dioxane, 5 mL) was stirred at room temperature for 16 hours and then concentrated to afford 3-(3,9-diazaspiro[5.5]undecane-3-yl)pentane-1,5-diol (462 mg, 100% yield) which was used crude in subsequent transformations. LCMS (ESI): Calc'd for C₁₄H₂₉N₂O₂ [M+H]⁺ 257.2; found 257.2.

### Step 4:3-(9-(4-((tert-butyldimethylsilyl)oxy)butyl)-3,9-diazaspiro[5.5]undecane-3-yl)pentane-1,5-diol

This compound was prepared according to the general alkylation procedure as described in Example 1 (Step 3). 3-(3,9-Diazaspiro[5.5]undecane-3-yl)pentane-1,5-diol (462 mg, 1.40 mmol) and (4-bromobutoxy) (tert-butyl)dimethylsilane (375 mg, 1.40 mmol) provided 3-(9-(4-((tert-butyldimethylsilyl)oxy)butyl)-3,9-diazaspiro[5.5]undecane-3-yl)pentane-1,5-diol (420 mg, 68% yield) as a colorless oil. LCMS (ESI): Calc'd for C₂₄H₅₁N₂O₃Si [M+H]⁺ 443.4; found 443.4.

### Step 5:3-(9-(4-((tert-butyldimethylsilyl)oxy)butyl)-3,9-diazaspiro[5.5]undecan-3-yl)pentane-1,5-diyl bis(2-hexyldecanoate)

This compound was prepared according to the general acylation procedure as described in Example 1 (Step 4). 3-(9-(4-((tert-Butyldimethylsilyl)oxy)butyl)-3,9-diazaspiro[5.5]undecane-3-yl)pentane-1,5-diol (370 mg, 0.84 mmol) and 2-hexyldecanoic acid (A13) (536 mg, 2.09 mmol) provided 3-(9-(4-((tert-butyldimethylsilyl)oxy)butyl)-3,9-diazaspiro[5.5]undecan-3-yl)pentane-1,5-diyl bis(2-hexyldecanoate) (760 mg, 99% yield) as a light-yellow oil.

### Step 6:3-(9-(4-hydroxybutyl)-3,9-diazaspiro[5.5]undecan-3-yl)pentane-1,5-diyl bis(2-hexyldecanoate)

This compound was prepared according to the general HCl-mediated tertbutydimethyl silyl deprotection procedure as described in Example 1 (Step 5). 3-(9-(4-((tert-Butyldimethylsilyl)oxy)butyl)-3,9-diazaspiro[5.5]undecan-3-yl)pentane-1,5-diyl bis(2-hexyldecanoate) (180 mg, 0.22 mmol) provided 3-(9-(4-hydroxybutyl)-3,9-diazaspiro[5.5]-undecan-3-yl)pentane-1,5-diyl bis(2-hexyldecanoate) (145 mg, 80% yield) as a light-yellow oil. ¹H NMR (400 MHZ, CD₃OD) δ ppm 4.30-4.18 (m, 2H), 4.15-4.05 (m, 2H), 3.64-3.51 (m, 2H), 2.84-2.70 (m, 1H), 2.57-2.29 (m, 12H), 1.96-1.84 (m, 2H), 1.66-1.42 (m, 22H), 1.36-1.22 (m, 40H), 0.97-0.81 (m, 12H). LCMS (ESI): Calc'd for C₅₀H₉₇N₂O₅ [M+H]⁺ 805.73; found 805.7.

### Example 16

### 2-(7-(5-hydroxypentyl)-2,7-diazaspiro[4.4]nonan-2-yl)propane-1,3-diyl bis(2-hexyldecanoate) (16)

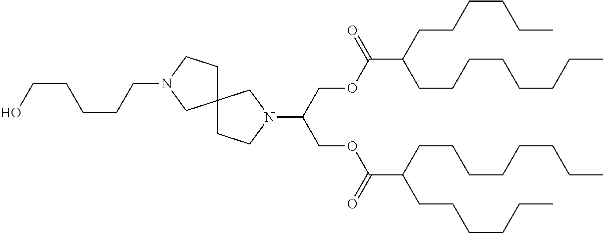 [SMILES: CCCCCCCCC(CCCCCC)C(=O)OCC(COC(=O)C(CCCCCC)CCCCCCCC)N1CCC2(CCN(CCCCCO)C2)C1]

### Step 1: 2-(7-(5-((tert-butyldimethylsilyl)oxy) pentyl)-2,7-diazaspiro[4.4]nonan-2-yl)propane-1,3-diol

This compound was prepared according to the general alkylation procedure as described in Example 1 (Step 3). 2-(2,7-Diazaspiro[4.4]nonan-2-yl)propane-1,3-diol (Example 12, step 2) (3.4 g, 12.44 mmol) and ((5-bromopentyl)oxy) (tert-butyl)dimethylsilane (3.5 g, 12.4 mmol) afforded 2-(7-(5-((tert-butyldimethylsilyl)oxy) pentyl)-2,7-diazaspiro[4.4]nonan-2-yl)propane-1,3-diol (2 g, 37% yield) as a yellow oil. LCMS (ESI): Calc'd for C₂₁H₄₅N₂O₃Si [M+H]⁺ 401.3; found 401.3.

### Step 2: 2-(7-(5-((tert-butyldimethylsilyl)oxy)pentyl)-2,7-diazaspiro[4.4]nonan-2-yl)propane-1,3-diyl bis(2-hexyldecanoate)

This compound was prepared according to the general acylation procedure as described in Example 1 (Step 4). 2-(7-(5-((tert-Butyldimethylsilyl)oxy) pentyl)-2,7-diazaspiro[4.4]nonan-2-yl)propane-1,3-diol (440 mg, 1.10 mmol) and 2-hexyldecanoic acid (A13) (704 mg, 2.75 mmol) afforded 2-(7-(5-((tert-butyldimethylsilyl)oxy) pentyl)-2,7-diazaspiro[4.4]nonan-2-yl)propane-1,3-diyl bis(2-hexyldecanoate) (950 mg, 87%) as a colorless oil. ¹H NMR (400 MHz, CD₃OD) δ 4.26-4.20 (m, 4H), 3.64 (t, J=6.2 Hz, 2H), 2.91-2.62 (m, 11H), 2.40-2.33 (m, 2H), 2.01-1.78 (m, 4H), 1.60-1.55 (m, 8H), 1.49-1.40 (m, 4H), 1.31-1.27 (m, 42H), 0.92-0.87 (m, 21H), 0.05 (s, 6H).

### Step 3: 2-(7-(5-hydroxypentyl)-2,7-diazaspiro[4.4]nonan-2-yl)propane-1,3-diyl bis(2-hexyldecanoate)

This compound was prepared according to the general TBAF-mediated tertbutyldimethyl silyl deprotection procedure as described in Example 4 (Step 2). 2-(7-(5-((tert-Butyldimethylsilyl)oxy) pentyl)-2,7-diazaspiro[4.4]nonan-2-yl)propane-1,3-diyl bis(2-hexyldecanoate) (850 mg, 0.98 mmol) afforded 2-(7-(5-hydroxypentyl)-2,7-diazaspiro[4.4]nonan-2-yl)propane-1,3-diyl bis(2-hexyldecanoate) (451 mg, 56% yield) as a colorless oil. ¹H NMR (400 MHz, CD₃OD) δ ppm 4.16-4.33 (m, 4H), 3.51-3.65 (m, 2H), 2.77-2.90 (m, 4H), 2.64-2.75 (m, 4H), 2.57-2.63 (m, 1H), 2.49-2.56 (m, 2H), 2.33-2.45 (m, 2H), 1.76-2.00 (m, 4H), 1.54-1.69 (m, 8H), 1.45-1.53 (m, 4H), 1.38-1.44 (m, 2H), 1.34-1.39 (m, 40H), 0.83-1.00 (m, 12H). LCMS (ESI): Calc'd for C₄₇H₉₁N₂O₅ [M+H]⁺ 763.68; found 763.9.

### Example 17

### 2-(2-(5-hydroxypentyl)-2,8-diazaspiro[4.5]decan-8-yl)propane-1,3-diyl bis(2-hexyldecanoate) (17)

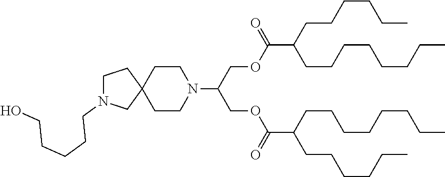 [SMILES: CCCCCCCCC(CCCCCC)C(=O)OCC(COC(=O)C(CCCCCC)CCCCCCCC)N1CCC2(CCN(CCCCCO)C2)CC1]

### Step 1:2-(2-(5-((tert-butyldimethylsilyl)oxy) pentyl)-2,8-diazaspiro[4.5]decan-8-yl)propane-1,3-diol

This compound was prepared according to the general alkylation protocol as described in Example 1 (Step 3). 2-(2,8-Diazaspiro[4.5]decan-8-yl)propane-1,3-diol (Example 6, step 2) (3.5 g, 12.2 mmol) and ((5-bromopentyl)oxy) (tert-butyl)dimethylsilane (3.43 g, 12.2 mmol) provided 2-(2-(5-((tert-butyldimethylsilyl)oxy) pentyl)-2,8-diazaspiro[4.5]decan-8-yl)propane-1,3-diol (2.33 g, 46% yield). LCMS (ESI): Calc'd for C₂₂H₄₇N₂O₅Si [M+H]⁺ 415.3; found 415.2.

### Step 2: 2-(2-(5-((tert-butyldimethylsilyl)oxy)pentyl)-2,8-diazaspiro[4.5]decan-8-yl)propane-1,3-diyl bis(2-hexyldecanoate)

This compound was prepared according to the general acylation procedure as described in Example 1 (Step 4). 2-(2-(5-((tert-Butyldimethylsilyl)oxy) pentyl)-2,8-diazaspiro[4.5]decan-8-yl)propane-1,3-diol (800 mg, 1.93 mmol) and 2-hexyldecanoic acid (A13) (1.24 g, 4.82 mmol) provided 2-(2-(5-((tert-butyldimethylsilyl)oxy) pentyl)-2,8-diazaspiro[4.5]decan-8-yl)propane-1,3-diyl bis(2-hexyldecanoate) (1.2 g, 70% yield) as a light-yellow oil. LCMS (ESI): Calc'd for C₅₄H₁₀₈N₂O₅Si [M+2H]²⁺446.4; found 446.5.

### Step 3: 2-(2-(5-hydroxypentyl)-2,8-diazaspiro[4.5]decan-8-yl)propane-1,3-diyl bis(2-hexyldecanoate)

This compound was prepared according to the general HCl-mediated tertbutyldimethyl silyl deprotection procedure as described in Example 1 (Step 5). 2-(2-(5-((tert-Butyldimethylsilyl)oxy) pentyl)-2,8-diazaspiro[4.5]decan-8-yl)propane-1,3-diyl bis(2-hexyldecanoate) (1.2 g, 1.35 mmol) provided 2-(2-(5-hydroxypentyl)-2,8-diazaspiro[4.5]decan-8-yl)propane-1,3-diyl bis(2-hexyldecanoate) (500 mg, 48% yield) as a light-yellow oil. ¹H NMR (400 MHz, CD₃OD) δ ppm 4.26-4.40 (m, 2H), 4.08-4.17 (m, 2H), 3.48-3.60 (m, 2H), 2.96-3.07 (m, 1H), 2.57-2.73 (m, 6H), 2.42-2.51 (m, 4H), 2.29-2.41 (m, 2H), 1.53-1.68 (m, 14H), 1.42-1.51 (m, 4H), 1.20-1.39 (m, 42H), 0.85-0.94 (m, 12H). LCMS (ESI): Calc'd for C₄₈H₉₃N₂O₅ [M+H]⁺ 777.70; found 777.9.

### Example 18

### 2-(7-(5-hydroxypentyl)-2,7-diazaspiro[3.5]nonan-2-yl)propane-1,3-diyl bis(2-heptylnonanoate) (18)

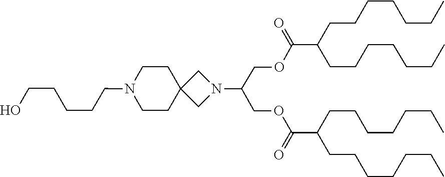 [SMILES: CCCCCCCC(CCCCCCC)C(=O)OCC(COC(=O)C(CCCCCCC)CCCCCCC)N1CC2(CCN(CCCCCO)CC2)C1]

### Step 1: 2-(7-(5-((tert-butyldimethylsilyl)oxy)pentyl)-2,7-diazaspiro[3.5]nonan-2-yl)propane-1,3-diol

This compound was prepared according to the general alkylation procedure as described in Example 1 (Step 3). 2-(2,7-Diazaspiro[3.5]nonan-2-yl)propane-1,3-diol (Example 13, step 2) (1.10 g, 4.0 mmol) and ((5-bromopentyl)oxy) (tert-butyl)dimethylsilane (1.19 g, 4.23 mmol) provided 2-(7-(5-((tert-butyldimethylsilyl)oxy) pentyl)-2,7-diazaspiro[3.5]nonan-2-yl)propane-1,3-diol (1.4 g, 64% yield) as a yellow oil. LCMS (ESI): Calc'd for C₂₁H₄₅N₂O₃Si [M+H]⁺ 401.3; found 401.3.

### Step 2: 2-(7-(5-((tert-butyldimethylsilyl)oxy) pentyl)-2,7-diazaspiro[3.5]nonan-2-yl)propane-1,3-diyl bis(2-heptylnonanoate)

This compound was prepared according to the general acylation procedure described in Example 1 (Step 4). 2-(7-(5-((tert-Butyldimethylsilyl)oxy) pentyl)-2,7-diazaspiro[3.5]nonan-2-yl)propane-1,3-diol (380 mg, 0.95 mmol) and 2-heptylnonanoic acid (A12) (632 mg, 2.47 mmol) provided 2-(7-(5-((tert-butyldimethylsilyl)oxy) pentyl)-2,7-diazaspiro[3.5]nonan-2-yl)propane-1,3-diyl bis(2-heptylnonanoate) (865 mg, 92% yield) as a colorless oil. LCMS (ESI): Calc'd for C₅₃H₁₀₆N₂O₅Si [M+2H]²⁺ 439.4; found 439.5.

### Step 3:2-(7-(5-hydroxypentyl)-2,7-diazaspiro[3.5]nonan-2-yl)propane-1,3-diyl bis(2-heptylnonanoate)

This compound was prepared according to the general TBAF-mediated tertbutyldimethyl silyl deprotection procedure described in Example 4 (Step 2). 2-(7-(5-((tert-Butyldimethylsilyl)oxy) pentyl)-2,7-diazaspiro[3.5]nonan-2-yl)propane-1,3-diyl bis(2-heptylnonanoate) (760 mg, 0.87 mmol) provided 2-(7-(5-hydroxypentyl)-2,7-diazaspiro[3.5]-nonan-2-yl)propane-1,3-diyl bis(2-heptylnonanoate) (426 mg, 57% yield) as a colorless oil. 1H NMR (400 MHz, CD₃OD) δ ppm 4.09-4.16 (m, 2H), 4.00-4.07 (m, 2H), 3.52-3.61 (m, 2H), 3.16-3.22 (m, 4H), 2.60-2.80 (m, 5H), 2.31-2.44 (m, 2H), 1.80-1.98 (m, 4H), 1.52-1.68 (m, 8H), 1.38-1.52 (m, 6H), 1.23-1.35 (m, 42H), 0.88-0.93 (m, 12H). LCMS (ESI): Calc'd for C₄₇H₉₁N₂O₅ [M+H]⁺ 763.68; found 763.7.

### Example 19

### 2-(9-(4-hydroxybutyl)-3,9-diazaspiro[5.5]undecan-3-yl)pentane-1,5-diyl bis(2-hexyldecanoate) (19)

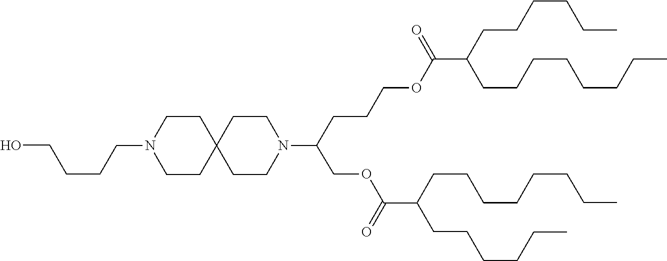 [SMILES: CCCCCCCCC(CCCCCC)C(=O)OCCCC(COC(=O)C(CCCCCC)CCCCCCCC)N1CCC2(CCN(CCCCO)CC2)CC1]

### Step 1: dimethyl 2-(9-(tert-butoxycarbonyl)-3,9-diazaspiro[5.5]undecan-3-yl)pentanedioate

Dimethyl 2-bromopentanedioate (612 mg, 2.56 mmol), Cs₂CO₃ (1.59 g, 4.87 mmol), and NaI (36.5 mg, 0.24 mmol) were added to a solution of tert-butyl 3,9-diazaspiro[5.5]undecane-3-carboxylate (620 mg, 2.44 mmol) in DMF (12 mL), and the mixture was stirred at 110° C. for 3 hours. The mixture was then diluted with EtOAc, washed with H₂O and brine, dried over Na₂SO₄. And filtered. The filtrate was concentrated in vacuo, and the residue was purified by silica gel chromatography to provide dimethyl 2-(9-(tert-butoxycarbonyl)-3,9-diazaspiro[5.5]undecan-3-yl)pentanedioate (950 mg, 71% yield) as a colorless oil. LCMS (ESI): Calc'd for C₂₁H₃₇N₂O₆ [M+H]⁺ 413.3; found 413.3.

### Step 2: tert-butyl 9-(1,5-dihydroxypentan-2-yl)-3,9-diazaspiro[5.5]undecane-3-carboxylate

CaCl₂) (307 mg, 2.76 mmol) was added to a solution of dimethyl 2-(9-(tert-butoxycarbonyl)-3,9-diazaspiro[5.5]undecan-3-yl)pentanedioate (950 mg, 2.3 mmol) in THF (10 mL) and EtOH (10 mL) and then NaBH₄ (590 mg, 15.6 mmol) was slowly at 0° C. The reaction mixture was warmed to room temperature and stirred for 16 hours. The mixture was then quenched with saturated ammonium chloride and concentrated in vacuo to dryness. The residue was partitioned between DCM:iPrOH (3:1) and H₂O. The organic layer was separated, washed with brine, dried over Na₂SO₄, and filtered. The filtrate was concentrated, and the residue was purified by silica gel chromatography to provide tert-butyl 9-(1,5-dihydroxypentan-2-yl)-3,9-diazaspiro[5.5]undecane-3-carboxylate (700 mg, 85% yield) as a white gum. LCMS (ESI): Calc'd for C₁₉H₃₇N₂O₄ [M+H]⁺ 357.3; found 357.3.

### Step 3:2-(3,9-diazaspiro[5.5]undecan-3-yl)pentane-1,5-diol

HCl (1M, 5 mL) was added to a solution of tert-butyl 9-(1,5-dihydroxypentan-2-yl)-3,9-diazaspiro[5.5]undecane-3-carboxylate (647 mg, 1.96 mmol) in MeOH (5 mL), and the mixture was stirred at room temperature for 2 hours. The mixture was then concentrated in vacuo to give 2-(3,9-diazaspiro[5.5]undecan-3-yl)pentane-1,5-diol (647 mg, 100% yield) as a white solid. LCMS (ESI): Calc'd for C₁₄H₂₉N₂O₂ [M+H]⁺ 257.2; found 257.1.

### Step 4:2-(9-(4-((tert-butyldimethylsilyl)oxy)butyl)-3,9-diazaspiro[5.5]undecan-3-yl)pentane-1,5-diol

This compound was prepared according to the general alkylation procedure as described in Example 1 (Step 3). 2-(3,9-Diazaspiro[5.5]undecan-3-yl)pentane-1,5-diol (647 mg, 1.96 mmol) and (4-bromobutoxy) (tert-butyl)dimethylsilane (525 mg, 1.96 mmol) provided 2-(9-(4-((tert-butyldimethylsilyl)oxy)butyl)-3,9-diazaspiro[5.5]undecan-3-yl)pentane-1,5-diol (600 mg, 69% yield) as a colorless oil. LCMS (ESI): Calc'd for C₂₄H₅₁N₂O₃Si [M+H]⁺ 443.4; found 443.5.

### Step 5: 2-(9-(4-((tert-butyldimethylsilyl)oxy)butyl)-3,9-diazaspiro[5.5]undecan-3-yl)pentane-1,5-diyl bis(2-hexyldecanoate)

This compound was prepared according to the general acylation procedure as described in Example 1 (Step 4). 2-(9-(4-((tert-Butyldimethylsilyl)oxy)butyl)-3,9-diazaspiro[5.5]undecan-3-yl)pentane-1,5-diol (500 mg, 1.13 mmol) and 2-hexyldecanoic acid (A13) (724 mg, 2.82 mmol) provided 2-(9-(4-((tert-butyldimethylsilyl)oxy)butyl)-3,9-diazaspiro[5.5]undecan-3-yl)pentane-1,5-diyl bis(2-hexyldecanoate) (1.0 g, 96% yield) as a light-yellow oil. LCMS (ESI): Calc'd for C₅₆H₁₁₁N₂O₅Si [M+H]⁺ 919.8; found 919.5.

### Step 6: 2-(9-(4-hydroxybutyl)-3,9-diazaspiro[5.5]undecan-3-yl)pentane-1,5-diyl bis(2-hexyldecanoate)

This compound was prepared according to the general TBAF-mediated tertbutyldimethyl silyl deprotection procedure as described in Example 4 (Step 2). 2-(9-(4-((tert-Butyldimethylsilyl)oxy)butyl)-3,9-diazaspiro[5.5]undecan-3-yl)pentane-1,5-diyl bis(2-hexyl-decanoate) (950 mg, 1.03 mmol) provided 2-(9-(4-hydroxybutyl)-3,9-diazaspiro[5.5]undecan-3-yl)pentane-1,5-diyl bis(2-hexyldecanoate) (239 mg, 29% yield) as a light-yellow oil. ¹H NMR (400 MHz, CD₃OD) δ ppm 4.29-4.19 (m, 1H), 4.17-4.04 (m, 3H), 3.61-3.48 (m, 2H), 2.83-2.61 (m, 3H), 2.59-2.44 (m, 6H), 2.44-2.31 (m, 4H), 1.84-1.41 (m, 24H), 1.39-1.21 (m, 40H), 0.96-0.81 (m, 12H). LCMS (ESI): Calc'd for C₅₀H₉₇N₂O₅ [M+H]⁺ 805.73; found 806.0.

### Example 20

### 2-(2-(4-hydroxybutyl)-2,7-diazaspiro[3.5]nonan-7-yl)propane-1,3-diyl bis(2-heptylnonanoate) (20)

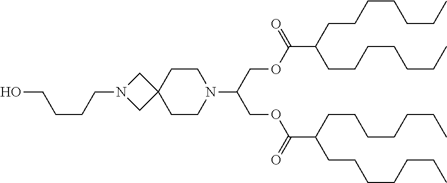 [SMILES: CCCCCCCC(CCCCCCC)C(=O)OCC(COC(=O)C(CCCCCCC)CCCCCCC)N1CCC2(CC1)CN(CCCCO)C2]

### Step 1: tert-butyl 7-(2,2-dimethyl-1,3-dioxan-5-yl)-2,7-diazaspiro[3.5]nonane-2-carboxylate

This compound was prepared according to the general reductive amination procedure as described in Example 3 (Step 1). tert-Butyl 2,7-diazaspiro[3.5]nonane-2-carboxylate (8.0 g, 35.4 mmol) and 2,2-dimethyl-1,3-dioxan-5-one (5.98 g, 46.0 mmol) provided tert-butyl 7-(2,2-dimethyl-1,3-dioxan-5-yl)-2,7-diazaspiro[3.5]nonane-2-carboxylate (7.2 g, 60% yield) as a yellow solid. LCMS (ESI): Calc'd for C₁₈H₃₃N₂O₄ [M+H]⁺ 341.2; found 341.1.

### Step 2: 2-(2,7-diazaspiro[3.5]nonan-7-yl)propane-1,3-diol

This compound was prepared according to the general acetal deprotection/boc-deprotection protocol as described in Example 3 (Step 2). tert-butyl 7-(2,2-Dimethyl-1,3-dioxan-5-yl)-2,7-diazaspiro[3.5]nonane-2-carboxylate (3.0 g, 8.8 mmol) provided 2-(2,7-diazaspiro[3.5]nonan-7-yl)propane-1,3-diol (2.1 g, 87% yield) as a yellow oil. LCMS (ESI): Calc'd for C₁₀H₂₁N₂O₂ [M+H]⁺ 201.1; found 201.1.

### Step 3: 2-(2-(4-((tert-butyldimethylsilyl)oxy)butyl)-2,7-diazaspiro[3.5]nonan-7-yl)propane-1,3-diol

This compound was prepared according to the general alkylation procedure as described in Example 1 (Step 3). 2-(2,7-Diazaspiro[3.5]nonan-7-yl)propane-1,3-diol (2.0 g, 9.99 mmol) and (4-bromobutoxy) (tert-butyl)dimethylsilane (2.67 g, 9.99 mmol) provided 2-(2-(4-((tert-butyldimethylsilyl)oxy)butyl)-2,7-diazaspiro[3.5]nonan-7-yl)propane-1,3-diol (1.35 g, 35% yield) as a colorless oil. LCMS (ESI): Calc'd for C₂₀H₄₃N₂O₃Si [M+H]⁺ 387.3; found 387.3.

### Step 4: 2-(2-(4-((tert-butyldimethylsilyl)oxy)butyl)-2,7-diazaspiro[3.5]nonan-7-yl)propane-1,3-diyl bis(2-heptylnonanoate)

This compound was prepared according to the general acylation procedure as described in Example 1 (Step 4). 2-(2-(4-((tert-Butyldimethylsilyl)oxy)butyl)-2,7-diazaspiro[3.5]nonan-7-yl)propane-1,3-diol (500 mg, 1.29 mmol) and 2-heptylnonanoic acid (A12) (829 mg, 3.23 mmol) provided 2-(2-(4-((tert-butyldimethylsilyl)oxy)butyl)-2,7-diazaspiro[3.5]nonan-7-yl)propane-1,3-diyl bis(2-heptylnonanoate) (800 mg, 72% yield) as a yellow solid. LCMS (ESI): Calc'd for C₅₂H₁₀₃N₂O₅Si [M+H]⁺ 863.8; found 863.9.

### Step 5: 2-(2-(4-hydroxybutyl)-2,7-diazaspiro[3.5]nonan-7-yl)propane-1,3-diyl bis(2-heptylnonanoate)

This compound was prepared according to the general TBAF-mediated tertbutyldimethyl silyl deprotection procedure as described in Example 4 (Step 2). 2-(2-(4-((tert-Butyldimethylsilyl)oxy)butyl)-2,7-diazaspiro[3.5]nonan-7-yl)propane-1,3-diyl bis(2-heptylnonanoate) (750 mg, 1.02 mmol) provided 2-(2-(4-hydroxybutyl)-2,7-diazaspiro[3.5]nonan-7-yl)propane-1,3-diyl bis(2-heptylnonanoate) (342 mg, 45% yield) as a colorless oil. ¹H NMR (400 MHz, CD₃OD) δ 4.31 (dd, J=11.6, 6.1 Hz, 2H), 4.10 (dd, J=11.6, 5.4 Hz, 2H), 3.54 (t, J=6.1 Hz, 2H), 3.11 (s, 4H), 3.00 (p, J=5.7 Hz, 1H), 2.63-2.61 (m, 4H), 2.56 (t, J=7.3 Hz, 2H), 2.36 (tt, J=9.4, 5.1 Hz, 2H), 1.75-1.73 (m, 4H), 1.63-1.53 (m, 6H), 1.48-1.42 (m, 6H), 1.29 (brs, 40H), 0.91 (m, 12H). LCMS (ESI): Calc'd for C₄₆H₈₉N₂O₅ [M+H]⁺ 749.7; found 749.8.

### Example 21

### 2-(9-(5-hydroxypentan-2-yl)-3,9-diazaspiro[5.5]undecan-3-yl)propane-1,3-diyl bis(2-hexyldecanoate) (21)

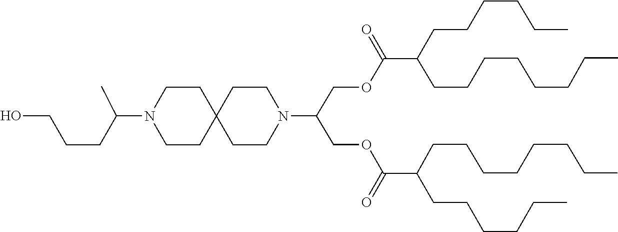 [SMILES: CCCCCCCCC(CCCCCC)C(=O)OCC(COC(=O)C(CCCCCC)CCCCCCCC)N1CCC2(CCN(C(C)CCCO)CC2)CC1]

### Step 1: 2-(9-(5-((tert-butyldimethylsilyl)oxy) pentan-2-yl)-3,9-diazaspiro[5.5]undecan-3-yl)propane-1,3-diol

TEA (2 g, 19.9 mmol) followed by 5-((tert-butyldimethylsilyl)oxy) pentan-2-one (1.72 g, 7.96 mmol) were added to a solution of 2-(3,9-diazaspiro[5.5]undecan-3-yl)propane-1,3-diol (Example 3, Step 2) (2 g, 6.64 mmol) in MeOH (40 mL). After stirring at room temperature for 3 hours, acetic acid (0.4 mL) followed by NaBH₃CN (1.25 g, 19.9 mmol) were added, and the mixture was stirred at room temperature for 16 hours. The reaction mixture was then filtered, and the MeOH was removed in vacuo. The residue was partitioned between DCM and saturated NaHCO₃. The organic layer was dried over Na₂SO₄ and filtered. The filtrate was concentrated in vacuo, and the crude product was purified by silica gel chromatography to provide 2-(9-(5-((tert-butyldimethylsilyl)oxy) pentan-2-yl)-3,9-diazaspiro[5.5]undecan-3-yl)propane-1,3-diol (500 mg, 16% yield) as a yellow oil. LCMS (ESI): Calc'd for C₂₃H₄₉N₂O₃Si [M+H]⁺ 429.4; found 429.2.

### Step 2: 2-(9-(5-((tert-butyldimethylsilyl)oxy) pentan-2-yl)-3,9-diazaspiro[5.5]undecan-3-yl)propane-1,3-diyl bis(2-hexyldecanoate)

This compound was prepared according to the general acylation procedure as described in Example 1 (Step 4). 2-(9-(5-((tert-Butyldimethylsilyl)oxy) pentan-2-yl)-3,9-diazaspiro[5.5]-undecan-3-yl)propane-1,3-diol (450 mg, 1.05 mmol) and 2-hexyldecanoic acid (A13) (673 mg, 2.62 mmol) provided 2-(9-(5-((tert-butyldimethylsilyl)oxy) pentan-2-yl)-3,9-diazaspiro[5.5]-undecan-3-yl)propane-1,3-diyl bis(2-hexyldecanoate) (650 mg, 62% yield) as a yellow oil. LCMS (ESI): Calc'd for C₅₅H₁₀₉N₂O₅Si [M+H]⁺ 905.8; found 905.5.

### Step 3: 2-(9-(5-hydroxypentan-2-yl)-3,9-diazaspiro[5.5]undecan-3-yl)propane-1,3-diyl bis(2-hexyldecanoate)

This compound was prepared according to the general TBAF-mediated tertbutyldimethyl silyl deprotection as described in Example 4 (Step 2). 2-(9-(5-((tert-Butyldimethylsilyl)oxy) pentan-2-yl)-3,9-diazaspiro[5.5]undecan-3-yl)propane-1,3-diyl bis(2-hexyldecanoate) (600 mg, 0.663 mmol) provided 2-(9-(5-hydroxypentan-2-yl)-3,9-diazaspiro[5.5]undecan-3-yl)propane-1,3-diyl bis(2-hexyldecanoate) (197 mg, 38% yield) as a colorless oil. ¹H NMR (400 MHZ, CD₃OD) δ 4.33-4.30 (m, 2H), 4.17-4.13 (m, 2H), 3.63-3.49 (m, 2H), 3.61-3.50 (m, 1H), 2.70-2.66 (m, 7H), 2.60-2.54 (m, 2H), 2.37 (tt, J=9.4, 5.1 Hz, 2H), 1.76-1.54 (m, 10H), 1.54-1.41 (m, 8H), 1.29 (s, 42H), 1.08 (d, J=6.6 Hz, 3H), 0.94-0.89 (m, 12H). LCMS (ESI): Calc'd for C₄₉H₉₅N₂O₅ [M+H]⁺ 791.7; found 791.9.

The following Examples 22-50 were prepared in a similar manner to the specified Example and other Examples described herein employing the corresponding carboxylic acid stated.

TABLE 3

| Ex | Lipid | Analytical Data |
|---|---|---|
| 22 | According to Example 6 with A12. 2-(2-(4-hydroxybutyl)-2,8- diazaspiro[4.5]decan-8-yl)propane-1,3-diyl bis(2-heptylnonanoate) (22) 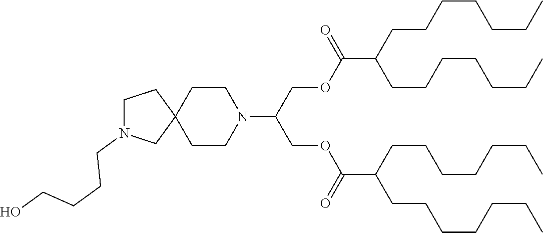 [SMILES: CCCCCCCC(CCCCCCC)C(=O)OCC(COC(=O)C(CCCCCCC)CCCCCCC)N1CCC2(CCN(CCCCO)C2)CC1] | ¹H NMR (400 MHz, CDCl₃) δ 4.28 (dd, J = 11.5, 6.4 Hz, 2H), 4.04 (dd, J = 11.5, 5.5 Hz, 2H), 3.56 (br s, 2H), 2.95-3.04 (m, 1H), 2.63 (br s, 2H), 2.58 (t, J = 5.4 Hz, 4H), 2.46 (d, J = 19.1 Hz, 4H), 2.32 (tt, J = 8.8, 5.4 Hz, 2H), 1.70-1.51 (m, 14H), 1.43 (m, 5H), 1.25 (m, 40H), 0.88 (t, J = 6.7 Hz, 12H). LCMS (ESI): Calc'd for C₄₇H₉₁N₂O₅ [M + H]⁺ 763.68; found 763.9. |
| 23 | According to Example 3 with A13, 2-(9-(4-hydroxybutyl)-3,9- diazaspiro[5.5]undecan-3-yl)propane-1,3-diyl bis(2-hexyldecanoate) (23) 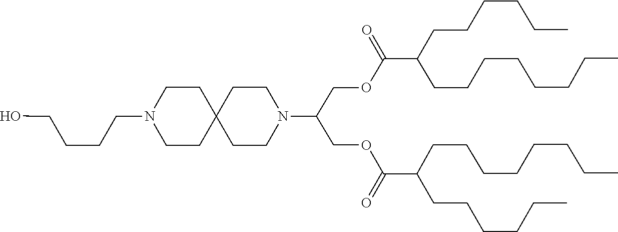 [SMILES: CCCCCCCCC(CCCCCC)C(=O)OCC(COC(=O)C(CCCCCC)CCCCCCCC)N1CCC2(CCN(CCCCO)CC2)CC1] | ¹H NMR (400 MHz, CDCl₃) δ ppm 4.24- 4.37 (m, 2 H), 4.02- 4.12 (m, 2 H), 3.56- 3.64 (m, 2 H), 2.94- 3.04 (m, 1 H), 2.41- 2.66 (m, 10 H), 2.28- 2.39 (m, 3 H), 1.66- 1.76 (m, 4 H), 1.54- 1.64 (m, 8 H), 1.38- 1.50 (m, 8 H), 1.26 (m, 40 H), 0.85-0.91 (m, 12 H). LCMS (ESI): Calc'd for C₄₈H₉₃N₂O₅ [M + H]⁺ 777.7; found 777.6. |
| 24 | According to Example 3 with A20. 2-(9-(4-hydroxybutyl)-3,9- diazaspiro[5.5]undecan-3-yl)propane-1,3-diyl bis(2-octyldecanoate) (24) 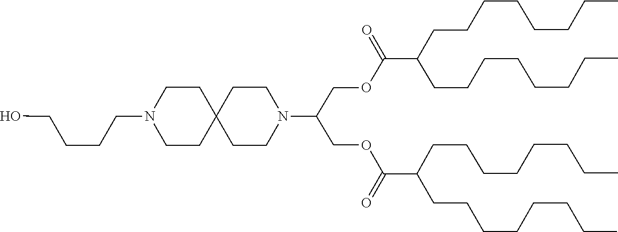 [SMILES: CCCCCCCCC(CCCCCCCC)C(=O)OCC(COC(=O)C(CCCCCCCC)CCCCCCCC)N1CCC2(CCN(CCCCO)CC2)CC1] | ¹H NMR (400 MHz, CDCl₃) δ 4.29 (dd, J = 11.5, 6.3 Hz, 2H), 4.05 (dd, J = 11.5, 5.5 H, 2H), 3.59 (t, J = 4.9 Hz, 2H), 2.97 (p, J = 6.0 Hz, 1H), 2.60 (t, J = 5.5 Hz, 6H), 2.52 (br s, 2H), 2.32 (tt, J = 8.9, 5.5 Hz, 2H), 1.77-1.64 (m, 4H), 1.63-1.51 (m, 8H), 1.50-1.36 (m, 8H), 1.33-1.17 (m, 51H), 0.87 (t, J = 6.7, 12H). LCMS (ESI): Calc'd for C₅₂H₁₀₁N₂O₅ [M + H]⁺ 833.76; found 833.7. |
| 25 | According to Example 5 with A12. 2-(9-(5-hydroxypentyl)-3,9- diazaspiro[5.5]undecan-3-yl)propane-1,3-diyl bis(2-heptylnonanoate) (25) 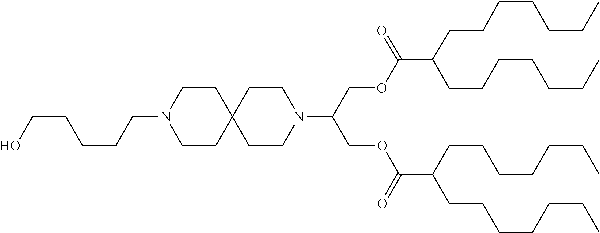 [SMILES: CCCCCCCC(CCCCCCC)C(=O)OCC(COC(=O)C(CCCCCCC)CCCCCCC)N1CCC2(CCN(CCCCCO)CC2)CC1] | ¹H NMR (400 MHz, CDCl₃) δ 4.29 (dd, J = 11.5, 6.3 Hz, 2H), 4.06 (dd, J = 11.5, 5.4 Hz, 2H), 3.63 (t, J = 6.3 Hz, 2H), 2.97 (p, J = 5.9 Hz, 1H), 2.59 (t, J = 5.4 Hz, 4H), 2.47- 2.36 (m, 6H), 2.35-2.26 (m, 3H), 1.63-1.50 (m, 12H), 1.47-1.34 (m, 10H), 1.32-1.18 (m, 40H), 0.87 (t, J = 6.7 Hz, 12H). LCMS (ESI): Calc'd for C₄₉H₉₅N₂O₅ [M + H]⁺ 791.72; found 791.7. |
| 26 | According to Example 3 with A14. 2-(9-(4-hydroxybutyl)-3,9- diazaspiro[5.5]undecan-3-yl)propane-1,3-diyl bis(2-heptyldecanoate) (26) 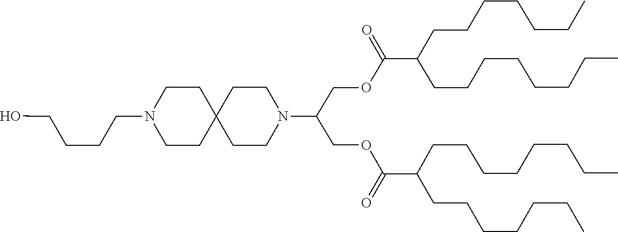 [SMILES: CCCCCCCCC(CCCCCCC)C(=O)OCC(COC(=O)C(CCCCCCC)CCCCCCCC)N1CCC2(CCN(CCCCO)CC2)CC1] | ¹H NMR (400 MHz, CDCl₃) δ 4.31 (dd, J = 11.5, 6.3 Hz, 2H), 4.07 (dd, J = 11.5, 5.5 Hz, 2H), 3.62 (t, J = 5.1 Hz, 2H), 2.99 (p, J = 5.9 Hz, 1H), 2.73-2.65 (m, 2H), 2.63-2.58 (m, 6H), 2.33 (tt, J = 8.8, 5.4 Hz, 2H), 1.82-1.74 (m, 2H), 1.72-1.54 (m, 11H), 1.52-1.36 (m, 9H), 1.33-1.20 (m, 45H), 0.89 (t, J = 6.7 Hz, 12H). LCMS (ESI): Calc'd for C₅₀H₉₇N₂O₅ [M + H]⁺ 805.73; found 805.6. |
| 27 | According to Example 3 with A15. 2-(9-(4-hydroxybutyl)-3,9- diazaspiro[5.5]undecan-3-yl)propane-1,3-diyl bis(2-butyldecanoate) (27) 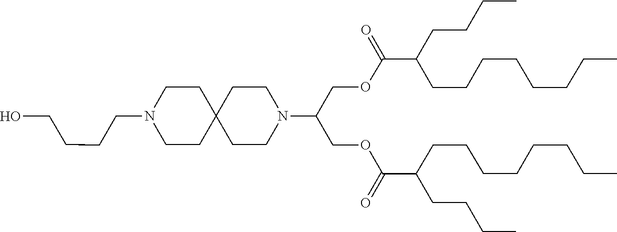 [SMILES: CCCCCCCCC(CCCC)C(=O)OCC(COC(=O)C(CCCC)CCCCCCCC)N1CCC2(CCN(CCCCO)CC2)CC1] | ¹H NMR (400 MHz, CDCl₃) δ 4.32 (ddd, J = 11.5, 6.4, 1.8 Hz, 2H), 4.08 (ddd, J = 11.6, 5.5, 2.1 Hz, 2H), 3.61 (t, J = 4.9 Hz, 2H), 3.00 (p, J = 5.9 Hz, 1H), 2.62 (t, J = 5.4 Hz, 4H), 2.51 (br s, 2H), 2.34 (tt, J = 8.7, 5.3 Hz, 2H), 1.80-1.66 (m, 5H), 1.60 (h, J = 7.4, 6.3 Hz, 8H), 1.52-1.40 (m, 9H), 1.36-1.20 (m, 34H), 0.89 (t, J = 6.9 Hz, 12H). LCMS (ESI): Calc'd for C₄₄H₈₅N₂O₅ [M + H]⁺ 721.64; found 721.6. |
| 28 | According to Example 3 with A20. 2-(9-(4-hydroxybutyl)-3,9- diazaspiro[5.5]undecan-3-yl)propane-1,3-diyl bis(3-hexylundecanoate) (28) 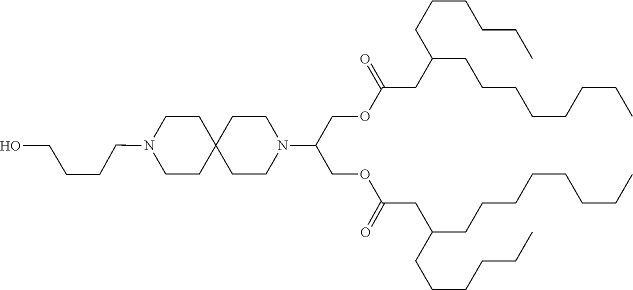 [SMILES: CCCCCCCCC(CCCCCC)CC(=O)OCC(COC(=O)CC(CCCCCC)CCCCCCCC)N1CCC2(CCN(CCCCO)CC2)CC1] | 1H NMR (400 MHz, CDCl₃) δ 4.30 (dd, J = 11.5, 6.2 Hz, 2H), 4.08 (dd, J = 11.5, 5.7 Hz, 2H), 3.61-3.56 (m, 2H), 2.98 (p, J = 5.9 Hz, 1H), 2.61 (t, J = 5.4 Hz, 4H), 2.56-2.41 (m, 6H), 2.25 (d, J = 6.8 Hz, 4H), 1.88-1.80 (m, 2H), 1.73-1.66 (m, 4H), 1.56 (t, J = 5.7 Hz, 4H), 1.47 (t, J = 5.4 Hz, 4H), 1.37- 1.22 (m, 49H), 0.90 (t, J = 6.7 Hz, 12H). LCMS (ESI: Calc'd for C₅₀H₉₇N₂O₅ [M + H]⁺ 805.73; found 805.6. |
| 29 | According to Example 3 with A6. 2-(9-(4-hydroxybutyl)-3,9- diazaspiro[5.5]undecan-3-yl)propane-1,3-diyl bis(2-pentyldecanoate) (29) 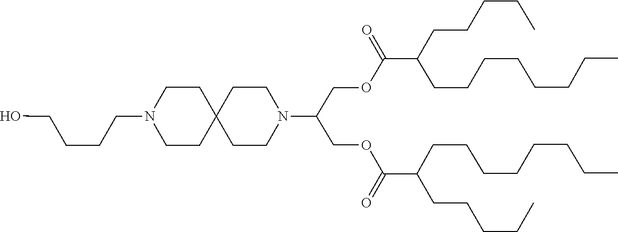 [SMILES: CCCCCCCCC(CCCCC)C(=O)OCC(COC(=O)C(CCCCC)CCCCCCCC)N1CCC2(CCN(CCCCO)CC2)CC1] | ¹H NMR (400 MHz, CD₃OD) δ 4.32 (ddd, J = 11.6, 6.0, 4.1 Hz, 2H), 4.15 (ddd, J = 11.6, 5.4 3.8 Hz, 2H), 3.56 (t, J = 5.9 Hz, 2H), 3.01 (q, J = 5.7 Hz, 1H), 2.67 (t, J = 5.5 Hz, 4H), 2.56-2.48 (m, 3H), 2.44 (t, J = 7.1 Hz, 2H), 2.37 (tt, J = 9.4, 5.2 Hz, 2H), 1.67-1.41 (m, 20H), 1.37-1.21 (m, 37H), 0.90 (t, J = 6.7 Hz, 12H). LCMS (ESI): Calc'd for C₄₆H₈₉N₂O₅ [M + H]⁺ 749.67, found 749.7. |
| 30 | According to Example 3 with A4. 2-(9-(4-hydroxybutyl)-3,9- diazaspiro[5.5]undecan-3-yl)propane-1,3-diyl bis(2-(cyclobutylmethyl)decanoate) (30) 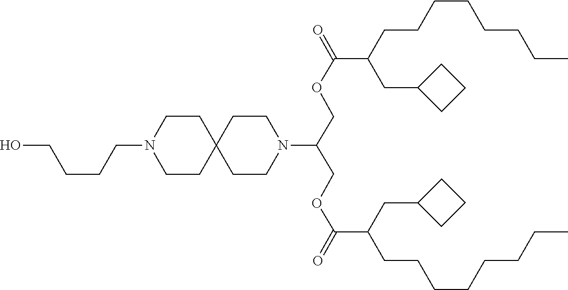 [SMILES: CCCCCCCCC(CC1CCC1)C(=O)OCC(COC(=O)C(CCCCCCCC)CC1CCC1)N1CCC2(CCN(CCCCO)CC2)CC1] | ¹H NMR (400 MHz, CD₃OD) δ 4.32 (ddd, J = 11.6, 6.0, 3.8 Hz, 2H), 4.14 (dt, J = 10.7, 4.7 Hz, 2H), 3.57 (t, J = 6.0 Hz, 2H), 3.02 (p, J = 5.7 Hz, 1H), 2.69 (t, J = 5.5 Hz, 4H), 2.65- 2.57 (m, 4H), 2.53 (t, J = 7.5 Hz, 2H), 2.38- 2.23 (m, 4H), 2.13- 1.96 (m, 4H), 1.94- 1.51 (m, 27H), 1.49- 1.41 (m, 2H), 1.36- 1.22 (m, 23H), 0.91 (t, J = 6.7 Hz, 6H). LCMS (ESI): Calc'd for C₄₆H₈₅N₂O₅ [M + H]⁺ 745.64, found 745.7. |
| 31 | According to Example 3 with A7. 2-(9-(4-hydroxybutyl)-3,9- diazaspiro[5.5]undecan-3-yl)propane-1,3-diyl bis(2-heptyltetradecanoate) (31) 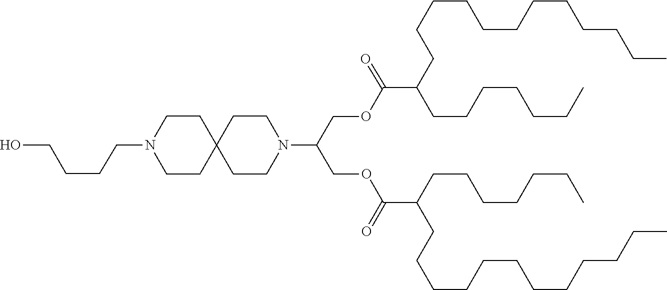 [SMILES: CCCCCCCCCCCCC(CCCCCCC)C(=O)OCC(COC(=O)C(CCCCCCC)CCCCCCCCCCCC)N1CCC2(CCN(CCCCO)CC2)CC1] | ¹H NMR (400 MHz, CDCl₃) δ 4.31 (dd, J = 11.5, 6.3 Hz, 2H), 4.08 (dd, J = 11.5, 5.4 Hz, 2H), 3.60- 3.54 (m, 2H), 2.99 (p, J = 5.9 Hz, 1H), 2.61 (t, J = 5.4 Hz, 4H), 2.50-2.28 (m, 8H), 1.71-1.39 (m, 26H), 1.34-1.18 (m, 55H), 0.89 (t, J = 6.9, 2.0 Hz, 12H). LCMS (ESI): Calc'd for C₅₈H₁₁₄N₂O₅ [M + 2H]²⁺ 459.43, found 459.5. |
| 32 | According to Example 3 with A8. 2-(9-(4-hydroxybutyl)-3,9- diazaspiro[5.5]undecan-3-yl)propane-1,3-diyl bis(2-(cyclopentylmethyl)decanoate) (32) 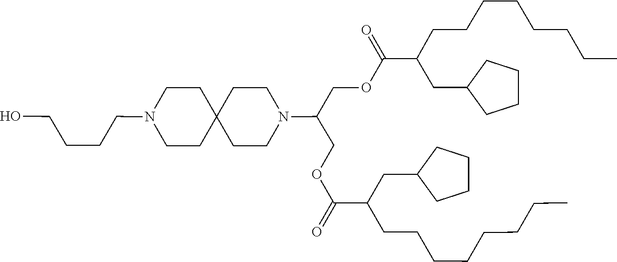 [SMILES: CCCCCCCCC(CC1CCCC1)C(=O)OCC(COC(=O)C(CCCCCCCC)CC1CCCC1)N1CCC2(CCN(CCCCO)CC2)CC1] | ¹H NMR (400 MHz, CD₃OD) δ 4.31 (ddd, J = 11.7, 6.0, 2.3 Hz, 2H), 4.19- 4.12 (m, 2H), 3.55 (t, J = 5.8 Hz, 2H), 3.05-2.98 (m, 1H), 2.67 (t, J = 5.6 Hz, 4H), 2.52-2.35 (m, 8H), 1.88-1.41 (m, 34H), 1.35-1.24 (m, 24H), 1.16-1.03 (m, 4H), 0.91 (t, J = 6.6 Hz, 6H). LCMS: Calc'd for C₄₈H₈₉N₂O₅ [M + H]⁺ 773.67, found 773.5. |
| 33 | According to Example 12 with A13. 2-(7-(4-hydroxybutyl)-2,7- diazaspiro[4.4]nonan-2-yl)propane-1,3-diyl bis(2-hexyldecanoate) (33) 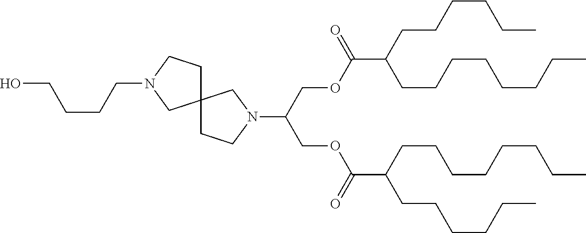 [SMILES: CCCCCCCCC(CCCCCC)C(=O)OCC(COC(=O)C(CCCCCC)CCCCCCCC)N1CCC2(CCN(CCCCO)C2)C1] | ¹H NMR (400 MHz, CD₃OD) δ 4.14-4.33 (m, 4H), 3.52-3.60 (m, 2H), 2.74-2.87 (m, 4H), 2.62-2.72 (m, 4H), 2.48-2.61 (m, 3H), 2.31-2.44 (m, 2H), 1.74-1.96 (m, 4H), 1.53-1.68 (m, 8H), 1.41-1.52 (m, 4H), 1.24-1.34 (m, 40H), 0.86-0.93 (m, 12H). LCMS (ESI): Calc'd for C₄₆H₈₉N₂O₅ [M + H]⁺ 749.67; found 749.8. |
| 34 | According to Example 13 with A13. 2-(7-(4-hydroxybutyl)-2,7- diazaspiro [3.5]nonan-2-yl)propane-1,3-diyl bis(2-hexyldecanoate) (34) 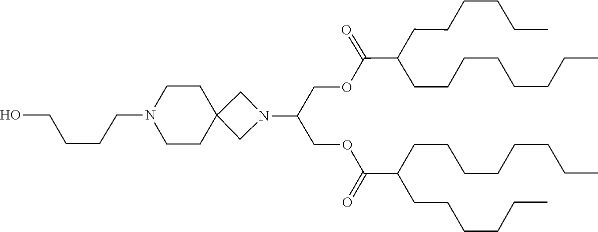 [SMILES: CCCCCCCCC(CCCCCC)C(=O)OCC(COC(=O)C(CCCCCC)CCCCCCCC)N1CC2(CCN(CCCCO)CC2)C1] | ¹H NMR (400 MHz, CD₃OD) δ ppm 4.08-4.17 (m, 2H), 3.98-4.07 (m, 2H), 3.52-3.62 (m, 2H), 3.17 (s, 4H), 2.67- 2.82 (m, 1H), 2.21- 2.63 (m, 8H), 1.73- 1.90 (m, 4H), 1.53- 1.68 (m, 8H), 1.42- 1.52 (m, 4H), 1.22- 1.36 (m, 40H), 0.84- 0.97 (m, 12H). LCMS (ESI): Calc'd for C₄₆H₈₉N₂O₅ [M + H]⁺ 749.67, found 749.8. |
| 35 | According to Example 3 with A9. 2-(9-(4-hydroxybutyl)-3,9- diazaspiro[5.5]undecan-3-yl)propane-1,3-diyl bis(2-(cyclopent-3-en-1-ylmethyl)decanoate) 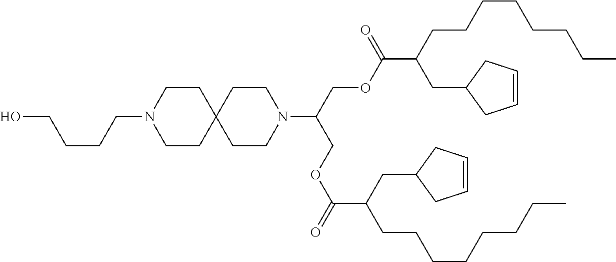 [SMILES: CCCCCCCCC(CC1CC=CC1)C(=O)OCC(COC(=O)C(CCCCCCCC)CC1CC=CC1)N1CCC2(CCN(CCCCO)CC2)CC1] | 1H NMR (400 MHz, CD₃OD) δ ppm 5.56-5.75 (m, 4H), 4.27-4.42 (m, 2H), 4.05-4.22 (m, 2H), 3.49-3.68 (m, 2H), 2.97-3.07 (m, 1H), 2.61-2.74 (m, 4H), 2.34-2.57 (m, 12H), 2.13-2.26 (m, 2H), 1.88-2.03 (m, 4H), 1.70-1.83 (m, 2H), 1.43-1.67 (m, 18H), 1.25-1.35 (m, 24H), 0.85-0.98 (m, 6H). LCMS (ESI): Calc'd for C₄₈H₈₅N₂O₅ [M + H]⁺ 769.64; found 769.7. |
| 36 | According to Example 3 with A10. 2-(9-(4-hydroxybutyl)-3,9- diazaspiro[5.5]undecan-3-yl)propane-1,3-diyl bis(2-(2-cyclobutylethyl)decanoate) (36) 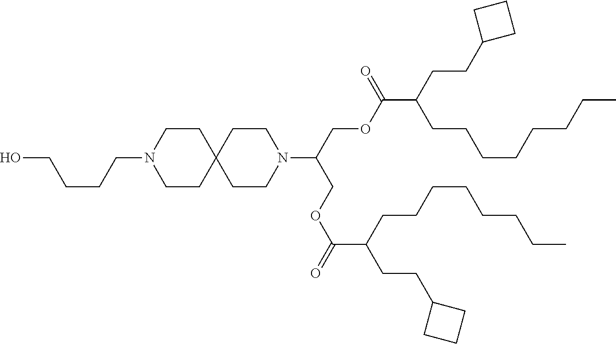 [SMILES: CCCCCCCCC(CCC1CCC1)C(=O)OCC(COC(=O)C(CCCCCCCC)CCC1CCC1)N1CCC2(CCN(CCCCO)CC2)CC1] | ¹H NMR (400 MHz, CD₃OD) δ ppm 4.27-4.40 (m, 2H), 4.08-4.22 (m, 2H), 3.50-3.67 (m, 2H), 2.94-3.12 (m, 1H), 2.63-2.72 (m, 4H), 2.43-2.55 (m, 4H), 2.29-2.42 (m, 4H), 2.17-2.29 (m, 2H), 1.99-2.11 (m, 4H), 1.75-1.95 (m, 4H), 1.40-1.68 (m, 23H), 1.35-1.42 (m, 6H), 1.24-1.33 (m, 23H), 0.84-0.97 (m, 6H). LCMS (ESI): Calc'd for C₄₈H₈₉N₂O₅ [M + H]⁺ 773.67; found 773.9. |
| 37 | According to Example 3 with A11. 2-(9-(4-hydroxybutyl)-3,9- diazaspiro[5.5]undecan-3-yl)propane-1,3-diyl bis(2-(cyclohexylmethyl)decanoate) (37) 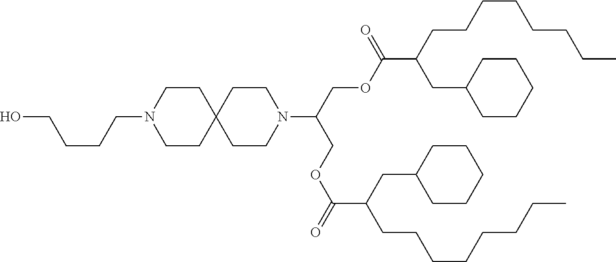 [SMILES: CCCCCCCCC(CC1CCCCC1)C(=O)OCC(COC(=O)C(CCCCCCCC)CC1CCCCC1)N1CCC2(CCN(CCCCO)CC2)CC1] | ¹H NMR (400 MHz, CD₃OD) δ ppm 4.26-4.37 (m, 2H), 4.10-4.19 (m, 2H), 3.47-3.60 (m, 2H), 2.93-3.08 (m, 1H), 2.61-2.76 (m, 4H), 2.43-2.56 (m, 6H), 2.33-2.42 (m, 2H), 1.77-1.87 (m, 2H), 1.34-1.76 (m, 28H), 0.93-1.33 (m, 36H), 0.88-0.92 (s, 6H). LCMS (ESI): Calc'd for C₅₀H₉₃N₂O₅ [M + H]⁺ 801.70; found 801.7. |
| 38 | According to Example 38 with A21. 2-(9-(4-hydroxybutyl)-3,9- diazaspiro[5.5]undecan-3-yl)propane-1,3-diyl bis(4,5-dibutylnonanoate) (38) 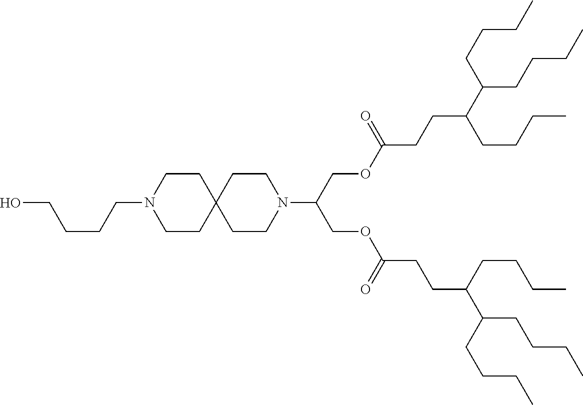 [SMILES: CCCCC(CCCC)C(CCCC)CCC(=O)OCC(COC(=O)CCC(CCCC)C(CCCC)CCCC)N1CCC2(CCN(CCCCO)CC2)CC1] | ¹H NMR (400 MHz, CD₃OD) δ ppm 4.25-4.43 (m, 2H) 4.06-4.21 (m, 2H) 3.50-3.60 (m, 2H) 2.97-3.07 (m, 1H) 2.61-2.73 (m, 4H) 2.42-2.54 (m, 4H) 2.36-2.42 (m, 2H) 2.23-2.35 (m, 4H) 1.42-1.70 (m, 16H) 1.14-1.37 (m, 40H) 0.85-0.98 (m, 18H). LCMS (ESI): Calc'd for C₅₀H₉₇N₂O₅ [M + H]⁺ 805.73; found 805.7. |
| 39 | According to Example 3 with A22. 2-(9-(4-hydroxybutyl)-3,9- diazaspiro[5.5]undecan-3-yl)propane-1,3-diyl bis(3,3-dibutylnonanoate) (39) 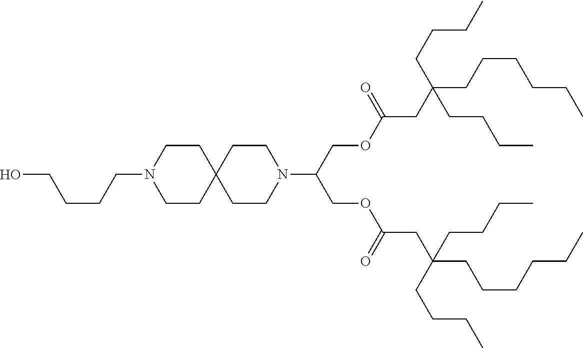 [SMILES: CCCCCCC(CCCC)(CCCC)CC(=O)OCC(COC(=O)CC(CCCC)(CCCC)CCCCCC)N1CCC2(CCN(CCCCO)CC2)CC1] | ¹H NMR (400 MHz, CD₃OD) δ ppm 4.28-4.40 (m, 2H), 4.00-4.15 (m, 2H), 3.46-3.63 (m, 2H), 2.89-3.01 (m, 1H), 2.38-2.81 (m, 10H), 2.24 (s, 4H), 1.44- 1.75 (m, 12H), 1.14- 1.41 (m, 44H), 0.70- 1.06 (m, 18H). LCMS (ESI): Calc'd for C₅₀H₉₇N₂O₅ [M + H]⁺ 805.73; found 805.8. |
| 40 | According to Example 3 with A23. 2-(9-(4-hydroxybutyl)-3,9- diazaspiro[5.5]undecan-3-yl)propane-1,3-diyl bis(4-heptylundecanoate) (40) 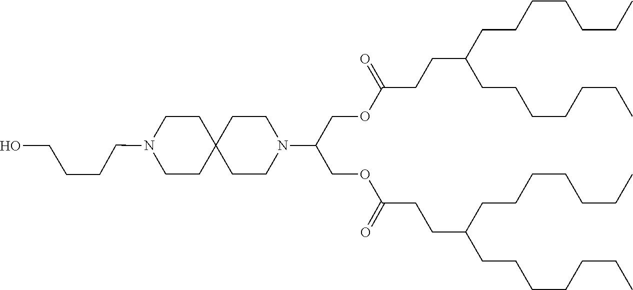 [SMILES: CCCCCCCC(CCCCCCC)CCC(=O)OCC(COC(=O)CCC(CCCCCCC)CCCCCCC)N1CCC2(CCN(CCCCO)CC2)CC1] | ¹H NMR (400 MHz, CDCl₃) δ 4.28 (dd, J = 11.4, 6.1 Hz, 2H), 4.07 (dd, J = 11.4, 5.8 Hz, 2H), 3.56-3.54 (m, 2H), 2.98 (q, J = 6.0 Hz, 1H), 2.60- 2.57 (m, 4H), 2.49- 2.32 (m, 6H), 2.31- 2.23 (m, 4H), 1.70- 1.64 (m, 4H), 1.60- 1.42 (m, 10H), 1.24 (m, 52H), 0.90-0.86 (m, 12H). LCMS (ESI): Calc'd for C₅₂H₁₀₁N₂O₅ [M + H]⁺ 833.76; found 833.8. |
| 41 | According to Example 16 with A12. 2-(7-(5-hydroxypentyl)-2,7- diazaspiro [4.4]nonan-2-yl)propane-1,3-diyl bis(2-heptylnonanoate) (41) 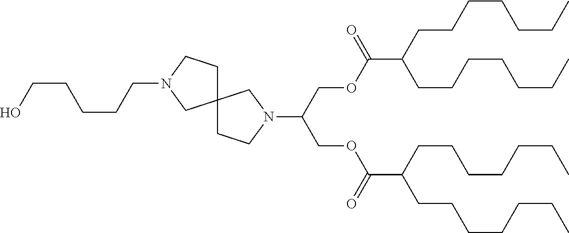 [SMILES: CCCCCCCC(CCCCCCC)C(=O)OCC(COC(=O)C(CCCCCCC)CCCCCCC)N1CCC2(CCN(CCCCCO)C2)C1] | ¹H NMR (400 MHz, CD₃OD) δ 4.32-4.19 (m, 4H), 3.58 (t, J = 6.5 Hz, 2H), 2.91- 2.63 (m, 9H), 2.62- 2.54 (m, 2H), 2.45- 2.34 (m, 2H), 2.00- 1.78 (m, 4H), 1.69- 1.38 (m, 14H), 1.38- 1.26 (m, 40H), 0.92 (t, 12H). LCMS (ESI): Calc'd for C₄₇H₉₁N₂O₅ [M + H]⁺ 763.69, found 763.8. |
| 42 | According to Example 7 with A18 and A13. 3-(decanoyloxy)-2-(9-(4-hydroxybutyl)-3,9- diazaspiro[5.5]undecan-3-yl)propyl 2- hexyldecanoate (42) 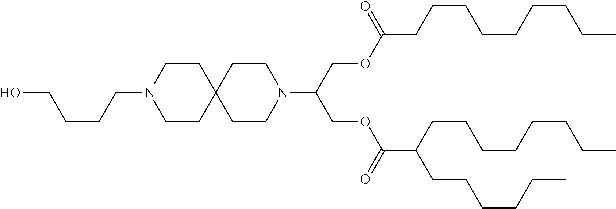 [SMILES: CCCCCCCCCC(=O)OCC(COC(=O)C(CCCCCC)CCCCCCCC)N1CCC2(CCN(CCCCO)CC2)CC1] | 1H NMR (400 MHz, CD₃OD) δ ppm 4.25-4.38 (m, 2H), 4.09-4.21 (m, 2H), 3.51-3.61 (m, 2H), 2.95-3.06 (m, 1H), 2.57-2.82 (m, 8H), 2.49-2.56 (m, 2H), 2.28-2.40 (m, 3H), 1.54-1.79 (m, 12H), 1.41-1.54 (m, 6H), 1.16-1.40 (m, 32H), 0.68-1.02 (m, 9H). LCMS (ESI): Calc'd for C₄₂H₈₁N₂O₅ [M + H]⁺ 693.61; found 693.7. |
| 43 | According to Example 7 with A19 and A13. 3-((2-hexyldecanoyl)oxy)-2-(9-(4- hydroxybutyl)-3,9-diazaspiro[5.5]undecan-3- yl)propyl (Z)-dodec-5-enoate (43) 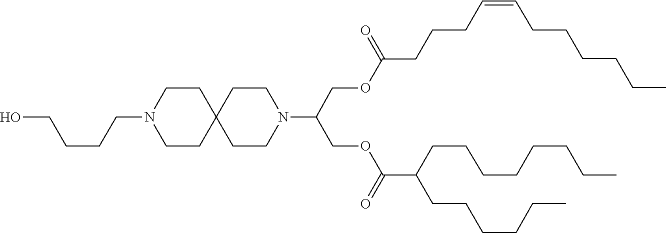 [SMILES: CCCCCC/C=C\CCCC(=O)OCC(COC(=O)C(CCCCCC)CCCCCCCC)N1CCC2(CCN(CCCCO)CC2)CC1] | ¹H NMR (400 MHz, CD₃OD) δ ppm 5.26-5.49 (m, 2H), 4.25-4.40 (m, 2H), 4.09-4.22 (m, 2H), 3.48-3.61 (m, 2H), 2.95-3.09 (m, 1H), 2.60-2.74 (m, 4H), 2.42-2.55 (m, 4H), 2.30-2.42 (m, 5H), 1.99-2.15 (m, 4H), 1.45-1.71 (m, 18H), 1.22-1.35 (m, 28H), 0.75-1.05 (m, 9H). LCMS (ESI): Calc'd for C₄₄H₈₃N₂O₅ [M + H]⁺ 719.62; found 719.7. |
| 44 | According to Example 7 with A4 and A13. 3-((2-(cyclobutylmethyl)decanoyl)oxy)-2-(9-(4- hydroxybutyl)-3,9-diazaspiro[5.5]undecan-3- yl)propyl 2-hexyldecanoate (44) 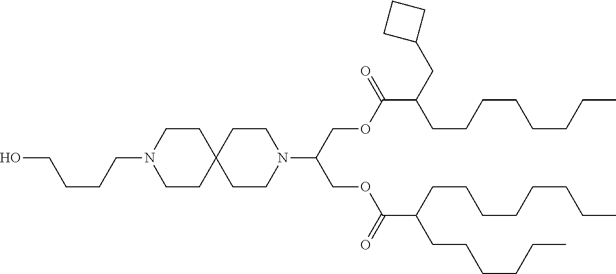 [SMILES: CCCCCCCCC(CCCCCC)C(=O)OCC(COC(=O)C(CCCCCCCC)CC1CCC1)N1CCC2(CCN(CCCCO)CC2)CC1] | ¹H NMR (400 MHz, CD₃OD) δ ppm 4.24-4.39 (m, 2H), 4.08-4.22 (m, 2H), 3.50-3.61 (m, 2H), 2.96-3.09 (m, 1H), 2.62-2.75 (m, 4H), 2.43-2.54 (m, 4H), 2.23-2.42 (m, 5H), 1.96-2.11 (m, 2H), 1.76-1.92 (m, 2H), 1.41-1.75 (m, 22H), 1.23-1.38 (m, 32H), 0.79-0.98 (m, 9H). LCMS (ESI): Calc'd for C₄₇H₈₉N₂O₅ [M + H]⁺ 761.67; found 761.8. |
| 45 | According to Example 3 with A15 and A13. 3-((2-hexyldecanoyl)oxy)-2-(9-(4- hydroxybutyl)-3,9-diazaspiro[5.5]undecan-3- yl)propyl 2-butylundecanoate (45) 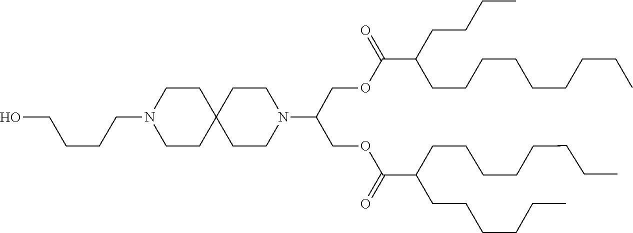 [SMILES: CCCCCCCCCC(CCCC)C(=O)OCC(COC(=O)C(CCCCCC)CCCCCCCC)N1CCC2(CCN(CCCCO)CC2)CC1] | ¹H NMR (500 MHz, CD₃OD) δ ppm 4.26-4.39 (m, 2H), 4.08-4.21 (m, 2H), 3.50-3.60 (m, 2H), 2.95-3.11 (m, 1H), 2.63-2.73 (m, 4H), 2.51-2.56 (m, 3H), 2.42-2.47 (m, 2H), 2.33-2.40 (m, 2H), 1.54-1.68 (m, 12H), 1.43-1.52 (m, 8H), 1.13-1.39 (m, 40H), 0.83-0.96 (m, 12H). LCMS (ESI): Calc'd for C₄₇H₉₁N₂O₅ [M + H]⁺ 763.68; found 763.6. |
| 46 | According to Example 7 with A15 and A17. 3-((4,5-dibutylnonanoyl)oxy)-2-(9-(4- hydroxybutyl)-3,9-diazaspiro[5.5]undecan-3- yl)propyl palmitate (46) 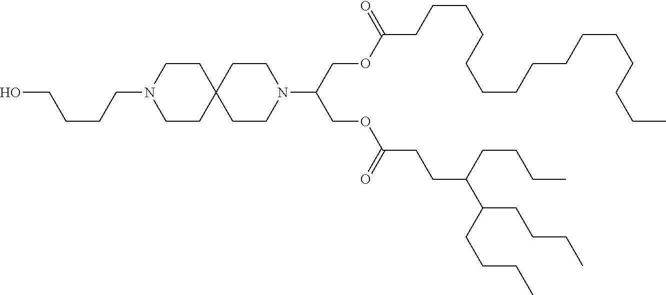 [SMILES: CCCCCCCCCCCCCCCC(=O)OCC(COC(=O)CCC(CCCC)C(CCCC)CCCC)N1CCC2(CCN(CCCCO)CC2)CC1] | ¹H NMR (400 MHz, CD₃OD) δ ppm 4.23-4.45 (m, 2H), 4.07-4.22 (m, 2H), 3.51-3.61 (m, 2H), 2.96-3.06 (m, 1H), 2.61-2.73 (m, 4H), 2.39-2.55 (m, 6H), 2.26-2.36 (m, 4H), 1.43-1.70 (m, 16H), 1.15-1.38 (m, 44H), 0.84-0.99 (m, 12H). LCMS (ESI): Calc'd for C₄₉H₉₅N₂O₅ [M + H]⁺ 791.72; found 791.7. |
| 47 | According to Example 3 with A12. 3-(9-(4-hydroxybutyl)-3,9- diazaspiro[5.5]undecan-3-yl)pentane-1,5-diyl bis(2-heptylnonanoate) (47) 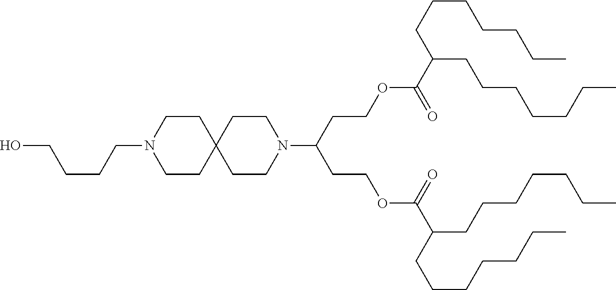 [SMILES: CCCCCCCC(CCCCCCC)C(=O)OCCC(CCOC(=O)C(CCCCCCC)CCCCCCC)N1CCC2(CCN(CCCCO)CC2)CC1] | ¹H NMR (400 MHz, CD₃OD) δ ppm 4.18-4.30 (m, 2H), 4.05-4.15 (m, 2H), 3.51-3.64 (m, 2H), 2.70-2.84 (m, 1H), 2.29-2.57 (m, 12H), 1.84-1.96 (m, 2H), 1.42-1.66 (m, 22H), 1.22-1.36 (m, 40H), 0.81-0.97 (m, 12H). LCMS (ESI): Calc'd for C₅₀H₉₇N₂O₅ [M + H]⁺ 805.73; found 805.7. |
| 48 | According to Example 17 with A12. 2-(2-(5-hydroxypentyl)-2,8- diazaspiro[4.5]decan-8-yl)propane-1,3-diyl bis(2-heptylnonanoate) (48) 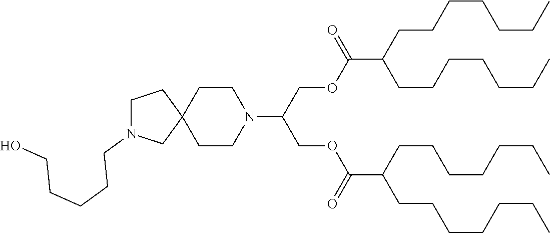 [SMILES: CCCCCCCC(CCCCCCC)C(=O)OCC(COC(=O)C(CCCCCCC)CCCCCCC)N1CCC2(CCN(CCCCCO)C2)CC1] | ¹H NMR (400 MHz, CD₃OD) δ ppm 4.27-4.37 (m, 2H), 4.06-4.18 (m, 2H), 3.49-3.63 (m, 2H), 2.95-3.07 (m, 1H), 2.72-2.83 (m, 2H), 2.63-2.71 (m, 4H), 2.51-2.61 (m, 4H), 2.29-2.42 (m, 2H), 1.67-1.75 (m, 2H), 1.53-1.67 (m, 12H), 1.39-1.52 (m, 6H), 1.24-1.37 (m, 40H), 0.83-0.97 (m, 12H). LCMS (ESI): Calc'd for C₄₈H₉₃N₂O₅ [M + H]⁺ 777.70; found 777.9. |
| 49 | According to Example 18 with A13. 2-(7-(5-hydroxypentyl)-2,7- diazaspiro [3.5]nonan-2-yl)propane-1,3-diyl bis(2-hexyldecanoate) (49) 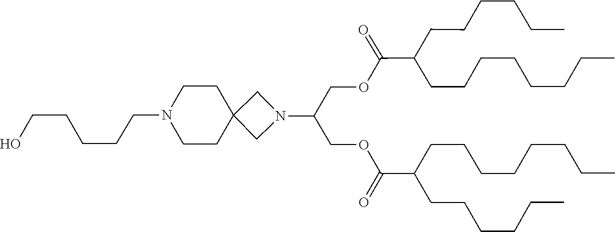 [SMILES: CCCCCCCCC(CCCCCC)C(=O)OCC(COC(=O)C(CCCCCC)CCCCCCCC)N1CC2(CCN(CCCCCO)CC2)C1] | 1H NMR (400 MHz, CD₃OD) δ ppm 4.09-4.16 (m, 2H), 4.00-4.07 (m, 2H), 3.52-3.61 (m, 2H), 3.16-3.22 (m, 4H), 2.60-2.80 (m, 5H), 2.31-2.44 (m, 2H), 1.80-1.98 (m, 4H), 1.52-1.68 (m, 8H), 1.38-1.52 (m, 6H), 1.23-1.35 (m, 42H), 0.88-0.93 (m, 12H). LCMS (ESI): Calc'd for C₄₇H₉₁N₂O₅ [M + H]⁺ 763.68; found 763.7. |
| 50 | According to Example 20 with A20. 2-(2-(4-hydroxybutyl)-2,7- diazaspiro[3.5]nonan-7-yl)propane-1,3-diyl bis(2-hexyldecanoate) (50) 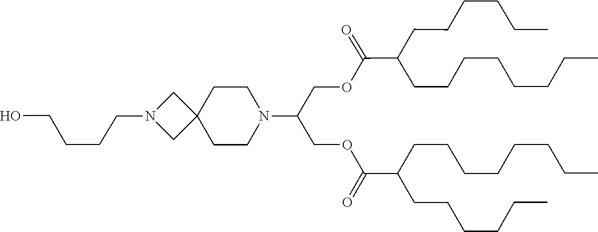 [SMILES: CCCCCCCCC(CCCCCC)C(=O)OCC(COC(=O)C(CCCCCC)CCCCCCCC)N1CCC2(CC1)CN(CCCCO)C2] | ¹H NMR (400 MHz, CD₃OD) δ 4.30 (ddd, J = 11.6, 6.2, 2.1 Hz, 2H), 4.10 (ddd, J = 11.6, 5.5, 1.8 Hz, 2H), 3.53 (t, J = 6.1 Hz, 2H), 3.06 (s, 4H), 3.01 (q, J = 5.7 Hz, 1H), 2.62 (m, 4H), 2.54-2.50 (m, 2H), 2.40-2.32 (m, 2H), 1.75-1.72 (m, 4H), 1.64-1.54 (m, 6H), 1.49-1.41 (m, 6H), 1.29 (m, 40H), 0.92-0.89 (m, 12H). LCMS (ESI): Calc'd for C₄₆H₈₉N₂O₅ [M + H]⁺ 749.7; found 749.8. |

### Example 51

### rac-(2R,3R)-8-(4-Hydroxybutyl)-8-azaspiro[4.5]decane-2,3-diyl bis(2-heptylnonanoate) (51)

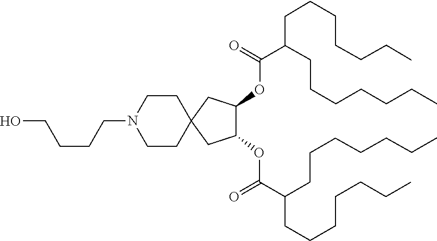 [SMILES: CCCCCCCC(CCCCCCC)C(=O)O[C@@H]1CC2(CCN(CCCCO)CC2)C[C@H]1OC(=O)C(CCCCCCC)CCCCCCC]

### Step 1: tert-butyl 4,4-diallyl-3,5-dioxopiperidine-1-carboxylate

K₂CO₃ (64.8 g, 469 mmol), 3-bromoprop-1-ene (42.6 g, 352 mmol), and tetrabutylammonium hydrogensulfate (19.9 g, 58.6 mmol) were added to a solution of tert-butyl 3,5-dioxopiperidine-1-carboxylate (25.0 g, 117.24 mmol) in MeCN (400 mL). The reaction mixture was stirred at 25° C. for 10 hours. At that time, the mixture was concentrated, and the residue was purified by silica gel chromatography to provide tert-butyl 4,4-diallyl-3,5-dioxopiperidine-1-carboxylate (15.0 g, 43.6% yield). ¹H NMR (400 MHZ, CDCl₃) δ 5.62-5.47 (m, 2H), 5.10-5.00 (m, 4H), 4.14 (br s, 4H), 2.55-2.48 (m, 4H), 1.47 (s, 9H). LCMS (ESI): Calc'd for C₁₁H₁₅NO₂ [M+H-Boc]⁺ 194.11, found 194.0.

### Step 2: tert-butyl 6,10-dioxo-8-azaspiro[4.5]dec-2-ene-8-carboxylate

Under a nitrogen atmosphere, Grubb's catalyst 2^{nd} generation (2.17 g, 2.56 mmol) was added to the solution of tert-butyl 4,4-diallyl-3,5-dioxopiperidine-1-carboxylate (15.0 g, 51.1 mmol) in DCM (200 mL). The mixture was stirred at room temperature for 12 hours. At that time, the mixture was concentrated, and the residue was purified by silica gel chromatography affording tert-butyl 6,10-dioxo-8-azaspiro[4.5]dec-2-ene-8-carboxylate (10.5 g, 77% yield) as a white solid. ¹H NMR (400 MHZ, CDCl₃) δ 5.51 (s, 2H), 4.32 (br s, 4H), 2.87 (br s, 4H), 1.51 (s, 9H).

### Step 3: tert-butyl (6Z,10E)-6,10-bis(2-tosylhydrazineylidene)-8-azaspiro[4.5]dec-2-ene-8-carboxylate

TsNHNH₂ (16.2 g, 87.1 mmol) was added to a solution of tert-butyl 6,10-dioxo-8-azaspiro[4.5]dec-2-ene-8-carboxylate (10.5 g, 39.58 mmol) in MeOH (250 mL), and the mixture was stirred at 65° C. for 16 hours. The solid was collected by filtration to provide tert-butyl (6Z,10E)-6,10-bis(2-tosylhydrazineylidene)-8-azaspiro[4.5]dec-2-ene-8-carboxylate (15.45 g, 64.9% yield) as a white powder. ¹H NMR (400 MHZ, CD₃OD) δ 7.79 (d, J=8.1 Hz, 4H), 7.40 (d, J=8.0 Hz, 4H), 5.41 (s, 2H), 4.35-4.28 (m, 4H), 2.69-2.60 (m, 6H), 2.45 (s, 6H), 1.48 (s, 9H).

### Step 4: tert-butyl 8-azaspiro[4.5]dec-2-ene-8-carboxylate

Na(CN)BH₃ (11.10 g, 177 mmol) and TsOH (7.61 g, 44.2 mmol) were added to a solution of tert-butyl (6Z,10E)-6,10-bis(2-tosylhydrazineylidene)-8-azaspiro[4.5]dec-2-ene-8-carboxylate (13.3 g, 22.1 mmol) in a 1:1 mixture of DMF (80.0 mL) and sulfolane (80.0 mL), and the mixture was stirred at 115° C. for 5 hours. At that time, the mixture was concentrated to remove DMF. H₂O (200 mL) was added to the residue, and the resulting mixture was extracted with EtOAc (100 mL×4). The combined organic layers were dried over Na₂SO₄ and filtered. The filtrate was concentrated in vacuo, and the residue was purified by silica gel chromatography to give tert-butyl 8-azaspiro[4.5]dec-2-ene-8-carboxylate (1.89 g, 36% yield) as a colorless oil. ¹H NMR (400 MHz, CDCl₃) δ 5.62 (s, 2H), 3.43-3.35 (m, 4H), 2.20 (s, 4H), 1.54-1.48 (m, 4H), 1.46 (s, 9H). LCMS (ESI): Calc'd for C₁₀H₁₆NO₂ [M-tBu+H]⁺ 182.11, found 182.1.

### Step 5: tert-butyl 6-oxaspiro[bicyclo[3.1.0]hexane-3,4′-piperidine]-1′-carboxylate

To a solution of tert-butyl 8-azaspiro[4.5]dec-2-ene-8-carboxylate (1.89 g, 7.96 mmol) in DCM (80.0 mL) was added NaHCO₃ (1.47 g, 17.5 mmol) followed by MCPBA (3.78 g, 17.5 mmol), and the mixture was stirred at room temperature for 12 hours. The mixture was quenched with saturated Na₂SO₃ (100 mL). The aqueous layer was extracted with with DCM (30 ml×4). The combined organic layers were washed with brine (60 mL), dried over Na₂SO₄, filtered, and concentrated in vacuo. The residue was purified by silica gel column chromatography to provide tert-butyl 6-oxaspiro[bicyclo[3.1.0]hexane-3,4′-piperidine]-1′-carboxylate (1.22 g, 61% yield) as a colorless oil. ¹H NMR (400 MHZ, CDCl₃) δ 3.51 (s, 2H), 3.38-3.27 (m, 4H), 2.02 (d, J=14.5 Hz, 2H), 1.60-1.48 (m, 4H), 1.44 (s, 11H). LCMS (ESI): Calc'd for C₁₀H₁₆NO₃ [M-tBu+H]⁺198.11, found 198.1.

### Step 6: rac-(2R,3R)-8-azaspiro[4.5]decane-2,3-diol

A solution of 2M H₂SO₄ (14.0 mL) was added to a solution of tert-butyl 6-oxaspiro[bicyclo[3.1.0]hexane-3,4′-piperidine]-1′-carboxylate (1.22 g, 4.83 mmol) in 1,4-dioxane (14.0 mL) at room temperature, and the resulting solution was stirred at room temperature for 16 hours. saturated Na₂CO₃ (20 mL) was added to the mixture until no gas was generated, and then the mixture was brought to pH 8 by the addition of 2N NaOH. The mixture was concentrated in vacuo to give a white solid which was taken up in MeOH (25 mL) and stirred at room temperature for 16 h. The solids were removed by filtration, and the filtrate was concentrated in vacuo. The residue from the filtrate was triturated at room temperature with DCM and the solid was collected by filtration to provide 4 g of the boc-protected diol as a white solid which was further processed to provide the titled compound vide infra. The filtrate was concentrated in vacuo to give the desired aminodiol (440 mg) as a light-yellow solid.

The boc-protected diol from the previous section (white solid, 4 g) was stirred in MeOH (20 mL) at room temperature for an additional 18 hours, residual solids were removed by filtration and the filtrate was concentrated in vacuo. The resulting solid was combined with the previous batch (440 mg) to afford rac-(2R,3R)-8-azaspiro[4.5]decane-2,3-diol (827 mg, 99% yield) as a light-yellow solid. This material was used crude in the subsequent transformation. LCMS (ESI): Calc'd for C₉H₁₈NO₂ [M+H]⁺ 172.13, found 172.1.

### Step 7: rac-(2R,3R)-8-(4-((tert-butyldimethylsilyl)oxy)butyl)-8-azaspiro[4.5]decane-2,3-diol

This compound was prepared according to the general alkylation procedure as described in Example 1 (Step 3). rac-(2R,3R)-8-azaspiro[4.5]decane-2,3-diol (827.0 mg, 4.83 mmol) provided rac-(2R,3R)-8-(4-((tert-butyldimethylsilyl)oxy)butyl)-8-azaspiro[4.5]decane-2,3-diol (769.7 mg, 44.6% yield) as a yellow gum. ¹H NMR (400 MHZ, CDCl₃) δ 4.04-3.98 (m, 2H), 3.61 (t, J=6.0 Hz, 2H), 2.53-2.26 (m, 7H), 2.00-1.90 (m, 2H), 1.72-1.35 (m, 11H), 0.88 (s, 9H), 0.04 (s, 6H). LCMS (ESI): Calc'd for C₁₉H₄₀NO₃Si [M+H]⁺ 358.27, found 358.2.

### Step 8: rac-(2R,3R)-8-(4-((tert-butyldimethylsilyl)oxy)butyl)-8-azaspiro[4.5]decane-2,3-diyl bis(2-heptylnonanoate)

This compound was prepared according to the general acylation procedure as described in Example 1 (Step 4). rac-(2R,3R)-8-(4-((tert-Butyldimethylsilyl)oxy)butyl)-8-azaspiro[4.5]decane-2,3-diol (770.0 mg, 2.15 mmol) provided rac-(2R,3R)-8-(4-((tert-butyldimethylsilyl)oxy)butyl)-8-azaspiro[4.5]decane-2,3-diyl bis(2-heptylnonanoate) (1.52 g, 84.7% yield) as a light-yellow oil. ¹H NMR (400 MHZ, CDCl₃) δ 5.14-5.11 (m, 2H), 3.61-3.58 (m, 2H), 2.55-2.33 (m, 5H), 2.32-2.20 (m, 3H), 2.06 (dd, J=14.2, 6.2 Hz, 2H), 1.71-1.35 (m, 20H), 1.32-1.20 (m, 40H), 0.89-0.83 (m, 19H), 0.03 (d, J=1.9 Hz, 6H). LCMS (ESI): Calc'd for C₅₁H₁₀₀NO₅Si [M+H]⁺ 834.73, found 834.5.

### Step 9: rac-(2R,3R)-8-(4-hydroxybutyl)-8-azaspiro[4.5]decane-2,3-diyl bis(2-heptylnonanoate)

This compound was prepared according to the general tert-butyldimethylsilyl deprotection procedure as described in Example 1 (Step 5). rac-(2R,3R)-8-(4-((tert-Butyldimethylsilyl)-oxy)butyl)-8-azaspiro[4.5]decane-2,3-diyl bis(2-heptylnonanoate) (1.52 g, 1.82 mmol) yielded rac-(2R,3R)-8-(4-hydroxybutyl)-8-azaspiro[4.5]decane-2,3-diyl bis(2-heptylnonanoate) (682 mg, 62% yield) as a colorless oil. ¹H NMR (400 MHZ, CDCl₃) δ ppm 6.27-7.11 (m, 1H), 5.13 (br t, J=4.13 Hz, 2H), 3.43-3.65 (m, 2H), 2.17-2.59 (m, 1H), 2.15-2.77 (m, 7H), 1.98-2.11 (m, 2H), 1.60-1.72 (m, 8H), 1.51-1.59 (m, 6H), 1.37-1.46 (m, 4H), 1.24 (br s, 40H), 0.79-0.92 (m, 12H). LCMS (ESI): Calc'd for C₄₅H₈₆NO₅ [M+H]+ 720.64, found 720.9.

### Example 52

### rac-(2R,3R)-8-(2-hydroxyethyl)-8-azaspiro[4.5]decane-2,3-diyl bis(2-heptylnonanoate) (52)

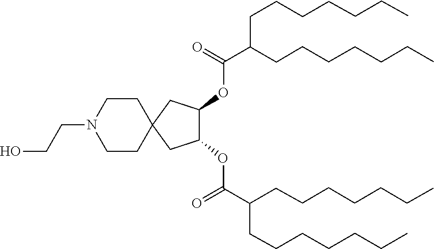 [SMILES: CCCCCCCC(CCCCCCC)C(=O)O[C@@H]1CC2(CCN(CCO)CC2)C[C@H]1OC(=O)C(CCCCCCC)CCCCCCC]

### Step 1: rac-(2R,3R)-8-(2-((tert-butyldimethylsilyl)oxy)ethyl)-8-azaspiro[4.5]decane-2,3-diol

This compound was prepared according to the general acylation procedure as described in Example 1 (Step 4). rac-(2R,3R)-8-azaspiro[4.5]decane-2,3-diol (Example 51, step 6) (1.0 g, 5.84 mmol) and (2-bromoethoxy) (tert-butyl)dimethylsilane (1.40 g, 5.84 mmol) afforded rac-(2R,3R)-8-(2-((tert-butyldimethylsilyl)oxy) ethyl)-8-azaspiro[4.5]decane-2,3-diol (800 mg, 42% yield). LCMS (ESI): Calc'd for C₁₇H₃₆NO₃Si [M+H]⁺ 330.24; found 330.1.

### Step 2: rac-(2R,3R)-8-(2-((tert-butyldimethylsilyl)oxy) ethyl)-8-azaspiro[4.5]decane-2,3-diyl bis(2-heptylnonanoate)

This compound was prepared according to the general acylation procedure as described in Example 1 (Step 4). rac-(2R,3R)-8-(2-((tert-butyldimethylsilyl)oxy)ethyl)-8-azaspiro[4.5]decane-2,3-diol (800 mg, 2.43 mmol) and 2-heptylnonanoic acid (A12) (1.37 g, 5.34 mmol) provided rac-(2R,3R)-8-(2-((tert-butyldimethylsilyl)oxy) ethyl)-8-azaspiro[4.5]decane-2,3-diyl bis(2-heptylnonanoate) that was contaminated with residual 2-heptylnonanoic acid (1.0 g, 51% yield).

### Step 3: rac-(2R,3R)-8-(2-hydroxyethyl)-8-azaspiro[4.5]decane-2,3-diyl bis(2-heptylnonanoate)

This compound was prepared according to the general HCl-mediated tert-butyldimethylsilyl deprotection as described in Example 1 (Step 5). rac-(2R,3R)-8-(2-((tert-butyldimethylsilyl)oxy)ethyl)-8-azaspiro[4.5]decane-2,3-diyl bis(2-heptylnonanoate) (1.0 g, 1.24 mmol) provided rac-(2R,3R)-8-(2-hydroxyethyl)-8-azaspiro[4.5]decane-2,3-diyl bis(2-heptylnonanoate) (205 mg, 44% yield) as a colorless oil. ¹H NMR (400 MHZ, CDCl₃) δ 5.14-5.12 (m, 2H), 3.73 (m, 2H), 2.70-2.63 (m, 6H), 2.32-2.25 (m, 2H), 2.10 (dd, J=14.1, 6.2 Hz, 2H), 1.63-1.55 (m, 7H), 1.48-1.37 (m, 5H), 1.25 (m, 42H), 0.88 (t, J=6.7 Hz, 12H). LCMS (ESI): Calc'd for C₄₃H₈₂NO₅ [M+H]⁺ 692.6; found 692.6.

### Example 53

### rac-(7R,8R)-2-(3-hydroxypropyl)-2-azaspiro[4.4]nonane-7,8-diyl bis(2-heptylnonanoate) (53)

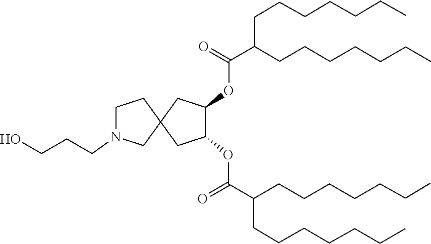 [SMILES: CCCCCCCC(CCCCCCC)C(=O)O[C@@H]1CC2(CCN(CCCO)C2)C[C@H]1OC(=O)C(CCCCCCC)CCCCCCC]

### Step 1: 2-(3-(benzyloxy)propyl)-2-azaspiro[4.4]non-7-en-1-one

A solution of 2-azaspiro[4.4]non-7-en-1-one (2.89 g, 21.1 mmol) in DMF (80 mL) was added dropwise to a mixture of NaH (1.26 g, 31.6 mmol) in DMF (80 mL) at 0° C. After stirring at 0° C. for 30 minutes, ((3-bromopropoxy) methyl)benzene (4.83 g, 21.1 mmol) and NaI (316 mg, 2.11 mmol) were added. The mixture was heated to 80° C. and stirred for 16 hours. The mixture was then quenched with saturated NH₄Cl. The aqueous layer was separated and extracted with EtOAc. The combined organic layers were washed with brine, dried over Na₂SO₄, and filtered. The filtrate was concentrated in vacuo, and the crude product was purified by silica gel chromatography to afford 2-(3-(benzyloxy)propyl)-2-azaspiro[4.4]non-7-en-1-one (3.7 g, 82% yield) as a light-yellow oil. LCMS (ESI): Calc'd for C₁₈H₂₄NO₂ [M+H]⁺ 286.2; found 286.1.

### Step 2: 1′-(3-(benzyloxy)propyl)-6-oxaspiro[bicyclo[3.1.0]hexane-3,3′-pyrrolidin]-2′-one

MCPBA (4.47 g, 14.7 mmol) and NaHCO₃ (2.72 g, 32.4 mmol) were added to a solution of 2-(3-(benzyloxy) propyl)-2-azaspiro[4.4]non-7-en-1-one (3.7 g, 12.97 mmol) in DCM (70 mL). After stirring at room temperature for 2 hours, the mixture was partitioned between saturated Na₂S₂O₃ and DCM. The combined organic layers were washed with brine, dried over Na₂SO₄ and filtered. The filtrate was concentrated in vacuo, and the crude product was purified by silica gel chromatography to provide 1′-(3-(benzyloxy)propyl)-6-oxaspiro[bicyclo[3.1.0]hexane-3,3′-pyrrolidin]-2′-one (3.8 g, 97%) as a light-yellow oil. LCMS (ESI): Calc'd for C₁₈H₂₄NO₃ [M+H]⁺ 302.2; found 302.2.

### Step 3: rac-(7R,8R)-2-(3-(benzyloxy) propyl)-7,8-dihydroxy-2-azaspiro[4.4]nonan-1-one

A solution of 1′-(3-(benzyloxy) propyl)-6-oxaspiro[bicyclo[3.1.0]hexane-3,3′-pyrrolidin]-2′-one (3.8 g, 12.61 mmol) and H₂SO₄ (2M, 20 mL) in dioxane (40 mL) was stirred at room temperature for 4 hours. The mixture was then quenched with saturated NaHCO₃. The aqueous phase was extracted with EtOAc. The combined organic layers were dried over Na₂SO₄ and filtered. The filtrate was concentrated in vacuo. The crude residue was purified by silica gel chromatography to afford rac-(7R,8R)-2-(3-(benzyloxy)propyl)-7,8-dihydroxy-2-azaspiro[4.4]-nonan-1-one (1.9 g, 47%) as a light-yellow oil. LCMS (ESI): Calc'd for C₁₈H₂₆NO₄ [M+H]⁺ 320.2; found 320.2.

### Step 4: rac-(7R,8R)-2-(3-(benzyloxy) propyl)-2-azaspiro[4.4]nonane-7,8-diyl bis(2-heptylnonanoate)

This compound was prepared according to the general acylation procedure as described in Example 1 (Step 4). rac-(7R,8R)-2-(3-(benzyloxy) propyl)-7,8-dihydroxy-2-azaspiro[4.4]nonan-1-one (650 mg, 2.13 mmol) and 2-heptylnonanoic acid (A12) (1.64 g, 6.38 mmol) afforded rac-(7R,8R)-2-(3-(benzyloxy) propyl)-2-azaspiro[4.4]nonane-7,8-diyl bis(2-heptylnonanoate) (1.6 g, 96% yield) as a light-yellow oil. LCMS (ESI): Calc'd for C₅₀H₈₈NO₅ [M+H]⁺ 782.7; found 782.7.

### Step 5: rac-(7R,8R)-2-(3-hydroxypropyl)-2-azaspiro[4.4]nonane-7,8-diyl bis(2-heptylnonanoate)

Pd(OH)₂ (215 mg, 1.53 mmol) and 10% Pd/C (163 mg, 0.15 mmol) were added to a solution of rac-(7R,8R)-2-(3-(benzyloxy) propyl)-2-azaspiro[4.4]nonane-7,8-diyl bis(2-heptylnonanoate) (1.2 g, 1.53 mmol) in THF (15 mL), and the mixture was stirred under an atmosphere of H₂ (50 psi) at 60° C. for 72 hours. The reaction mixture was filtered. The filtrate was concentrated in vacuo, and the residue was purified by silica gel chromatography to afford rac-(7R,8R)-2-(3-hydroxypropyl)-2-azaspiro[4.4]nonane-7,8-diyl bis(2-heptylnonanoate) (219 mg, 21% yield) as a light-yellow oil. ¹H NMR (400 MHZ, CD₃OD) δ 5.02-5.23 (m, 2H), 3.69-3.54 (m, 2H), 2.72-2.50 (m, 6H), 2.40-2.12 (m, 4H), 1.98-1.82 (m, 2H), 1.81-1.66 (m, 4H), 1.65-1.53 (m, 4H), 1.51-1.41 (m, 4H), 1.39-1.20 (m, 40H), 0.97-0.81 (m, 12H). LCMS (ESI): Calc'd for C₄₃H₈₂NO₅ [M+H]⁺ 692.61; found 692.7.

### Example 54

### rac-(2R,3R)-8-(3-hydroxypropyl)-8-azaspiro[4.5]decane-2,3-diyl bis(2-heptylnonanoate) (54)

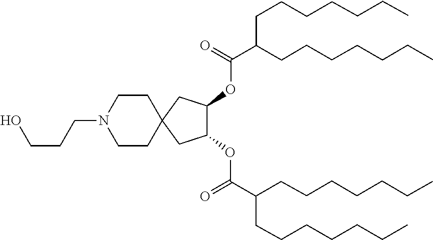 [SMILES: CCCCCCCC(CCCCCCC)C(=O)O[C@@H]1CC2(CCN(CCCO)CC2)C[C@H]1OC(=O)C(CCCCCCC)CCCCCCC]

### Step 1: rac-(2R,3R)-8-(3-((tert-butyldimethylsilyl)oxy) propyl)-8-azaspiro[4.5]decane-2,3-diol

This compound was prepared according to the general alkylation procedure as described in Example 1 (Step 3). rac-(2R,3R)-8-azaspiro[4.5]decane-2,3-diol (Example 51, step 6) (1.0 g, 5.84 mmol) and (3-bromopropoxy)(tert-butyl)dimethylsilane provided rac-(2R,3R)-8-(3-((tert-butyldimethylsilyl)oxy)propyl)-8-azaspiro[4.5]decane-2,3-diol (200 mg, 10% yield) as a yellow oil. ¹H NMR (400 MHZ, CDCl₃) δ 4.03 (s, 2H), 3.65 (t, J=6.2 Hz, 2H), 2.40 (brs, 4H), 1.97 (dd, J=13.6, 6.0 Hz, 2H), 1.76 (m, 4H), 1.53-1.44 (m, 8H), 0.88 (s, 9H), 0.04 (s, 6H). LCMS (ESI): Calc'd for C₁₈H₃₈NO₃Si [M+H]⁺ 344.3, found 344.2.

### Step 2: rac-(2R,3R)-8-(3-((tert-butyldimethylsilyl)oxy) propyl)-8-azaspiro[4.5]decane-2,3-diyl bis(2-heptylnonanoate)

This compound was prepared according to the general acylation procedure as described in Example 1 (Step 4). rac-(2R,3R)-8-(3-((tert-butyldimethylsilyl)oxy) propyl)-8-azaspiro[4.5]-decane-2,3-diol (200 mg, 0.582 mmol) and 2-heptylnonanoic acid (A12) (328 mg, 1.28 mmol) provided rac-(2R,3R)-8-(3-((tert-butyldimethylsilyl)oxy) propyl)-8-azaspiro[4.5]decane-2,3-diyl bis(2-heptylnonanoate) (170 mg, 36%) as a colorless oil.

### Step 3: rac-(2R,3R)-8-(3-hydroxypropyl)-8-azaspiro[4.5]decane-2,3-diyl bis(2-heptylnonanoate)

This compound was prepared according to the general HCl-mediated tert-butyldimethylsilyl deprotection as described in Example 1 (Step 5). rac-(2R,3R)-8-(3-((tert-butyldimethylsilyl)oxy) propyl)-8-azaspiro[4.5]decane-2,3-diyl bis(2-heptylnonanoate) (450 mg, 0.56 mmol) afforded rac-(2R,3R)-8-(3-hydroxypropyl)-8-azaspiro[4.5]decane-2,3-diyl bis(2-heptylnonanoate) (292 mg, 33% yield) as a colorless oil. ¹H NMR (400 MHZ, CDCl₃) δ 5.13 (t, J=4.8 Hz, 2H), 3.80 (t, J=5.2 Hz, 2H), 2.60 (br s, 2H), 2.28 (tt, J=8.8, 5.4 Hz, 2H), 2.06 (dd, J=14.3, 6.3 Hz, 2H), 1.78-1.49 (m, 16H), 1.46-1.36 (m, 5H), 1.31-1.20 (m, 40H), 0.87 (t, J=6.7 Hz, 12H). LCMS (ESI): Calc'd for C₄₄H₈₄NO₅ [M+H]⁺ 706.63, found 706.6.

### Example 55

### rac-(2R,3R)-8-(3-((2-(methylamino)-3,4-dioxocyclobut-1-en-1-yl)amino)propyl)-8-azaspiro[4.5]decane-2,3-diyl bis(2-heptylnonanoate) (55)

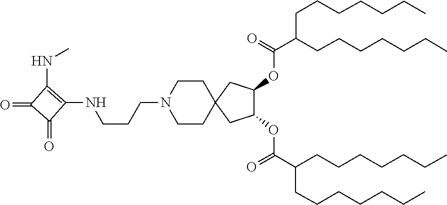 [SMILES: CCCCCCCC(CCCCCCC)C(=O)O[C@@H]1CC2(CCN(CCCNc3c(NC)c(=O)c3=O)CC2)C[C@H]1OC(=O)C(CCCCCCC)CCCCCCC]

### Step 1: rac-tert-butyl (tert-butoxycarbonyl) (3-((2R,3R)-2,3-dihydroxy-8-azaspiro[4.5]decan-8-yl)propyl) carbamate

This compound was prepared according to the general alkylation using tert-butyl (3-bromopropyl) (tert-butoxycarbonyl) carbamate as described in Example 9 (Step 1). rac-(2R,3R)-8-azaspiro[4.5]decane-2,3-diol (Example 51, step 6) (700 mg, 3.37 mmol) provided rac-tert-butyl (tert-butoxycarbonyl) (3-((2R,3R)-2,3-dihydroxy-8-azaspiro[4.5]decan-8-yl)propyl) carbamate (320 mg, 22%) as a colorless oil. LCMS (ESI): Calc'd for C₂₂H₄₁N₂O₈ [M+H]⁺ 429.3; found 429.2.

### Step 2: rac-(2R,3R)-8-(3-(bis(tert-butoxycarbonyl)amino)propyl)-8-azaspiro[4.5]decane-2,3-diyl bis(2-heptylnonanoate)

This compound was prepared according to the general acylation procedure as described in Example 1 (Step 4). rac-tert-butyl (tert-butoxycarbonyl) (3-((2R,3R)-2,3-dihydroxy-8-azaspiro[4.5]decan-8-yl)propyl) carbamate (320 mg, 0.747 mmol) and 2-heptylnonanoic acid (A12) (395 mg, 1.54 mmol) provided rac-(2R,3R)-8-(3-(bis(tert-butoxycarbonyl)amino)propyl)-8-azaspiro[4.5]decane-2,3-diyl bis(2-heptylnonanoate) (620 mg, 45% yield) as a colorless oil. LCMS (ESI): Calc'd for C₅₄H₁₀₁N₂O₈ [M+H]⁺ 905.8; found 905.5.

### Step 3: rac-(2R,3R)-8-(3-aminopropyl)-8-azaspiro[4.5]decane-2,3-diyl bis(2-heptylnonanoate)

This compound was prepared according to the general HCl-mediated boc deprotection as described in Example 1 (Step 5). rac-(2R,3R)-8-(3-(bis(tert-butoxycarbonyl)amino)propyl)-8-azaspiro[4.5]decane-2,3-diyl bis(2-heptylnonanoate) (620 mg, 0.685 mmol) afforded rac-(2R,3R)-8-(3-aminopropyl)-8-azaspiro[4.5]decane-2,3-diyl bis(2-heptylnonanoate) (533 mg, 100%) as a colorless solid. LCMS (ESI): Calc'd for C₄₄H₈₅N₂O₄ [M+H]⁺ 705.6; found 705.5.

### Step 4: rac-(2R,3R)-8-(3-((2-(methylamino)-3,4-dioxocyclobut-1-en-1-yl)amino)propyl)-8-azaspiro[4.5]decane-2,3-diyl bis(2-heptylnonanoate)

This compound was prepared according to the general squaramide formation using 3-ethoxy-4-(methylamino) cyclobut-3-ene-1,2-dione as describe in Example 8 (Step 4). rac-(2R,3R)-8-(3-Aminopropyl)-8-azaspiro[4.5]decane-2,3-diyl bis(2-heptylnonanoate) (533 mg, 0.685 mmol) provided rac-(2R,3R)-8-(3-((2-(methylamino)-3,4-dioxocyclobut-1-en-1-yl)amino)propyl)-8-azaspiro[4.5]decane-2,3-diyl bis(2-heptylnonanoate) (164 mg, 29% yield) as a white solid. ¹H NMR (400 MHZ, CDCl₃) δ 5.17-5.11 (m, 2H), 3.75-3.66 (m, 2H), 3.34 (d, J=5.1 Hz, 3H), 3.18 (br s, 1H), 2.33-2.23 (m, 3H), 2.19-2.07 (m, 5H), 1.90 (br s, 1H), 1.69-1.48 (m, 12H), 1.48-1.36 (m, 6H), 1.31-1.21 (m, 40H), 0.88 (t, J=6.8 Hz, 12H). LCMS (ESI): Calc'd for C₄₉H₈₈N₃O₆ [M+H]⁺ 814.66, found 814.6.

### Example 56

### rac-(2R,3R)-8-(5-hydroxypentyl)-8-azaspiro[4.5]decane-2,3-diyl bis(2-heptylnonanoate) (56)

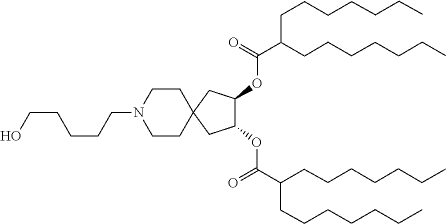 [SMILES: CCCCCCCC(CCCCCCC)C(=O)O[C@@H]1CC2(CCN(CCCCCO)CC2)C[C@H]1OC(=O)C(CCCCCCC)CCCCCCC]

### Step 1: rac-(2R,3R)-8-(5-((tert-butyldimethylsilyl)oxy) pentyl)-8-azaspiro[4.5]decane-2,3-diol

This compound was prepared according to the general alkylation procedure as described in Example 1 (Step 3). rac-(2R,3R)-8-Azaspiro[4.5]decane-2,3-diol (Example 51, step 6) (1.00 g, 5.84 mmol) and ((5-bromopentyl)oxy) (tert-butyl)dimethylsilane (1.72 g, 6.13 mmol) provided rac-(2R,3R)-8-(5-((tert-butyldimethylsilyl)oxy) pentyl)-8-azaspiro[4.5]decane-2,3-diol (0.81 g, 37% yield) as a light-yellow oil. LCMS (ESI): Calc'd for C₂₀H₄₂NO₃Si [M+H]⁺ 372.29, found 372.1.

### Step 2: rac-(2R,3R)-8-(5-((tert-butyldimethylsilyl)oxy) pentyl)-8-azaspiro[4.5]decane-2,3-diyl bis(2-heptylnonanoate)

This compound was prepared according to the general acylation procedure as described in Example 1 (Step 4). rac-(2R,3R)-8-(5-((tert-butyldimethylsilyl)oxy) pentyl)-8-azaspiro[4.5]-decane-2,3-diol (0.100 g, 0.269 mmol) and 2-heptylnonanoic acid (A12) (0.172 g, 0.673 mmol) provided rac-(2R,3R)-8-(5-((tert-butyldimethylsilyl)oxy) pentyl)-8-azaspiro[4.5]decane-2,3-diyl bis(2-heptylnonanoate) (0.10 g, 44% yield) as a light-yellow oil. ¹H NMR (400 MHZ, CDCl₃) δ 5.13 (t, J=4.6 Hz, 2H), 3.58 (t, J=6.4 Hz, 2H), 2.53 (br s, 2H), 2.42 (t, J=8.0 Hz, 2H), 2.32-2.19 (m, 3H), 2.09-2.01 (m, 2H), 1.66 (br s, 3H), 1.61-1.47 (m, 10H), 1.45-1.36 (m, 6H), 1.32-1.21 (m, 42H), 0.90-0.82 (m, 21H), 0.03 (s, 6H).

### Step 3: rac-(2R,3R)-8-(5-hydroxypentyl)-8-azaspiro[4.5]decane-2,3-diyl bis(2-heptylnonanoate)

This compound was prepared according to the general HCl-mediated tertbutyldimethyl silyl deprotection as described in Example 1 (Step 5). rac-(2R,3R)-8-(5-((tert-butyldimethylsilyl)oxy) pentyl)-8-azaspiro[4.5]decane-2,3-diyl bis(2-heptylnonanoate) (0.790 g, 0.931 mmol) provided rac-(2R,3R)-8-(5-hydroxypentyl)-8-azaspiro[4.5]decane-2,3-diyl bis(2-heptylnonanoate) hydrochloride salt (0.30 g, 44%) as a light-yellow oil. ¹H NMR (400 MHZ, CDCl₃) δ 12.17 (s, 1H), 5.13 (d, J=35.3 Hz, 2H), 3.67 (t, J=6.0 Hz, 2H), 3.53-3.41 (m, 2H), 2.91 (t, J=8.3 Hz, 2H), 2.65 (br s, 2H), 2.45 (br s, 2H), 2.28 (br s, 2H), 2.15 (dd, J=4.3, 6.7 Hz, 2H), 1.99-1.89 (m, 2H), 1.63-1.56 (m, 6H), 1.53-1.36 (m, 10H), 1.29-1.19 (m, 40H), 0.88 (t, J=6.7 Hz, 12H). LCMS (ESI): Calc'd for C₄₆H₈₈NO₅ [M+H]⁺ 734.66, found 734.6.

Examples 57 and 58 were prepared through chiral separation of rac-(2R,3R)-8-(4-hydroxybutyl)-8-azaspiro[4.5]decane-2,3-diyl bis(2-heptylnonanoate) obtained in Example 51 using the following conditions: Chiral Technologies IG 250 mm×30 mm, 5 μm column; mobile phase A: CO₂ (70%), mobile phase B: 2-propanol containing 0.2% isopropylamine; 40° C., 80 mL/min.

TABLE 4

| Ex | Lipid | Analytical Data |
|---|---|---|
| 57 | (2R*,3R*)-8-(4-hydroxybutyl)-8-azaspiro[4.5]decane-2,3-diyl bis(2- heptylnonanoate) (57) 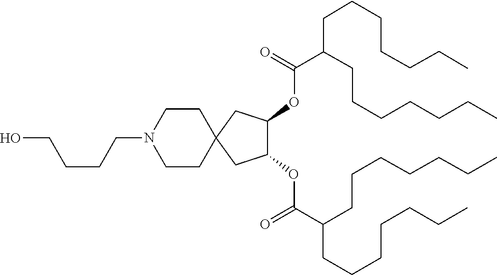 [SMILES: CCCCCCCC(CCCCCCC)C(=O)O[C@@H]1CC2(CCN(CCCCO)CC2)C[C@H]1OC(=O)C(CCCCCCC)CCCCCCC] | ¹H NMR (400 MHz, CDCl₃) δ 5.15 (t, J = 4.8 Hz, 2H), 3.78-3.70 (m, 1H), 3.64-3.57 (m, 2H), 2.71-2.39 (m, 4H), 2.30 (tt, J = 9.0, 5.3 Hz, 2H), 2.15-2.04 (m, 2H), 1.83-1.65 (m, 7H), 1.64-1.51 (m, 7 H), 1.48-1.36 (m, 4H), 1.33-1.19 (m, 42H), 0.89 (t, J = 6.7 Hz, 12H). LCMS (ESI): Calc'd for C₄₅H₈₆NO₅ [M + H]⁺ 720.64, found 721.0. First eluting isomer |
| 58 | (2S*,3S*)-8-(4-hydroxybutyl)-8-azaspiro[4.5]decane-2,3-diyl bis(2- heptylnonanoate) (58) 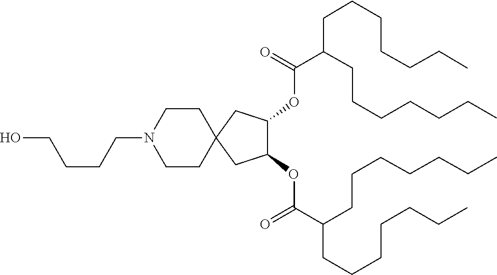 [SMILES: CCCCCCCC(CCCCCCC)C(=O)O[C@H]1CC2(CCN(CCCCO)CC2)C[C@@H]1OC(=O)C(CCCCCCC)CCCCCCC] | ¹H NMR (400 MHz, CDCl₃) δ 5.15 (t, J = 4.8 Hz, 2H), 3.78-3.70 (m, 1H), 3.64-3.57 (m, 2H), 2.71-2.39 (m, 4H), 2.30 (tt, J = 9.0, 5.3 Hz, 2H), 2.15-2.04 (m, 2H), 1.83-1.65 (m, 7H), 1.64-1.51 (m, 7H), 1.48-1.36 (m, 4H), 1.33-1.19 (m, 42H), 0.89 (t, J = 6.7 Hz, 12H). LCMS (ESI): Calc'd for C₄₅H₈₆NO₅ [M + H]⁺ 720.64, found 721.0. Second eluting isomer |

The following Examples 59-70 were prepared in a similar manner to Example 51 and other examples described herein employing the stated carboxylic acid.

TABLE 5

| Ex | Lipid | Analytical Data |
|---|---|---|
| 59 | According to Example 51 with A20. rac-(2R,3R)-8-(4-hydroxybutyl)-8-azaspiro[4.5]decane-2,3-diyl bis(2-octyldecanoate) (59) 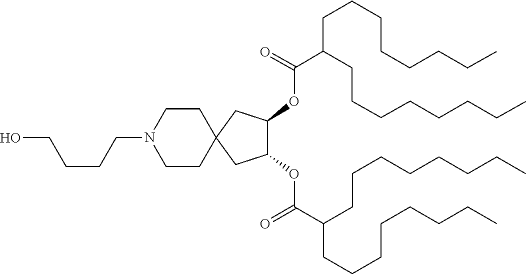 [SMILES: CCCCCCCCC(CCCCCCCC)C(=O)O[C@@H]1CC2(CCN(CCCCO)CC2)C[C@H]1OC(=O)C(CCCCCCCC)CCCCCCCC] | ¹H NMR (400 MHz, CD₃OD) δ 5.15 (t, J = 4.7 Hz, 2H), 4.58 (s, 1H), 3.56 (t, J = 5.9 Hz, 2H), 2.53 (br s, 2H), 2.42 (t, J = 6.9 Hz, 2H), 2.33 (tt, J = 9.5, 5.1 Hz, 2H), 2.09 (dd, J = 13.9, 6.5 Hz, 2H), 1.67 (t, J = 5.5 Hz, 4H), 1.64-1.52 (m, 10H), 1.50-1.39 (m, 4H), 1.33-1.26 (m, 50H), 0.93-0.87 (m, 12H). LCMS (SI): Calc'd for C₄₉H₉₄NO₅ [M + H]⁺ 776.70, found 776.6. |
| 60 | According to Example 51 with A7. rac-(2R,3R)-8-(4-hydroxybutyl)-8-azaspiro[4.5]decane-2,3-diyl bis(2- heptyltetradecanoate) (60) 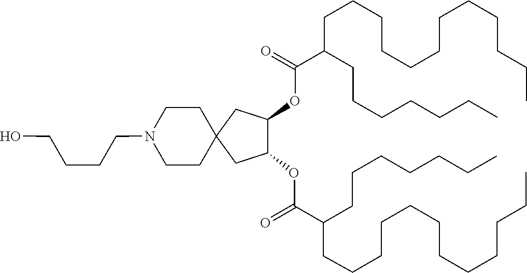 [SMILES: CCCCCCCCCCCCC(CCCCCCC)C(=O)O[C@@H]1CC2(CCN(CCCCO)CC2)C[C@H]1OC(=O)C(CCCCCCC)CCCCCCCCCCCC] | ¹H NMR (400 MHz, CDCl₃) δ 5.11 (t, J = 4.4 Hz, 2H), 3.53 (s, 2H), 2.48 (br s, 2H), 2.37 (s, 2H), 2.25 (td, J = 8.9, 4.5 Hz, 2H), 2.04 (dd, J = 14.1, 6.3 Hz, 2H), 1.68-1.61 (m, 7H), 1.59-1.46 (m, 7H), 1.45-1.33 (m, 5H), 1.29-1.18 (m, 62H), 0.85 (t, J = 6.7 Hz, 12H). LCMS (ESI): Calc'd for C₅₅H₁₀₆NO₅ [M + H]⁺ 860.80, found 860.7. |
| 61 | According to Example 51 with A13. rac-(2R,3R)-8-(4-hydroxybutyl)-8-azaspiro[4.5]decane-2,3-diyl bis(2-hexyldecanoate) (61) 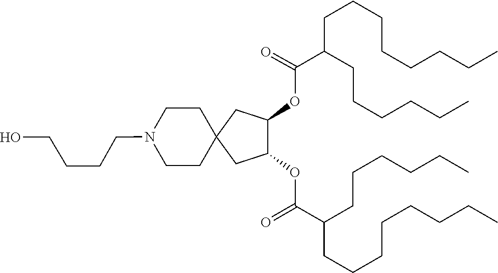 [SMILES: CCCCCCCCC(CCCCCC)C(=O)O[C@@H]1CC2(CCN(CCCCO)CC2)C[C@H]1OC(=O)C(CCCCCC)CCCCCCCC] | ¹H NMR (400 MHz, CDCl₃) δ 5.13 (t, J = 4.6 Hz, 2H), 3.58 (t, J = 4.7 Hz, 2H), 2.44 (br s, 4H), 2.28 (tt, J = 8.7, 5.4 Hz, 2H), 2.07 (dd, J = 14.1, 6.3 Hz, 2H), 1.77-1.62 (m, 7H), 1.61-1.49 (m, 7H), 1.47-1.34 (m, 5H), 1.29-1.19 (m, 42H), 0.87 (t, J = 6.6 Hz, 12H). LSMS (ESI): Calc'd for C₄₅H₈₆NO₅ [M + H]⁺ 720.64, found 720.6 |
| 62 | According to Example 51 with A2. rac-O,O′-((2R,3R)-8-(4-hydroxybutyl)-8-azaspiro[4.5]decane-2,3-diyl) di(pentadecan-8- yl) disuccinate (62) 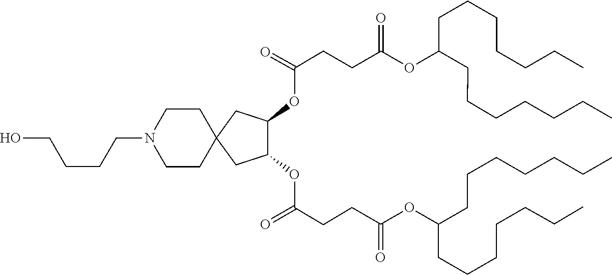 [SMILES: CCCCCCCC(CCCCCCC)OC(=O)CCC(=O)O[C@@H]1CC2(CCN(CCCCO)CC2)C[C@H]1OC(=O)CCC(=O)OC(CCCCCCC)CCCCCCC] | ¹H NMR (400 MHz, CD₃OD) δ ppm 5.07-5.24 (m, 2H), 3.51-3.61 (m, 2H), 2.34-2.69 (m, 14H), 1.97-2.15 (m, 2H), 1.49-1.71 (m, 18H), 1.14- 1.44 (m, 42H), 0.77-1.02 (m, 12H). LCMS (ESI): Calc'd for C₅₁H₉₄NO₉ [M + H]⁺ 864.69; found 864.7. |
| 63 | According to Example 51 with A1 rac-O′¹,O¹-((2R,3R)-8-(4-hydroxybutyl)-8-azaspiro[4.5]decane-2,3-diyl) 7,7′-di(pentadecan-8- yl) di(heptanedioate) (63) 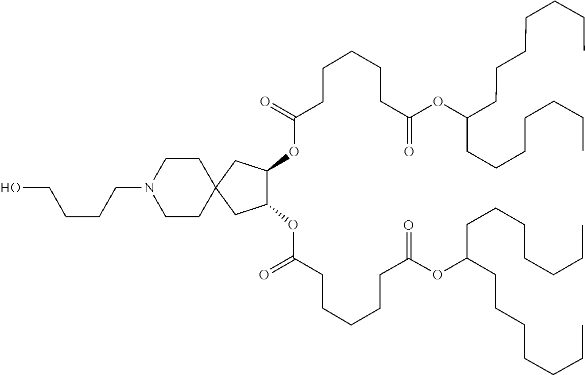 [SMILES: CCCCCCCC(CCCCCCC)OC(=O)CCCCCC(=O)O[C@@H]1CC2(CCN(CCCCO)CC2)C[C@H]1OC(=O)CCCCCC(=O)OC(CCCCCCC)CCCCCCC] | ¹H NMR (400 MHz, CD₃OD) δ ppm 5.09-5.20 (m, 2H), 3.50-3.60 (m, 2H), 2.44-2.56 (m, 3H), 2.36-2.43 (m, 3H), 2.29-2.35 (m, 8H), 2.00-2.14 (m, 2H), 1.53-1.67 (m, 26H), 1.25-1.38 (m, 46H), 0.87-0.94 (m, 12H). LCMS (ESI): Calc'd for C₅₇H₁₀₆NO₉ [M + H]⁺ 948.78; found 948.7. |
| 64 | According to Example 51 with A3. rac-(((2R,3R)-8-(4-hydroxybutyl)-8-azaspiro[4.5]decane-2,3-diyl)bis(oxy))bis(6-oxohexane-6,1- diyl) bis(2-heptylnonanoate) (64) 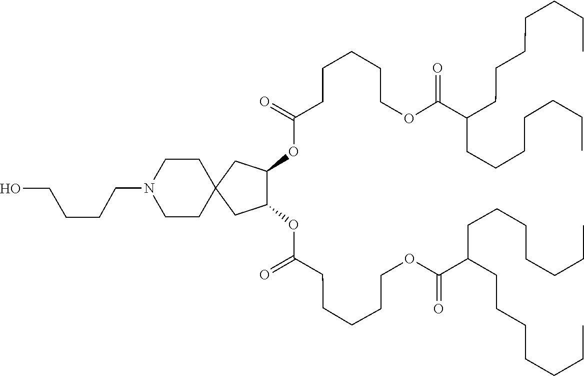 [SMILES: CCCCCCCC(CCCCCCC)C(=O)OCCCCCC(=O)O[C@@H]1CC2(CCN(CCCCO)CC2)C[C@H]1OC(=O)CCCCCOC(=O)C(CCCCCCC)CCCCCCC] | ¹H NMR (400 MHz, CD₃OD) δ 5.14 (t, J = 5.0 Hz, 2H), 4.09 (t, J = 6.4 Hz, 4H), 3.56 (t, J = 5.9 Hz, 2H), 2.48 (brs, 2H), 2.39-2.29 (m, 8H), 2.07 (dd, J = 13.9, 6.5 Hz, 2H), 1.66-1.56 (m, 22H), 1.47-1.40 (m, 8H), 1.32-1.27 (m, 42H), 0.91-0.89 (m, 12H). LCMS (ESI): Calc'd for C₅₇H₁₀₆NO₉ [M + H]⁺ 948.8; found 948.9. |
| 65 | According to Example 51 with A6. rac-(2R,3R)-8-(4-hydroxybutyl)-8-azaspiro[4.5]decane-2,3-diyl bis(2-pentyldecanoate) (65) 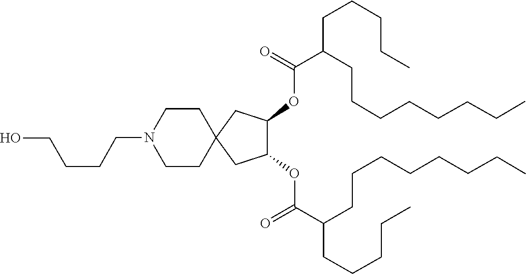 [SMILES: CCCCCCCCC(CCCCC)C(=O)O[C@@H]1CC2(CCN(CCCCO)CC2)C[C@H]1OC(=O)C(CCCCC)CCCCCCCC] | ¹H NMR (400 MHz, CD₃OD) δ 5.16 (t, J = 4.8 Hz, 2H), 3.58 (t, J = 6.0 Hz, 2H), 2.52 (br s, 2H), 2.43 (t, J = 6.8 Hz, 2H), 2.35 (ddd, J = 14.0, 9.0, 5.0, 2H), 2.11 (dd, J = 14.0, 7.3 Hz, 2H), 1.68 (t, J = 5.2 Hz, 4H), 1.65- 1.53 (m, 10H), 1.52-1.42 (m, 4H), 1.40-1.23 (m, 38H), 0.92 (t, J = 6.4, 12H). LCMS (ESI): Calc'd for C₄₃H₈₂NO₅ [M + H]⁺ 692.62, found 692.8. |
| 66 | According to Example 51 with A14. rac-(2R,3R)-8-(4-hydroxybutyl)-8-azaspiro[4.5]decane-2,3-diyl bis(2-heptyldecanoate) (66) 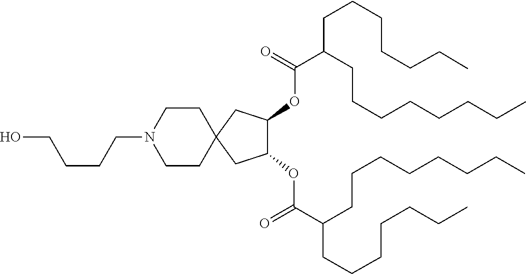 [SMILES: CCCCCCCCC(CCCCCCC)C(=O)O[C@@H]1CC2(CCN(CCCCO)CC2)C[C@H]1OC(=O)C(CCCCCCC)CCCCCCCC] | ¹H NMR (400 MHz, CD₃OD) δ 5.14 (t, J = 4.4 Hz, 2H), 3.55 (t, J = 5.6 Hz, 2H), 2.49 (br s, 2H), 2.41 (t, J = 7.2 Hz, 2H), 2.36-2.28 (m, 2H), 2.08 (dd, J = 16.0, 7.0 Hz, 2H), 1.66 (t, J = 5.6 Hz, 4H), 1.64-1.51 (m, 10H), 1.50- 1.40 (m, 4H), 1.38-1.20 (m, 46H), 0.90 (t, J = 6.8 Hz, 12H). LCMS (ESI): Calc'd for C₄₇H₉₀NO₅ [M + H]⁺ 748.68, found 748.9. |
| 67 | According to Example 51 with A24. rac-(2R,3R)-8-(4-hydroxybutyl)-8-azaspiro[4.5]decane-2,3-diyl bis(2-hexylundecanoate) (67) 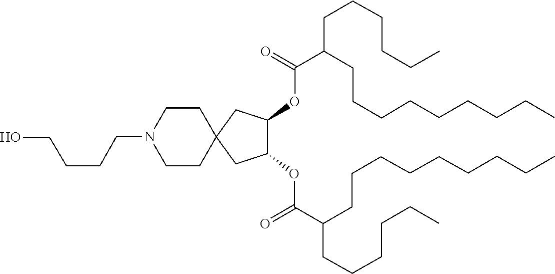 [SMILES: CCCCCCCCCC(CCCCCC)C(=O)O[C@@H]1CC2(CCN(CCCCO)CC2)C[C@H]1OC(=O)C(CCCCCC)CCCCCCCCC] | ¹H NMR (400 MHz, CD₃OD) δ 5.15 (t, J = 4.6 Hz, 2H), 3.56 (t, J = 6.0 Hz, 2H), 2.53 (br s, 2H), 2.43 (t, J = 7.2 Hz, 2H), 3.45 (ddd, J = 13.9, 9.7, 4.5, 2H), 2.09 (dd, J = 13.8, 6,4 Hz, 2H), 1.67 (t, J = 5.4 Hz, 4H), 1.65-1.52 (m, 10H), 1.51-1.40 (m, 4H), 1.38-1.21 (m, 46H), 0.91 (t, J = 7.0 Hz, 12H). LCMS (ESI): Calc'd for C₄₇H₉₀NO₅ [M + H]⁺ 748.68, found 748.9. |
| 68 | According to Example 51 with A25. rac-(2R,3R)-8-(4-hydroxybutyl)-8-azaspiro[4.5]decane-2,3-diyl bis(2- hexylnonanoate) (68) 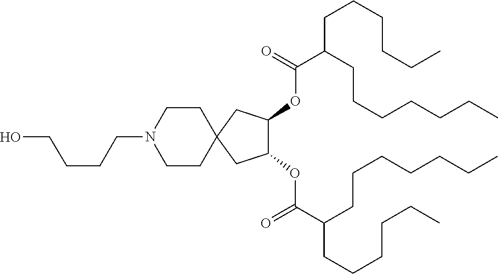 [SMILES: CCCCCCCC(CCCCCC)C(=O)O[C@@H]1CC2(CCN(CCCCO)CC2)C[C@H]1OC(=O)C(CCCCCC)CCCCCCC] | ¹H NMR (400 MHz, CDCl₃) δ 5.20- 5.08 (m, 2H), 3.67-3.46 (m, 2H), 2.60-2.34 (m, 4H), 2.33-2.25 (m, 2H), 2.12-2.02 (m, 2H), 1.75-1.62 (m, 8H), 1.60-1.52 (m, 6H), 1.47-1.38 (m, 4H), 1.32-1.18 (br s, 38H), 0.91- 0.85 (m, 12H). LCMS (ESI): Calc'd for C₄₃H₈₂NO₅ [M + H]⁺ 692.61; found 692.9. |
| 69 | According to Example 51 with A16. rac-(2R,3R)-8-(4-hydroxybutyl)-8-azaspiro[4.5]decane-2,3-diyl bis(2-butyldecanoate) (69) 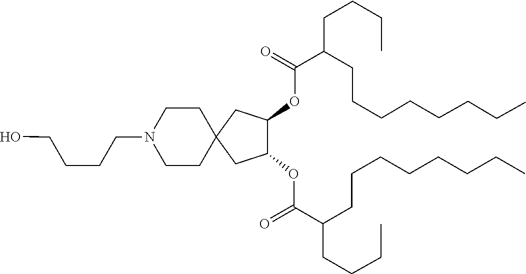 [SMILES: CCCCCCCCC(CCCC)C(=O)O[C@@H]1CC2(CCN(CCCCO)CC2)C[C@H]1OC(=O)C(CCCC)CCCCCCCC] | ¹H NMR (400 MHz, CD₃OD) δ 5.19- 5.11 (m, 2H), 3.61-3.52 (m, 2H), 2.55-2.44 (m, 2H), 2.40-2.30 (m, 4H), 2.12-2.03 (m, 2H), 1.68-1.54 (m, 14H), 1.54-1.38 (m, 6H), 1.34- 1.24 (m, 32H), 0.93-0.88 (m, 12H). LCMS (ESI): Calc'd for C₄₁H₇₈NO₅ [M + H]⁺ 664.58; found 664.8. |
| 70 | According to Example 51 with A13/A26. rac-(2R,3R)-3-((2-ethylnonanoyl)oxy)-8-(4-hydroxybutyl)-8-azaspiro[4.5]decan-2- yl 2-hexyldecanoate (70) 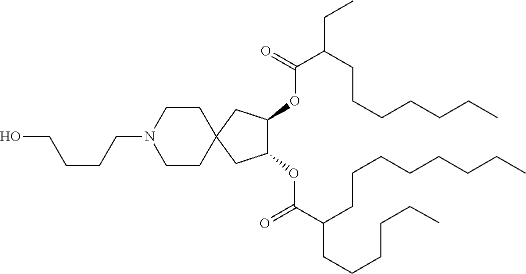 [SMILES: CCCCCCCCC(CCCCCC)C(=O)O[C@@H]1CC2(CCN(CCCCO)CC2)C[C@H]1OC(=O)C(CC)CCCCCCC] | ¹H NMR (400 MHz, CD₃OD) δ 5.20-5.10 (m, 2H), 3.61-3.51 (m, 2H), 2.57-2.48 (m, 2H), 2.45-2.38 (m, 2H), 2.37-2.31 (m, 1H), 2.29-2.22 (m, 1H), 2.14-2.04 (m, 2H), 1.70- 1.65 (m, 4H), 1.63-1.52 (m, 10H), 1.50-1.39 (m, 4H), 1.37-1.19 (m, 32H), 0.98-0.85 (m, 12H). LCMS (ESI): Calc'd for C₄₀H₇₆NO₅ [M + H]⁺ 650.57; found 650.8. |

### Example 71

### bis(3-pentyloctyl) 3-(9-(4-hydroxybutyl)-3,9-diazaspiro[5.5]undecan-3-yl)pentanedioate (71)

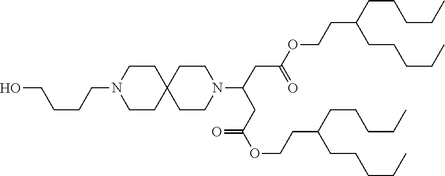 [SMILES: CCCCCC(CCCCC)CCOC(=O)CC(CC(=O)OCCC(CCCCC)CCCCC)N1CCC2(CCN(CCCCO)CC2)CC1]

### Step 1: bis(3-pentyloctyl) (E/Z)-pent-2-enedioate

3-Pentyloctan-1-ol (3.7 g, 18.4 mmol) and 1-(3-dimethylaminopropyl)-3-ethylcarbodiimide hydrochloride (4.42 g, 23.1 mmol) were added to a solution of glutaconic acid (1.0 g, 7.69 mmol) in DCE (80 mL). DMAP (376 mg, 3.07 mmol) was then added. The mixture was stirred at 65° C. for 16 hours and then filtered. The filtrate was concentrated in vacuo. The residue was partitioned between water and DCM. The organic layer was dried over Na₂SO₄ and filtered. The filtrate was concentrated in vacuo, and the residue was purified by silica gel chromatography to afford bis(3-pentyloctyl) (E/Z)-pent-2-enedioate (1.2 g, 32% yield) as a colorless oil. ¹H NMR (400 MHZ, CDCl₃) δ 6.99 (dt, J=15.7, 7.2 Hz, 1H), 5.97-5.87 (m, 1H), 4.17-4.09 (m, 4H), 3.22 (dd, J=7.2, 1.6 Hz, 2H), 1.65-1.53 (m, 4H), 1.34-1.17 (m, 34H), 0.89-0.83 (m, 12H).

### Step 2: tert-butyl 9-(4-(benzyloxy)butyl)-3,9-diazaspiro[5.5]undecane-3-carboxylate

This compound was prepared according to the general alkylation procedure as described in Example 1 (Step 3). tert-Butyl 3,9-diazaspiro[5.5]undecane-3-carboxylate (4.0 g, 20 mmol) and ((4-bromobutoxy) methyl)benzene (3.82 g, 15.7 mmol) afforded tert-butyl 9-(4-(benzyloxy)butyl)-3,9-diazaspiro[5.5]undecane-3-carboxylate (4.68 g, 70% yield) as a colorless oil. LCMS: Calc'd for C₂₅H₄₁N₂O₃ [M+H]⁺ 417.30, found 417.1.

### Step 3: 3-(4-(benzyloxy)butyl)-3,9-diazaspiro[5.5]undecane

A solution of tert-butyl 9-(4-(benzyloxy)butyl)-3,9-diazaspiro[5.5]undecane-3-carboxylate (2.0 g, 4.8 mmol) in 4M HCl/MeOH (18 mL) was stirred at room temperature for 4 hours. The mixture was adjusted to pH 7 using saturated NaHCO₃ and extracted with DCM/2-propanol (3:1). The organic layer was dried over Na₂SO₄ and filtered. The filtrate was concentrated to afford 3-(4-(benzyloxy)butyl)-3,9-diazaspiro[5.5]undecane which was used as is in the subsequent transformations. LCMS (ESI): Calc'd for C₂₀H₃₃N₂O [M+H]⁺ 317.25, found 317.0.

### Step 4: bis(3-pentyloctyl) 3-(9-(4-(benzyloxy)butyl)-3,9-diazaspiro[5.5]undecan-3-yl)pentanedioate

TEA (584 mg, 5.77 mmol) was added to a solution of bis(3-pentyloctyl) (E/Z)-pent-2-enedioate (1.0 g, 2.02 mmol) and 3-(4-(benzyloxy)butyl)-3,9-diazaspiro[5.5]undecane (609 mg, 1.92 mmol) in THF (5 mL), and the mixture was stirred at 80° C. for 16 hours. The mixture was extracted with EtOAc. The organic layer was dried over Na₂SO₄ and filtered. The filtrate was concentrated in vacuo, and the crude product was purified by silica gel chromatography to afford bis(3-pentyloctyl) 3-(9-(4-(benzyloxy)butyl)-3,9-diazaspiro[5.5]undecan-3-yl)pentanedioate (300 mg, 19% yield) as a yellow oil. LCMS (ESI): Calc'd for C₅₁H₉₁N₂O₅ [M+H]⁺ 811.68, found 812.4.

### Step 5: bis(3-pentyloctyl) 3-(9-(4-hydroxybutyl)-3,9-diazaspiro[5.5]undecan-3-yl)pentanedioate

Pd(OH)₂ (20%, 57.1 mg, 0.81 mmol) and Pd/C (10%, 43.3 mg, 0.047 mmol) was added to a solution of bis(3-pentyloctyl) 3-(9-(4-(benzyloxy)butyl)-3,9-diazaspiro[5.5]undecan-3-yl)pentanedioate (330 mg, 0.410 mmol) in THF (8 mL). The mixture was placed under an atmosphere of H₂ (50 psi) and stirred at 60° C. for 16 hours. The reaction was incomplete so an additional portion of Pd(OH)₂ (20%, 57.1 mg, 0.81 mmol) and Pd/C (10%, 43.3 mg, 0.047 mmol) was added. The mixture was returned to an atmosphere of H₂ (50 psi) and stirred at 60° C. for an additional 16 hours. The mixture was filtered and concentrated in vacuo, and the crude product was purified by silica gel chromatography to afford bis(3-pentyloctyl) 3-(9-(4-hydroxybutyl)-3,9-diazaspiro[5.5]undecan-3-yl)pentanedioate (110 mg, 38% yield) as a light yellow oil. ¹H NMR (400 MHz, CD₃OD) δ 4.10 (t, J=6.8 Hz, 4H), 3.57 (t, J=6.0 Hz, 2H), 3.54-3.47 (m, 1H), 2.73-2.54 (m, 8H), 2.54-2.48 (m, 4H), 2.42-2.33 (m, 2H), 1.71-1.53 (m, 12H), 1.51-1.42 (m, 6H), 1.37-1.27 (m, 32H), 0.96-0.88 (m, 12H). LCMS (ESI): Calc'd for C₄₄H₈₅N₂O₅ [M+H]⁺ 721.64; found 721.8.

The compounds shown in Tables 6A-6G are prophetic deuterated analogs (PDA) of Examples 3, 13, 22, 23, 25, 51, and 61, respectively. The PDAs are predicted based on the metabolic profile of Examples 3, 13, 22, 23, 25, 51, and 61. The position of the deuterated atom is indicated with “D” in each deuterated analog set forth in Tables 6A-6G.

TABLE 6A

| | |
|---|---|
| | Example 3 |
| 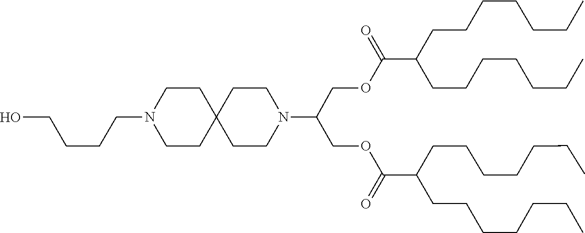 [SMILES: CCCCCCCC(CCCCCCC)C(=O)OCC(COC(=O)C(CCCCCCC)CCCCCCC)N1CCC2(CCN(CCCCO)CC2)CC1] | |

| | |
|---|---|
| 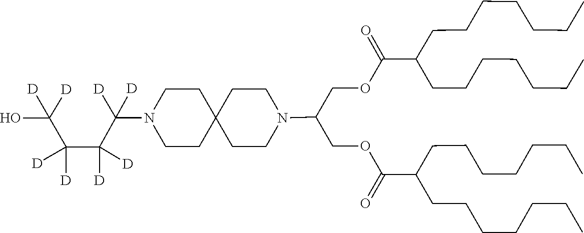 [SMILES: [2H]C([2H])(O)C([2H])([2H])C([2H])([2H])C([2H])([2H])N1CCC2(CCN(C(COC(=O)C(CCCCCCC)CCCCCCC)COC(=O)C(CCCCCCC)CCCCCCC)CC2)CC1] | Example 3-1-D |
| 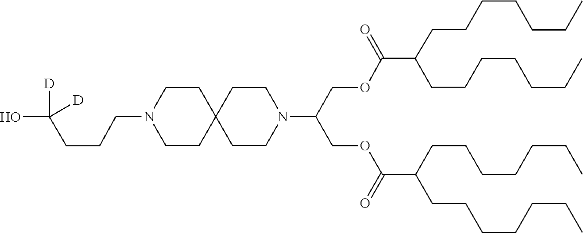 [SMILES: [2H]C([2H])(O)CCCN1CCC2(CC1)CCN(C(COC(=O)C(CCCCCCC)CCCCCCC)COC(=O)C(CCCCCCC)CCCCCCC)CC2] | Example 3-2-D |
| 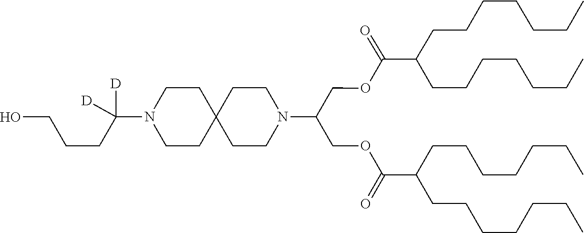 [SMILES: [2H]C([2H])(CCCO)N1CCC2(CCN(C(COC(=O)C(CCCCCCC)CCCCCCC)COC(=O)C(CCCCCCC)CCCCCCC)CC2)CC1] | Example 3-3-D |
| 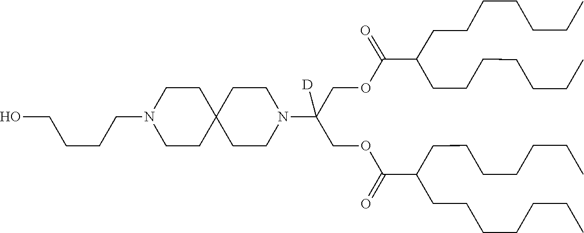 [SMILES: [2H]C(COC(=O)C(CCCCCCC)CCCCCCC)(COC(=O)C(CCCCCCC)CCCCCCC)N1CCC2(CCN(CCCCO)CC2)CC1] | Example 3-4-D |
| 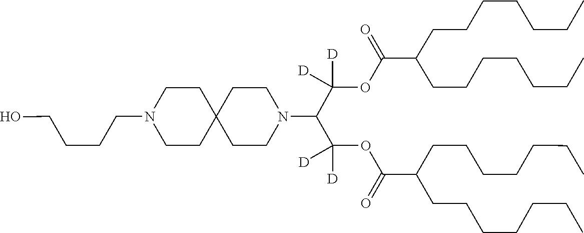 [SMILES: [2H]C([2H])(OC(=O)C(CCCCCCC)CCCCCCC)C(N1CCC2(CCN(CCCCO)CC2)CC1)C([2H])([2H])OC(=O)C(CCCCCCC)CCCCCCC] | Example 3-5-D |
| 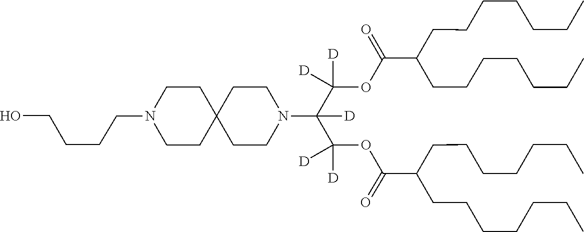 [SMILES: [2H]C([2H])(OC(=O)C(CCCCCCC)CCCCCCC)C([2H])(N1CCC2(CCN(CCCCO)CC2)CC1)C([2H])([2H])OC(=O)C(CCCCCCC)CCCCCCC] | Example 3-6-D |
| 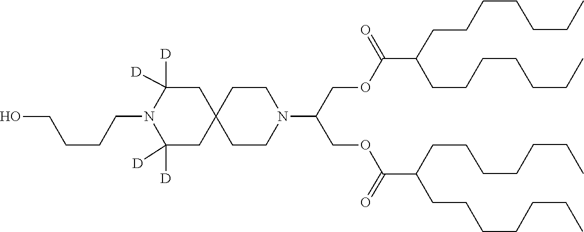 [SMILES: [2H]C1([2H])CC2(CCN(C(COC(=O)C(CCCCCCC)CCCCCCC)COC(=O)C(CCCCCCC)CCCCCCC)CC2)CC([2H])([2H])N1CCCCO] | Example 3-7-D |
| 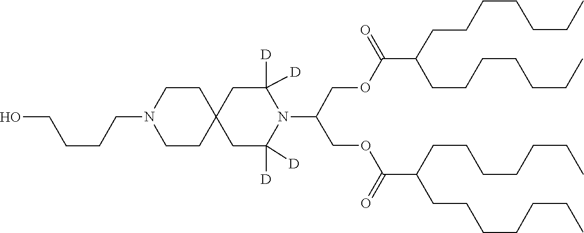 [SMILES: [2H]C1([2H])CC2(CCN(CCCCO)CC2)CC([2H])([2H])N1C(COC(=O)C(CCCCCCC)CCCCCCC)COC(=O)C(CCCCCCC)CCCCCCC] | Example 3-8-D |
| 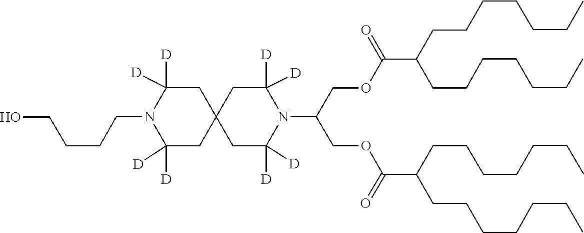 [SMILES: [2H]C1([2H])CC2(CC([2H])([2H])N1CCCCO)CC([2H])([2H])N(C(COC(=O)C(CCCCCCC)CCCCCCC)COC(=O)C(CCCCCCC)CCCCCCC)C([2H])([2H])C2] | Example 3-9-D |
| 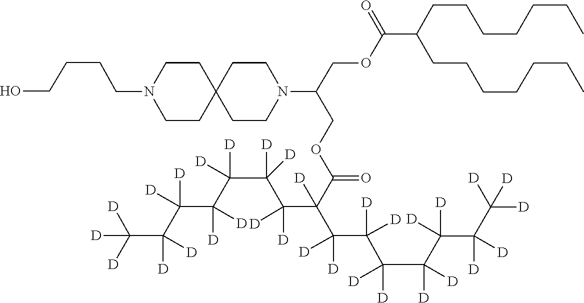 [SMILES: [2H]C([2H])([2H])C([2H])([2H])C([2H])([2H])C([2H])([2H])C([2H])([2H])C([2H])([2H])C([2H])([2H])C([2H])(C(=O)OCC(COC(=O)C(CCCCCCC)CCCCCCC)N1CCC2(CCN(CCCCO)CC2)CC1)C([2H])([2H])C([2H])([2H])C([2H])([2H])C([2H])([2H])C([2H])([2H])C([2H])([2H])C([2H])([2H])[2H]] | Example 3-10-D |
| 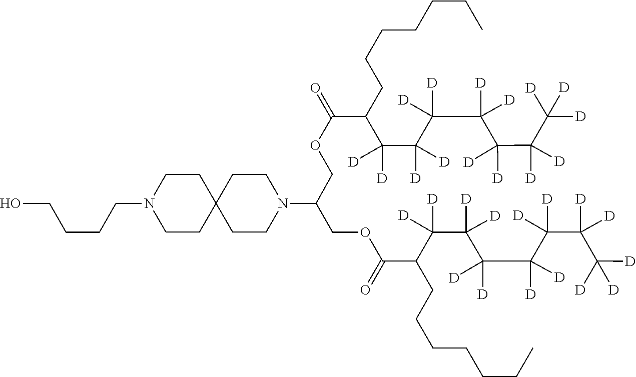 [SMILES: [2H]C([2H])([2H])C([2H])([2H])C([2H])([2H])C([2H])([2H])C([2H])([2H])C([2H])([2H])C([2H])([2H])C(CCCCCCC)C(=O)OCC(COC(=O)C(CCCCCCC)C([2H])([2H])C([2H])([2H])C([2H])([2H])C([2H])([2H])C([2H])([2H])C([2H])([2H])C([2H])([2H])[2H])N1CCC2(CCN(CCCCO)CC2)CC1] | Example 3-11-D |
| 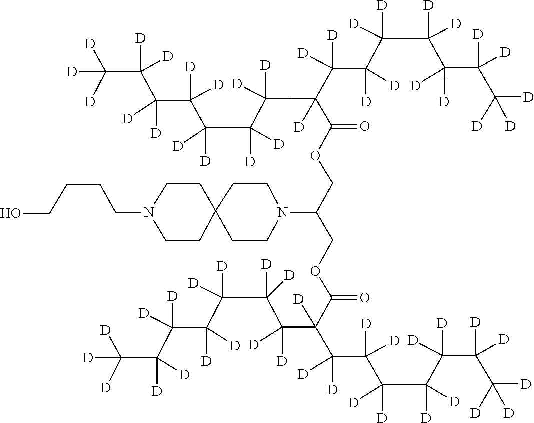 [SMILES: [2H]C([2H])([2H])C([2H])([2H])C([2H])([2H])C([2H])([2H])C([2H])([2H])C([2H])([2H])C([2H])([2H])C([2H])(C(=O)OCC(COC(=O)C([2H])(C([2H])([2H])C([2H])([2H])C([2H])([2H])C([2H])([2H])C([2H])([2H])C([2H])([2H])C([2H])([2H])[2H])C([2H])([2H])C([2H])([2H])C([2H])([2H])C([2H])([2H])C([2H])([2H])C([2H])([2H])C([2H])([2H])[2H])N1CCC2(CCN(CCCCO)CC2)CC1)C([2H])([2H])C([2H])([2H])C([2H])([2H])C([2H])([2H])C([2H])([2H])C([2H])([2H])C([2H])([2H])[2H]] | Example 3-12-D |
| 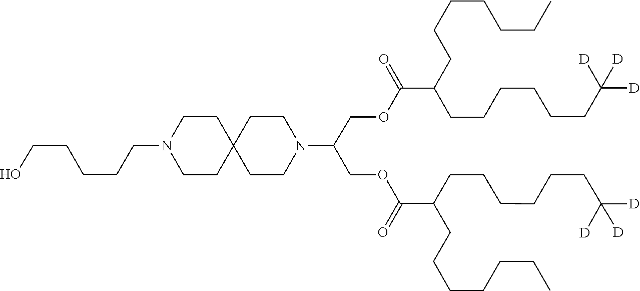 [SMILES: [2H]C([2H])([2H])CCCCCCC(CCCCCCC)C(=O)OCC(COC(=O)C(CCCCCCC)CCCCCCC([2H])([2H])[2H])N1CCC2(CCN(CCCCCO)CC2)CC1] | Example 3-13-D |
| 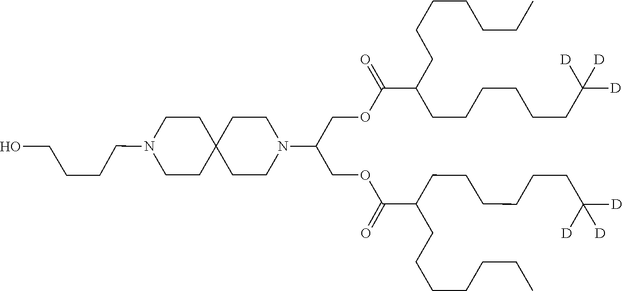 [SMILES: [2H]C([2H])([2H])CCCCCCC(CCCCCCC)C(=O)OCC(COC(=O)C(CCCCCCC)CCCCCCC([2H])([2H])[2H])N1CCC2(CCN(CCCCO)CC2)CC1] | Example 3-14-D |
| 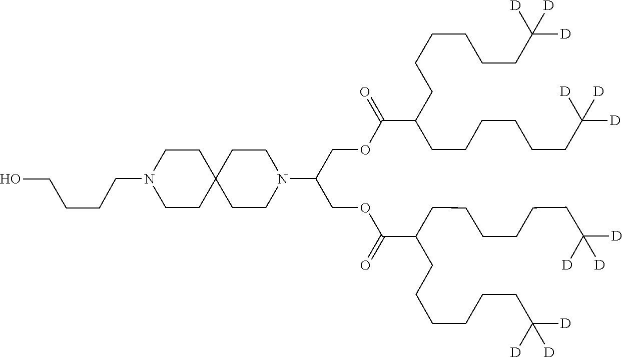 [SMILES: [2H]C([2H])([2H])CCCCCCC(CCCCCCC([2H])([2H])[2H])C(=O)OCC(COC(=O)C(CCCCCCC([2H])([2H])[2H])CCCCCCC([2H])([2H])[2H])N1CCC2(CCN(CCCCO)CC2)CC1] | Example 3-15-D |

TABLE 6B

| | |
|---|---|
| | Example 13 |
| 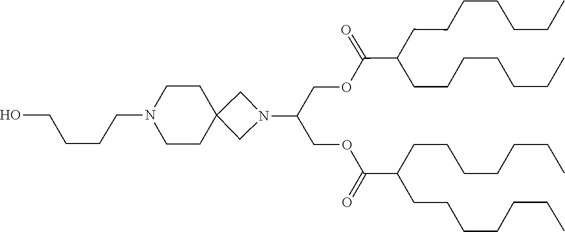 [SMILES: CCCCCCCC(CCCCCCC)C(=O)OCC(COC(=O)C(CCCCCCC)CCCCCCC)N1CC2(CCN(CCCCO)CC2)C1] | |

| | |
|---|---|
| 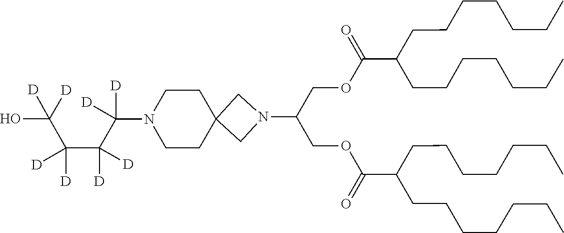 [SMILES: [2H]C([2H])(O)C([2H])([2H])C([2H])([2H])C([2H])([2H])N1CCC2(CC1)CN(C(COC(=O)C(CCCCCCC)CCCCCCC)COC(=O)C(CCCCCCC)CCCCCCC)C2] | Example 13-1-D |
| 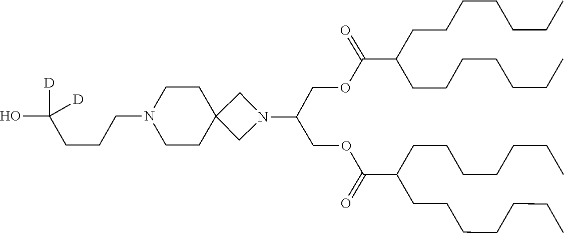 [SMILES: [2H]C([2H])(O)CCCN1CCC2(CC1)CN(C(COC(=O)C(CCCCCCC)CCCCCCC)COC(=O)C(CCCCCCC)CCCCCCC)C2] | Example 13-2-D |
| 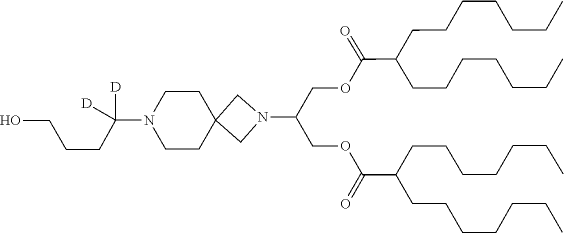 [SMILES: [2H]C([2H])(CCCO)N1CCC2(CC1)CN(C(COC(=O)C(CCCCCCC)CCCCCCC)COC(=O)C(CCCCCCC)CCCCCCC)C2] | Example 13-3-D |
| 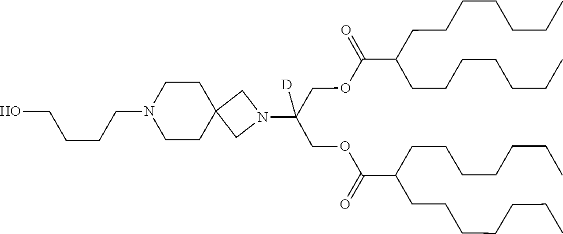 [SMILES: [2H]C(COC(=O)C(CCCCCCC)CCCCCCC)(COC(=O)C(CCCCCCC)CCCCCCC)N1CC2(CCN(CCCCO)CC2)C1] | Example 13-4-D |
| 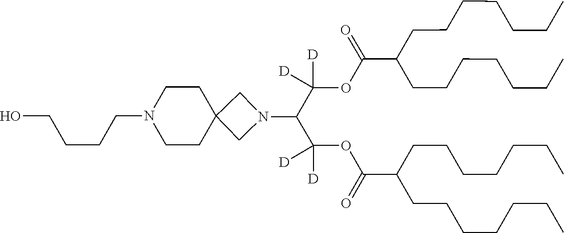 [SMILES: [2H]C([2H])(OC(=O)C(CCCCCCC)CCCCCCC)C(N1CC2(CCN(CCCCO)CC2)C1)C([2H])([2H])OC(=O)C(CCCCCCC)CCCCCCC] | Example 13-5-D |
| 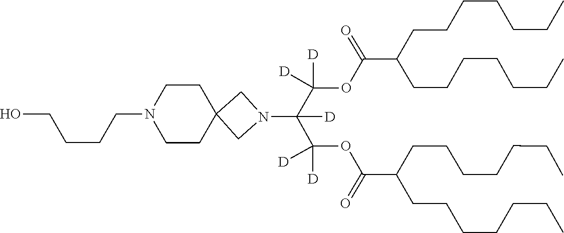 [SMILES: [2H]C([2H])(OC(=O)C(CCCCCCC)CCCCCCC)C([2H])(N1CC2(CCN(CCCCO)CC2)C1)C([2H])([2H])OC(=O)C(CCCCCCC)CCCCCCC] | Example 13-6-D |
| 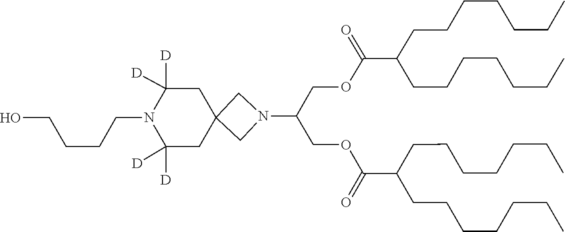 [SMILES: [2H]C1([2H])CC2(CN(C(COC(=O)C(CCCCCCC)CCCCCCC)COC(=O)C(CCCCCCC)CCCCCCC)C2)CC([2H])([2H])N1CCCCO] | Example 13-7-D |
| 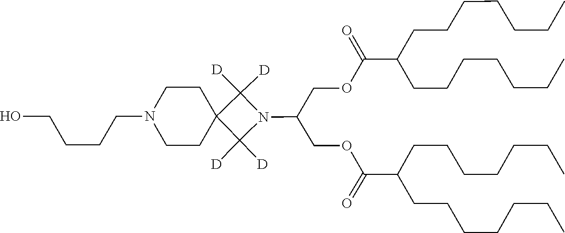 [SMILES: [2H]C1([2H])N(C(COC(=O)C(CCCCCCC)CCCCCCC)COC(=O)C(CCCCCCC)CCCCCCC)C([2H])([2H])C12CCN(CCCCO)CC2] | Example 13-8-D |
| 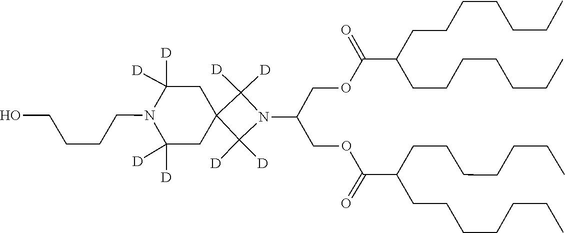 [SMILES: [2H]C1([2H])CC2(CC([2H])([2H])N1CCCCO)C([2H])([2H])N(C(COC(=O)C(CCCCCCC)CCCCCCC)COC(=O)C(CCCCCCC)CCCCCCC)C2([2H])[2H]] | Example 13-9-D |
| 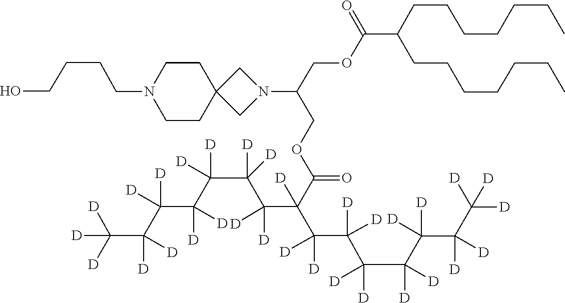 [SMILES: [2H]C([2H])([2H])C([2H])([2H])C([2H])([2H])C([2H])([2H])C([2H])([2H])C([2H])([2H])C([2H])([2H])C([2H])(C(=O)OCC(COC(=O)C(CCCCCCC)CCCCCCC)N1CC2(CCN(CCCCO)CC2)C1)C([2H])([2H])C([2H])([2H])C([2H])([2H])C([2H])([2H])C([2H])([2H])C([2H])([2H])C([2H])([2H])[2H]] | Example 13-10-D |
| 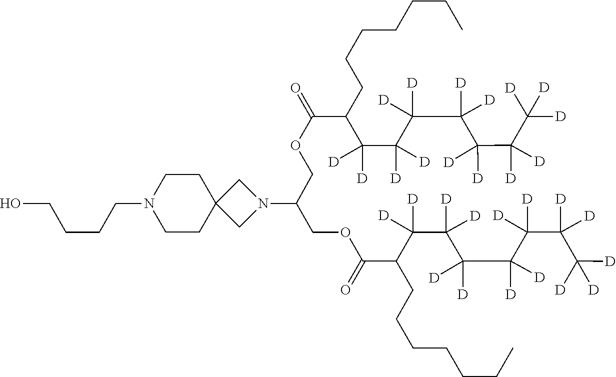 [SMILES: [2H]C([2H])([2H])C([2H])([2H])C([2H])([2H])C([2H])([2H])C([2H])([2H])C([2H])([2H])C([2H])([2H])C(CCCCCCC)C(=O)OCC(COC(=O)C(CCCCCCC)C([2H])([2H])C([2H])([2H])C([2H])([2H])C([2H])([2H])C([2H])([2H])C([2H])([2H])C([2H])([2H])[2H])N1CC2(CCN(CCCCO)CC2)C1] | Example 13-11-D |
| 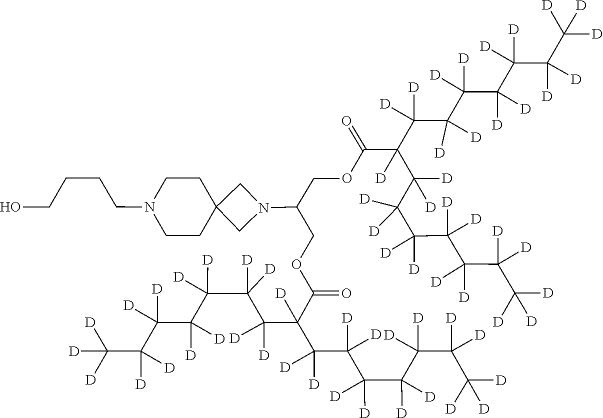 [SMILES: [2H]C([2H])([2H])C([2H])([2H])C([2H])([2H])C([2H])([2H])C([2H])([2H])C([2H])([2H])C([2H])([2H])C([2H])(C(=O)OCC(COC(=O)C([2H])(C([2H])([2H])C([2H])([2H])C([2H])([2H])C([2H])([2H])C([2H])([2H])C([2H])([2H])C([2H])([2H])[2H])C([2H])([2H])C([2H])([2H])C([2H])([2H])C([2H])([2H])C([2H])([2H])C([2H])([2H])C([2H])([2H])[2H])N1CC2(CCN(CCCCO)CC2)C1)C([2H])([2H])C([2H])([2H])C([2H])([2H])C([2H])([2H])C([2H])([2H])C([2H])([2H])C([2H])([2H])[2H]] | Example 13-12-D |
| 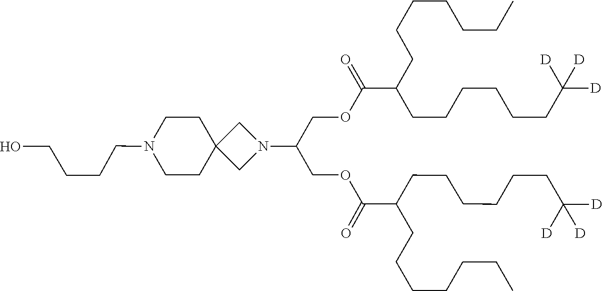 [SMILES: [2H]C([2H])([2H])CCCCCCC(CCCCCCC)C(=O)OCC(COC(=O)C(CCCCCCC)CCCCCCC([2H])([2H])[2H])N1CC2(CCN(CCCCO)CC2)C1] | Example 13-13-D |
| 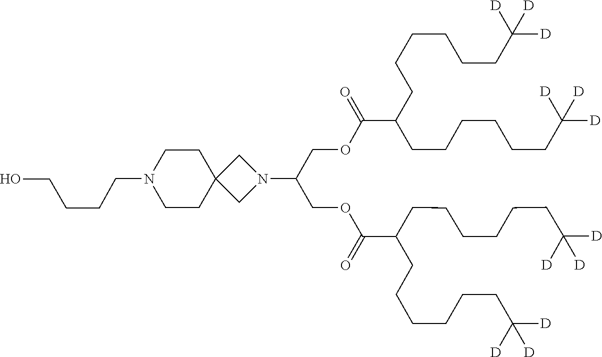 [SMILES: [2H]C([2H])([2H])CCCCCCC(CCCCCCC([2H])([2H])[2H])C(=O)OCC(COC(=O)C(CCCCCCC([2H])([2H])[2H])CCCCCCC([2H])([2H])[2H])N1CC2(CCN(CCCCO)CC2)C1] | Example 13-14-D |

TABLE 6C

| | |
|---|---|
| | Example 22 |
| 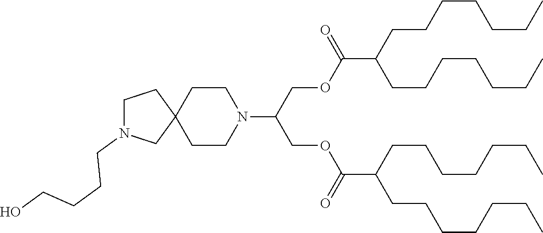 [SMILES: CCCCCCCC(CCCCCCC)C(=O)OCC(COC(=O)C(CCCCCCC)CCCCCCC)N1CCC2(CCN(CCCCO)C2)CC1] | |

| | |
|---|---|
| 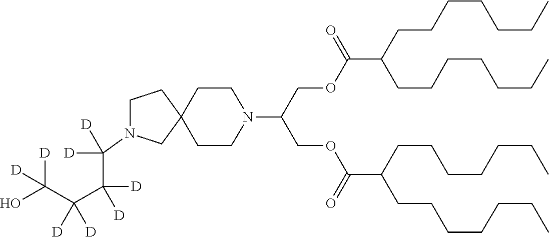 [SMILES: [2H]C([2H])(O)C([2H])([2H])C([2H])([2H])C([2H])([2H])N1CCC2(CCN(C(COC(=O)C(CCCCCCC)CCCCCCC)COC(=O)C(CCCCCCC)CCCCCCC)CC2)C1] | Example 22-1-D |
| 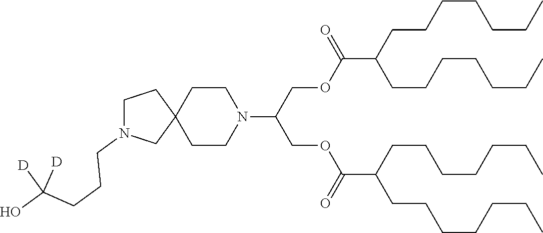 [SMILES: [2H]C([2H])(O)CCCN1CCC2(CCN(C(COC(=O)C(CCCCCCC)CCCCCCC)COC(=O)C(CCCCCCC)CCCCCCC)CC2)C1] | Example 22-2-D |
| 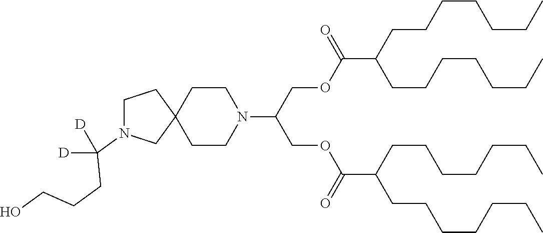 [SMILES: [2H]C([2H])(CCCO)N1CCC2(CCN(C(COC(=O)C(CCCCCCC)CCCCCCC)COC(=O)C(CCCCCCC)CCCCCCC)CC2)C1] | Example 22-3-D |
| 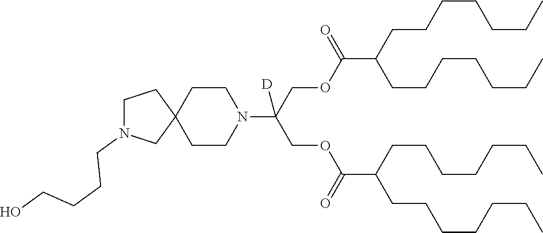 [SMILES: [2H]C(COC(=O)C(CCCCCCC)CCCCCCC)(COC(=O)C(CCCCCCC)CCCCCCC)N1CCC2(CCN(CCCCO)C2)CC1] | Example 22-4-D |
| 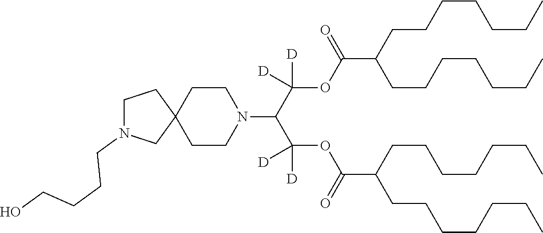 [SMILES: [2H]C([2H])(OC(=O)C(CCCCCCC)CCCCCCC)C(N1CCC2(CCN(CCCCO)C2)CC1)C([2H])([2H])OC(=O)C(CCCCCCC)CCCCCCC] | Example 22-5-D |
| 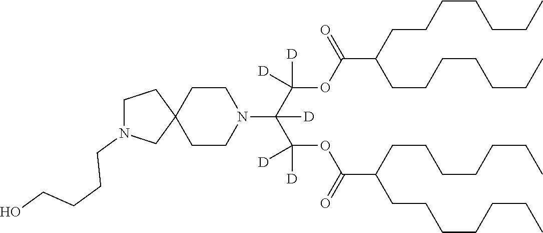 [SMILES: [2H]C([2H])(OC(=O)C(CCCCCCC)CCCCCCC)C([2H])(N1CCC2(CCN(CCCCO)C2)CC1)C([2H])([2H])OC(=O)C(CCCCCCC)CCCCCCC] | Example 22-6-D |
| 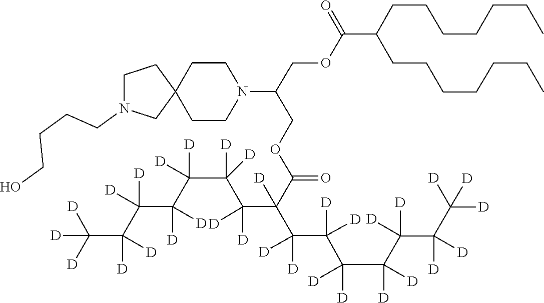 [SMILES: [2H]C([2H])([2H])C([2H])([2H])C([2H])([2H])C([2H])([2H])C([2H])([2H])C([2H])([2H])C([2H])([2H])C([2H])(C(=O)OCC(COC(=O)C(CCCCCCC)CCCCCCC)N1CCC2(CCN(CCCCO)C2)CC1)C([2H])([2H])C([2H])([2H])C([2H])([2H])C([2H])([2H])C([2H])([2H])C([2H])([2H])C([2H])([2H])[2H]] | Example 22-7-D |
| 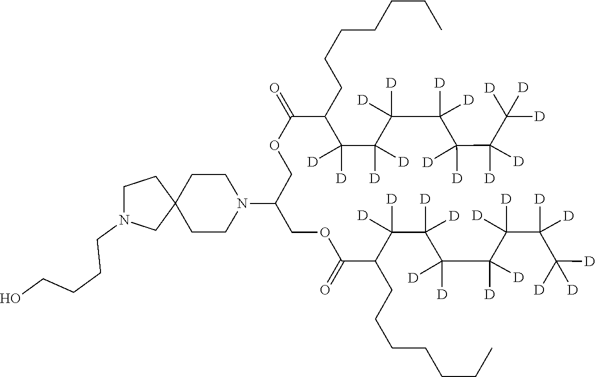 [SMILES: [2H]C([2H])([2H])C([2H])([2H])C([2H])([2H])C([2H])([2H])C([2H])([2H])C([2H])([2H])C([2H])([2H])C(CCCCCCC)C(=O)OCC(COC(=O)C(CCCCCCC)C([2H])([2H])C([2H])([2H])C([2H])([2H])C([2H])([2H])C([2H])([2H])C([2H])([2H])C([2H])([2H])[2H])N1CCC2(CCN(CCCCO)C2)CC1] | Example 22-8-D |
| 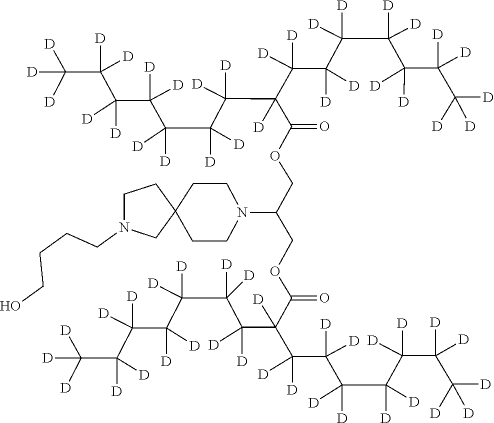 [SMILES: [2H]C([2H])([2H])C([2H])([2H])C([2H])([2H])C([2H])([2H])C([2H])([2H])C([2H])([2H])C([2H])([2H])C([2H])(C(=O)OCC(COC(=O)C([2H])(C([2H])([2H])C([2H])([2H])C([2H])([2H])C([2H])([2H])C([2H])([2H])C([2H])([2H])C([2H])([2H])[2H])C([2H])([2H])C([2H])([2H])C([2H])([2H])C([2H])([2H])C([2H])([2H])C([2H])([2H])C([2H])([2H])[2H])N1CCC2(CCN(CCCCO)C2)CC1)C([2H])([2H])C([2H])([2H])C([2H])([2H])C([2H])([2H])C([2H])([2H])C([2H])([2H])C([2H])([2H])[2H]] | Example 22-9-D |
| 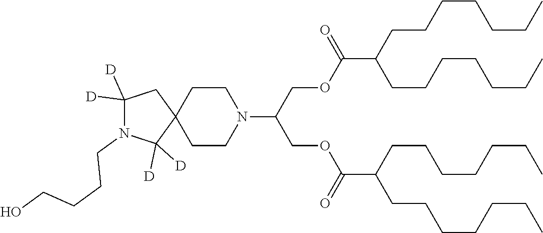 [SMILES: [2H]C1([2H])CC2(CCN(C(COC(=O)C(CCCCCCC)CCCCCCC)COC(=O)C(CCCCCCC)CCCCCCC)CC2)C([2H])([2H])N1CCCCO] | Example 22-10-D |
| 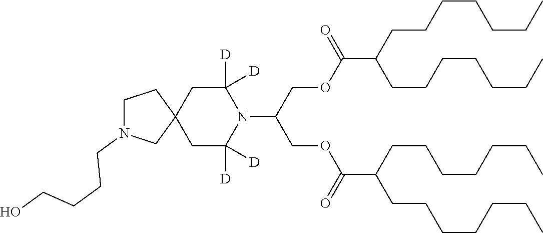 [SMILES: [2H]C1([2H])CC2(CCN(CCCCO)C2)CC([2H])([2H])N1C(COC(=O)C(CCCCCCC)CCCCCCC)COC(=O)C(CCCCCCC)CCCCCCC] | Example 22-11-D |
| 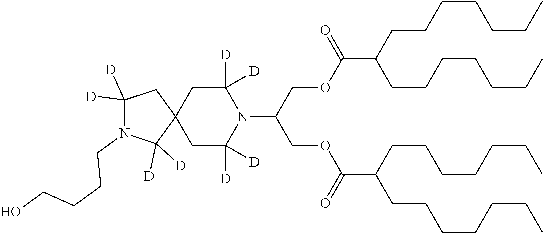 [SMILES: [2H]C1([2H])CC2(CC([2H])([2H])N1C(COC(=O)C(CCCCCCC)CCCCCCC)COC(=O)C(CCCCCCC)CCCCCCC)CC([2H])([2H])N(CCCCO)C2([2H])[2H]] | Example 22-12-D |
| 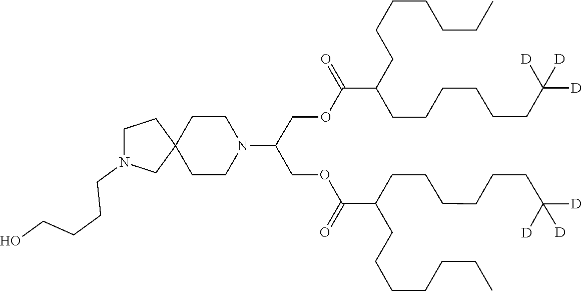 [SMILES: [2H]C([2H])([2H])CCCCCCC(CCCCCCC)C(=O)OCC(COC(=O)C(CCCCCCC)CCCCCCC([2H])([2H])[2H])N1CCC2(CCN(CCCCO)C2)CC1] | Example 22-13-D |
| 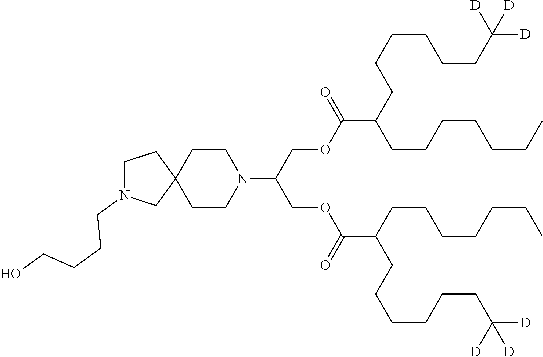 [SMILES: [2H]C([2H])([2H])CCCCCCC(CCCCCCC)C(=O)OCC(COC(=O)C(CCCCCCC)CCCCCCC([2H])([2H])[2H])N1CCC2(CCN(CCCCO)C2)CC1] | Example 22-14-D |
| 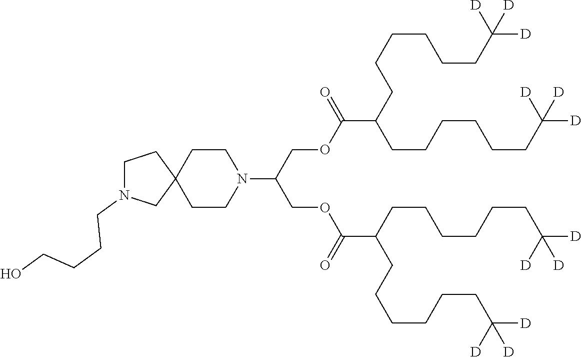 [SMILES: [2H]C([2H])([2H])CCCCCCC(CCCCCCC([2H])([2H])[2H])C(=O)OCC(COC(=O)C(CCCCCCC([2H])([2H])[2H])CCCCCCC([2H])([2H])[2H])N1CCC2(CCN(CCCCO)C2)CC1] | Example 22-15-D |

TABLE 6D

| | |
|---|---|
| | Example 23 |
| 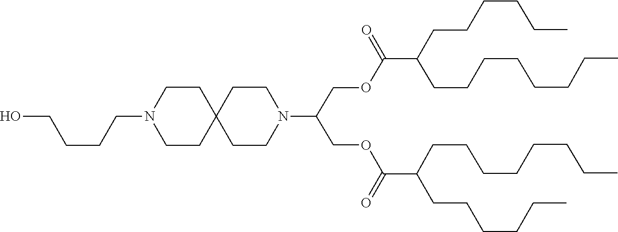 [SMILES: CCCCCCCCC(CCCCCC)C(=O)OCC(COC(=O)C(CCCCCC)CCCCCCCC)N1CCC2(CCN(CCCCO)CC2)CC1] | |

| | |
|---|---|
| 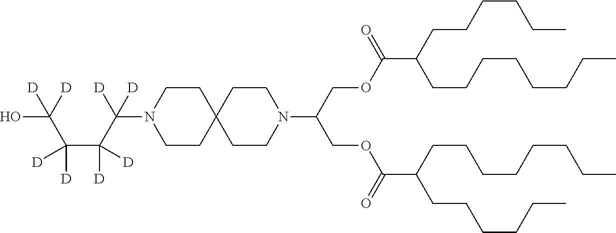 [SMILES: [2H]C([2H])(O)C([2H])([2H])C([2H])([2H])C([2H])([2H])N1CCC2(CCN(C(COC(=O)C(CCCCCC)CCCCCCCC)COC(=O)C(CCCCCC)CCCCCCCC)CC2)CC1] | Example 23-1-D |
| 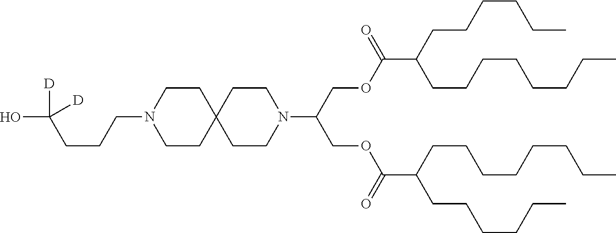 [SMILES: [2H]C([2H])(O)CCCN1CCC2(CC1)CCN(C(COC(=O)C(CCCCCC)CCCCCCCC)COC(=O)C(CCCCCC)CCCCCCCC)CC2] | Example 23-2-D |
| 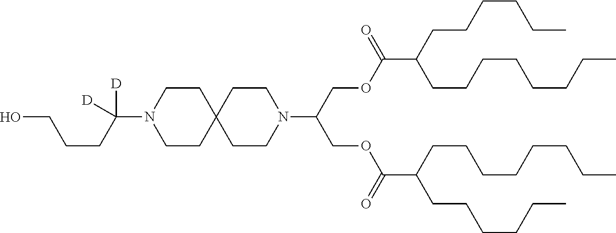 [SMILES: [2H]C([2H])(CCCO)N1CCC2(CCN(C(COC(=O)C(CCCCCC)CCCCCCCC)COC(=O)C(CCCCCC)CCCCCCCC)CC2)CC1] | Example 23-3-D |
| 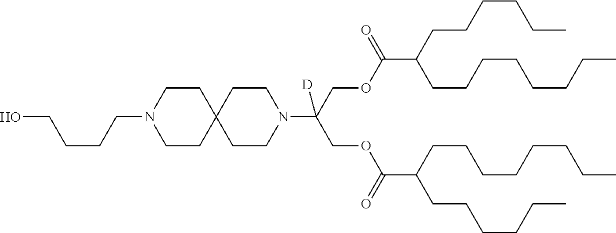 [SMILES: [2H]C(COC(=O)C(CCCCCC)CCCCCCCC)(COC(=O)C(CCCCCC)CCCCCCCC)N1CCC2(CCN(CCCCO)CC2)CC1] | Example 23-4-D |
| 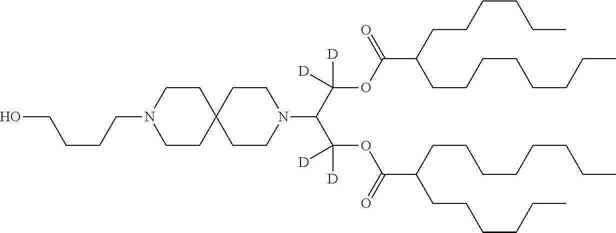 [SMILES: [2H]C([2H])(OC(=O)C(CCCCCC)CCCCCCCC)C(N1CCC2(CCN(CCCCO)CC2)CC1)C([2H])([2H])OC(=O)C(CCCCCC)CCCCCCCC] | Example 23-5-D |
| 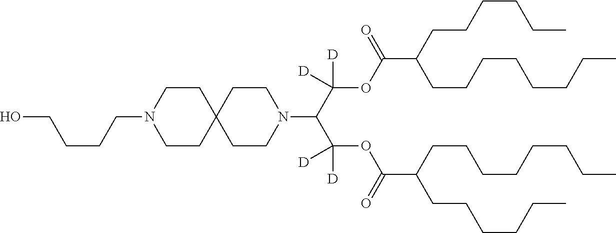 [SMILES: [2H]C([2H])(OC(=O)C(CCCCCC)CCCCCCCC)C(N1CCC2(CCN(CCCCO)CC2)CC1)C([2H])([2H])OC(=O)C(CCCCCC)CCCCCCCC] | Example 23-6-D |
| 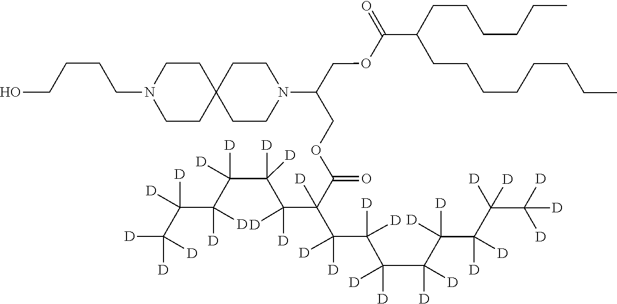 [SMILES: [2H]C([2H])([2H])C([2H])([2H])C([2H])([2H])C([2H])([2H])C([2H])([2H])C([2H])([2H])C([2H])([2H])C([2H])([2H])C([2H])(C(=O)OCC(COC(=O)C(CCCCCC)CCCCCCCC)N1CCC2(CCN(CCCCO)CC2)CC1)C([2H])([2H])C([2H])([2H])C([2H])([2H])C([2H])([2H])C([2H])([2H])C([2H])([2H])[2H]] | Example 23-7-D |
| 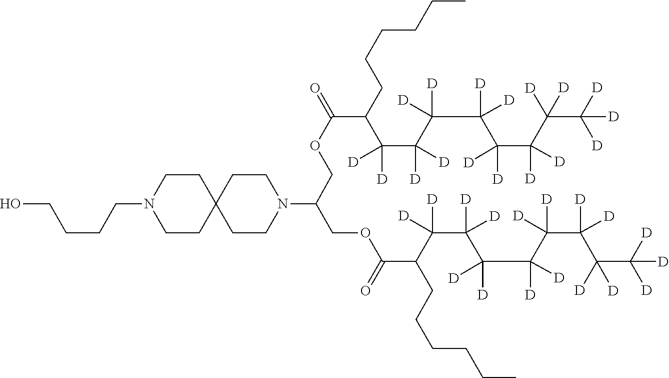 [SMILES: [2H]C([2H])([2H])C([2H])([2H])C([2H])([2H])C([2H])([2H])C([2H])([2H])C([2H])([2H])C([2H])([2H])C([2H])([2H])C(CCCCCC)C(=O)OCC(COC(=O)C(CCCCCC)C([2H])([2H])C([2H])([2H])C([2H])([2H])C([2H])([2H])C([2H])([2H])C([2H])([2H])C([2H])([2H])C([2H])([2H])[2H])N1CCC2(CCN(CCCCO)CC2)CC1] | Example 23-8-D |
| 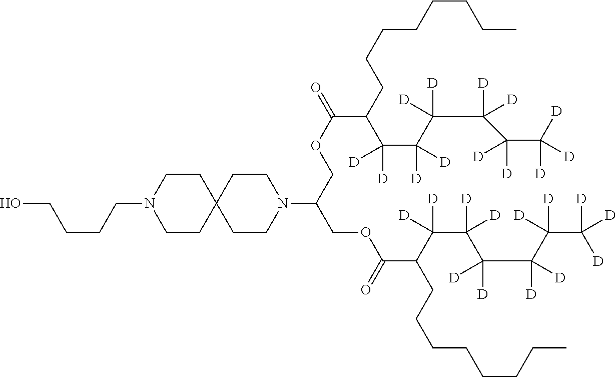 [SMILES: [2H]C([2H])([2H])C([2H])([2H])C([2H])([2H])C([2H])([2H])C([2H])([2H])C([2H])([2H])C(CCCCCCCC)C(=O)OCC(COC(=O)C(CCCCCCCC)C([2H])([2H])C([2H])([2H])C([2H])([2H])C([2H])([2H])C([2H])([2H])C([2H])([2H])[2H])N1CCC2(CCN(CCCCO)CC2)CC1] | Example 23-9-D |
| 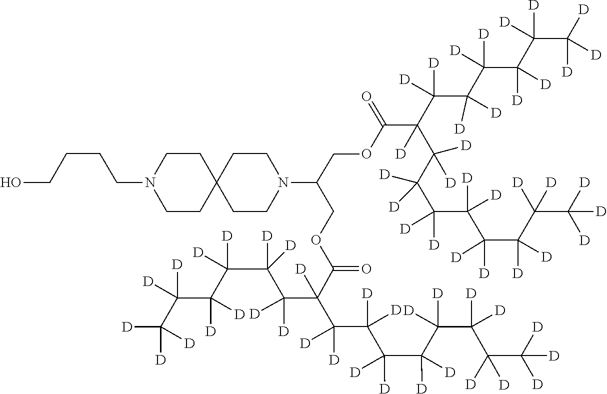 [SMILES: [2H]C([2H])([2H])C([2H])([2H])C([2H])([2H])C([2H])([2H])C([2H])([2H])C([2H])([2H])C([2H])([2H])C([2H])([2H])C([2H])(C(=O)OCC(COC(=O)C([2H])(C([2H])([2H])C([2H])([2H])C([2H])([2H])C([2H])([2H])C([2H])([2H])C([2H])([2H])[2H])C([2H])([2H])C([2H])([2H])C([2H])([2H])C([2H])([2H])C([2H])([2H])C([2H])([2H])C([2H])([2H])C([2H])([2H])[2H])N1CCC2(CCN(CCCCO)CC2)CC1)C([2H])([2H])C([2H])([2H])C([2H])([2H])C([2H])([2H])C([2H])([2H])C([2H])([2H])[2H]] | Example 23-10-D |
| 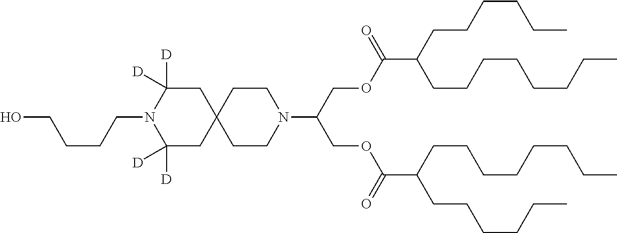 [SMILES: [2H]C1([2H])CC2(CCN(C(COC(=O)C(CCCCCC)CCCCCCCC)COC(=O)C(CCCCCC)CCCCCCCC)CC2)CC([2H])([2H])N1CCCCO] | Example 23-11-D |
| 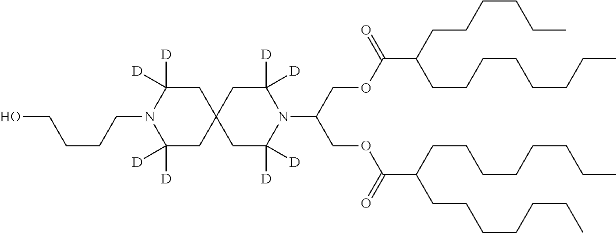 [SMILES: [2H]C1([2H])CC2(CC([2H])([2H])N1CCCCO)CC([2H])([2H])N(C(COC(=O)C(CCCCCC)CCCCCCCC)COC(=O)C(CCCCCCC)CCCCCCCC)C([2H])([2H])C2] | Example 23-12-D |
| 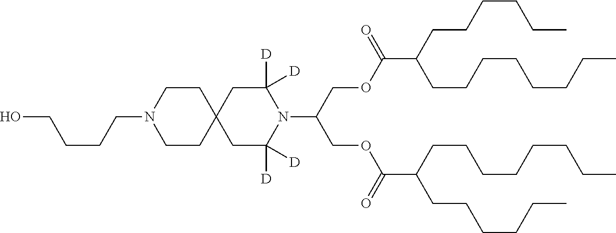 [SMILES: [2H]C1([2H])CC2(CCN(CCCCO)CC2)CC([2H])([2H])N1C(COC(=O)C(CCCCCC)CCCCCCCC)COC(=O)C(CCCCCC)CCCCCCCC] | Example 23-13-D |
| 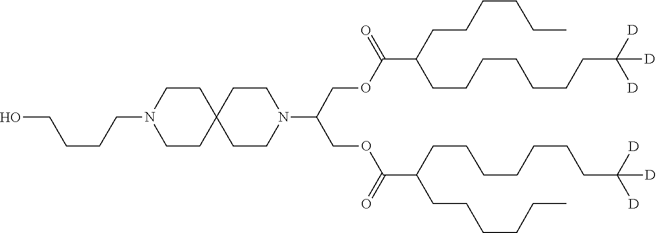 [SMILES: [2H]C([2H])([2H])CCCCCCCC(CCCCCC)C(=O)OCC(COC(=O)C(CCCCCC)CCCCCCCC([2H])([2H])[2H])N1CCC2(CCN(CCCCO)CC2)CC1] | Example 23-14-D |
| 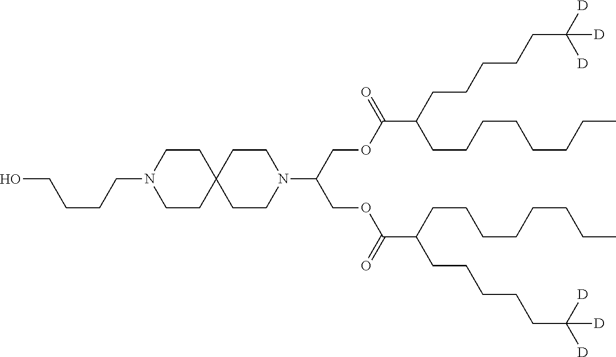 [SMILES: [2H]C([2H])([2H])CCCCCC(CCCCCCCC)C(=O)OCC(COC(=O)C(CCCCCCCC)CCCCCC([2H])([2H])[2H])N1CCC2(CCN(CCCCO)CC2)CC1] | Example 23-15-D |

TABLE 6E

| | |
|---|---|
| | Example 25 |
| 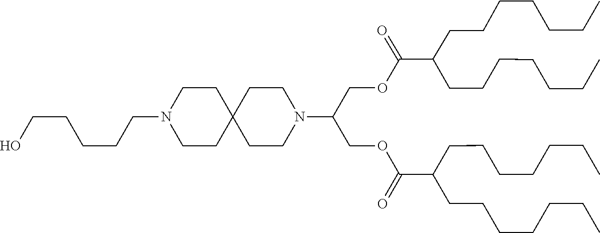 [SMILES: CCCCCCCC(CCCCCCC)C(=O)OCC(COC(=O)C(CCCCCCC)CCCCCCC)N1CCC2(CCN(CCCCCO)CC2)CC1] | |

| | |
|---|---|
| 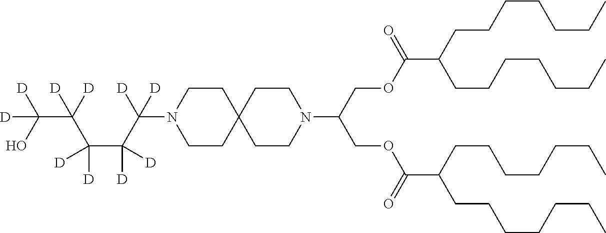 [SMILES: [2H]C([2H])(O)C([2H])([2H])C([2H])([2H])C([2H])([2H])C([2H])([2H])N1CCC2(CCN(C(COC(=O)C(CCCCCCC)CCCCCCC)COC(=O)C(CCCCCCC)CCCCCCC)CC2)CC1] | Example 25-1-D |
| 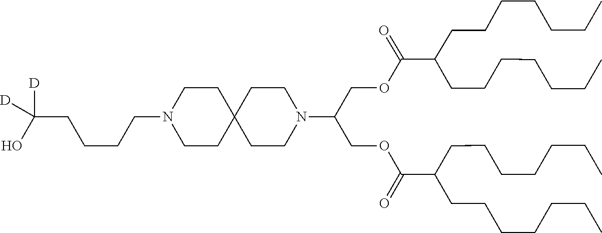 [SMILES: [2H]C([2H])(O)CCCCN1CCC2(CC1)CCN(C(COC(=O)C(CCCCCCC)CCCCCCC)COC(=O)C(CCCCCCC)CCCCCCC)CC2] | Example 25-2-D |
| 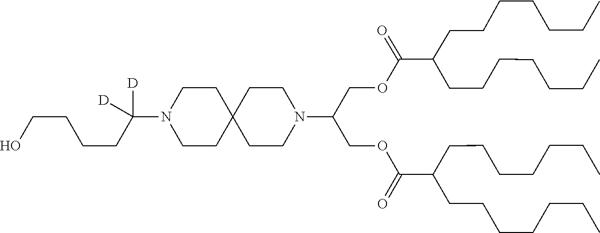 [SMILES: [2H]C([2H])(CCCCO)N1CCC2(CCN(C(COC(=O)C(CCCCCCC)CCCCCCC)COC(=O)C(CCCCCCC)CCCCCCC)CC2)CC1] | Example 25-3-D |
| 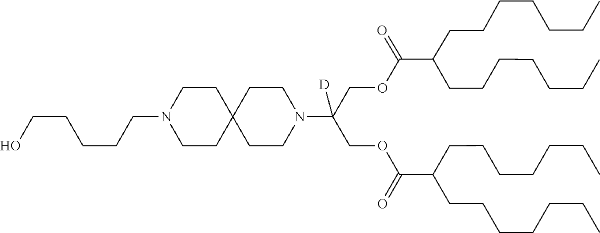 [SMILES: [2H]C(COC(=O)C(CCCCCCC)CCCCCCC)(COC(=O)C(CCCCCCC)CCCCCCC)N1CCC2(CCN(CCCCCO)CC2)CC1] | Example 25-4-D |
| 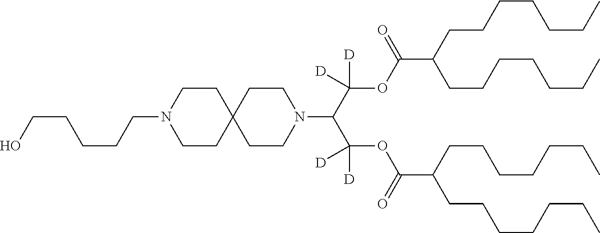 [SMILES: [2H]C([2H])(OC(=O)C(CCCCCCC)CCCCCCC)C(N1CCC2(CCN(CCCCCO)CC2)CC1)C([2H])([2H])OC(=O)C(CCCCCCC)CCCCCCC] | Example 25-4-D |
| 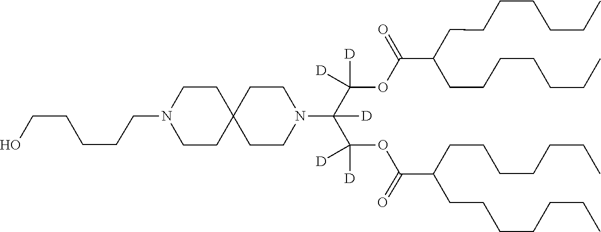 [SMILES: [2H]C([2H])(OC(=O)C(CCCCCCC)CCCCCCC)C([2H])(N1CCC2(CCN(CCCCCO)CC2)CC1)C([2H])([2H])OC(=O)C(CCCCCCC)CCCCCCC] | Example 25-5-D |
| 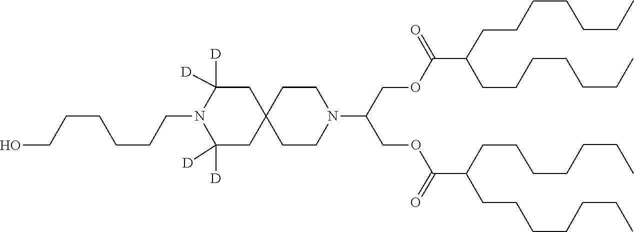 [SMILES: [2H]C1([2H])CC2(CCN(C(COC(=O)C(CCCCCCC)CCCCCCC)COC(=O)C(CCCCCCC)CCCCCCC)CC2)CC([2H])([2H])N1CCCCCCO] | Example 25-6-D |
| 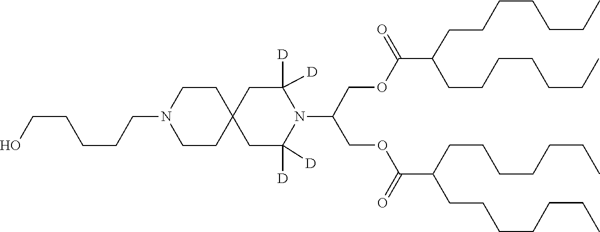 [SMILES: [2H]C1([2H])CC2(CCN(CCCCCO)CC2)CC([2H])([2H])N1C(COC(=O)C(CCCCCCC)CCCCCCC)COC(=O)C(CCCCCCC)CCCCCCC] | Example 25-7-D |
| 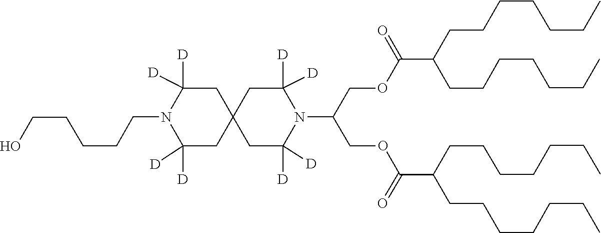 [SMILES: [2H]C1([2H])CC2(CC([2H])([2H])N1CCCCCO)CC([2H])([2H])N(C(COC(=O)C(CCCCCCC)CCCCCCC)COC(=O)C(CCCCCCC)CCCCCCC)C([2H])([2H])C2] | Example 25-8-D |
| 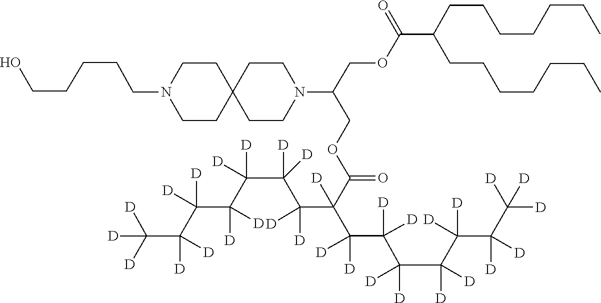 [SMILES: [2H]C([2H])([2H])C([2H])([2H])C([2H])([2H])C([2H])([2H])C([2H])([2H])C([2H])([2H])C([2H])([2H])C([2H])(C(=O)OCC(COC(=O)C(CCCCCCC)CCCCCCC)N1CCC2(CCN(CCCCCO)CC2)CC1)C([2H])([2H])C([2H])([2H])C([2H])([2H])C([2H])([2H])C([2H])([2H])C([2H])([2H])C([2H])([2H])[2H]] | Example 25-9-D |
| 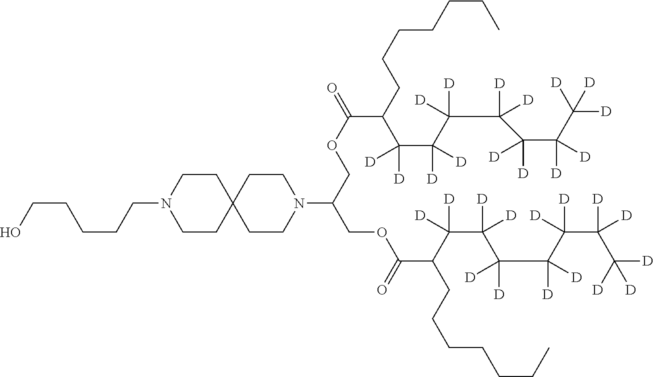 [SMILES: [2H]C([2H])([2H])C([2H])([2H])C([2H])([2H])C([2H])([2H])C([2H])([2H])C([2H])([2H])C([2H])([2H])C(CCCCCCC)C(=O)OCC(COC(=O)C(CCCCCCC)C([2H])([2H])C([2H])([2H])C([2H])([2H])C([2H])([2H])C([2H])([2H])C([2H])([2H])C([2H])([2H])[2H])N1CCC2(CCN(CCCCCO)CC2)CC1] | Example 25-10-D |
| 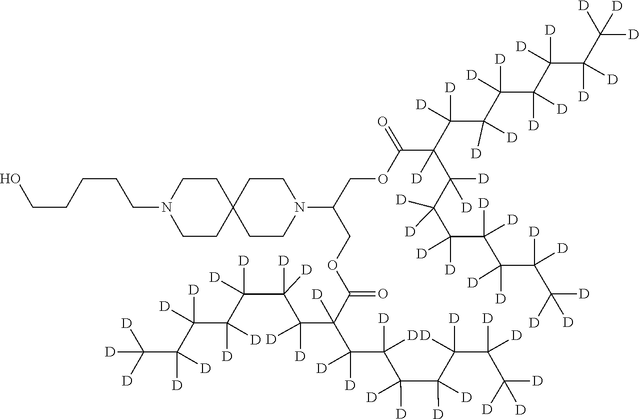 [SMILES: [2H]C([2H])([2H])C([2H])([2H])C([2H])([2H])C([2H])([2H])C([2H])([2H])C([2H])([2H])C([2H])([2H])C([2H])(C(=O)OCC(COC(=O)C([2H])(C([2H])([2H])C([2H])([2H])C([2H])([2H])C([2H])([2H])C([2H])([2H])C([2H])([2H])C([2H])([2H])[2H])C([2H])([2H])C([2H])([2H])C([2H])([2H])C([2H])([2H])C([2H])([2H])C([2H])([2H])C([2H])([2H])[2H])N1CCC2(CCN(CCCCCO)CC2)CC1)C([2H])([2H])C([2H])([2H])C([2H])([2H])C([2H])([2H])C([2H])([2H])C([2H])([2H])C([2H])([2H])[2H]] | Example 25-11-D |
| 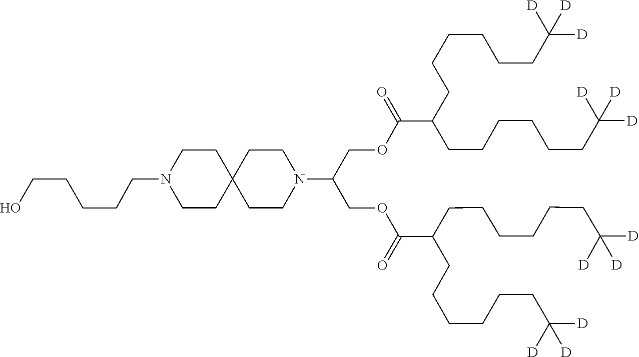 [SMILES: [2H]C([2H])([2H])CCCCCCC(CCCCCCC([2H])([2H])[2H])C(=O)OCC(COC(=O)C(CCCCCCC([2H])([2H])[2H])CCCCCCC([2H])([2H])[2H])N1CCC2(CCN(CCCCCO)CC2)CC1] | Example 25-12-D |
| 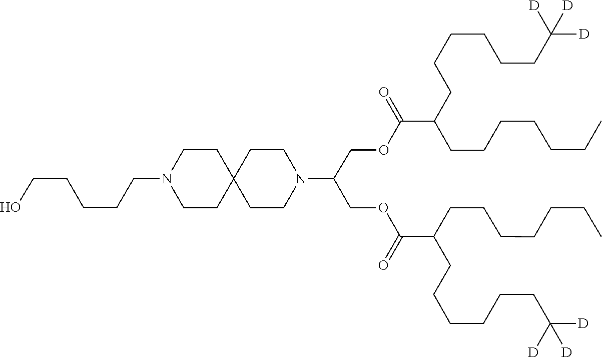 [SMILES: [2H]C([2H])([2H])CCCCCCC(CCCCCCC)C(=O)OCC(COC(=O)C(CCCCCCC)CCCCCCC([2H])([2H])[2H])N1CCC2(CCN(CCCCCO)CC2)CC1] | Example 25-13-D |
| 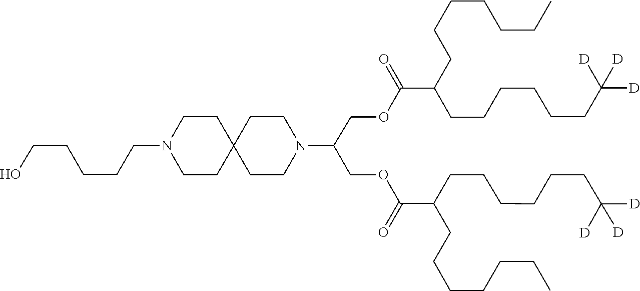 [SMILES: [2H]C([2H])([2H])CCCCCCC(CCCCCCC)C(=O)OCC(COC(=O)C(CCCCCCC)CCCCCCC([2H])([2H])[2H])N1CCC2(CCN(CCCCCO)CC2)CC1] | Example 25-14-D |

TABLE 6F

| | |
|---|---|
| | Example 51 |
| 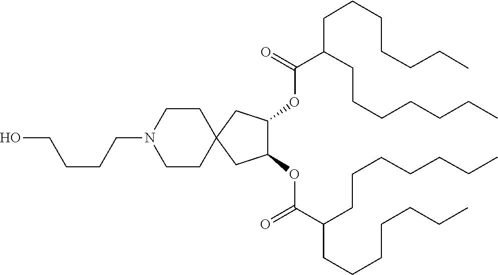 [SMILES: CCCCCCCC(CCCCCCC)C(=O)O[C@H]1CC2(CCN(CCCCO)CC2)C[C@@H]1OC(=O)C(CCCCCCC)CCCCCCC] | |

| | |
|---|---|
| 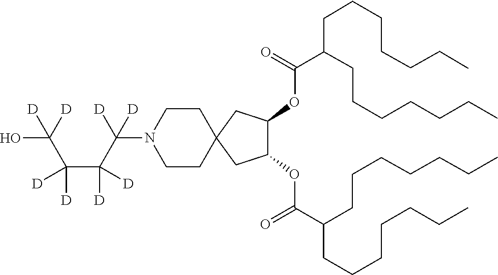 [SMILES: [2H]C([2H])(O)C([2H])([2H])C([2H])([2H])C([2H])([2H])N1CCC2(CC1)C[C@@H](OC(=O)C(CCCCCCC)CCCCCCC)[C@H](OC(=O)C(CCCCCCC)CCCCCCC)C2] | Example 51-1-D |
| 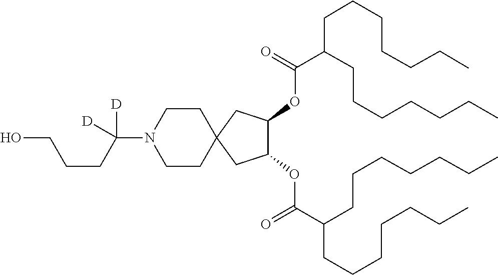 [SMILES: [2H]C([2H])(CCCO)N1CCC2(CC1)C[C@@H](OC(=O)C(CCCCCCC)CCCCCCC)[C@H](OC(=O)C(CCCCCCC)CCCCCCC)C2] | Example 51-2-D |
| 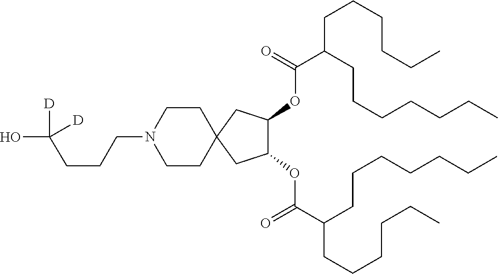 [SMILES: [2H]C([2H])(O)CCCN1CCC2(CC1)C[C@@H](OC(=O)C(CCCCCC)CCCCCCC)[C@H](OC(=O)C(CCCCCC)CCCCCCC)C2] | Example 51-3-D |
| 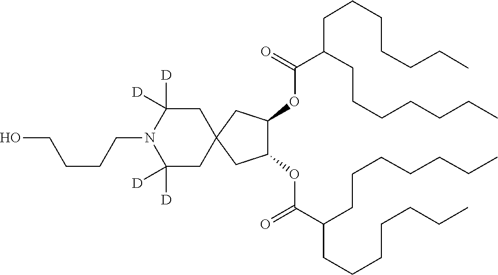 [SMILES: [2H]C1([2H])CC2(C[C@@H](OC(=O)C(CCCCCCC)CCCCCCC)[C@H](OC(=O)C(CCCCCCC)CCCCCCC)C2)CC([2H])([2H])N1CCCCO] | Example 51-4-D |
| 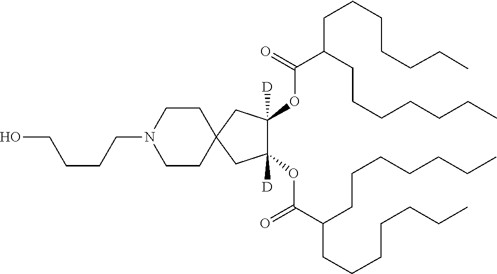 [SMILES: [2H][C@@]1(OC(=O)C(CCCCCCC)CCCCCCC)CC2(CCN(CCCCO)CC2)C[C@@]1([2H])OC(=O)C(CCCCCCC)CCCCCCC] | Example 51-5-D |
| 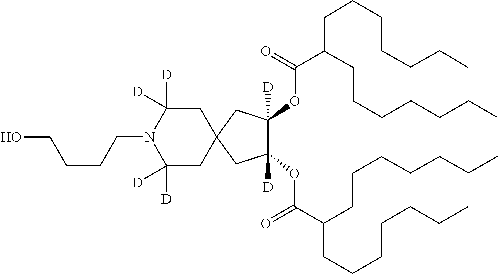 [SMILES: [2H]C1([2H])CC2(CC([2H])([2H])N1CCCCO)C[C@@]([2H])(OC(=O)C(CCCCCCC)CCCCCCC)[C@]([2H])(OC(=O)C(CCCCCCC)CCCCCCC)C2] | Example 51-6-D |
| 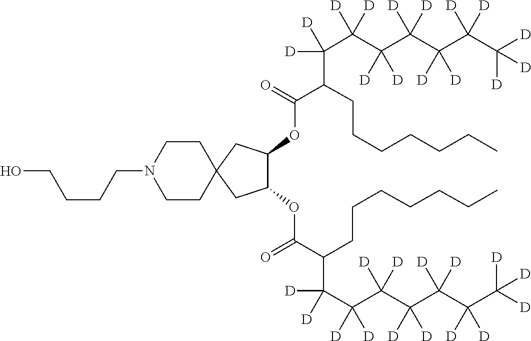 [SMILES: [2H]C([2H])([2H])C([2H])([2H])C([2H])([2H])C([2H])([2H])C([2H])([2H])C([2H])([2H])C([2H])([2H])C(CCCCCCC)C(=O)O[C@@H]1CC2(CCN(CCCCO)CC2)C[C@H]1OC(=O)C(CCCCCCC)C([2H])([2H])C([2H])([2H])C([2H])([2H])C([2H])([2H])C([2H])([2H])C([2H])([2H])C([2H])([2H])[2H]] | Example 51-7-D |
| 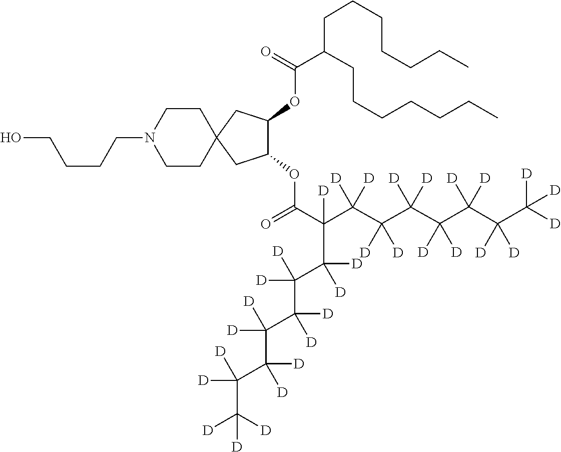 [SMILES: [2H]C([2H])([2H])C([2H])([2H])C([2H])([2H])C([2H])([2H])C([2H])([2H])C([2H])([2H])C([2H])([2H])C([2H])(C(=O)O[C@@H]1CC2(CCN(CCCCO)CC2)C[C@H]1OC(=O)C(CCCCCCC)CCCCCCC)C([2H])([2H])C([2H])([2H])C([2H])([2H])C([2H])([2H])C([2H])([2H])C([2H])([2H])C([2H])([2H])[2H]] | Example 51-8-D |
| 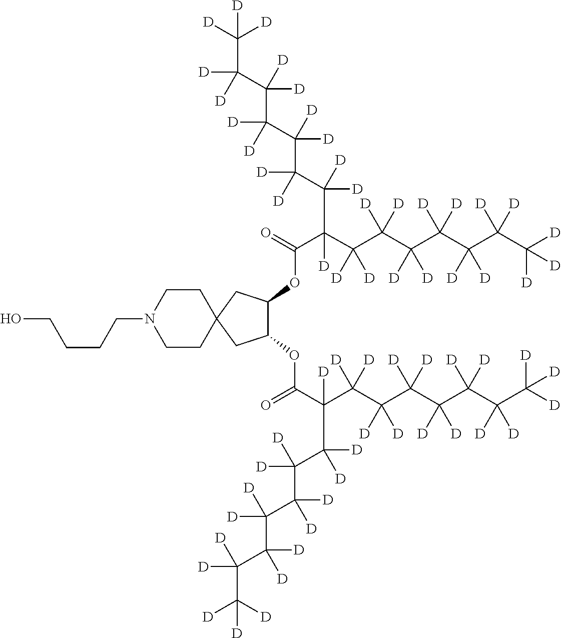 [SMILES: [2H]C([2H])([2H])C([2H])([2H])C([2H])([2H])C([2H])([2H])C([2H])([2H])C([2H])([2H])C([2H])([2H])C([2H])(C(=O)O[C@@H]1CC2(CCN(CCCCO)CC2)C[C@H]1OC(=O)C([2H])(C([2H])([2H])C([2H])([2H])C([2H])([2H])C([2H])([2H])C([2H])([2H])C([2H])([2H])C([2H])([2H])[2H])C([2H])([2H])C([2H])([2H])C([2H])([2H])C([2H])([2H])C([2H])([2H])C([2H])([2H])C([2H])([2H])[2H])C([2H])([2H])C([2H])([2H])C([2H])([2H])C([2H])([2H])C([2H])([2H])C([2H])([2H])C([2H])([2H])[2H]] | Example 51-9-D |
| 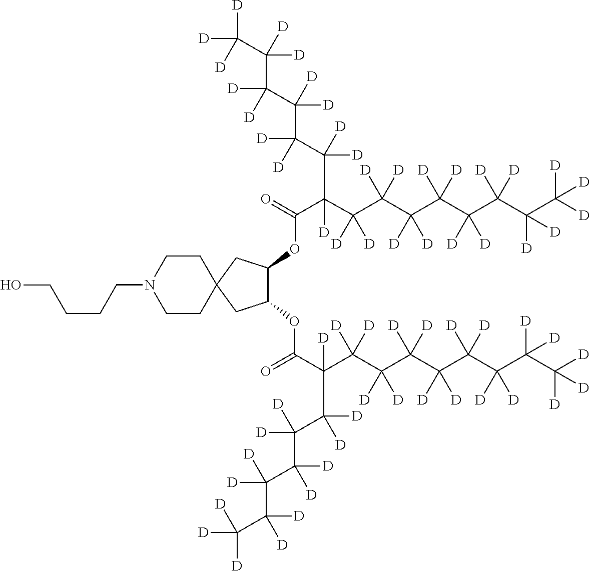 [SMILES: [2H]C([2H])([2H])C([2H])([2H])C([2H])([2H])C([2H])([2H])C([2H])([2H])C([2H])([2H])C([2H])([2H])C([2H])([2H])C([2H])(C(=O)O[C@@H]1CC2(CCN(CCCCO)CC2)C[C@H]1OC(=O)C([2H])(C([2H])([2H])C([2H])([2H])C([2H])([2H])C([2H])([2H])C([2H])([2H])C([2H])([2H])[2H])C([2H])([2H])C([2H])([2H])C([2H])([2H])C([2H])([2H])C([2H])([2H])C([2H])([2H])C([2H])([2H])C([2H])([2H])[2H])C([2H])([2H])C([2H])([2H])C([2H])([2H])C([2H])([2H])C([2H])([2H])C([2H])([2H])[2H]] | Example 51-10-D |
| 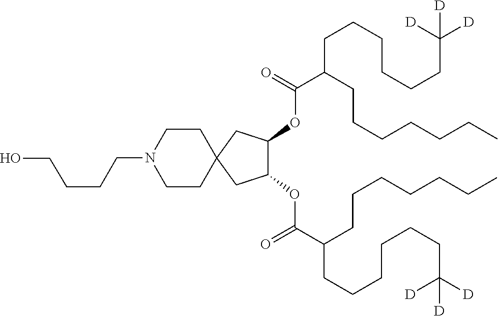 [SMILES: [2H]C([2H])([2H])CCCCCCC(CCCCCCC)C(=O)O[C@@H]1CC2(CCN(CCCCO)CC2)C[C@H]1OC(=O)C(CCCCCCC)CCCCCCC([2H])([2H])[2H]] | Example 51-11-D |
| 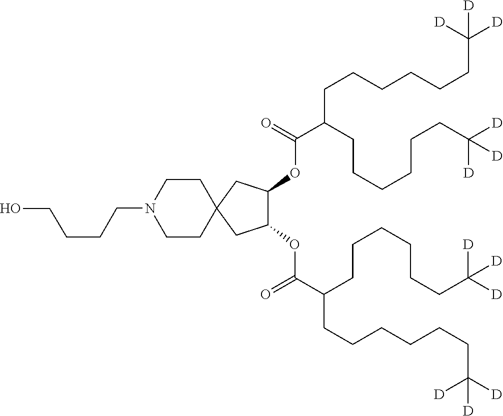 [SMILES: [2H]C([2H])([2H])CCCCCCC(CCCCCCC([2H])([2H])[2H])C(=O)O[C@@H]1CC2(CCN(CCCCO)CC2)C[C@H]1OC(=O)C(CCCCCCC([2H])([2H])[2H])CCCCCCC([2H])([2H])[2H]] | Example 51-12-D |
| 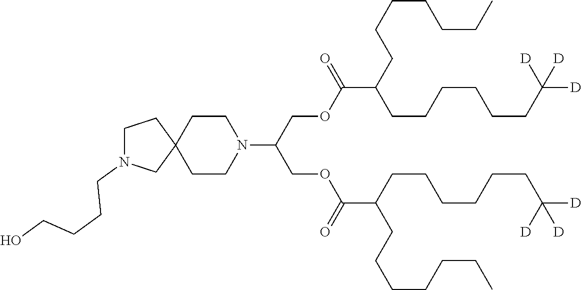 [SMILES: [2H]C([2H])([2H])CCCCCCC(CCCCCCC)C(=O)OCC(COC(=O)C(CCCCCCC)CCCCCCC([2H])([2H])[2H])N1CCC2(CCN(CCCCO)C2)CC1] | Example 51-13-D |

TABLE 6G

| | |
|---|---|
| | Example 61 |
| 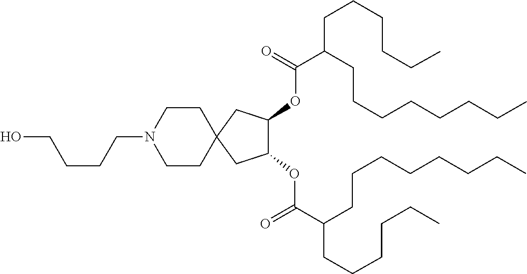 [SMILES: CCCCCCCCC(CCCCCC)C(=O)O[C@@H]1CC2(CCN(CCCCO)CC2)C[C@H]1OC(=O)C(CCCCCC)CCCCCCCC] | |

| | |
|---|---|
| 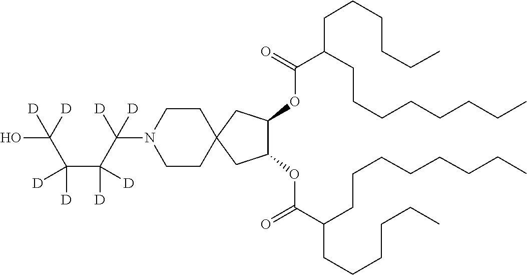 [SMILES: [2H]C([2H])(O)C([2H])([2H])C([2H])([2H])C([2H])([2H])N1CCC2(CC1)C[C@@H](OC(=O)C(CCCCCC)CCCCCCCC)[C@H](OC(=O)C(CCCCCC)CCCCCCCC)C2] | Example 61-1-D |
| 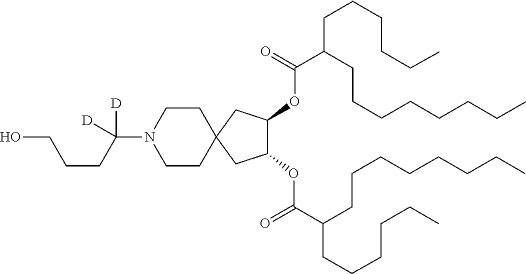 [SMILES: [2H]C([2H])(CCCO)N1CCC2(CC1)C[C@@H](OC(=O)C(CCCCCC)CCCCCCCC)[C@H](OC(=O)C(CCCCCC)CCCCCCCC)C2] | Example 61-2-D |
| 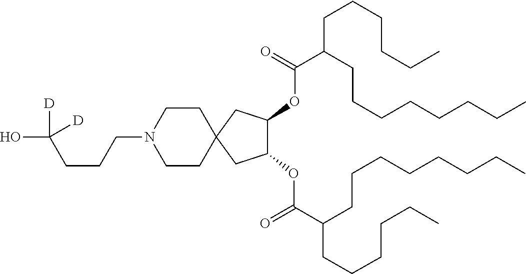 [SMILES: [2H]C([2H])(O)CCCN1CCC2(CC1)C[C@@H](OC(=O)C(CCCCCC)CCCCCCCC)[C@H](OC(=O)C(CCCCCC)CCCCCCCC)C2] | Example 61-3-D |
| 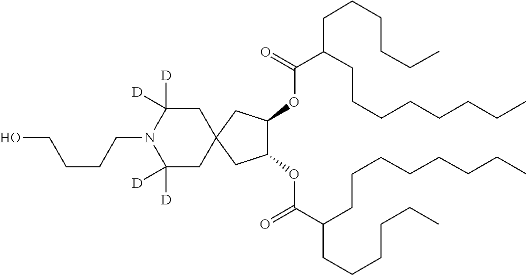 [SMILES: [2H]C1([2H])CC2(C[C@@H](OC(=O)C(CCCCCC)CCCCCCCC)[C@H](OC(=O)C(CCCCCC)CCCCCCCC)C2)CC([2H])([2H])N1CCCCO] | Example 61-4-D |
| 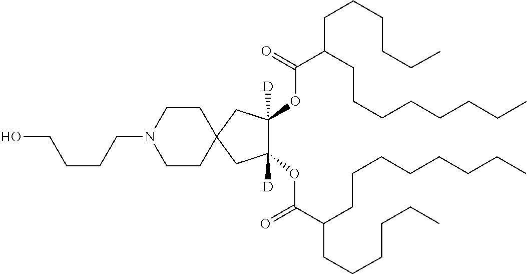 [SMILES: [2H][C@@]1(OC(=O)C(CCCCCC)CCCCCCCC)CC2(CCN(CCCCO)CC2)C[C@@]1([2H])OC(=O)C(CCCCCC)CCCCCCCC] | Example 61-5-D |
| 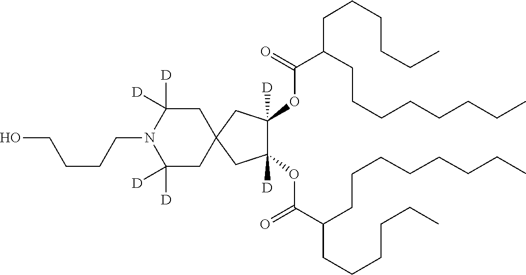 [SMILES: [2H]C1([2H])CC2(CC([2H])([2H])N1CCCCO)C[C@@]([2H])(OC(=O)C(CCCCCC)CCCCCCCC)[C@]([2H])(OC(=O)C(CCCCCC)CCCCCCCC)C2] | Example 61-6-D |
| 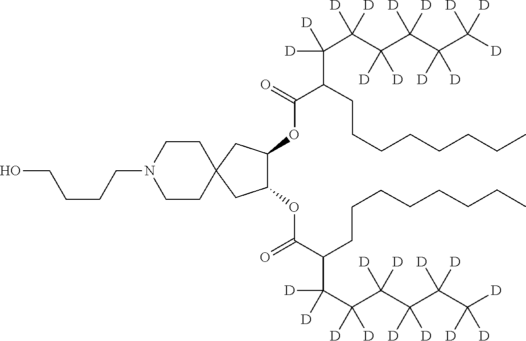 [SMILES: [2H]C([2H])([2H])C([2H])([2H])C([2H])([2H])C([2H])([2H])C([2H])([2H])C([2H])([2H])C(CCCCCCCC)C(=O)O[C@@H]1CC2(CCN(CCCCO)CC2)C[C@H]1OC(=O)C(CCCCCCCC)C([2H])([2H])C([2H])([2H])C([2H])([2H])C([2H])([2H])C([2H])([2H])C([2H])([2H])[2H]] | Example 61-7-D |
| 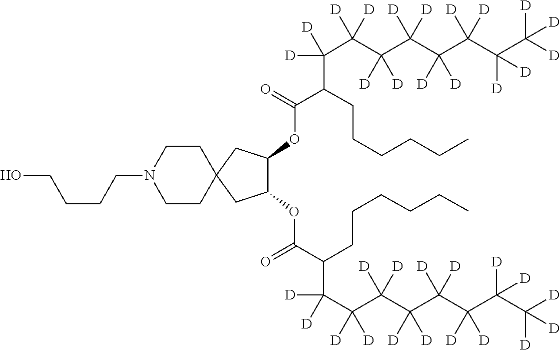 [SMILES: [2H]C([2H])([2H])C([2H])([2H])C([2H])([2H])C([2H])([2H])C([2H])([2H])C([2H])([2H])C([2H])([2H])C([2H])([2H])C(CCCCCC)C(=O)O[C@@H]1CC2(CCN(CCCCO)CC2)C[C@H]1OC(=O)C(CCCCCC)C([2H])([2H])C([2H])([2H])C([2H])([2H])C([2H])([2H])C([2H])([2H])C([2H])([2H])C([2H])([2H])C([2H])([2H])[2H]] | Example 61-8-D |
| 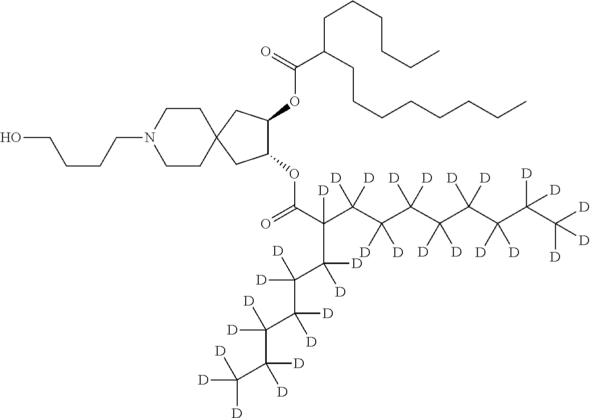 [SMILES: [2H]C([2H])([2H])C([2H])([2H])C([2H])([2H])C([2H])([2H])C([2H])([2H])C([2H])([2H])C([2H])([2H])C([2H])([2H])C([2H])(C(=O)O[C@@H]1CC2(CCN(CCCCO)CC2)C[C@H]1OC(=O)C(CCCCCC)CCCCCCCC)C([2H])([2H])C([2H])([2H])C([2H])([2H])C([2H])([2H])C([2H])([2H])C([2H])([2H])[2H]] | Example 61-9-D |
| 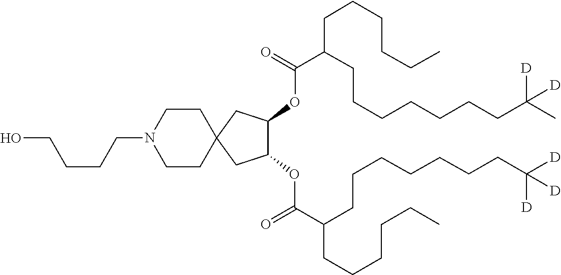 [SMILES: [2H]C([2H])([2H])CCCCCCCC(CCCCCC)C(=O)O[C@@H]1CC2(CCN(CCCCO)CC2)C[C@H]1OC(=O)C(CCCCCC)CCCCCCCC([2H])([2H])C] | Example 61-10-D |
| 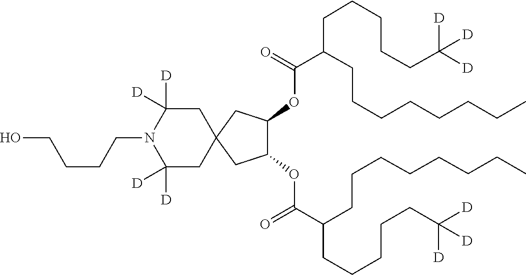 [SMILES: [2H]C([2H])([2H])CCCCCC(CCCCCCCC)C(=O)O[C@@H]1CC2(C[C@H]1OC(=O)C(CCCCCCCC)CCCCCC([2H])([2H])[2H])CC([2H])([2H])N(CCCCO)C([2H])([2H])C2] | Example 61-11-D |
| 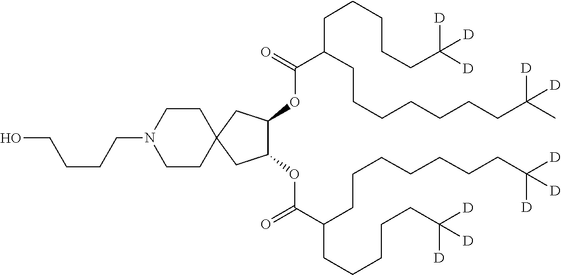 [SMILES: [2H]C([2H])([2H])CCCCCCCC(CCCCCC([2H])([2H])[2H])C(=O)O[C@@H]1CC2(CCN(CCCCO)CC2)C[C@H]1OC(=O)C(CCCCCCCC([2H])([2H])C)CCCCCC([2H])([2H])[2H]] | Example 61-12-D |

The examples of Tables 6A-6G may be synthesized employing methods analogous to those described in the examples above employing deuterated starting materials that are either commercially available or known in the literature. Also, various methods of metal-catalyzed hydrogen-deuterium exchange would be known to those skilled in the art that could be employed on the final lipid or intermediates there to, for example as described by Yamada et al. Adv. Synth. Catal. 2016, 358, 3277.

General methods/reviews for obtaining metabolite profile and identifying metabolites of a compound are described in: Dalvie, et al., “Assessment of Three Human in Vitro Systems in the Generation of Major Human Excretory and Circulating Metabolites,” Chemical Research in Toxicology, 2009, 22, 2, 357-368, tx8004357 (acs.org); King, R., “Biotransformations in Drug Metabolism,” Ch.3, Drug Metabolism Handbook Introduction, https://doi.org/10.1002/9781119851042.ch3; Wu, Y., et al, “Metabolite Identification in the Preclinical and Clinical Phase of Drug Development,” Current Drug Metabolish, 2021, 22, 11, 838-857, 10.2174/1389200222666211006104502; Godzien, J., et al, “Chapter Fifteen-Metabolite Annotation and Identification”.

Numerous publicly available and commercially available software tools are available to aid in the predictions of metabolic pathways and metabolites of compounds. Examples of such tools include, BioTransformer 3.0 (biotransformer.ca/new) which predicts the metabolic biotransformations of small molecules using a database of known metabolic reactions; MetaSite (moldiscovery.com/software/metasite/) which predicts metabolic transformations related to cytochrome P450 and flavin-containing monooxygenase mediated reactions in phase I metabolism; and Lhasa Meteor Nexus (lhasalimited.org/products/meteor-nexus.htm) offers prediction of metabolic pathways and metabolite structures using a range of machine learning models, which covers phase I and phase II biotransformations of small molecules.

The Examples set forth in Tables 6A-6G may afford certain therapeutic advantages resulting from reduced oxidative transformation, for example reduced dosage requirements, reduced CYP450 inhibition (competitive or time dependent), or an improvement in therapeutic index or tolerability.

A person with ordinary skill may make additional deuterated analogs of the Examples provided in Tables 6A-6G with different combinations of deuterated atom positions. Such additional deuterated analogs may provide similar therapeutic advantages that may be achieved by the deuterated analogs.

### Example 72

Preparation, Characterization, and Determination of Efficacy for Lipid Nanoparticle Formulations Containing Various Ionizable Lipids and mRNA (WISC HA modFlu)

The Examples are based on the influenza modRNA, unless specified otherwise. The influenza modRNA immunogenic composition is comprised of one or more nucleoside-modified mRNAs that encode the full-length HA glycoprotein derived from seasonal human influenza strains.

The specific construct (Wisconsin HA modRNA) is the only active ingredient in the immunogenic composition. In addition to the codon-optimized sequence encoding the antigen, the RNA contains common structural elements optimized for mediating high RNA stability and translational efficiency (5′-cap, 5′UTR, 3′-UTR, poly (A)-tail; see table and sequences below). Furthermore, an intrinsic signal peptide (sec) is part of the open reading frame and is translated as an N-terminal peptide. The RNA does not contain any uridines; instead of uridine, the modified N1-methylpseudouridine is used in RNA synthesis.

The specific constructs each comprise the elements shown below in Table 7:

TABLE 7
Construct Elements

| Element | Description | Position |
|---|---|---|
| 5′-cap | A modified 5′-cap1 structure (m⁷G⁺m3′-5′-ppp-5′-Am) | 1-2 |
| 5′-UTR | 5′-untranslated region derived from human alpha- | 3-54 |
| | globin RNA with an optimized Kozak sequence | |
| 3′-UTR | The 3′ untranslated region comprises two sequence | |
| | elements derived from the amino-terminal enhancer of | |
| | split (AES) mRNA and the mitochondrial encoded 12S | |
| | ribosomal RNA to confer RNA stability and high total | |
| | protein expression. | |
| poly(A) | A 110-nucleotide poly(A)-tail consisting of a stretch | |
| | of 30 adenosine residues, followed by a 10-nucleotide | |
| | linker sequence and another 70 adenosine residues. | |

### Sequences of Elements:

Cap and 5′-UTR: GAGAAψAAAC ψAGψAψψCψψ CψGGψCCCCA CAGACψCAGA GAGAACCCGC CACC (SEQ ID NO:1), where the bolded and underlined text corresponds to the 5 cap and the unmodified text corresponds to the 5′-UTR.

3′-UTR:
(SEQ ID NO: 2)
CΨCGAGCΨGGΨ ACΨGCAΨGCA CGCAAΨGCΨA GCΨGCCCCΨΨ
ΨCCCGΨCCΨG GGΨACCCCGA GΨCΨCCCCCG ACCΨCGGGΨC
CCAGGΨAΨGC ΨCCCACCΨCC ACCΨGCCCCA CΨCACCACCΨ
CΨGCΨAGΨΨC CAGACACCΨC CCAAGCACGC AGCAAΨGCAG
CΨCAAAACGC ΨΨAGCCΨAGC CACACCCCCA CGGGAAACAG
CAGΨGAΨΨAA CCΨΨΨAGCAA ΨAAACGAAAG ΨΨΨAACΨAAG
CΨAΨACΨAAC CCCAGGGΨΨG GΨCAAΨΨΨCG ΨGCCAGCCAC
ACCCΨGGAGC ΨAGC
Poly(A) tail:
(SEQ ID NO: 3)
AAAAAA AAAAAAAAAA AAAAAAAAAA AAAAGCAΨAΨ
GACΨAAAAAA AAAAAAAAAA AAAAAAAAAA AAAAAAAAAA
AAAAAAAAAA AAAAAAAAAA AAAAAAAAAA AAAA

As used herein, ψ represents 1-methyl-3′-pseudouridytyl.

The 5′-cap analog (m₂⁷·3′^{-OMe}Gppp(m₁2′^{-O})ApG) for production of RNA containing a cap1 structure is shown below.

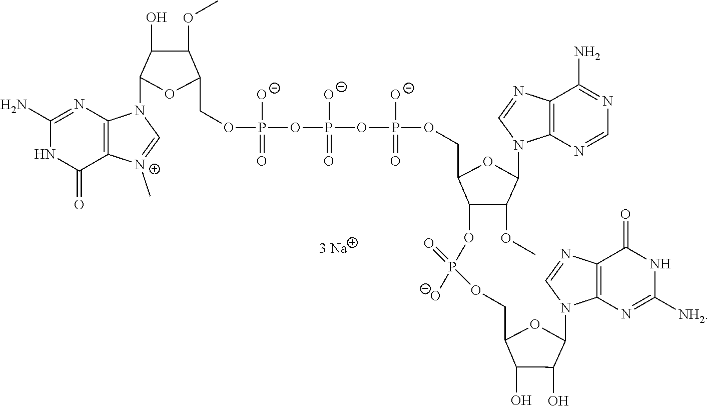 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O]

The above structure corresponds to Trilink's CleanCap AG (3′OMe)-m27,3′-OGppp (m12′-O) ApG. This molecule is identical to the natural RNA cap structure in that it starts with a guanosine methylated at N7 and is linked by a 5′ to 5′ triphosphate linkage to the first coded nucleotide of the transcribed RNA (in this case, an adenosine). This guanosine is also methylated at the 3′ hydroxyl of the ribose to mitigate possible reverse incorporation of the cap molecule. Finally, the 2′ hydroxyl of the ribose on the adenosine is methylated, conferring a Cap1 structure—by contrast, leaving this as a 2′ hydroxyl would give this a Cap0 structure. Cap1 structures should provide superior transcription to RNA's with a Cap0 structure in eukaryotes.

The influenza modRNA vaccine candidates may encode the HA protein derived from A/Wisconsin/588/2019 (H1N1), A/Cambodia/e0826360/2020 (H3N2), B/Washington/02/2019 (B/Victoria-lineage) and B/Phuket/3073/2013 (B/Yamagata lineage), which are the recommended vaccine strains for the cell culture-based influenza vaccines for the Northern Hemisphere 2021-2022 season. The number of A nucleotides present in the poly (A)-tail in the sequences preferably reflect how it would be in the final RNA after linearization with BspQ1 (or its isoschizomer): 30A-linker-70A. In the transcribed RNA with Cap 1 structure, the first two nucleotides in the mRNA sequence (AG) are actually provided by the CLEANCAP reagent and the 2′ hydroxyl of the ribose on the first adenosine is methylated.

The cap1 structure (e.g., containing a 2′-O-methyl group on the penultimate nucleoside of the 5′-end of the RNA chain) is incorporated into the drug substance by using a respective cap analog during in vitro transcription. For RNAs with modified uridine nucleotides, the cap1 structure is superior to other cap structures, since cap1 is not recognized by cellular factors such as IFIT1 and, thus, cap1-dependent translation is not inhibited by competition with eukaryotic translation initiation factor 4E. In the context of IFIT1 expression, mRNAs with a cap1 structure give higher protein expression levels.

In some preferred embodiments, the Influenza vaccine drug substance is a single-stranded, 5′-capped mRNA that is translated into the respective protein (the encoded antigen) which corresponds to the Hemagglutinin (HA) protein from Influenza strains either A/Wisconsin/588/2019 H1N1, A/Cambodia/e0826360/2020, B/Washington/02/2019 or B/Phuket/3073/2013. the general structure of the antigen-encoding RNA is determined by the respective nucleotide sequence of the DNA used as template for in vitro RNA transcription. In addition to the codon-optimized sequence encoding the antigen, the RNA contains common structural elements optimized for mediating high RNA stability and translational efficiency (5′-cap, 5′UTR, 3′-UTR, poly (A)-tail; see below).

The manufacturing process comprises RNA synthesis via an in vitro transcription (IVT) step followed by DNase I and proteinase K digestion steps, purification by ultrafiltration/diafiltration (UFDF), final filtration, dispense into an appropriate container, and storage at −20° C. A platform approach to the IVT, digestion, and purification process steps was used in the production of the four modRNAs. The mRNA clinical batches were prepared at a scale of 37.6 L starting volume for IVT. All the material was purified by a single 2-stage UFDF to produce mRNA drug substance.

Lipid nanoparticles were prepared and tested according to the general procedures described in U.S. Pat. No. 9,737,619 (PCT Pub. No. WO2015/199952) and U.S. Pat. No. 10,166,298 (PCT Pub. No. WO 2017/075531) and PCT Pub. No. WO2020/146805. The novel ionizable lipids of the invention, cholesterol, DSPC and PEG-Lipid were solubilized in Ethanol at a molar ratio of about 46.3:42.7:9.4:1.6. Lipid nanoparticles (LNP) were prepared at a total lipid to mRNA ratio of about 23:1. In short, the mRNA (WISC HA modFlu) was diluted in buffer. Syringe pumps were used to mix the lipid solution with the mRNA solution. The ethanol was removed, and external buffer replaced with another buffer (e.g., Tris) by dialysis. Finally, the lipid nanoparticle size and size distribution were determined by dynamic light scattering using an Unchained Labs Stunner (Unchained Labs, USA). RNA encapsulation efficiency was determined using the Quant-iT RiboGreen RNA assay (Life Technologies, USA). Briefly, LNPs were incubated with RiboGreen dye (200-fold dilution per manufactures' instruction) in the presence and absence of 1% Triton-X 100 and fluorescence intensities (Excitation/Emission: 485/528 nM) were measured for unencapsulated RNA and total RNA after release from LNPs by Triton-X 100.

Plated Hek293T or Hela cells were dosed with lipid nanoparticles in a total volume of 40 μl cell culture media and incubated overnight at 37° C. and 5% CO₂. After fixation with 4% paraformaldehyde in PBS cells were washed in PBS containing 0.3% Triton-X100 and 3% BSA (w/v), followed by nuclear staining with Hoechst and antibody staining for the encoded gene of interest. Nuclear count and identification of cells stained positive for gene of interest was done on an Opera Phenix high content imager (PerkinElmer, USA).

TABLE 8

| | | | | EC₅₀ | EC₅₀ |
|---|---|---|---|---|---|
| Ionizable lipid | Size | | | Hek293T | Hela |
| (Example Number) | (nm) | PDI | EE % | (ng/well) | (ng/well) |
| 1 | 76 | 0.04 | 99 | 13.9 | 18.3 |
| 2 | 93 | 0.06 | 99 | 14.5 | 28.1 |
| 3 | 152 | 0.07 | 99 | 11.9 | 18.4 |
| 51 | 72 | 0.03 | 96 | 5.3 | 10.7 |

### Example 73

Preparation, Characterization, and Determination of Efficacy for Lipid Nanoparticle Formulations Containing Various Ionizable Lipids and mRNA (CA HA modFlu mRNA)

The specific construct (California HA modRNA) is the only active ingredient in the immunogenic composition. In addition to the codon-optimized sequence encoding the antigen, the RNA contains common structural elements optimized for mediating high RNA stability and translational efficiency (5′-cap, 5′UTR, 3′-UTR, poly (A)-tail; see table and sequences described in Example 72). Furthermore, an intrinsic signal peptide (sec) is part of the open reading frame and is translated as an N-terminal peptide. The RNA does not contain any uridines; instead of uridine, the modified N1-methylpseudouridine is used in RNA synthesis.

Lipid nanoparticles were prepared and tested according to the general procedures described in U.S. Pat. No. 9,737,619 (PCT Pub. No. WO2015/199952) and U.S. Pat. No. 10,166,298 (PCT Pub. No. WO 2017/075531) and PCT Pub. No. WO2020/146805. The novel ionizable lipids of the invention, cholesterol, distearoylphosphatidylcholine (DSPC) and ALC-0159 2-[(polyethylene glycol)-2000]-N,N ditetradecylacetamide were solubilized in ethanol at a molar ratio of about 46.3:42.7:9.4:1.6. Lipid nanoparticles were made by mixing the lipids containing organic phase with the mRNA containing aqueous phase using an N:P ratio of about 6:1. The mRNA was diluted in buffer. Syringe pumps were used to mix the ethanolic lipid solution with the mRNA aqueous solution. The ethanol was then removed, and the external buffer replaced with another buffer (e.g., Tris) by dialysis. Finally, the lipid nanoparticles were filtered.

Total RNA concentration and encapsulation efficiency was assessed using the RiboGreen RNA quantitation assay. RNA integrity pre and post formulation was monitored by Agilent Fragment Analyzer capillary gel electrophoresis. Malvern Zetasizer dynamic light scattering (DLS) was used to determine LNP size and polydispersity index (PDI). In vitro expression of the novel ionizable lipid nanoparticles was assessed in HEK293F and Hela cells similar to described in Example 72. Nuclear count and identification of cells stained positive for gene of interest was done on an Opera Phenix high content imager (PerkinElmer, USA).

TABLE 9

| | | | | EC₅₀ | EC₅₀ |
|---|---|---|---|---|---|
| Ionizable lipid | Size | | | Hek293F | Hela |
| (Example Number) | (nm) | PDI | EE % | (ng/well) | (ng/well) |
| 1 | 78 | 0.050 | 100 | 1.7 | 1.2 |
| 2 | 92 | 0.040 | 100 | 2.5 | 1.6 |
| 3 | 90 | 0.040 | 100 | 2.8 | 1.7 |
| 4 | 97 | 0.170 | 98 | 46.9 | >50 |
| 5 | 136 | 0.040 | 100 | 1.32 | 4.3 |
| 6 | 133 | 0.080 | 100 | 0.42 | 1.1 |
| 7 | 84 | 0.130 | 99 | 20.4 | 2.0 |
| 8 | 68 | 0.101 | 100 | 4.1 | 2.5 |
| 9 | 96 | 0.070 | 99 | 2.6 | 4.2 |
| 10 | 109 | 0.054 | 99 | 1.3 | 1.5 |
| 11 | 121 | 0.080 | 99 | 3.7 | 13.8 |
| 12 | 105 | 0.042 | 100 | 0.22 | 0.44 |
| 13 | 131 | 0.086 | 99 | 0.36 | 1.6 |
| 14 | 114 | 0.036 | 100 | 1.5 | 2.5 |
| 15 | 110 | 0.120 | 100 | 4.0 | 2.7 |
| 16 | 134 | 0.090 | 99 | 0.11 | 0.18 |
| 17 | 149 | 0.053 | 100 | 0.64 | 1.3 |
| 18 | 149 | 0.060 | 99 | 0.57 | 1.4 |
| 19 | 153 | 0.140 | 99 | 1.4 | 0.89 |
| 20 | 105 | 0.073 | 98 | 0.22 | 0.28 |
| 21 | 159 | 0.064 | 99 | 0.28 | 0.41 |
| 22 | 107 | 0.065 | 99 | 0.14 | 0.39 |
| 23 | 144 | 0.090 | 100 | 0.47 | 1.2 |
| 24 | 82 | 0.072 | 99 | 0.48 | 0.72 |
| 25 | 137 | 0.040 | 99 | 0.42 | 0.61 |
| 26 | 96 | 0.030 | 100 | 1.6 | 1.8 |
| 27 | 74 | 0.095 | 97 | 35.9 | 12.5 |
| 28 | 96 | 0.025 | 99 | 9.9 | 6.4 |
| 29 | 130 | 0.050 | 100 | 0.82 | 1.0 |
| 30 | 102 | 0.030 | 99 | 12.6 | 3.3 |
| 31 | 121 | 0.150 | 99 | 1.7 | 2.9 |
| 32 | 132 | 0.060 | 100 | 0.96 | 0.91 |
| 33 | 115 | 0.090 | 99 | 0.22 | 0.29 |
| 34 | 130 | 0.040 | 99 | 2.1 | 3.8 |
| 35 | 102 | 0.040 | 99 | 21.3 | 1.4 |
| 36 | 136 | 0.059 | 99 | 1.9 | 2.4 |
| 37 | 143 | 0.070 | 99 | 0.67 | 0.90 |
| 38 | 146 | 0.100 | 100 | 28.1 | 7.0 |
| 39 | 134 | 0.080 | 100 | 4.1 | 3.4 |
| 40 | 117 | 0.080 | 100 | 5.6 | 3.6 |
| 41 | 119 | 0.060 | 99 | 0.20 | 0.30 |
| 42 | 100 | 0.080 | 99 | 42.7 | >50 |
| 43 | 89 | 0.111 | 99 | 33.3 | 14.2 |
| 44 | 119 | 0.080 | 99 | 0.28 | 0.30 |
| 45 | 117 | 0.060 | 99 | 1.1 | 1.1 |
| 46 | 92 | 0.090 | 99 | 24.6 | 1.7 |
| 47 | 147 | 0.080 | 99 | 5.9 | 4.6 |
| 48 | 127 | 0.093 | 100 | 0.76 | 1.0 |
| 49 | 160 | 0.110 | 99 | 0.41 | 1.4 |
| 50 | 120 | 0.046 | 99 | 0.56 | 0.74 |
| 51 | 58 | 0.030 | 99 | 0.27 | 0.64 |
| 52 | 66 | 0.088 | 99 | 3.3 | 11.0 |
| 53 | 89 | 0.090 | 94 | 2.3 | 29.5 |
| 54 | 71 | 0.101 | 98 | 1.5 | 3.6 |
| 55 | 65 | 0.124 | 100 | 21.4 | 45.0 |
| 56 | 81 | 0.063 | 98 | 0.60 | 0.96 |
| 57 | 74 | 0.118 | 99 | 2.9 | 4.7 |
| 58 | 79 | 0.125 | 98 | 1.6 | 1.7 |
| 59 | 80 | 0.091 | 97 | 2.3 | 34.6 |
| 60 | 94 | 0.117 | 98 | 1.6 | >50 |
| 61 | 77 | 0.094 | 99 | 1.0 | 1.6 |
| 62 | 161 | 0.090 | 99 | 0.36 | 1.0 |
| 63 | 107 | 0.060 | 99 | 0.39 | 0.58 |
| 64 | 110 | 0.056 | 99 | 0.64 | 0.69 |
| 65 | 123 | 0.038 | 97 | 0.35 | 0.57 |
| 66 | 78 | 0.146 | 97 | 1.1 | 2.2 |
| 67 | 89 | 0.127 | 96 | 1.4 | 2.0 |
| 70 | 116 | 0.110 | 98 | 3.0 | 1.2 |

### Example 74

### In Vivo Study

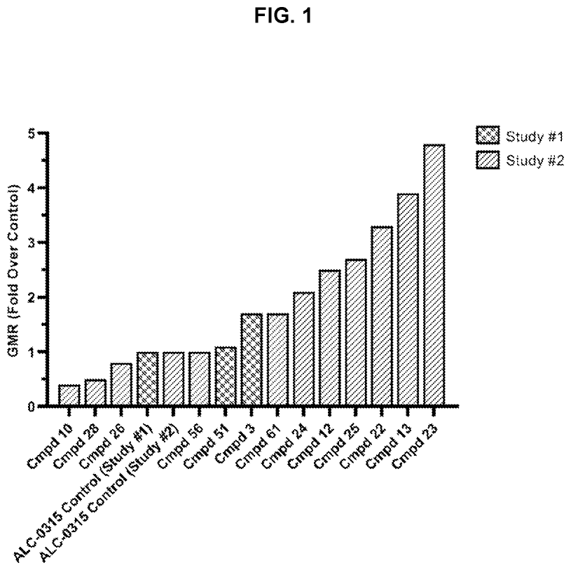

Female Balb/c mice (9-11 weeks, 10 mice per group) were immunized with a 0.2 microgram dose of modRNA Flu HA/California encapsulated with different LNP materials administered as a 50 microliter intramuscular injection on Day 0 (prime) and 28 (boost). All LNPs were formulated in 10 mM Tris, 300 mM Sucrose, pH 7.4. Sera collected on Day 21 post prime and Day 42 (14 days post boost) were evaluated by serology testing to quantitatively measure functional antibodies in serum that neutralize influenza virus activity. Geometric mean neutralization titers are reported as the reciprocal of the dilution that results in 50% reduction in infection when compared to a no serum control. Resulting neutralization titers were normalized to LNP formulated using ALC-0315 (((4-hydroxybutyl) azanediyl)bis(hexane-6,1-diyl) bis(2-hexyldecanoate)) as the ionizable lipid in order to provide the geometric mean ratio. The results are shown in FIG. 1.

TABLE 10
LNP

| | |
|---|---|
| LNP Formulation | Molar Ratio of Ionizable |
| | lipid:Cholesterol:DSPC:ALC-0159 = |
| | 50:38.5:10:1.5 |
| | NP ratio = 6 |
| LNP Formulation | 10 mM Tris, 300 mM Sucrose, pH 7.4 |
| Matrix | |
| Description of | modRNA Flu HA/California |
| Antigen | |

TABLE 11
Study Schedule

| Day | Procedure |
|---|---|
| 0 | Vaccination #1 |
| 21 | Interim Bleed (Submandibular) |
| 28 | Vaccination #2 |
| 42 | Terminal Bleed (Cardiac Puncture) |

It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

1. A compound of Formula (I) 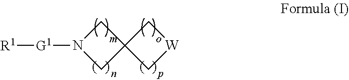
or a pharmaceutically acceptable salt, N-oxide, tautomer, or stereoisomer thereof, wherein
m, n, o, and p are each independently 1-3;
G¹ is C₁₋₁₂alkylene or C₂₋₁₂alkenylene;
R¹ is —N(R²)R³, —OR⁴, CN, —N(R⁴)(heteroaryl), —O(CH₂)_{q}OH, —(OCH₂CH₂)ᵣOH, —OC(═O)R⁵, —N(R⁴)C(═O)R⁵, —N(R⁴)S(O)₂R⁵, —N(R⁴)C(═O)N(R²)R³, —OC(═O)N(R²)R³, —N(R⁴)C(═O)OR⁵, —N(R⁴)C(═S)N(R²)R³, —N(R⁴)C(═NR⁶)N(R²)R³, or 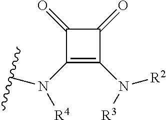
R² and R³ are each independently H, C₁₋₆alkyl, C₃₋₈cycloalkyl, or aryl; or R² and R³ together with the nitrogen atom to which they are attached form a heterocyclic ring;
R⁴ is H, C₁₋₆alkyl or C₃₋₈cycloalkyl;
R⁵ is C₁₋₆alkyl or C₃₋₈cycloalkyl optionally substituted by C₁₋₆alkyl;
R⁶ is H, CN, NO₂, C₁₋₆alkyl, OR⁵, S(O)₂R⁵, or S O)₂N(R²)R³;
q is 2-6;
r is 1-6;
W is 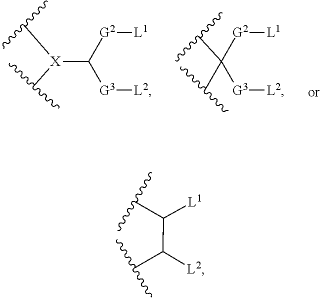
X is N or CH;
G² and G³ are each independently C₁₋₁₂alkylene or C₂₋₁₂alkenylene;
L¹ and L² are each independently —C(═O)OR⁷, —OC(═O)R⁷, —OC(═O)(CH₂)ᵣC(═O)OR⁷, —OC(═O)(CH₂)ᵣOC(═O)R⁷, —OC(═O)N(R⁴)R₇, —N(R⁴)C(═O)OR₇, —N(R⁴)C(═O)N(R⁴)R⁷, —OC(═O)OR⁷, or —S—SR⁷; and
R⁷ is C₆₋₂₄alkyl, C₆₋₂₄alkenyl, or C₆₋₂₄alkynyl where each is optionally substituted by F, C₁₋₆alkoxy, C₃₋₈cycloalkyl, or C₃₋₈cycloalkenyl, wherein the R⁷ of L¹ and L² may be the same or different.

2-4. (canceled)

5. The compound of claim 1, wherein
G¹ is C₁₋₁₂alkylene;
R¹ is —OH or 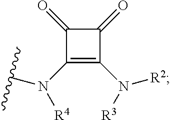
R² and R³ are each independently H, C₁₋₆alkyl, or C₃₋₈cycloalkyl; or R² and R³ together with the nitrogen atom to which they are attached form a heterocyclic ring;
R⁴ is H, C₁₋₆alkyl or C₃₋₈cycloalkyl;
G² and G³ are each independently C₁₋₁₂alkylene;
L¹ and L² are each —OC(═O)R¹; and
R⁷ is C₆₋₂₄alkyl, C₆₋₂₄alkenyl, or C₆₋₂₄alkynyl wherein each is optionally substituted by F, C₁₋₆alkoxy, C₃₋₈cycloalkyl, or C₃₋₈cycloalkenyl, wherein the R⁷ of L¹ and L² may be the same or different.

6. The compound of claim 1, wherein R⁷ has the following structure:

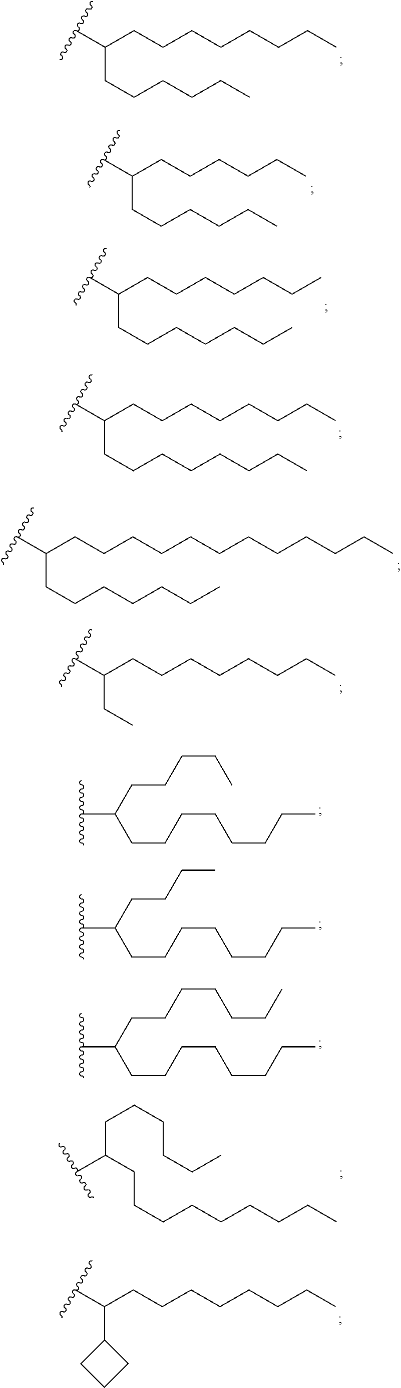

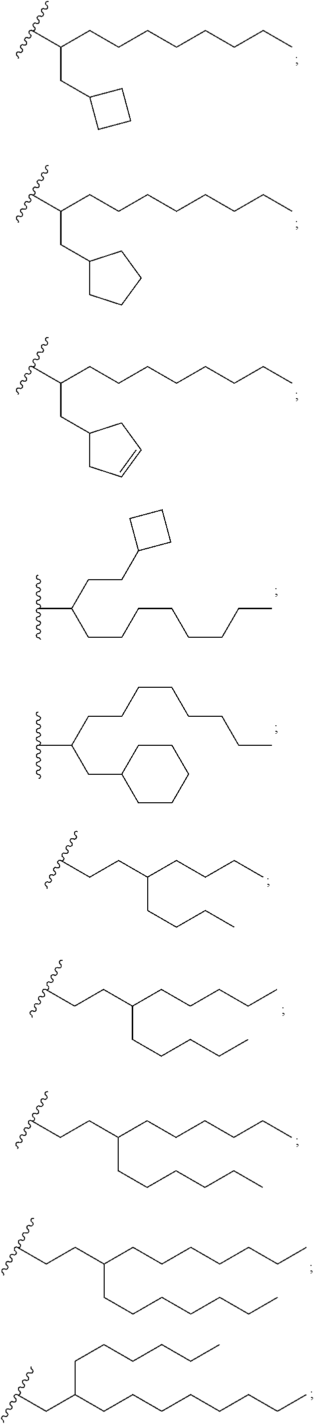

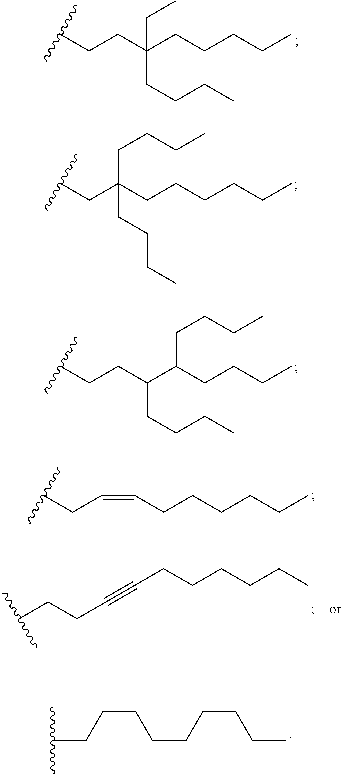

7. The compound of claim 1, wherein R¹ is OH.

8. The compound of claim 1, wherein R¹ is

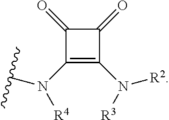

9. The compound of claim 1, wherein G¹ is C₂-C₅ alkylene.

10. The compound of claim 1, wherein G¹ is C₃-C₅ alkylene.

11. The compound of claim 1 selected from the group consisting of:
(3-(4-hydroxybutyl)-3-azaspiro[5.5]undecane-9,9-diyl)bis(methylene) bis(2-heptylnonanoate);
2-(3-(4-hydroxybutyl)-3-azaspiro[5.5]undecan-9-yl)propane-1,3-diyl bis(2-heptylnonanoate);
2-(9-(4-hydroxybutyl)-3,9-diazaspiro[5.5]undecan-3-yl)propane-1,3-diyl bis(2-heptylnonanoate);
2-(9-(4-hydroxybutyl)-3,9-diazaspiro[5.5]undecan-3-yl)propane-1,3-diyl bis(2-cyclobutyldecanoate);
2-(9-(5-hydroxypentyl)-3,9-diazaspiro[5.5]undecan-3-yl)propane-1,3-diyl bis(2-hexyldecanoate);
2-(2-(4-hydroxybutyl)-2,8-diazaspiro[4.5]decan-8-yl)propane-1,3-diyl bis(2-hexyldecanoate);
3-((2-hexyldecanoyl)oxy)-2-(9-(4-hydroxybutyl)-3,9-diazaspiro[5.5]undecan-3-yl)propyl palmitate;
2-(9-(3-((2-(methylamino)-3,4-dioxocyclobut-1-en-1-yl)amino)propyl)-3,9-diazaspiro[5.5]undecan-3-yl)propane-1,3-diyl bis(2-heptylnonanoate);
2-(9-(3-(1-methylcyclopropane-1-carboxamido)propyl)-3,9-diazaspiro[5.5]undecan-3-yl)propane-1,3-diyl bis(2-hexyldecanoate);
2-(9-(3-hydroxypropyl)-3,9-diazaspiro[5.5]undecan-3-yl)propane-1,3-diyl bis(2-heptylnonanoate);
2-(9-(3-(ethylsulfonamido)propyl)-3,9-diazaspiro[5.5]undecan-3-yl)propane-1,3-diyl bis(2-hexyldecanoate);
2-(7-(4-hydroxybutyl)-2,7-diazaspiro[4.4]nonan-2-yl)propane-1,3-diyl bis(2-heptylnonanoate);
2-(7-(4-hydroxybutyl)-2,7-diazaspiro[3.5]nonan-2-yl)propane-1,3-diyl bis(2-heptylnonanoate);
2-(8-(4-hydroxybutyl)-2,8-diazaspiro[4.5]decan-2-yl)propane-1,3-diyl bis(2-heptylnonanoate);
3-(9-(4-hydroxybutyl)-3,9-diazaspiro[5.5]undecan-3-yl)pentane-1,5-diyl bis(2-hexyldecanoate);
2-(7-(5-hydroxypentyl)-2,7-diazaspiro[4.4]nonan-2-yl)propane-1,3-diyl bis(2-hexyldecanoate);
2-(2-(5-hydroxypentyl)-2,8-diazaspiro[4.5]decan-8-yl)propane-1,3-diyl bis(2-hexyldecanoate);
2-(7-(5-hydroxypentyl)-2,7-diazaspiro[3.5]nonan-2-yl)propane-1,3-diyl bis(2-heptylnonanoate);
2-(9-(4-hydroxybutyl)-3,9-diazaspiro[5.5]undecan-3-yl)pentane-1,5-diyl bis(2-hexyldecanoate);
2-(2-(4-hydroxybutyl)-2,7-diazaspiro[3.5]nonan-7-yl)propane-1,3-diyl bis(2-heptylnonanoate);
2-(9-(5-hydroxypentan-2-yl)-3,9-diazaspiro[5.5]undecan-3-yl)propane-1,3-diyl bis(2-hexyldecanoate);
2-(2-(4-hydroxybutyl)-2,8-diazaspiro[4.5]decan-8-yl)propane-1,3-diyl bis(2-heptylnonanoate);
2-(9-(4-hydroxybutyl)-3,9-diazaspiro[5.5]undecan-3-yl)propane-1,3-diyl bis(2-hexyldecanoate);
2-(9-(4-hydroxybutyl)-3,9-diazaspiro[5.5]undecan-3-yl)propane-1,3-diyl bis(2-octyldecanoate);
2-(9-(5-hydroxypentyl)-3,9-diazaspiro[5.5]undecan-3-yl)propane-1,3-diyl bis(2-heptylnonanoate);
2-(9-(4-hydroxybutyl)-3,9-diazaspiro[5.5]undecan-3-yl)propane-1,3-diyl bis(2-heptyldecanoate);
2-(9-(4-hydroxybutyl)-3,9-diazaspiro[5.5]undecan-3-yl)propane-1,3-diyl bis(2-butyldecanoate);
2-(9-(4-hydroxybutyl)-3,9-diazaspiro[5.5]undecan-3-yl)propane-1,3-diyl bis(3-hexylundecanoate);
2-(9-(4-hydroxybutyl)-3,9-diazaspiro[5.5]undecan-3-yl)propane-1,3-diyl bis(2-pentyldecanoate);
2-(9-(4-hydroxybutyl)-3,9-diazaspiro[5.5]undecan-3-yl)propane-1,3-diyl bis(2-(cyclobutylmethyl)decanoate);
2-(9-(4-hydroxybutyl)-3,9-diazaspiro[5.5]undecan-3-yl)propane-1,3-diyl bis(2-heptyltetradecanoate);
2-(9-(4-hydroxybutyl)-3,9-diazaspiro[5.5]undecan-3-yl)propane-1,3-diyl bis(2-(cyclopentylmethyl)decanoate);
2-(7-(4-hydroxybutyl)-2,7-diazaspiro[4.4]nonan-2-yl)propane-1,3-diyl bis(2-hexyldecanoate);
2-(7-(4-hydroxybutyl)-2,7-diazaspiro[3.5]nonan-2-yl)propane-1,3-diyl bis(2-hexyldecanoate);
2-(9-(4-hydroxybutyl)-3,9-diazaspiro[5.5]undecan-3-yl)propane-1,3-diyl bis(2-(cyclopent-3-en-1-ylmethyl)decanoate);
2-(9-(4-hydroxybutyl)-3,9-diazaspiro[5.5]undecan-3-yl)propane-1,3-diyl bis(2-(2-cyclobutylethyl)decanoate);
2-(9-(4-hydroxybutyl)-3,9-diazaspiro[5.5]undecan-3-yl)propane-1,3-diyl bis(2-(cyclohexylmethyl)decanoate);
2-(9-(4-hydroxybutyl)-3,9-diazaspiro[5.5]undecan-3-yl)propane-1,3-diyl bis(4,5-dibutylnonanoate);
2-(9-(4-hydroxybutyl)-3,9-diazaspiro[5.5]undecan-3-yl)propane-1,3-diyl bis(3,3-dibutylnonanoate);
2-(9-(4-hydroxybutyl)-3,9-diazaspiro[5.5]undecan-3-yl)propane-1,3-diyl bis(4-heptylundecanoate);
2-(7-(5-hydroxypentyl)-2,7-diazaspiro[4.4]nonan-2-yl)propane-1,3-diyl bis(2-heptylnonanoate);
3-(decanoyloxy)-2-(9-(4-hydroxybutyl)-3,9-diazaspiro[5.5]undecan-3-yl)propyl 2-hexyldecanoate;
3-((2-hexyldecanoyl)oxy)-2-(9-(4-hydroxybutyl)-3,9-diazaspiro[5.5]undecan-3-yl)propyl (Z)-dodec-5-enoate;
3-((2-(cyclobutylmethyl)decanoyl)oxy)-2-(9-(4-hydroxybutyl)-3,9-diazaspiro[5.5]undecan-3-yl)propyl 2-hexyldecanoate;
3-((2-hexyldecanoyl)oxy)-2-(9-(4-hydroxybutyl)-3,9-diazaspiro[5.5]undecan-3-yl)propyl 2-butylundecanoate;
3-((4,5-dibutylnonanoyl)oxy)-2-(9-(4-hydroxybutyl)-3,9-diazaspiro[5.5]undecan-3-yl)propyl palmitate;
3-(9-(4-hydroxybutyl)-3,9-diazaspiro[5.5]undecan-3-yl)pentane-1,5-diyl bis(2-heptylnonanoate);
2-(2-(5-hydroxypentyl)-2,8-diazaspiro[4.5]decan-8-yl)propane-1,3-diyl bis(2-heptylnonanoate);
2-(7-(5-hydroxypentyl)-2,7-diazaspiro[3.5]nonan-2-yl)propane-1,3-diyl bis(2-hexyldecanoate);
2-(2-(4-hydroxybutyl)-2,7-diazaspiro[3.5]nonan-7-yl)propane-1,3-diyl bis(2-hexyldecanoate);
rac-(2R,3R)-8-(4-hydroxybutyl)-8-azaspiro[4.5]decane-2,3-diyl bis(2-heptylnonanoate);
rac-(2R,3R)-8-(2-hydroxyethyl)-8-azaspiro[4.5]decane-2,3-diyl bis(2-heptylnonanoate);
rac-(7R,8R)-2-(3-hydroxypropyl)-2-azaspiro[4.4]nonane-7,8-diyl bis(2-heptylnonanoate);
rac-(2R,3R)-8-(3-hydroxypropyl)-8-azaspiro[4.5]decane-2,3-diyl bis(2-heptylnonanoate);
rac-(2R,3R)-8-(3-((2-(methylamino)-3,4-dioxocyclobut-1-en-1-yl)amino)propyl)-8-azaspiro[4.5]decane-2,3-diyl bis(2-heptylnonanoate);
rac-(2R,3R)-8-(5-hydroxypentyl)-8-azaspiro[4.5]decane-2,3-diyl bis(2-heptylnonanoate);
(2R,3R)-8-(4-hydroxybutyl)-8-azaspiro[4.5]decane-2,3-diyl bis(2-heptylnonanoate);
(2S,3S)-8-(4-hydroxybutyl)-8-azaspiro[4.5]decane-2,3-diyl bis(2-heptylnonanoate);
rac-(2R,3R)-8-(4-hydroxybutyl)-8-azaspiro[4.5]decane-2,3-diyl bis(2-octyldecanoate);
rac-(2R,3R)-8-(4-hydroxybutyl)-8-azaspiro[4.5]decane-2,3-diyl bis(2-heptyltetradecanoate);
rac-(2R,3R)-8-(4-hydroxybutyl)-8-azaspiro[4.5]decane-2,3-diyl bis(2-hexyldecanoate);
rac-O,O′-((2R,3R)-8-(4-hydroxybutyl)-8-azaspiro[4.5]decane-2,3-diyl) di(pentadecan-8-yl) Disuccinate;
rac-O′¹,O¹-((2R,3R)-8-(4-hydroxybutyl)-8-azaspiro[4.5]decane-2,3-diyl) 7,7′-di(pentadecan-8-yl) di(heptanedioate);
rac-(((2R,3R)-8-(4-hydroxybutyl)-8-azaspiro[4.5]decane-2,3-diyl)bis(oxy))bis(6-oxohexane-6,1-diyl) bis(2-heptylnonanoate);
rac-(2R,3R)-8-(4-hydroxybutyl)-8-azaspiro[4.5]decane-2,3-diyl bis(2-pentyldecanoate);
rac-(2R,3R)-8-(4-hydroxybutyl)-8-azaspiro[4.5]decane-2,3-diyl bis(2-heptyldecanoate);
rac-(2R,3R)-8-(4-hydroxybutyl)-8-azaspiro[4.5]decane-2,3-diyl bis(2-hexylundecanoate);
rac-(2R,3R)-8-(4-hydroxybutyl)-8-azaspiro[4.5]decane-2,3-diyl bis(2-hexylnonanoate);
rac-(2R,3R)-8-(4-hydroxybutyl)-8-azaspiro[4.5]decane-2,3-diyl bis(2-butyldecanoate);
rac-(2R,3R)-3-((2-ethylnonanoyl)oxy)-8-(4-hydroxybutyl)-8-azaspiro[4.5]decan-2-yl 2-hexyldecanoate; and
bis(3-pentyloctyl) 3-(9-(4-hydroxybutyl)-3,9-diazaspiro[5.5]undecan-3-yl)pentanedioate;
or a pharmaceutically acceptable salt thereof.

12. The compound of claim 1 selected from the group consisting of;
2-(9-(4-hydroxybutyl)-3,9-diazaspiro[5.5]undecan-3-yl)propane-1,3-diyl bis(2-heptylnonanoate);
2-(7-(4-hydroxybutyl)-2,7-diazaspiro[3.5]nonan-2-yl)propane-1,3-diyl bis(2-heptylnonanoate);
2-(2-(4-hydroxybutyl)-2,8-diazaspiro[4.5]decan-8-yl)propane-1,3-diyl bis(2-heptylnonanoate);
2-(9-(4-hydroxybutyl)-3,9-diazaspiro[5.5]undecan-3-yl)propane-1,3-diyl bis(2-hexyldecanoate);
2-(9-(5-hydroxypentyl)-3,9-diazaspiro[5.5]undecan-3-yl)propane-1,3-diyl bis(2-heptylnonanoate);
rac-(2R,3R)-8-(4-hydroxybutyl)-8-azaspiro[4.5]decane-2,3-diyl bis(2-heptylnonanoate);
rac-(2R,3R)-8-(4-hydroxybutyl)-8-azaspiro[4.5]decane-2,3-diyl bis(2-hexyldecanoate);
2-(2-(4-hydroxybutyl)-2,8-diazaspiro[4.5]decan-8-yl)propane-1,3-diyl bis(2-hexyldecanoate);
2-(9-(3-hydroxypropyl)-3,9-diazaspiro[5.5]undecan-3-yl)propane-1,3-diyl bis(2-heptylnonanoate);
2-(9-(4-hydroxybutyl)-3,9-diazaspiro[5.5]undecan-3-yl)propane-1,3-diyl bis(3-hexylundecanoate);
2-(7-(4-hydroxybutyl)-2,7-diazaspiro[4.4]nonan-2-yl)propane-1,3-diyl bis(2-hexyldecanoate);
2-(7-(5-hydroxypentyl)-2,7-diazaspiro[3.5]nonan-2-yl)propane-1,3-diyl bis(2-hexyldecanoate);
2-(2-(4-hydroxybutyl)-2,7-diazaspiro[3.5]nonan-7-yl)propane-1,3-diyl bis(2-hexyldecanoate); and
rac-(2R,3R)-8-(5-hydroxypentyl)-8-azaspiro[4.5]decane-2,3-diyl bis(2-heptylnonanoate);
or a pharmaceutically acceptable salt thereof.

13-40. (canceled)

41. A pharmaceutical composition comprising a nucleic acid, at least one pharmaceutically acceptable excipient, and the compound according to claim 1, or a pharmaceutically acceptable salt thereof.

42. The pharmaceutical composition of claim 41, wherein the pharmaceutically acceptable excipient is selected from the group consisting of neutral lipids, steroids and polymer conjugated lipids.

43. The pharmaceutical composition of claim 42, wherein the pharmaceutical composition comprises 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), phophatidylethanolamines such as 1,2-Dioleoyl-sn-glycero-3-phosphoethanolamine (DOPE), sphingomyelins (SM), or a combination thereof.

44. The pharmaceutical composition of claim 42, wherein the steroid is cholesterol.

45. The pharmaceutical composition of claim 42, wherein the polymer conjugated lipid is a pegylated lipid.

46. The pharmaceutical composition of claim 45, wherein the pegylated lipid is PEG-DAG, PEG-PE, PEG-S-DAG, PEG-cer, PEG-DMG, 2-[(polyethylene glycol)-2000]-N,N-ditetradecylacetamide (ALC-0159) or a PEG dialkyoxypropylcarbamate.

47. The pharmaceutical composition of claim 41, wherein the nucleic acid is RNA.

48. The pharmaceutical composition of claim 47, wherein the RNA is messenger RNA.

49. (canceled)

50. A method for administering a nucleic acid to a subject in need thereof, the method comprising preparing or providing the pharmaceutical composition of claim 41, and administering the pharmaceutical composition to the subject.

51-53. (canceled)
